(19) United States
(12) Patent Application Publication
(10) Pub. No.: US 2026/0167614 A1
(43) Pub. Date: Jun. 18, 2026

(54) IDENTIFICATION OF SMALL MOLECULES THAT RECRUIT AND ACTIVATE RNASE L

(71) Applicant: UNIVERSITY OF FLORIDA RESEARCH FOUNDATION, INCORPORATED, Gainesville, FL (US)

(72) Inventor: Matthew D. Disney, Gainesville, FL (US)

(73) Assignee: UNIVERSITY OF FLORIDA RESEARCH FOUNDATION, INCORPORATED, Gainesville, FL (US)

(21) Appl. No.: 19/126,779

(22) PCT Filed: Nov. 2, 2023
(86) PCT No.: PCT/US2023/078482

§ 371 Date: May 2, 2025

Related U.S. Application Data
(60) Provisional application No. 63/422,389, filed on Nov. 3, 2022.

(51) Int. Cl. C07D 333/20; A61K 31/381; A61K 31/496; A61P 35/00; C07D 409/14; C12N 15/10

(52) U.S. Cl. CPC C07D 333/20; A61K 31/381; A61K 31/496; A61P 35/00; C07D 409/14; C12N 15/1093

(57) ABSTRACT

The present disclosure provides compounds of the formulae herein (e.g., Formulae (I) and (II)), and pharmaceutically acceptable salts, solvates, hydrates, polymorphs, co-crystals, tautomers, stereoisomers, isotopically labeled compounds, and prodrugs thereof, which bind RNase L and/or bind an RNA target, allowing for cleavage of the RNA target. The present disclosure also provides pharmaceutical compositions and kits comprising the compounds, or pharmaceutically acceptable salts, solvates, hydrates, polymorphs, co-crystals, tautomers, stereoisomers, isotopically labeled compounds, or prodrugs thereof, and methods of treating or preventing diseases by administering to a subject in need thereof the compounds, or pharmaceutically acceptable salts, solvates, hydrates, polymorphs, co-crystals, tautomers, stereoisomers, isotopically labeled compounds, or prodrugs thereof, or pharmaceutical compositions thereof.

### CROSS-REFERENCE TO RELATED APPLICATIONS

This application claims the benefit of and priority under 35 U.S.C. § 119(e) to U.S. Provisional Application No. 63/422,389, filed Nov. 3, 2022, titled IDENTIFICATION OF SMALL MOLECULES THAT RECRUIT AND ACTIVATE RNASE L, the contents of which are incorporated herewith by reference in their entirety.

### GOVERNMENT SUPPORT

This invention was made with government support under grant numbers R01 CA249180 and R35 NS 116846, awarded by the National Institutes of Health. The government has certain rights in the invention.

### BACKGROUND OF THE INVENTION

Heterobifunctional molecules can affect various disease processes by facilitating interactions between two biomolecules, where one modulates the function of the other. For example, proteolysis targeting chimeras (ProTACs) comprise a molecule that binds to a target protein and another that binds to E3 ubiquitin ligase to facilitate protein degradation via the proteosome.¹ Likewise, an RNA-binding small molecule and a ribonuclease-recruiting compound comprise ribonuclease targeting chimeras (RiboTACs), eliminating the target RNA by induced proximity (FIG. 1).² RiboTACs are a small molecule equivalent of antisense and short interfering (si)RNA-based approaches, however rather than recognize RNA sequence, they recognize RNA structure.²⁻³ RiboTACs may be more amenable to medicinal chemistry optimization than oligonucleotide-based modalities, and can eliminate messenger RNAs (mRNAs) that encode difficult to target proteins, creating possible new avenues to target disease.

RiboTACs have been designed by first identifying a structure-specific RNA-binding compound. Design can be guided by lead identification software, Inforna,⁴ which relies on RNA-small molecule binding partners,⁵ microarray-based screening,⁶ or medicinal chemistry optimization informed by structural modeling experiments,⁷⁻¹⁰ among other strategies. The structure-specific compound is then conjugated to a heterocyclic small molecule recruiter of monomeric latent ribonuclease (RNase L),¹¹ causing the enzyme to dimerize and activating its enzymatic activity. The linker separating the two modules can also be optimized, positioning RNase L near a preferred substrate, for example UNN.¹²

### SUMMARY OF THE INVENTION

In the present disclosure, a DNA-encoded library (DEL)¹³ is used as a starting point for identifying scaffolds that bind monomeric RNase L, and subsequently determining which compounds can dimerize and activate the ribonuclease. DEL screening has previously been used to identify the protein or enzyme-binding small molecule in ProTACs.¹⁴ The present disclosure expands this technology to identify small molecules that bind the effector protein (e.g., RNase L), the other component of such heterobifunctional compounds.

Accordingly, in one aspect, the present disclosure provides compounds of Formula (I):

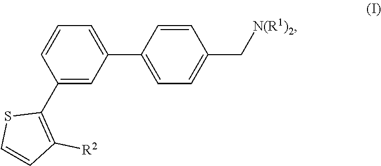

or pharmaceutically acceptable salts, solvates, hydrates, polymorphs, co-crystals, tautomers, stereoisomers, isotopically labeled compounds, or prodrugs thereof, wherein R¹ and R² are as defined herein.

In another aspect, the present disclosure provides compounds of Formula:

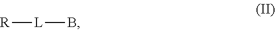

or pharmaceutically acceptable salts, solvates, hydrates, polymorphs, co-crystals, tautomers, stereoisomers, isotopically labeled compounds, or prodrugs thereof, wherein:
R is an RNase L recruiter of Formula (III):

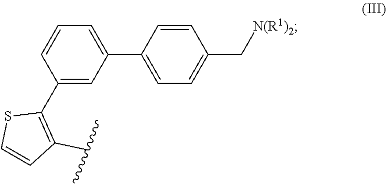

L is a linker; and S
B is an RNA binder; (III);
   wherein R¹ is as defined herein.

In another aspect, the present disclosure provides pharmaceutical compositions comprising a compound disclosed herein. In some embodiments, the pharmaceutical composition comprises an excipient.

In another aspect, the present disclosure provides methods of binding RNase L in a subject in need thereof or in a cell, tissue, or biological sample in a subject in need thereof or in a cell, tissue, or biological sample, comprising administering to the subject in need thereof or contacting the cell, tissue, or biological sample with an effective amount of a provided compound or pharmaceutical composition. In certain embodiments, the cell, tissue, or biological sample is in vivo. In certain embodiments, the cell, tissue, or biological sample is in vitro. In certain embodiments, binding RNase L comprises activating RNase L. In certain embodiments, binding RNase L comprises inducing RNase L dimerization.

In another aspect, the present disclosure provides methods of binding an RNA target in a subject in need thereof or in a cell, tissue, or biological sample in a subject in need thereof or in a cell, tissue, or biological sample, comprising administering to the subject in need thereof or contacting the cell, tissue, or biological sample with an effective amount of a provided compound or pharmaceutical composition. In certain embodiments, the cell, tissue, or biological sample is in vivo. In certain embodiments, the cell, tissue, or biological sample is in vitro. In certain embodiments, the method further comprises recruiting RNase L. In certain embodiments, the method further comprises activating RNase L. In certain embodiments, the method further comprises inducing RNase L dimerization. In certain embodiments, the method comprises decreasing an amount of the RNA target. In certain embodiments, the method comprises cleaving the RNA target. In certain embodiments, the RNA target is a precursor to microRNA-21 (pre-miR-21). In certain embodiments, the method further comprises downregulating mature microRNA-21 (miR-21) (e.g. miR-21-5p).

In another aspect, the present disclosure provides methods of inhibiting cell proliferation or inducing apoptosis in a subject in need thereof or in a cell, tissue, or biological sample in a subject in need thereof or in a cell, tissue, or biological sample, comprising administering to the subject in need thereof or contacting the cell, tissue, or biological sample with an effective amount of a provided compound or pharmaceutical composition. In certain embodiments, the cell, tissue, or biological sample is in vivo. In certain embodiments, the cell, tissue, or biological sample is in vitro.

In another aspect, the present disclosure provides methods of treating or preventing a disease in a subject in need thereof, comprising administering to the subject in need thereof a provided compound or pharmaceutical composition. In certain embodiments, the disease is associated with microRNA-21 (miR-21) (e.g., proliferative disease, kidney disease).

In another aspect, the present disclosure provides methods of identifying a binder of an effector protein, comprising screening the effector protein against a DNA-encoded library (DEL) to identify a set of compounds specifically enriched for binding to the effector protein; synthesizing one or more compounds from the set of compounds specifically enriched for binding to the effector protein without an encoding DNA tag; determining effector protein activation by the one or more compounds from the set of compounds specifically enriched for binding to the effector protein synthesized without the encoding DNA tag; and identifying the one or more compounds from the set of compounds specifically enriched for binding to the effector protein synthesized without the encoding DNA tag as a binder of the effector protein. In certain embodiments, determining effector protein activation comprises an in vitro assay. In certain embodiments, the in vitro assay is fluorescence-based.

In another aspect, the present disclosure provides methods of preparing a compound of Formula (II-a):

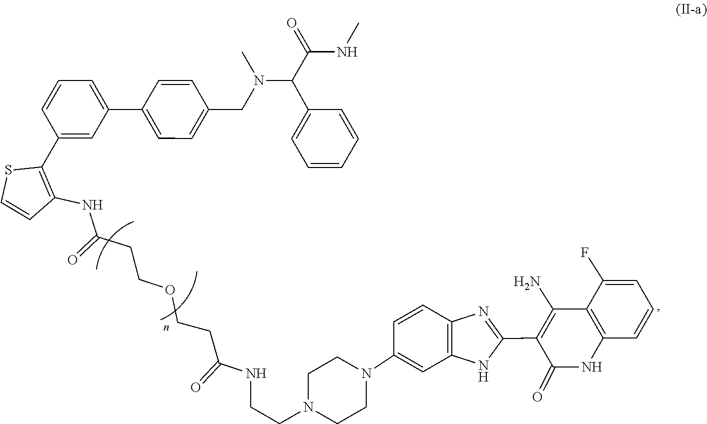

or a pharmaceutically acceptable salt, solvate, hydrate, polymorph, co-crystal, tautomer, stereoisomer, isotopically labeled compound, or prodrug thereof.

In another aspect, the present disclosure provides kits comprising a provided compound or pharmaceutical composition disclosed herein and instructions for its use.

It should be appreciated that the foregoing concepts, and the additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying drawings.

### BRIEF DESCRIPTION OF THE DRAWINGS

FIG. 1 shows ribonuclease-targeting chimeras (RiboTACs) target RNA structure, rather than sequence, to catalyze proximity-induced degradation of a target. RiboTACs can bind to a target structure and recruit a ribonuclease, RNase L, to degrade the RNA. Antisense-oligonucleotides (ASOs) can target RNA sequence, recruiting RNase H to the RNA-DNA heteroduplex to degrade the RNA target.

FIGS. 2A-2D show an RNase L recruiter identified by screening a DNA-encoded library (DEL). FIG. 2A shows a selection and hit validation scheme for screening the DEL library (left) and schematic depicting refinement of DEL hits to afford the four lead compounds selected for further study (>300-fold enrichment for RNase L binding, >25 copy number) (right). FIG. 2B shows Markush structures of chemical building blocks in the most highly enriched chemical family enriched by RNase L. X and R represent arbitrary substituents while A represents either C, O, S or N. FIG. 2C shows structures of previously reported RNase L recruiter 1, validated RNase L recruiters 2 and 3 from the DEL screening, an analog of 2 lacking the alkyl chain (4), and analogs of 4 with a polyethylene glycol linker and t-Bu (5) or H (6). FIG. 2D shows compounds 1-6 induce dose-dependent cleavage of a fluorescently labeled RNA displaying preferential RNase L cleavage sites, indicating these compounds dimerize and activate RNase L in vitro. Data are reported as “Relative RNA Cleavage”, normalizing fluorescence to the highest level of cleavage observed—upon addition of 50 μM of 6, which was set to 1. ****, p<0.0001 as determined by an unpaired t-test. Data are reported as the mean±SD.

FIGS. 3A-3D show that RiboTAC 7 binds to and cleaves pre-miR-21 in vitro. FIG. 3A shows schematic representation of mature miR-21 biogenesis and associated downstream pathology. FIG. 3B shows the structure of RiboTAC 7. FIG. 3C shows RiboTAC 7 dose-dependently cleaves a premiR-21 construct dually labeled 5′ 6-FAM and 3′ BHQ (n=3). FIG. 3D shows RiboTAC 7 dose-dependently and selectively induces co-immunoprecipitation of 32P-pre-miR-21 compared to premiR-21 mutant in which the RNA-binding modules binding site was mutated from an A-bulge to a base-pair (n=4). *, p<0.05; **, p<0.01; ***, p<0.001; ****, p<0.0001 as determined by a one-way ANOVA with multiple comparisons. Data are reported as the mean±SD.

FIGS. 4A-4F show that RiboTAC 7 alleviates miR-21-associated pathologies in triple negative breast cancer cells. FIG. 4A shows the effect of RiboTAC 7 on mature miR-21 levels in MDA-MB-231 cells modified by CRISPR with either a control guide RNA (“Control sgRNA; express RNase L; left side) or with an RNase L-targeting sgRNA (“RNase L-Targeting sgRNA; right side), as determined by RT-qPCR (n=3 for both cell lines). FIG. 4B shows the effect of RiboTAC 7 on pre-miR-21 levels in MDA-MB-231 cells modified by CRISPR with either a control guide RNA (“Control sgRNA; expresses RNase L; left side) or with an RNase L-targeting sgRNA (“RNase L-Targeting sgRNA; right side), as determined by RT-qPCR (n=3 for both cell lines). FIG. 4C shows results of miRNA profiling experiment showing miR-21 is selectively down-regulated by RiboTAC 7 (n=377 miRNAs; 3 biological replicates). miR-101-3p, miR-363-3p, miR-345-5p, miR-378a-3p, and miR-135b-5p represent miRs that are commonly dysregulated in TNBC. FIG. 4D shows a representative Western blot image of PDCD4 protein abundance upon treatment of MDA-MB-231 cells modified by CRISPR with the control sgRNA cells (express RNase L) with RIBOTAC 7 (n=3) (left), and quantification of Western blots (n=3) (right). FIG. 4E shows representative images of the invasion of MDAMB-231 cells modified by CRISPR with the control sgRNA cells (express RNase L), a miR-21-associated phenotype, with or without RiboTAC 7-treatment, as determined by a Boyden chamber assay. FIG. 4F shows quantification of Boyden chamber invasion assays (n=3, average of 4 images per biological replicate). *, p<0.05; **, p<0.01; ***, p<0.001; ****, p<0.0001, as determined by a one-way ANOVA with multiple comparisons. Data are reported as the mean±SD.

FIGS. 5A-5B show secondary validation of DEL screening hits via an RNA cleavage assay. FIG. 5A shows a schematic depicting the in vitro RNA cleavage assay used to validate DEL screening hits as activators of RNase L. Activation is measured by an increase in fluorescence, generated as the RNA is cleaved. FIG. 5B shows compounds 1-6 induce dose-dependent cleavage of the RNA, indicating these compounds dimerize and activate RNase L. Data are reported as “Relative RNA Cleavage”, normalizing fluorescence to the highest level of cleavage observed—upon addition of 50 μM of 6, which was set to 1. “WuXi Hit 3” and “WuXi Hit 4” were not further pursued as they failed to induce RNA cleavage to a greater extent than control compound 1. ****, p<0.0001 as determined by an unpaired t-test. Data are reported as the mean±SD.

FIGS. 6A-6C show compound 2 induces dimerization of RNase L and 6 binds to it in vitro. FIG. 6A shows a representative Western blot showing RNase L dimerization upon incubation with 2 (left), and quantification of the dimerization assay (n=2) (right). FIG. 6B shows saturation transfer difference (STD-) NMR spectroscopy confirms selective binding of 6 to RNase L. ¹H NMR spectrum of 6 is shown as a reference (top). STD-NMR spectrum of 6, recorded in the presence of RNase L (middle) or lysozyme (bottom). FIG. 6C shows circular dichroism spectra for RNase L in the presence and absence of 6.

FIGS. 7A-7B show RiboTAC 7 selectively binds pre-miR-21, as measured by changes in intrinsic fluorescence of the RiboTAC. FIG. 7A shows the structure of the pre-miR-21 RNA construct used for binding studies (left), and representative binding curve for RiboTAC 7 and pre-miR-21, which binds with an affinity of K_{d} of 4.6±2.1 μM (n=3 independent experiments, each with 3 technical replicates) (right). FIG. 7B shows the structure of the pre-miR-21 mutant in which the U- and A- (Dicer site) bulges are base-paired (left), and representative binding curve for RiboTAC 7 and the mutant premiR-21 in which no saturable binding was observed (K_{d}>100 μM; n=2 independent experiments, each with 3 technical replicates) (right).

FIGS. 8A-8B show in vitro C-Chem-CLIP indicates RiboTAC 7 directly engages pre-miR-21. FIG. 8A shows structures of Chem-CLIP probe 8 and the control Chem-CLIP probe 9.¹ FIG. 8B shows percentage of 32P-labeled pre-miR-21 pulled-down by 8 when treated in competition with 7. *, p<0.05; **, p<0.01; ***, p<0.001; and ****, p<0.0001, as determined by a one-way ANOVA with multiple comparisons. Data are reported as mean±SD.

FIG. 9 shows RNase L recruiter 6 does not induce ternary complex formation in vitro. Compound 6 was unable to co-immunoprecipitate pre-miR-21 or the pre-miR-21 mutant with RNase L as it lacks the pre-miR-21-binding module (Dovitinib). Data are reported as mean±SD.

FIGS. 10A-10B show RiboTAC 7 is cell permeable and induces pre-miR-21 degradation within 48 h of treatment. FIG. 10A shows RiboTAC 7 has a cellular uptake half-life of 2.4 h and reached a concentration of 3.6±1.2 μM inside cells after 48 h treatment with 7 (5 μM) in culture medium (n=6). FIG. 10B shows RiboTAC 7 (5 μM) induces pre-miR-21 degradation after 48 h of treatment (n=3). *, p<0.05; **, p<0.01; ***, p<0.001; and ****, p<0.0001, as determined by a one-way ANOVA with multiple comparisons. Data are reported as mean±SD.

FIGS. 11A-11D show proteome-wide analysis of the effects of RiboTAC 7 and an LNA antagomir targeting miR-21-5p in MDA-MB-231 cells modified with CRISPR and a control guide RNA. FIG. 11A shows a volcano plot showing proteome-wide changes induced by 7 (5 μM; n=3). FIG. 11B shows a cumulative distribution plot shows an increase in the abundance of miR-21-5p-regulated proteins upon treatment with 7. Proteins regulated by miR-21-5p were predicted by TargetScanHuman v7.22 (n=384). As a control, the abundance of proteins regulated by let-7b-5p were also investigated. Proteins regulated by let-7b-5p were predicted by TargetScanHuman v7.22 (n=1207). Targets with context++ scores<−0.1 were used in the calculations. FIG. 11C shows a volcano plot showing proteome-wide changes induced by the miR-21-5p-targeting LNA (0.1 μM; n=3). (The LNA Power Inhibitor was purchased from Qiagen, catalog #YI00199006-DFA.) FIG. 11D shows a cumulative distribution plot shows an increase in the abundance of miR-21-5p regulated proteins upon treatment with the antagomir but not let-7b-5p.

FIGS. 12A-12C show RiboTAC 7 rescues an invasive phenotype in healthy breast epithelial cells forced to express pre-miR-21. FIG. 12A shows sequences and structures of pre-miR-21 and its mutant expressed in MCF-10A cells by transfection of the corresponding plasmid. FIG. 12B shows representative microscopic images of invasive MCF-10A cells transfected with WT pre-miR-21 or mutant premiR-21, with or without treatment with RiboTAC 7. Vehicle is 0.1% (v/v) DMSO (n=3, average of four fields of view per biological replicate). FIG. 12C shows quantification of the number of invasive cells transfected with WT pre-miR-21 or mutant pre-miR-21, with or without treatment with RiboTAC 7. *, p<0.05; **, p<0.01; ***, p<0.001; ****, p<0.0001, as determined by an unpaired t-test with Welch's correction. Data are reported as mean±SD.

FIGS. 13A-13D show RiboTAC 7 does not inhibit RTKs, as assayed by measuring the phosphorylation of ERK. FIG. 13A shows representative Western blot showing pERK versus ERK abundance upon treatment of CRISPR-Control sgRNA MDA-MB-231 cells with Dovitinib, as a positive control (n=3). FIG. 13B shows quantification of Western blot in A (n=3). FIG. 13C shows representative Western blot showing pERK versus ERK abundance upon treatment of CRISPR-Control sgRNA MDA-MB-231 cells with 7 (n=3). FIG. 13D shows quantification of Western blot in C (n=3). *, p<0.05; **, p<0.01; ***, p<0.001; ****, p<0.0001, as determined by an unpaired t-test with Welch's correction. Data are reported as mean±SD.

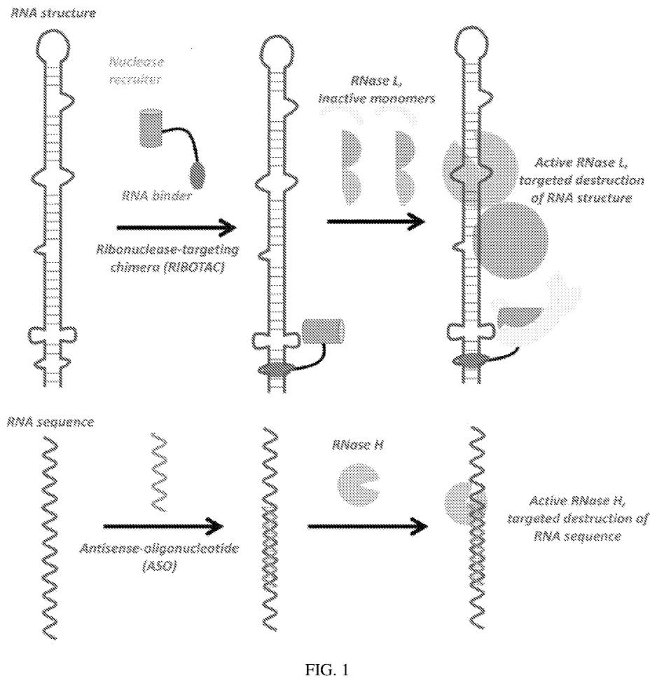

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Michael B. Smith, March's Advanced Organic Chemistry, 7^{th} Edition, John Wiley & Sons, Inc., New York, 2013; Richard C. Larock, Comprehensive Organic Transformations, John Wiley & Sons, Inc., New York, 2018; and Carruthers, Some Modern Methods of Organic Synthesis, 3^{rd} Edition, Cambridge University Press, Cambridge, 1987.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high-pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, NY, 1962); and Wilen, S. H., Tables of Resolving Agents and Optical Resolutions p. 268 (E.L. Eliel, Ed., Univ. of Notre Dame Press, Notre Dame, IN 1972). The invention additionally encompasses compounds as individual isomers substantially free of other isomers, and alternatively, as mixtures of various isomers. The term “isomers” is intended to include diastereoisomers, enantiomers, regioisomers, structural isomers, rotational isomers, tautomers, and the like. All such isomers of such compounds herein are expressly included in the present invention.

When a range of values (“range”) is listed, it encompasses each value and sub-range within the range. A range is inclusive of the values at the two ends of the range unless otherwise provided. For example “C₁₋₆ alkyl” encompasses, C₁, C₂, C₃, C₄, C₅, C₆, C₁₋₆, C₁₋₅, C₁₋₄, C₁₋₃, C₁₋₂, C₂₋₆, C₂₋₅, C₂, C₂₋₃, C₃₋₆, C₃₋₅, C₃₋₄, C₄₋₆, C₄₋₅, and C₅₋₆ alkyl.

The term “aliphatic” refers to alkyl, alkenyl, alkynyl, and carbocyclic groups. Likewise, the term “heteroaliphatic” refers to heteroalkyl, heteroalkenyl, heteroalkynyl, and heterocyclic groups.

The term “alkyl” refers to a radical of a straight-chain or branched saturated hydrocarbon group having from 1 to 20 carbon atoms (“C₁₋₂₀ alkyl”). In some embodiments, an alkyl group has 1 to 12 carbon atoms (“C₁₋₁₂ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, isobutyl), pentyl (C₅) (e.g., n-pentyl, 3-pentanyl, amyl, neopentyl, 3-methyl-2-butanyl, tert-amyl), and hexyl (C₆) (e.g., n-hexyl). Additional examples of alkyl groups include n-heptyl (C₇), n-octyl (C₈), n-dodecyl (C₁₂), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₂ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu or s-Bu), unsubstituted isobutyl (i-Bu)). In certain embodiments, the alkyl group is a substituted C₁₋₁₂ alkyl (such as substituted C₁₋₆ alkyl, e.g., —CH₂F, —CHF₂, —CF₃, —CH₂CH₂F, —CH₂CHF₂, —CH₂CF₃, or benzyl (Bn)).

The term “haloalkyl” is a substituted alkyl group, wherein one or more of the hydrogen atoms are independently replaced by a halogen, e.g., fluoro, bromo, chloro, or iodo. “Perhaloalkyl” is a subset of haloalkyl and refers to an alkyl group wherein all of the hydrogen atoms are independently replaced by a halogen, e.g., fluoro, bromo, chloro, or iodo. In some embodiments, the haloalkyl moiety has 1 to 20 carbon atoms (“C₁₋₂₀ haloalkyl”). In some embodiments, the haloalkyl moiety has 1 to 10 carbon atoms (“C₁₋₁₀ haloalkyl”). In some embodiments, the haloalkyl moiety has 1 to 9 carbon atoms (“C₁₋₉ haloalkyl”). In some embodiments, the haloalkyl moiety has 1 to 8 carbon atoms (“C₁₋₈ haloalkyl”). In some embodiments, the haloalkyl moiety has 1 to 7 carbon atoms (“C₁₋₇ haloalkyl”). In some embodiments, the haloalkyl moiety has 1 to 6 carbon atoms (“C₁₋₆ haloalkyl”). In some embodiments, the haloalkyl moiety has 1 to 5 carbon atoms (“C₁₋₅ haloalkyl”). In some embodiments, the haloalkyl moiety has 1 to 4 carbon atoms (“C₁₋₄ haloalkyl”). In some embodiments, the haloalkyl moiety has 1 to 3 carbon atoms (“C₁₋₃ haloalkyl”). In some embodiments, the haloalkyl moiety has 1 to 2 carbon atoms (“C₁₋₂ haloalkyl”). In some embodiments, all of the haloalkyl hydrogen atoms are independently replaced with fluoro to provide a “perfluoroalkyl” group. In some embodiments, all of the haloalkyl hydrogen atoms are independently replaced with chloro to provide a “perchloroalkyl” group. Examples of haloalkyl groups include —CHF₂, —CH₂F, —CF₃, —CH₂CF₃, —CF₂CF₃, —CF₂CF₂CF₃, —CCl₃, —CFCl₂, —CF₂Cl, and the like.

The term “heteroalkyl” refers to an alkyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkyl group refers to a saturated group having from 1 to 20 carbon atoms and 1 or more heteroatoms within the parent chain (“heteroC₁₋₂₀ alkyl”). In certain embodiments, a heteroalkyl group refers to a saturated group having from 1 to 12 carbon atoms and 1 or more heteroatoms within the parent chain (“heteroC₁₋₁₂ alkyl”). In some embodiments, a heteroalkyl group is a saturated group having 1 to 11 carbon atoms and 1 or more heteroatoms within the parent chain (“heteroC₁₋₁₁ alkyl”). In some embodiments, a heteroalkyl group is a saturated group having 1 to 10 carbon atoms and 1 or more heteroatoms within the parent chain (“heteroC₁₋₁₀ alkyl”). In some embodiments, a heteroalkyl group is a saturated group having 1 to 9 carbon atoms and 1 or more heteroatoms within the parent chain (“heteroC₁₋₉ alkyl”). In some embodiments, a heteroalkyl group is a saturated group having 1 to 8 carbon atoms and 1 or more heteroatoms within the parent chain (“heteroC₁₋₈ alkyl”). In some embodiments, a heteroalkyl group is a saturated group having 1 to 7 carbon atoms and 1 or more heteroatoms within the parent chain (“heteroC₁₋₇ alkyl”). In some embodiments, a heteroalkyl group is a saturated group having 1 to 6 carbon atoms and 1 or more heteroatoms within the parent chain (“heteroC₁₋₆ alkyl”). In some embodiments, a heteroalkyl group is a saturated group having 1 to 5 carbon atoms and 1 or 2 heteroatoms within the parent chain (“heteroC₁₋₅ alkyl”). In some embodiments, a heteroalkyl group is a saturated group having 1 to 4 carbon atoms and for 2 heteroatoms within the parent chain (“heteroC₁₋₄ alkyl”). In some embodiments, a heteroalkyl group is a saturated group having 1 to 3 carbon atoms and 1 heteroatom within the parent chain (“heteroC₁₋₃ alkyl”). In some embodiments, a heteroalkyl group is a saturated group having 1 to 2 carbon atoms and 1 heteroatom within the parent chain (“heteroC₁₋₂ alkyl”). In some embodiments, a heteroalkyl group is a saturated group having 1 carbon atom and 1 heteroatom (“heteroC₁ alkyl”). In some embodiments, a heteroalkyl group is a saturated group having 2 to 6 carbon atoms and 1 or 2 heteroatoms within the parent chain (“heteroC₂₋₆ alkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₁₂ alkyl. In certain embodiments, the heteroalkyl group is a substituted heteroC₁₋₁₂ alkyl.

The term “alkenyl” refers to a radical of a straight-chain or branched hydrocarbon group having from 2 to 20 carbon atoms and one or more carbon-carbon double bonds (e.g., 1, 2, 3, or 4 double bonds). In some embodiments, an alkenyl group has 2 to 20 carbon atoms (“C₂₋₂₀ alkenyl”). In some embodiments, an alkenyl group has 2 to 12 carbon atoms (“C₂₋₁₂ alkenyl”). In some embodiments, an alkenyl group has 2 to 11 carbon atoms (“C₂₋₁₁alkenyl”). In some embodiments, an alkenyl group has 2 to 10 carbon atoms (“C₂₋₁₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C₂₋₂₀ alkenyl. In certain embodiments, the alkenyl group is a substituted C₂₋₂₀ alkenyl. In an alkenyl group, a C═C double bond for which the stereochemistry is not specified

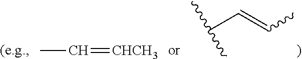

may be in the (E)- or (Z)-configuration.

The term “heteroalkenyl” refers to an alkenyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkenyl group refers to a group having from 2 to 20 carbon atoms, at least one double bond, and 1 or more heteroatoms within the parent chain (“heteroC₂₋₂₀ alkenyl”). In certain embodiments, a heteroalkenyl group refers to a group having from 2 to 12 carbon atoms, at least one double bond, and 1 or more heteroatoms within the parent chain (“heteroC₂₋₁₂ alkenyl”). In certain embodiments, a heteroalkenyl group refers to a group having from 2 to 11 carbon atoms, at least one double bond, and 1 or more heteroatoms within the parent chain (“heteroC₂₋₁₁ alkenyl”). In certain embodiments, a heteroalkenyl group refers to a group having from 2 to 10 carbon atoms, at least one double bond, and 1 or more heteroatoms within the parent chain (“heteroC₂₋₁₀ alkenyl”). In some embodiments, a heteroalkenyl group has 2 to 9 carbon atoms at least one double bond, and 1 or more heteroatoms within the parent chain (“heteroC₂₋₉ alkenyl”). In some embodiments, a heteroalkenyl group has 2 to 8 carbon atoms, at least one double bond, and 1 or more heteroatoms within the parent chain (“heteroC₂₋₈ alkenyl”). In some embodiments, a heteroalkenyl group has 2 to 7 carbon atoms, at least one double bond, and 1 or more heteroatoms within the parent chain (“heteroC₂₋₇ alkenyl”). In some embodiments, a heteroalkenyl group has 2 to 6 carbon atoms, at least one double bond, and 1 or more heteroatoms within the parent chain (“heteroC₂₋₆ alkenyl”). In some embodiments, a heteroalkenyl group has 2 to 5 carbon atoms, at least one double bond, and 1 or 2 heteroatoms within the parent chain (“heteroC₂₋₅ alkenyl”). In some embodiments, a heteroalkenyl group has 2 to 4 carbon atoms, at least one double bond, and 1 or 2 heteroatoms within the parent chain (“heteroC₂₋₄ alkenyl”). In some embodiments, a heteroalkenyl group has 2 to 3 carbon atoms, at least one double bond, and 1 heteroatom within the parent chain (“heteroC₂₋₃ alkenyl”). In some embodiments, a heteroalkenyl group has 2 carbon atoms, at least one double bond, and 1 heteroatom within the parent chain (“heteroC₂ alkenyl”). In some embodiments, a heteroalkenyl group has 2 to 6 carbon atoms, at least one double bond, and 1 or 2 heteroatoms within the parent chain (“heteroC₂₋₆ alkenyl”). Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted heteroC₂₋₂₀ alkenyl. In certain embodiments, the heteroalkenyl group is a substituted heteroC₂₋₂₀ alkenyl.

The term “alkynyl” refers to a radical of a straight-chain or branched hydrocarbon group having from 2 to 20 carbon atoms and one or more carbon-carbon triple bonds (e.g., 1, 2, 3, or 4 triple bonds) (“C₁₋₂₀ alkynyl”). In some embodiments, an alkynyl group has 2 to 10 carbon atoms (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted C₂₋₂₀ alkynyl. In certain embodiments, the alkynyl group is a substituted C₂₋₂₀ alkynyl.

The term “heteroalkynyl” refers to an alkynyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkynyl group refers to a group having from 2 to 20 carbon atoms, at least one triple bond, and 1 or more heteroatoms within the parent chain (“heteroC₂₋₂₀ alkynyl”). In certain embodiments, a heteroalkynyl group refers to a group having from 2 to 10 carbon atoms, at least one triple bond, and 1 or more heteroatoms within the parent chain (“heteroC₂₋₁₀ alkynyl”). In some embodiments, a heteroalkynyl group has 2 to 9 carbon atoms, at least one triple bond, and 1 or more heteroatoms within the parent chain (“heteroC₂₋₉ alkynyl”). In some embodiments, a heteroalkynyl group has 2 to 8 carbon atoms, at least one triple bond, and 1 or more heteroatoms within the parent chain (“heteroC₂₋₈ alkynyl”). In some embodiments, a heteroalkynyl group has 2 to 7 carbon atoms, at least one triple bond, and 1 or more heteroatoms within the parent chain (“heteroC₂₋₇ alkynyl”). In some embodiments, a heteroalkynyl group has 2 to 6 carbon atoms, at least one triple bond, and 1 or more heteroatoms within the parent chain (“heteroC₂₋₆ alkynyl”). In some embodiments, a heteroalkynyl group has 2 to 5 carbon atoms, at least one triple bond, and 1 or 2 heteroatoms within the parent chain (“heteroC₂₋₅ alkynyl”). In some embodiments, a heteroalkynyl group has 2 to 4 carbon atoms, at least one triple bond, and 1 or 2 heteroatoms within the parent chain (“heteroC₂₋₄ alkynyl”). In some embodiments, a heteroalkynyl group has 2 to 3 carbon atoms, at least one triple bond, and 1 heteroatom within the parent chain (“heteroC₂₋₃ alkynyl”). In some embodiments, a heteroalkynyl group has 2 carbon atoms, at least one triple bond, and 1 heteroatom within the parent chain (“heteroC₂ alkynyl”). In some embodiments, a heteroalkynyl group has 2 to 6 carbon atoms, at least one triple bond, and 1 or 2 heteroatoms within the parent chain (“heteroC₂₋₆ alkynyl”). Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted heteroC₂₋₂₀ alkynyl. In certain embodiments, the heteroalkynyl group is a substituted heteroC₂₋₂₀ alkynyl.

The term “carbocyclyl” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In some embodiments, a carbocyclyl group has 3 to 14 ring carbon atoms (“C₃₋₁₄ carbocyclyl”). In some embodiments, a carbocyclyl group has 3 to 13 ring carbon atoms (“C₃₋₁₃ carbocyclyl”). In some embodiments, a carbocyclyl group has 3 to 12 ring carbon atoms (“C₃₋₁₂ carbocyclyl”). In some embodiments, a carbocyclyl group has 3 to 11 ring carbon atoms (“C₃₋₁₁ carbocyclyl”). In some embodiments, a carbocyclyl group has 3 to 10 ring carbon atoms (“C₃₋₁₀ carbocyclyl”). In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 4 to 6 ring carbon atoms (“C₄₋₆ carbocyclyl”). In some embodiments, a carbocyclyl group has 5 to 6 ring carbon atoms (“C₅₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. Exemplary C₃₋₈ carbocyclyl groups include the aforementioned C₃₋₁₀ carbocyclyl groups as well as cycloundecyl (C₁₁), spiro[5.5]undecanyl (C₁₁), cyclododecyl (C₁₂), cyclododecenyl (C₁₂), cyclotridecane (C₁₃), cyclotetradecane (C₁₄), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₄ carbocyclyl. In certain embodiments, the carbocyclyl group is a substituted C₃₋₁₄ carbocyclyl.

In some embodiments, “carbocyclyl” is a monocyclic, saturated carbocyclyl group having from 3 to 14 ring carbon atoms (“C₃₋₁₄ cycloalkyl”). In some embodiments, a cycloalkyl group has 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 4 to 6 ring carbon atoms (“C₄₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₄ cycloalkyl. In certain embodiments, the cycloalkyl group is a substituted C₃₋₁₄ cycloalkyl. In certain embodiments, the carbocyclyl includes 0, 1, or 2 C═C double bonds in the carbocyclic ring system, as valency permits.

The term “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl. In certain embodiments, the heterocyclyl is substituted or unsubstituted, 3- to 7-membered, monocyclic heterocyclyl, wherein 1, 2, or 3 atoms in the heterocyclic ring system are independently oxygen, nitrogen, or sulfur, as valency permits.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

Exemplary 3-membered heterocyclyl groups containing 1 heteroatom include azirdinyl, oxiranyl, and thiiranyl. Exemplary 4-membered heterocyclyl groups containing 1 heteroatom include azetidinyl, oxetanyl, and thietanyl. Exemplary 5-membered heterocyclyl groups containing 1 heteroatom include tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include dioxolanyl, oxathiolanyl and dithiolanyl. Exemplary 5-membered heterocyclyl groups containing 3 heteroatoms include triazolinyl, oxadiazolinyl, and thiadiazolinyl. Exemplary 6-membered heterocyclyl groups containing 1 heteroatom include piperidinyl, tetrahydropyranyl, dihydropyridinyl, and thianyl. Exemplary 6-membered heterocyclyl groups containing 2 heteroatoms include piperazinyl, morpholinyl, dithianyl, and dioxanyl. Exemplary 6-membered heterocyclyl groups containing 3 heteroatoms include triazinyl. Exemplary 7-membered heterocyclyl groups containing 1 heteroatom include azepanyl, oxepanyl and thiepanyl. Exemplary 8-membered heterocyclyl groups containing 1 heteroatom include azocanyl, oxecanyl and thiocanyl. Exemplary bicyclic heterocyclyl groups include indolinyl, isoindolinyl, dihydrobenzofuranyl, dihydrobenzothienyl, tetra-hydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

The term “aryl” refers to a radical of a monocyclic or polycyclic (e.g., bicyclic or tricyclic) 4n+2 aromatic ring system (e.g., having 6, 10, or 14 it electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents. In certain embodiments, the aryl group is an unsubstituted C₆₋₁₄ aryl. In certain embodiments, the aryl group is a substituted C₆₋₁₄ aryl.

“Aralkyl” is a subset of “alkyl” and refers to an alkyl group substituted by an aryl group, wherein the point of attachment is on the alkyl moiety.

The term “heteroaryl” refers to a radical of a 5-14 membered monocyclic or polycyclic (e.g., bicyclic, tricyclic) 4n+2 aromatic ring system (e.g., having 6, 10, or 14 π electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, e.g., either the ring bearing a heteroatom (e.g., 2-indolyl) or the ring that does not contain a heteroatom (e.g., 5-indolyl). In certain embodiments, the heteroaryl is substituted or unsubstituted, 5- or 6-membered, monocyclic heteroaryl, wherein 1, 2, 3, or 4 atoms in the heteroaryl ring system are independently oxygen, nitrogen, or sulfur. In certain embodiments, the heteroaryl is substituted or unsubstituted, 9- or 10-membered, bicyclic heteroaryl, wherein 1, 2, 3, or 4 atoms in the heteroaryl ring system are independently oxygen, nitrogen, or sulfur.

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

Exemplary 5-membered heteroaryl groups containing 1 heteroatom include pyrrolyl, furanyl, and thiophenyl. Exemplary 5-membered heteroaryl groups containing 2 heteroatoms include imidazolyl, pyrazolyl, oxazolyl, isoxazolyl, thiazolyl, and isothiazolyl. Exemplary 5-membered heteroaryl groups containing 3 heteroatoms include triazolyl, oxadiazolyl, and thiadiazolyl. Exemplary 5-membered heteroaryl groups containing 4 heteroatoms include tetrazolyl. Exemplary 6-membered heteroaryl groups containing 1 heteroatom include pyridinyl. Exemplary 6-membered heteroaryl groups containing 2 heteroatoms include pyridazinyl, pyrimidinyl, and pyrazinyl. Exemplary 6-membered heteroaryl groups containing 3 or 4 heteroatoms include triazinyl and tetrazinyl, respectively. Exemplary 7-membered heteroaryl groups containing 1 heteroatom include azepinyl, oxepinyl, and thiepinyl. Exemplary 5,6-bicyclic heteroaryl groups include indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include naphthyridinyl, pteridinyl, quinolinyl, isoquinolinyl, cinnolinyl, quinoxalinyl, phthalazinyl, and quinazolinyl. Exemplary tricyclic heteroaryl groups include phenanthridinyl, dibenzofuranyl, carbazolyl, acridinyl, phenothiazinyl, phenoxazinyl, and phenazinyl.

“Heteroaralkyl” is a subset of “alkyl” and refers to an alkyl group substituted by a heteroaryl group, wherein the point of attachment is on the alkyl moiety.

The term “unsaturated bond” refers to a double or triple bond.

The term “unsaturated” or “partially unsaturated” refers to a moiety that includes at least one double or triple bond.

The term “saturated” or “fully saturated” refers to a moiety that does not contain a double or triple bond, e.g., the moiety only contains single bonds.

Affixing the suffix “-ene” to a group indicates the group is a divalent moiety, e.g., alkylene is the divalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

A group is optionally substituted unless expressly provided otherwise. The term “optionally substituted” refers to being substituted or unsubstituted. In certain embodiments, alkyl, alkenyl, alkynyl, heteroalkyl, heteroalkenyl, heteroalkynyl, carbocyclyl, heterocyclyl, aryl, and heteroaryl groups are optionally substituted. “Optionally substituted” refers to a group which is substituted or unsubstituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” heteroalkenyl, “substituted” or “unsubstituted” heteroalkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds and includes any of the substituents described herein that results in the formation of a stable compound. The present invention contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety. The invention is not limited in any manner by the exemplary substituents described herein.

Exemplary carbon atom substituents include halogen, —CN, —NO₂, —N₃, —SO₂H, —SO₃H, —OH, —OR^{aa}, —ON(R^{bb})₂, —N(R^{bb})₂, —N(R^{bb})₃—X⁻, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃ —C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR^{cc})₂, —OP(═O)(R^{aa})₂, —OP(═O)(OR^{cc})₂, —P(═O)(N(R^{bb})₂)₂, —OP(═O)(N(R^{bb})₂)₂, —NR^{bb}P(═O)(R^{aa})₂, —NR^{bb}P(═O)(OR^{cc})₂, —NR^{bb}P(═O)(N(R^{bb})₂)₂, —P(R^{cc})₂, —P(OR^{cc})₂, —P(R^{cc})₃⁺X⁻, —P(OR^{cc})₃+X⁻, —P(R^{cc})₄, —P(OR)₄, —OP(R)₂, —OP(R^{cc})₃+X⁻, —OP(OR)₂, —OP(OR^{cc})₃+X⁻, —OP(R^{cc})₄, —OP(OR^{cc})₄, —B(R^{aa})₂, —B(OR^{cc})₂, —BR^{aa}(OR^{cc}), C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;
or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc};
wherein:
each instance of R^{aa} is, independently, selected from C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆-14 aryl, and 5-14 membered heteroaryl, or two R^{aa} groups are joined to form a 3-14 membered heterocyclyl or 5-14 membered heteroaryl ring, wherein each of the alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{bb} is, independently, selected from hydrogen, —OH, —OR, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S^{cc})SR^{cc}, —P(═O)(R^{aa})₂, —P(═O)(OR^{cc})₂, —P(═O)(N(R^{cc})₂)₂, C₁₋₂₀ alkyl, C₁₋₂₀ perhaloalkyl, C₁₋₂₀ alkenyl, C₁₋₂₀ alkynyl, heteroC₁₋₂₀alkyl, heteroC₁₋₂₀alkenyl, heteroC₁₋₂₀alkynyl, C₃₋₁₀ carbocyclyl, 3-14 membered heterocyclyl, C₆₋₁₄ aryl, and 5-14 membered heteroaryl, or two R groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{cc} is, independently, selected from hydrogen, C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}—, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, C₁₋₁₀ alkyl, C₁₋₁₀ perhaloalkyl, C₁₋₁₀ alkenyl, C₁₋₁₀ alkynyl, heteroC₁₋₁₀alkyl, heteroC₁₋₁₀alkenyl, heteroC₁₋₁₀alkynyl, C₃₋₁₀ carbocyclyl, 3-10 membered heterocyclyl, C₆₋₁₀ aryl, and 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R⁹⁹ groups, or two geminal R^{dd} substituents are joined to form ═O or ═S; wherein X⁻ is a counterion;
each instance of R^{ee} is, independently, selected from C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
each instance of R^{ff} is, independently, selected from hydrogen, C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, 3-₁₀ membered heterocyclyl, C₆-10 aryl, and 5-10 membered heteroaryl, or two R^{ff} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)N₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃ —C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, C₆₋₁₀ aryl, 3-10 membered heterocyclyl, or 5-10 membered heteroaryl; or two geminal R^{gg} substituents can be joined to form ═O or ═S; and each X⁻ is a counterion.

In certain embodiments, each carbon atom substituent is independently halogen, substituted (e.g., substituted with one or more halogen) or unsubstituted C₁₋₆ alkyl, —OR^{aa}, —SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂. In certain embodiments, each carbon atom substituent is independently halogen, substituted (e.g., substituted with one or more halogen) or unsubstituted C₁₋₁₀ alkyl, —OR^{aa}, —SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂, wherein R^{aa} is hydrogen, substituted (e.g., substituted with one or more halogen) or unsubstituted C₁₋₁₀ alkyl, an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted (e.g., substituted with one or more halogen) or unsubstituted C₁₋₁₀ alkyl, or a nitrogen protecting group (e.g., Bn, Boc, Cbz, Fmoc, trifluoroacetyl, triphenylmethyl, acetyl, or Ts). In certain embodiments, each carbon atom substituent is independently halogen, substituted (e.g., substituted with one or more halogen) or unsubstituted C₁₋₆ alkyl, —OR^{aa}, —SR, —N(R^{bb})₂, —CN, —SCN, or —NO₂. In certain embodiments, each carbon atom substituent is independently halogen, substituted (e.g., substituted with one or more halogen moieties) or unsubstituted C₁₋₁₀ alkyl, —OR^{aa}, —SR^{aa}, —N(R^{bb})₂, —CN, —SCN, or —NO₂, wherein R^{aa} is hydrogen, substituted (e.g., substituted with one or more halogen) or unsubstituted C₁₋₁₀ alkyl, an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R is independently hydrogen, substituted (e.g., substituted with one or more halogen) or unsubstituted C₁₋₁₀ alkyl, or a nitrogen protecting group (e.g., Bn, Boc, Cbz, Fmoc, trifluoroacetyl, triphenylmethyl, acetyl, or Ts).

In certain embodiments, the molecular weight of a carbon atom substituent is lower than 250, lower than 200, lower than 150, lower than 100, or lower than 50 g/mol. In certain embodiments, a carbon atom substituent consists of carbon, hydrogen, fluorine, chlorine, bromine, iodine, oxygen, sulfur, nitrogen, and/or silicon atoms. In certain embodiments, a carbon atom substituent consists of carbon, hydrogen, fluorine, chlorine, bromine, iodine, oxygen, sulfur, and/or nitrogen atoms. In certain embodiments, a carbon atom substituent consists of carbon, hydrogen, fluorine, chlorine, bromine, and/or iodine atoms. In certain embodiments, a carbon atom substituent consists of carbon, hydrogen, fluorine, and/or chlorine atoms.

The term “halo” or “halogen” refers to fluorine (fluoro, —F), chlorine (chloro, —Cl), bromine (bromo, —Br), or iodine (iodo, —I).

The term “hydroxyl” or “hydroxy” refers to the group —OH. The term “substituted hydroxyl” or “substituted hydroxyl,” by extension, refers to a hydroxyl group wherein the oxygen atom directly attached to the parent molecule is substituted with a group other than hydrogen, and includes groups selected from —OR^{aa}, —ON(R^{bb})₂, —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OSi(R^{aa})₃, —OP(R^{cc})₂, —OP(R^{cc})₃⁺X⁻, —OP(OR^{cc})₂, —OP(OR^{cc})₃⁺X⁻, —OP(═O)(R^{aa})₂, —OP(═O)(OR^{cc})₂, and —OP(═O)(N(R^{bb}))₂, wherein X⁻, R^{aa}, R^{bb}, and R^{cc} are as defined herein.

The term “thiol” or “thio” refers to the group —SH. The term “substituted thiol” or “substituted thio,” by extension, refers to a thiol group wherein the sulfur atom directly attached to the parent molecule is substituted with a group other than hydrogen, and includes groups selected from —SR^{aa}, —S═SR^{cc}, —SC(═S)SR^{aa}, —SC(═S)OR^{aa}, —SC(═S) N(R^{bb})₂, —SC(═O)SR^{aa}, —SC(═O)OR^{aa}, —SC(═O)N(R^{bb})₂, and —SC(═O)R^{aa}, wherein R^{aa} and R^{cc} are as defined herein.

The term “amino” refers to the group —NH₂. The term “substituted amino,” by extension, refers to a monosubstituted amino, a disubstituted amino, or a trisubstituted amino. In certain embodiments, the “substituted amino” is a monosubstituted amino or a disubstituted amino group.

The term “mono substituted amino” refers to an amino group wherein the nitrogen atom directly attached to the parent molecule is substituted with one hydrogen and one group other than hydrogen, and includes groups selected from —NH(R^{bb}), —NHC(═O)R^{aa}, —NHCO₂R^{aa}, —NHC(═O)N(R^{bb})₂, —NHC(═NR^{bb})N(R^{bb})₂, —NHSO₂R^{aa}, —NHP(═O)(OR^{cc})₂, and —NHP(═O)(N(R^{bb})₂)₂, wherein R^{aa}, R^{bb} and R^{cc} are as defined herein, and wherein R^{bb} of the group —NH(R^{bb}) is not hydrogen.

The term “disubstituted amino” refers to an amino group wherein the nitrogen atom directly attached to the parent molecule is substituted with two groups other than hydrogen, and includes groups selected from —N(R^{bb})₂, —NR^{bb} C(═O)R^{aa}, —NR^{bb}CO₂R^{aa}, —NR^{bb}C(═O)N(R^{bb})₂, —NR^{bb}C(═NR^{bb})N(R^{bb})₂, —NR^{bb}SO₂R^{aa}, —NR^{bb}P(═O)(OR^{cc})₂, and —NR^{bb}P(═O)(N(R^{bb})₂)₂, wherein R^{aa}, R^{bb}, and R^{cc} are as defined herein, with the proviso that the nitrogen atom directly attached to the parent molecule is not substituted with hydrogen.

The term “trisubstituted amino” refers to an amino group wherein the nitrogen atom directly attached to the parent molecule is substituted with three groups, and includes groups selected from —N(R^{bb})₃ and —N(R^{bb})₃⁺X⁻, wherein R^{bb} and X⁻ are as defined herein.

The term “sulfonyl” refers to a group selected from —SO₂N(R^{bb})₂, —SO₂R^{aa}, and —SO₂OR^{aa}, wherein R^{aa} and R^{bb} are as defined herein.

The term “sulfinyl” refers to the group —S(═O)R^{aa}, wherein R^{aa} is as defined herein.

The term “acyl” refers to a group having the general formula —C(═O)R^{xi} —C(═O)OR^{X1}, —C(═O)—O—C(═O)R^{X1}, —C(═O)SR^{X1}, —C(═O)N(R^{X1})₂, —C(═S)R^{X1}, —C(═S)N(R^{X1})₂, and —C(═S)S(R^{X1}), —C(═NR^{X1})R^{X1}, —C(═NR^{X1})OR^{X1}, —C(═NR^{X1})SR^{X1}, and —C(═NR^{X1})N(R^{X1})₂, wherein R^{X1} is hydrogen; halogen; substituted or unsubstituted hydroxyl; substituted or unsubstituted thiol; substituted or unsubstituted amino; substituted or unsubstituted acyl, cyclic or acyclic, substituted or unsubstituted, branched or unbranched aliphatic; cyclic or acyclic, substituted or unsubstituted, branched or unbranched heteroaliphatic; cyclic or acyclic, substituted or unsubstituted, branched or unbranched alkyl; cyclic or acyclic, substituted or unsubstituted, branched or unbranched alkenyl; substituted or unsubstituted alkynyl; substituted or unsubstituted aryl, substituted or unsubstituted heteroaryl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, mono- or di-aliphaticamino, mono- or di-heteroaliphaticamino, mono- or di-alkylamino, mono- or di-heteroalkylamino, mono- or di-arylamino, or mono- or di-heteroarylamino; or two R^{X1} groups taken together form a 5- to 6-membered heterocyclic ring. Exemplary acyl groups include aldehydes (—CHO), carboxylic acids (—CO₂H), ketones, acyl halides, esters, amides, imines, carbonates, carbamates, and ureas. Acyl substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “carbonyl” refers to a group wherein the carbon directly attached to the parent molecule is sp² hybridized, and is substituted with an oxygen, nitrogen or sulfur atom, e.g., a group selected from ketones (—C(═O)R^{aa}), carboxylic acids (—CO₂H), aldehydes (—CHO), esters (—CO₂R^{aa}, —C(═O)SR^{aa}, —C(═S)SR^{aa}), amides (—C(═O)N(R^{bb})₂, —C(═O)NR^{bb}SO₂R^{aa}, —C(═S)N(R^{bb})₂), and imines (—C(═NR^{bb})R^{aa}, —C(═NR^{bb})OR^{aa}), —C(═NR^{bb})N(R^{bb})₂), wherein R^{aa} and R^{bb} are as defined herein.

The term “silyl” refers to the group —Si(R^{aa})₃, wherein R^{aa} is as defined herein.

The term “phosphino” refers to the group —P(R^{cc})₂, wherein R^{cc} is as defined herein.

The term “phosphono” refers to the group —(P═O)(OR^{cc})₂, wherein R^{aa}and R^{cc} are as defined herein.

The term “phosphoramido” refers to the group —O(P═O)(N(R^{bb})₂)₂, wherein each R is as defined herein.

The term “oxo” refers to the group ═O, and the term “thiooxo” refers to the group ═S.

Nitrogen atoms can be substituted or unsubstituted as valency permits, and include primary, secondary, tertiary, and quaternary nitrogen atoms. Exemplary nitrogen atom substituents include hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(OR^{cc})₂, —P(═O)(R^{aa})₂, —P(═O)(N(R^{cc})₂)₂, C₁₋₂₀ alkyl, C₁₋₂₀ perhaloalkyl, C₁₋₂₀ alkenyl, C₁₋₂₀ alkynyl, hetero C₁₋₂₀ alkyl, hetero C₁₋₂₀ alkenyl, hetero C₁₋₂₀ alkynyl, C₃₋₁₀ carbocyclyl, 3-14 membered heterocyclyl, C₆₋₁₄ aryl, and 5-14 membered heteroaryl, or two R^{cc} groups attached to an N atom are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

In certain embodiments, each nitrogen atom substituent is independently substituted (e.g., substituted with one or more halogen) or unsubstituted C₁₋₆ alkyl, —C(═O)R^{aa}, —CO₂R^{aa}, or a nitrogen protecting group. In certain embodiments, each nitrogen atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a nitrogen protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each nitrogen atom substituent is independently substituted (e.g., substituted with one or more halogen) or unsubstituted C₁₋₆ alkyl or a nitrogen protecting group.

In certain embodiments, the substituent present on the nitrogen atom is a nitrogen protecting group (also referred to herein as an “amino protecting group”). Nitrogen protecting groups include —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₁₋₂₀ alkenyl, C₁₋₂₀ alkynyl, hetero C₁₋₂₀ alkyl, hetero C₁₋₂₀ alkenyl, hetero C₁₋₂₀ alkynyl, C₃₋₁₀carbocyclyl, 3-₁₄ membered heterocyclyl, C₆-14 aryl, and 5-14 membered heteroaryl groups, wherein each alkyl, alkenyl, alkynyl, heteroalkyl, heteroalkenyl, heteroalkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc}and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

For example, in certain embodiments, at least one nitrogen protecting group is an amide group (e.g., a moiety that include the nitrogen atom to which the nitrogen protecting groups (e.g., —C(═O)R^{aa}) is directly attached). In certain such embodiments, each nitrogen protecting group, together with the nitrogen atom to which the nitrogen protecting group is attached, is independently selected from the group consisting of formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivatives, benzamide, p-phenylbenzamide, o-nitophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivatives, o-nitrobenzamide, and o-(benzoyloxymethyl)benzamide.

In certain embodiments, at least one nitrogen protecting group is a carbamate group (e.g., a moiety that includes the nitrogen atom to which the nitrogen protecting groups (e.g., —C(═O)OR^{aa}) is directly attached). In certain such embodiments, each nitrogen protecting group, together with the nitrogen atom to which the nitrogen protecting group is attached, is independently selected from the group consisting of methyl carbamate, ethyl carbamate, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC or Boc), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

In certain embodiments, at least one nitrogen protecting group is a sulfonamide group (e.g., a moiety that include the nitrogen atom to which the nitrogen protecting groups (e.g., —S(═O)₂R^{aa}) is directly attached). In certain such embodiments, each nitrogen protecting group, together with the nitrogen atom to which the nitrogen protecting group is attached, is independently selected from the group consisting of p-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

In certain embodiments, each nitrogen protecting group, together with the nitrogen atom to which the nitrogen protecting group is attached, is independently selected from the group consisting of phenothiazinyl-(10)-acyl derivatives, N′-p-toluenesulfonylaminoacyl derivatives, N′-phenylaminothioacyl derivatives, N-benzoylphenylalanyl derivatives, N-acetylmethionine derivatives, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivatives, N-diphenylborinic acid derivatives, N—[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys). In some embodiments, two instances of a nitrogen protecting group together with the nitrogen atoms to which the nitrogen protecting groups are attached are N,N′-isopropylidenediamine.

In certain embodiments, at least one nitrogen protecting group is Bn, Boc, Cbz, Fmoc, trifluoroacetyl, triphenylmethyl, acetyl, or Ts.

In certain embodiments, each oxygen atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or an oxygen protecting group. In certain embodiments, each oxygen atom substituents is independently substituted (e.g., substituted with one or more halogen) or unsubstituted C₁₋₆ alkyl, —C(═O)R^{aa}, —CO₂R^{aa}, —C(═O)N(R^{bb})₂, or an oxygen protecting group, wherein R^{aa} is hydrogen, substituted (e.g., substituted with one or more halogen) or unsubstituted C₁₋₁₀ alkyl, or an oxygen protecting group when attached to an oxygen atom; and each R is independently hydrogen, substituted (e.g., substituted with one or more halogen) or unsubstituted C₁₋₁₀ alkyl, or a nitrogen protecting group. In certain embodiments, each oxygen atom substituent is independently substituted (e.g., substituted with one or more halogen) or unsubstituted C₁₋₆ alkyl or an oxygen protecting group.

In certain embodiments, the substituent present on an oxygen atom is an oxygen protecting group (also referred to herein as an “hydroxyl protecting group”). Oxygen protecting groups include —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂ —P(═O)(OR^{cc})₂, and —P(═O)(N(R^{bb})₂)₂, wherein X⁻, R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

In certain embodiments, each oxygen protecting group, together with the oxygen atom to which the oxygen protecting group is attached, is selected from the group consisting of methyl, methoxymethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl (PMB), 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, a-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 4,4′-Dimethoxy-3′″-[N-(imidazolylmethyl)]trityl Ether (IDTr-OR), 4,4′-Dimethoxy-3′″-[N-(imidazolylethyl)carbamoyl]trityl Ether (IETr-OR), 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), methyl carbonate, 9-fluorenylmethyl carbonate (Fmoc), ethyl carbonate, 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), isobutyl carbonate, vinyl carbonate, allyl carbonate, t-butyl carbonate (BOC or Boc), p-nitrophenyl carbonate, benzyl carbonate, p-methoxybenzyl carbonate, 3,4-dimethoxybenzyl carbonate, o-nitrobenzyl carbonate, p-nitrobenzyl carbonate, S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl carbonate (MTMEC-OR), 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, a-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, at least one oxygen protecting group is silyl, TBDPS, TBDMS, TIPS, TES, TMS, MOM, THP, t-Bu, Bn, allyl, acetyl, pivaloyl, or benzoyl.

In certain embodiments, each sulfur atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a sulfur protecting group. In certain embodiments, each sulfur atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a sulfur protecting group, wherein R is hydrogen, substituted (e.g., substituted with one or more halogen) or unsubstituted C₁₋₁₀ alkyl, or an oxygen protecting group when attached to an oxygen atom; and each R is independently hydrogen, substituted (e.g., substituted with one or more halogen) or unsubstituted C₁₋₁₀ alkyl, or a nitrogen protecting group. In certain embodiments, each sulfur atom substituent is independently substituted (e.g., substituted with one or more halogen) or unsubstituted C₁₋₆ alkyl or a sulfur protecting group.

In certain embodiments, the substituent present on a sulfur atom is a sulfur protecting group (also referred to as a “thiol protecting group”). In some embodiments, each sulfur protecting group is selected from the group consisting of —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa} —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

In certain embodiments, the molecular weight of a substituent is lower than 250, lower than 200, lower than 150, lower than 100, or lower than 50 g/mol. In certain embodiments, a substituent consists of carbon, hydrogen, fluorine, chlorine, bromine, iodine, oxygen, sulfur, nitrogen, and/or silicon atoms. In certain embodiments, a substituent consists of carbon, hydrogen, fluorine, chlorine, bromine, iodine, oxygen, sulfur, and/or nitrogen atoms. In certain embodiments, a substituent consists of carbon, hydrogen, fluorine, chlorine, bromine, and/or iodine atoms. In certain embodiments, a substituent consists of carbon, hydrogen, fluorine, and/or chlorine atoms. In certain embodiments, a substituent comprises 0, 1, 2, or 3 hydrogen bond donors. In certain embodiments, a substituent comprises 0, 1, 2, or 3 hydrogen bond acceptors.

A “counterion” or “anionic counterion” is a negatively charged group associated with a positively charged group in order to maintain electronic neutrality. An anionic counterion may be monovalent (e.g., including one formal negative charge). An anionic counterion may also be multivalent (e.g., including more than one formal negative charge), such as divalent or trivalent. Exemplary counterions include halide ions (e.g., F⁻, Cl⁻, Br⁻, I⁻), NO₃⁻, ClO₄⁻, OH⁻, H₂PO₄⁻, HCO₃⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), carboxylate ions (e.g., acetate, propanoate, benzoate, glycerate, lactate, tartrate, glycolate, gluconate, and the like), BF₄⁻, PF₄⁻, PF₆⁻, AsF₆⁻, SbF₆⁻, B[3,5-(CF₃)₂C₆H₃]₄]⁻, B(C₆F₅)₄⁻, BPh₄⁻, Al(OC(CF₃)₃)₄⁻, and carborane anions (e.g., CB₁₁H₁₂⁻ or (HCB₁₁Me₅Br₆)⁻). Exemplary counterions which may be multivalent include CO₃²⁻, HPO₄²⁻, PO₄³⁻, B₄O₇²⁻, SO₄²⁻, S₂O₃²⁻, carboxylate anions (e.g., tartrate, citrate, fumarate, maleate, malate, malonate, gluconate, succinate, glutarate, adipate, pimelate, suberate, azelate, sebacate, salicylate, phthalates, aspartate, glutamate, and the like), and carboranes.

A “leaving group” (LG) is an art-understood term referring to an atomic or molecular fragment that departs with a pair of electrons in heterolytic bond cleavage, wherein the molecular fragment is an anion or neutral molecule. As used herein, a leaving group can be an atom or a group capable of being displaced by a nucleophile. See e.g., Smith, March Advanced Organic Chemistry 6th ed. (501-502). Exemplary leaving groups include, but are not limited to, halo (e.g., fluoro, chloro, bromo, iodo) and activated substituted hydroxyl groups (e.g., —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OP(R^{cc})₂, —OP(R^{cc})₃, —OP(═O)₂R^{aa}, —OP(═O)(R^{aa})₂, —OP(═O)(OR^{cc})₂, —OP(═O)₂N(R^{bb})₂, and —OP(═O)(NR^{bb})₂, wherein R^{aa}, R^{bb}, and R^{cc} are as defined herein). Additional examples of suitable leaving groups include, but are not limited to, halogen alkoxycarbonyloxy, aryloxycarbonyloxy, alkanesulfonyloxy, arenesulfonyloxy, alkyl-carbonyloxy (e.g., acetoxy), arylcarbonyloxy, aryloxy, methoxy, N,O-dimethylhydroxylamino, pixyl, and haloformates. In some embodiments, the leaving group is a sulfonic acid ester, such as toluenesulfonate (tosylate, -OTs), methanesulfonate (mesylate, -OMs), p-bromobenzenesulfonyloxy (brosylate, -OBs), —OS(═O)₂(CF₂)₃CF₃ (nonaflate, -ONf), or trifluoromethanesulfonate (triflate, -OTf). In some embodiments, the leaving group is a brosylate, such as p-bromobenzenesulfonyloxy. In some embodiments, the leaving group is a nosylate, such as 2-nitrobenzenesulfonyloxy. In some embodiments, the leaving group is a sulfonate-containing group. In some embodiments, the leaving group is a tosylate group. In some embodiments, the leaving group is a phosphineoxide (e.g., formed during a Mitsunobu reaction) or an internal leaving group such as an epoxide or cyclic sulfate. Other non-limiting examples of leaving groups are water, ammonia, alcohols, ether moieties, thioether moieties, zinc halides, magnesium moieties, diazonium salts, and copper moieties.

Use of the phrase “at least one instance” refers to 1, 2, 3, 4, or more instances, but also encompasses a range, e.g., for example, from 1 to 4, from 1 to 3, from 1 to 2, from 2 to 4, from 2 to 3, or from 3 to 4 instances, inclusive.

A “non-hydrogen group” refers to any group that is defined for a particular variable that is not hydrogen.

These and other exemplary substituents are described in more detail in the Detailed Description, Examples, and Claims. The invention is not limited in any manner by the above exemplary listing of substituents.

As used herein, the term “salt” refers to any and all salts and encompasses pharmaceutically acceptable salts. The term “salt” refers to ionic compounds that result from the neutralization reaction of an acid and a base. A salt is composed of one or more cations (positively charged ions) and one or more anions (negative ions) so that the salt is electrically neutral (without a net charge). Salts of the compounds of this disclosure include those derived from inorganic and organic acids and bases. Examples of acid addition salts are salts of an amino group formed with inorganic acids, such as hydrochloric acid, hydrobromic acid, phosphoric acid, sulfuric acid, and perchloric acid, or with organic acids, such as acetic acid, oxalic acid, maleic acid, tartaric acid, citric acid, succinic acid, or malonic acid or by using other methods known in the art such as ion exchange. Other salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate, hippurate, and the like. Salts derived from appropriate bases include alkali metal, alkaline earth metal, ammonium, and N⁺(C₁₋₄ alkyl)₄ salts. Representative alkali or alkaline earth metal salts include sodium, lithium, potassium, calcium, magnesium, and the like. Further salts include ammonium, quaternary ammonium, and amine cations formed using counterions such as halide, hydroxide, carboxylate, sulfate, phosphate, nitrate, lower alkyl sulfonate, and aryl sulfonate.

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. Pharmaceutically acceptable salts of the compounds of this disclosure include those derived from suitable inorganic and organic acids and bases. Examples of pharmaceutically acceptable, nontoxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N⁺(C₁₋₄ alkyl)₄⁻ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

The term “solvate” refers to forms of the compound, or a salt thereof, that are associated with a solvent, usually by a solvolysis reaction. This physical association may include hydrogen bonding. Conventional solvents include water, methanol, ethanol, acetic acid, DMSO, THF, diethyl ether, and the like. The compounds described herein may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolatable solvates. Representative solvates include hydrates, ethanolates, and methanolates.

The term “hydrate” refers to a compound that is associated with water. Typically, the number of the water molecules contained in a hydrate of a compound is in a definite ratio to the number of the compound molecules in the hydrate. Therefore, a hydrate of a compound may be represented, for example, by the general formula R·x H₂O, wherein R is the compound, and x is a number greater than 0. A given compound may form more than one type of hydrate, including, e.g., monohydrates (x is 1), lower hydrates (x is a number greater than 0 and smaller than 1, e.g., hemihydrates (R·0.5 H₂O)), and polyhydrates (x is a number greater than 1, e.g., dihydrates (R·2H₂O) and hexahydrates (R·6H₂O)).

The term “polymorph” refers to a crystalline form of a compound (or a salt, hydrate, or solvate thereof). All polymorphs have the same elemental composition. Different crystalline forms usually have different X-ray diffraction patterns, infrared spectra, melting points, density, hardness, crystal shape, optical and electrical properties, stability, and solubility. Recrystallization solvent, rate of crystallization, storage temperature, and other factors may cause one crystal form to dominate. Various polymorphs of a compound can be prepared by crystallization under different conditions.

The term “co-crystal” refers to a crystalline structure comprising at least two different components (e.g., a compound and an acid), wherein each of the components is independently an atom, ion, or molecule. In certain embodiments, none of the components is a solvent. In certain embodiments, at least one of the components is a solvent. A co-crystal of a compound and an acid is different from a salt formed from a compound and the acid. In the salt, a compound is complexed with the acid in a way that proton transfer (e.g., a complete proton transfer) from the acid to a compound easily occurs at room temperature. In the co-crystal, however, a compound is complexed with the acid in a way that proton transfer from the acid to a herein does not easily occur at room temperature. In certain embodiments, in the co-crystal, there is substantially no proton transfer from the acid to a compound. In certain embodiments, in the co-crystal, there is partial proton transfer from the acid to a compound. Co-crystals may be useful to improve the properties (e.g., solubility, stability, and ease of formulation) of a compound.

The term “tautomers” or “tautomeric” refers to two or more interconvertible compounds resulting from at least one formal migration of a hydrogen atom and at least one change in valency (e.g., a single bond to a double bond, a triple bond to a single bond, or vice versa). The exact ratio of the tautomers depends on several factors, including temperature, solvent, and pH. Tautomerizations (i.e., the reaction providing a tautomeric pair) may catalyzed by acid or base. Exemplary tautomerizations include keto-to-enol, amide-to-imide, lactam-to-lactim, enamine-to-imine, and enamine-to-(a different enamine) tautomerizations.

Compounds that have the same molecular formula but differ in the nature or sequence of bonding of their atoms or the arrangement of their atoms in space are termed “isomers.” Isomers that differ in the arrangement of their atoms in space are termed “stereoisomers.”

Stereoisomers that are not mirror images of one another are termed “diastereomers,” and those that are non-superimposable mirror images of each other are termed “enantiomers.” When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture.”

The term “isotopically labeled compound” refers to a derivative of a compound that only structurally differs from the compound in that at least one atom of the derivative includes at least one isotope enriched above (e.g., enriched 3—, 10-, 30-, 100-, 300-, 1,000-, 3,000- or 10,000-fold above) its natural abundance, whereas each atom of the compound includes isotopes at their natural abundances. In certain embodiments, the isotope enriched above its natural abundance is ²H. In certain embodiments, the isotope enriched above its natural abundance is ¹³C, ¹⁵N, or ¹⁸O.

The term “prodrugs” refers to compounds that have cleavable groups and become by solvolysis or under physiological conditions the compounds described herein, which are pharmaceutically active in vivo. Such examples include choline ester derivatives and the like, N-alkylmorpholine esters and the like. Other derivatives of the compounds described herein have activity in both their acid and acid derivative forms, but in the acid sensitive form often offer advantages of solubility, tissue compatibility, or delayed release in the mammalian organism (see, Bundgaard, H., Design of Prodrugs, pp. 7-9, 21-24, Elsevier, Amsterdam 1985). Prodrugs include, for example, esters prepared by reaction of the parent acid with a suitable alcohol, or amides prepared by reaction of the parent acid compound with a substituted or unsubstituted amine, or acid anhydrides, or mixed anhydrides.

The terms “pharmaceutical composition,” “composition,” and “formulation” are used interchangeably.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. The non-human animal may be a male or female at any stage of development. The non-human animal may be a transgenic animal or genetically engineered animal. The term “patient” refers to a human subject in need of treatment of a disease.

The term “biological sample” refers to any sample including tissue samples (such as tissue sections and needle biopsies of a tissue); cell samples (e.g., cytological smears (such as Pap or blood smears) or samples of cells obtained by microdissection); samples of whole organisms (such as samples of yeasts or bacteria); or cell fractions, fragments or organelles (such as obtained by lysing cells and separating the components thereof by centrifugation or otherwise). Other examples of biological samples include blood, serum, urine, semen, fecal matter, cerebrospinal fluid, interstitial fluid, mucous, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample.

The term “administer,” “administering,” or “administration” refers to implanting, absorbing, ingesting, injecting, inhaling, or otherwise introducing a compound described herein, or a pharmaceutical composition thereof, in or on a subject.

The terms “treatment,” “treat,” and “treating” refer to reversing, alleviating, delaying the onset of, or inhibiting the progress of a disease described herein. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms and/or in light of exposure to a pathogen). Treatment may also be continued after symptoms have resolved, for example, to delay or prevent recurrence.

The term “prevent,” “preventing,” or “prevention” refers to a prophylactic treatment of a subject who is not and was not with a disease but is at risk of developing the disease or who was with a disease, is not with the disease, but is at risk of regression of the disease. In certain embodiments, the subject is at a higher risk of developing the disease or at a higher risk of regression of the disease than an average healthy member of a population. In some embodiments, the subject is at risk of developing a disease or condition due to environmental factors (e.g., exposure to the sun).

An “effective amount” of a compound described herein refers to an amount sufficient to elicit the desired biological response. An effective amount of a compound described herein may vary depending on such factors as the desired biological endpoint, severity of side effects, disease, or disorder, the identity, pharmacokinetics, and pharmacodynamics of the particular compound, the condition being treated, the mode, route, and desired or required frequency of administration, the species, age and health or general condition of the subject. In certain embodiments, an effective amount is a therapeutically effective amount. In certain embodiments, an effective amount is a prophylactic treatment. In certain embodiments, an effective amount is the amount of a compound described herein in a single dose. In certain embodiments, an effective amount is the combined amounts of a compound described herein in multiple doses. In certain embodiments, the desired dosage is delivered three times a day, two times a day, once a day, every other day, every third day, every week, every two weeks, every three weeks, or every four weeks. In certain embodiments, the desired dosage is delivered using multiple administrations (e.g., two, three, four, five, six, seven, eight, nine, ten, eleven, twelve, thirteen, fourteen, or more administrations).

In certain embodiments, an effective amount of a compound for administration one or more times a day to a 70 kg adult human comprises about 0.0001 mg to about 3000 mg, about 0.0001 mg to about 2000 mg, about 0.0001 mg to about 1000 mg, about 0.001 mg to about 1000 mg, about 0.01 mg to about 1000 mg, about 0.1 mg to about 1000 mg, about 1 mg to about 1000 mg, about 1 mg to about 100 mg, about 10 mg to about 1000 mg, or about 100 mg to about 1000 mg, of a compound per unit dosage form.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

A “therapeutically effective amount” of a compound described herein is an amount sufficient to provide a therapeutic benefit in the treatment of a condition or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms, signs, or causes of the condition, and/or enhances the therapeutic efficacy of another therapeutic agent. In certain embodiments, a therapeutically effective amount is an amount sufficient for treating a disease or disorder associated with associated with an RNA target in a subject in need thereof. In certain embodiments, a therapeutically effective amount is an amount sufficient for treating a disease or disorder associated with associated with microRNA-21 (miR-21) (e.g., proliferative disease, kidney disease) in a subject in need thereof. In certain embodiments, a therapeutically effective amount is an amount effective for treating a disease or disorder associated with microRNA-21 (miR-21) (e.g., proliferative disease, kidney disease) in a subject in need thereof. In certain embodiments, a therapeutically effective amount is an amount effective for binding a precursor to microRNA-21 (pre-miR-21) in a subject in need thereof or in a cell, tissue, or biological sample (e.g., by at least about 10%, at least about 20%, at least about 30%, at least about 40%, at least about 50%, at least about 60%, at least about 70%, at least about 80%, at least about 90%, at least 95%, at least 98%, at least 99%, or at least about 100%). In certain embodiments, a therapeutically effective amount is an amount effective for downregulating Eye mature microRNA-21 (miR-21) (e.g., by at least about 10%, at least about 20%, at least about 30%, at least about 40%, at least about 50%, at least about 60%, at least about 70%, at least about 80%, at least about 90%, at least 95%, at least 98%, at least 99%, or at least about 100%). In certain embodiments, a therapeutically effective amount is an amount effective for decreasing an amount of pre-miR-21 (e.g., by at least about 10%, at least about 20%, at least about 30%, at least about 40%, at least about 50%, at least about 60%, at least about 70%, at least about 80%, at least about 90%, at least 95%, at least 98%, at least 99%, or at least about 100%). In certain embodiments, a therapeutically effective amount is an amount effective for cleaving pre-miR-21 (e.g., by at least about 10%, at least about 20%, at least about 30%, at least about 40%, at least about 50%, at least about 60%, at least about 70%, at least about 80%, at least about 90%, at least 95%, at least 98%, at least 99%, or at least about 100%). In certain embodiments, a therapeutically effective amount is an amount effective for downregulating mature microRNA-21 (miR-21) (e.g. miR-21-5p). In certain embodiments, miR-21 is downregulated (e.g., by at least about 10%, at least about 20%, at least about 30%, at least about 40%, at least about 50%, at least about 60%, at least about 70%, at least about 80%, at least about 90%, at least 95%, at least 98%, at least 99%, or at least about 100%). In certain embodiments, a therapeutically effective amount is an amount effective for decreasing an amount of miR-21 (e.g., by at least about 10%, at least about 20%, at least about 30%, at least about 40%, at least about 50%, at least about 60%, at least about 70%, at least about 80%, at least about 90%, at least 95%, at least 98%, at least 99%, or at least about 100%). In certain embodiments, a therapeutically effective amount is an amount effective for increasing an amount of programmed cell death protein 4 (PDCD4) (e.g., by at least about 10%, at least about 20%, at least about 30%, at least about 40%, at least about 50%, at least about 60%, at least about 70%, at least about 80%, at least about 90%, at least about 100%, at least about 150%, at least about 200%, at least about 300%, at least about 400%, at least about 500%, at least about 600%, at least about 700%, at least about 800%, at least about 900%, or at least about 1000%).

A “prophylactically effective amount” of a compound is an amount sufficient to prevent a condition, or one or more signs and/or symptoms associated with the condition or prevent its recurrence. In certain embodiments, the prophylactically effective amount is an amount that improves overall prophylaxis and/or enhances the prophylactic efficacy of another prophylactic agent. In certain embodiments, a prophylactically effective amount is an amount effective for preventing a disease or disorder associated with an RNA target in a subject in need thereof. In certain embodiments, a prophylactically effective amount is an amount effective for preventing a disease or disorder associated with microRNA-21 (miR-21) (e.g., proliferative disease, kidney disease) in a subject in need thereof. In certain embodiments, a prophylactically effective amount is an amount effective for reducing the risk of developing a disease or disorder associated with microRNA-21 (miR-21) (e.g., proliferative disease, kidney disease) in a subject in need thereof. In certain embodiments, a prophylactically effective amount is an amount effective for binding a precursor to microRNA-21 (pre-miR-21) in a subject in need thereof or in a cell, tissue, or biological sample (e.g., by at least about 10%, at least about 20%, at least about 30%, at least about 40%, at least about 50%, at least about 60%, at least about 70%, at least about 80%, at least about 90%, at least 95%, at least 98%, at least 99%, or at least about 100%). In certain embodiments, a prophylactically effective amount is an amount effective for downregulating mature microRNA-21 (miR-21) (e.g., by at least about 10%, at least about 20%, at least about 30%, at least about 40%, at least about 50%, at least about 60%, at least about 70%, at least about 80%, at least about 90%, at least 95%, at least 98%, at least 99%, or at least about 100%). In certain embodiments, a prophylactically effective amount is an amount effective for decreasing an amount of pre-miR-21 (e.g., by at least about 10%, at least about 20%, at least about 30%, at least about 40%, at least about 50%, at least about 60%, at least about 70%, at least about 80%, at least about 90%, at least 95%, at least 98%, at least 99%, or at least about 100%). In certain embodiments, a prophylactically effective amount is an amount effective for cleaving pre-miR-21 (e.g., by at least about 10%, at least about 20%, at least about 30%, at least about 40%, at least about 50%, at least about 60%, at least about 70%, at least about 80%, at least about 90%, at least 95%, at least 98%, at least 99%, or at least about 100%). In certain embodiments, a prophylactically effective amount is an amount effective for downregulating mature microRNA-21 (miR-21) (e.g. miR-21-5p). In certain embodiments, miR-21 is downregulated (e.g., by at least about 10%, at least about 20%, at least about 30%, at least about 40%, at least about 50%, at least about 60%, at least about 70%, at least about 80%, at least about 90%, at least 95%, at least 98%, at least 99%, or at least about 100%). In certain embodiments, a prophylactically effective amount is an amount effective for decreasing an amount of miR-21 (e.g., by at least about 10%, at least about 20%, at least about 30%, at least about 40%, at least about 50%, at least about 60%, at least about 70%, at least about 80%, at least about 90%, at least 95%, at least 98%, at least 99%, or at least about 100%). In certain embodiments, a prophylactically effective amount is an amount effective for increasing an amount of programmed cell death protein 4 (PDCD4) (e.g., by at least about 10%, at least about 20%, at least about 30%, at least about 40%, at least about 50%, at least about 60%, at least about 70%, at least about 80%, at least about 90%, at least about 100%, at least about 150%, at least about 200%, at least about 300%, at least about 400%, at least about 500%, at least about 600%, at least about 700%, at least about 800%, at least about 900%, or at least about 1000%).

The term “cancer” refers to a class of diseases characterized by the development of abnormal cells that proliferate uncontrollably and have the ability to infiltrate and destroy normal body tissues. See e.g., Stedman's Medical Dictionary, 25th ed.; Hensyl ed.; Williams & Wilkins: Philadelphia, 1990. Exemplary cancers include, but are not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; ocular cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenstrom's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendoctrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva). In some embodiments, cancer is skin cancer (e.g., basal-cell skin cancer, squamous-cell skin cancer, or melanoma). The compounds disclosed herein may also be useful in treating inflammation associated with cancer.

The term “kidney disease” refers to a disorder of at least one kidney in a human, wherein the disorder compromises or impairs the function of the kidney(s). In some embodiments, kidney disease is characterized physiologically by the leakage of protein into the urine, or by the excretion of nitrogenous waste. In some embodiments, kidney disease results from a primary pathology of the kidney, such as injury to the glomerulus or tubule, or from damage to another organ, such as the pancreas, which adversely affects the ability of the kidney to perform biological functions, such as the retention of protein. Thus, kidney disease in the human can be the direct or indirect effect of a disease condition which may affect other organs. Exemplary kidney diseases include Abderhalden-Kaufmann-Lignac syndrome (Nephropathic Cystinosis), Abdominal Compartment Syndrome, Acetaminophen-induced Nephrotoxicity, Acute Kidney Failure/Acute Kidney Injury, Acute Lobar Nephronia, Acute Phosphate Nephropathy, Acute Tubular Necrosis, Adenine Phosphoribosyltransferase Deficiency, Adenovirus Nephritis, Alagille Syndrome, Alport Syndrome, Amyloidosis, ANCA Vasculitis Related to Endocarditis and Other Infections, Angiomyolipoma, Analgesic Nephropathy, Anorexia Nervosa and Kidney Disease, Angiotensin Antibodies and Focal Segmental Glomerulosclerosis, Antiphospholipid Syndrome, Anti-TNF-α Therapy-related Glomerulonephritis, APOL1 Mutations, Apparent Mineralocorticoid Excess Syndrome, Aristolochic Acid Nephropathy, Chinese Herbal Nephropathy, Balkan Endemic Nephropathy, Arteriovenous Malformations and Fistulas of the Urologic Tract, Autosomal Dominant Hypocalcemia, Bardet-Biedl Syndrome, Bartter Syndrome, Bath Salts and Acute Kidney Injury, Beer Potomania, Beeturia, β-Thalassemia Renal Disease, Bile Cast Nephropathy, BK Polyoma Virus Nephropathy in the Native Kidney, Bladder Rupture, Bladder Sphincter Dyssynergia, Bladder Tamponade, Border-Crossers' Nephropathy, Bourbon Virus and Acute Kidney Injury, Burnt Sugarcane Harvesting and Acute Renal Dysfunction, Byetta and Renal Failure, C1q Nephropathy, C3 Glomerulopathy, C3 Glomerulopathy with Monoclonal Gammopathy, C4 Glomerulopathy, Calcineurin Inhibitor Nephrotoxicity, Callilepsis Laureola Poisoning, Cannabinoid Hyperemesis Acute Renal Failure, Cardiorenal syndrome, Carfilzomib-Indiced Renal Injury, CFHR5 nephropathy, Charcot-Marie-Tooth Disease with Glomerulopathy, Chinese Herbal Medicines and Nephrotoxicity, Cherry Concentrate and Acute Kidney Injury, Cholesterol Emboli, Churg-Strauss syndrome, Chyluria, Ciliopathy, Cocaine and the Kidney, Cold Diuresis, Colistin Nephrotoxicity, Collagenofibrotic Glomerulopathy, Collapsing Glomerulopathy, Collapsing Glomerulopathy Related to CMV, Combination Antiretroviral (cART) Related-Nephropathy, Congenital Anomalies of the Kidney and Urinary Tract (CAKUT), Congenital Nephrotic Syndrome, Congestive Renal Failure, Conorenal syndrome (Mainzer-Saldino Syndrome or Saldino-Mainzer Disease), Contrast Nephropathy, Copper Sulphate Intoxication, Cortical Necrosis, Crizotinib-related Acute Kidney Injury, Cryocrystalglobulinemia, Cryoglobuinemia, Crystalglobulin-Induced Nephropathy, Crystal-Induced Acute Kidney injury, Crystal-Storing Histiocytosis, Cystic Kidney Disease, Acquired, Cystinuria, Dasatinib-Induced Nephrotic-Range Proteinuria, Dense Deposit Disease (MPGN Type 2), Dent Disease (X-linked Recessive Nephrolithiasis), DHA Crystalline Nephropathy, Dialysis Disequilibrium Syndrome, Diabetes and Diabetic Kidney Disease, Diabetes Insipidus, Dietary Supplements and Renal Failure, Diffuse Mesangial Sclerosis, Diuresis, Djenkol Bean Poisoning (Djenkolism), Down Syndrome and Kidney Disease, Drugs of Abuse and Kidney Disease, Duplicated Ureter, EAST syndrome, Ebola and the Kidney, Ectopic Kidney, Ectopic Ureter, Edema, Swelling, Erdheim-Chester Disease, Fabry's Disease, Familial Hypocalciuric Hypercalcemia, Fanconi Syndrome, Fraser syndrome, Fibronectin Glomerulopathy, Fibrillary Glomerulonephritis and Immunotactoid Glomerulopathy, Fraley syndrome, Fluid Overload, Hypervolemia, Focal Segmental Glomerulosclerosis, Focal Sclerosis, Focal Glomerulosclerosis, Galloway Mowat syndrome, Giant Cell (Temporal) Arteritis with Kidney Involvement, Gestational Hypertension, Gitelman Syndrome, Glomerular Diseases, Glomerular Tubular Reflux, Glycosuria, Goodpasture Syndrome, Green Smoothie Cleanse Nephropathy, HANAC Syndrome, Harvoni (Ledipasvir with Sofosbuvir)-Induced Renal Injury, Hair Dye Ingestion and Acute Kidney Injury, Hantavirus Infection Podocytopathy, Heat Stress Nephropathy, Hematuria (Blood in Urine), Hemolytic Uremic Syndrome (HUS), Atypical Hemolytic Uremic Syndrome (aHUS), Hemophagocytic Syndrome, Hemorrhagic Cystitis, Hemorrhagic Fever with Renal Syndrome (HFRS, Hantavirus Renal Disease, Korean Hemorrhagic Fever, Epidemic Hemorrhagic Fever, Nephropathis Epidemica), Hemosiderinuria, Hemosiderosis related to Paroxysmal Nocturnal Hemoglobinuria and Hemolytic Anemia, Hepatic Glomerulopathy, Hepatic Veno-Occlusive Disease, Sinusoidal Obstruction Syndrome, Hepatitis C-Associated Renal Disease, Hepatocyte Nuclear Factor 1β-Associated Kidney Disease, Hepatorenal Syndrome, Herbal Supplements and Kidney Disease, High Altitude Renal Syndrome, High Blood Pressure and Kidney Disease, HIV-Associated Immune Complex Kidney Disease (HIVICK), HIV-Associated Nephropathy (HIVAN), HNF1B-related Autosomal Dominant Tubulointerstitial Kidney Disease, Horseshoe Kidney (Renal Fusion), Hunner's Ulcer, Hydroxychloroquine-induced Renal Phospholipidosis, Hyperaldosteronism, Hypercalcemia, Hyperkalemia, Hypermagnesemia, Hypernatremia, Hyperoxaluria, Hyperphosphatemia, Hypocalcemia, Hypocomplementemic Urticarial Vasculitic Syndrome, Hypokalemia, Hypokalemia-induced renal dysfunction, Hypokalemic Periodic Paralysis, Hypomagnesemia, Hyponatremia, Hypophosphatemia, Hypophosphatemia in Users of Cannabis, Hypertension, Hypertension, Monogenic, Iced Tea Nephropathy, Ifosfamide Nephrotoxicity, IgA Nephropathy, IgG4 Nephropathy, Immersion Diuresis, Immune-Checkpoint Therapy-Related Interstitial Nephritis, Infliximab-Related Renal Disease, Interstitial Cystitis, Painful Bladder Syndrome (Questionnaire), Interstitial Nephritis, Interstitial Nephritis, Karyomegalic, Ivemark's syndrome, JC Virus Nephropathy, Joubert Syndrome, Ketamine-Associated Bladder Dysfunction, Kidney Stones, Nephrolithiasis, Kombucha Tea Toxicity, Lead Nephropathy and Lead-Related Nephrotoxicity, Lecithin Cholesterol Acyltransferase Deficiency (LCAT Deficiency), Leptospirosis Renal Disease, Light Chain Deposition Disease, Monoclonal Immunoglobulin Deposition Disease, Light Chain Proximal Tubulopathy, Liddle Syndrome, Lightwood-Albright Syndrome, Lipoprotein Glomerulopathy, Lithium Nephrotoxicity, LMXIB Mutations Cause Hereditary FSGS, Loin Pain Hematuria, Lupus, Systemic Lupus Erythematosis, Lupus Kidney Disease, Lupus Nephritis, Lupus Nephritis with Antineutrophil Cytoplasmic Antibody Seropositivity, Lupus Podocytopathy, Lyme Disease-Associated Glomerulonephritis, Lysinuric Protein Intolerance, Lysozyme Nephropathy, Malarial Nephropathy, Malignancy-Associated Renal Disease, Malignant Hypertension, Malakoplakia, McKittrick-Wheelock Syndrome, MDMA (Molly; Ecstacy; 3,4-Methylenedioxymethamphetamine) and Kidney Failure, Meatal Stenosis, Medullary Cystic Kidney Disease, Urolodulin-Associated Nephropathy, Juvenile Hyperuricemic Nephropathy Type 1, Medullary Sponge Kidney, Megaureter, Melamine Toxicity and the Kidney, MELAS Syndrome, Membranoproliferative Glomerulonephritis, Membranous Nephropathy, Membranous-like Glomerulopathy with Masked IgG Kappa Deposits, MesoAmerican Nephropathy, Metabolic Acidosis, Metabolic Alkalosis, Methotrexate-related Renal Failure, Microscopic Polyangiitis, Milk-alkalai syndrome, Minimal Change Disease, Monoclonal Gammopathy of Renal Significance, Dysproteinemia, Mouthwash Toxicity, MUC1 Nephropathy, Multicystic dysplastic kidney, Multiple Myeloma, Myeloproliferative Neoplasms and Glomerulopathy, Nail-patella Syndrome, NARP Syndrome, Nephrocalcinosis, Nephrogenic Systemic Fibrosis, Nephroptosis (Floating Kidney, Renal Ptosis), Nephrotic Syndrome, Neurogenic Bladder, 9/11 and Kidney Disease, Nodular Glomerulosclerosis, Non-Gonococcal Urethritis, Nutcracker syndrome, Oligomeganephronia, Orofaciodigital Syndrome, Orotic Aciduria, Orthostatic Hypotension, Orthostatic Proteinuria, Osmotic Diuresis, Osmotic Nephrosis, Ovarian Hyperstimulation Syndrome, Oxalate Nephropathy, Page Kidney, Papillary Necrosis, Papillorenal Syndrome (Renal-Coloboma Syndrome, Isolated Renal Hypoplasia), PARN Mutations and Kidney Disease, Parvovirus B19 and the Kidney, The Peritoneal-Renal Syndrome, POEMS Syndrome, Posterior Urethral Valve, Podocyte Infolding Glomerulopathy, Post-infectious Glomerulonephritis, Post-streptococcal Glomerulonephritis, Post-infectious Glomerulonephritis, Atypical, Post-Infectious Glomerulonephritis (IgA-Dominant), Mimicking IgA Nephropathy, Polyarteritis Nodosa, Polycystic Kidney Disease, Posterior Urethral Valves, Post-Obstructive Diuresis, Preeclampsia, Propofol infusion syndrome, Proliferative Glomerulonephritis with Monoclonal IgG Deposits (Nasr Disease), Propolis (Honeybee Resin) Related Renal Failure, Proteinuria (Protein in Urine), Pseudohyperaldosteronism, Pseudohypobicarbonatemia, Pseudohypoparathyroidism, Pulmonary-Renal Syndrome, Pyelonephritis (Kidney Infection), Pyonephrosis, Pyridium and Kidney Failure, Radiation Nephropathy, Ranolazine and the Kidney, Refeeding syndrome, Reflux Nephropathy, Rapidly Progressive Glomerulonephritis, Renal Abscess, Peripnephric Abscess, Renal Agenesis, Renal Arcuate Vein Microthrombi-Associated Acute Kidney Injury, Renal Artery Aneurysm, Renal Artery Dissection, Spontaneous, Renal Artery Stenosis, Renal Cell Cancer, Renal Cyst, Renal Hypouricemia with Exercise-induced Acute Renal Failure, Renal Infarction, Renal Osteodystrophy, Renal Tubular Acidosis, Renin Mutations and Autosomal Dominant Tubulointerstitial Kidney Disease, Renin Secreting Tumors (Juxtaglomerular Cell Tumor), Reset Osmostat, Retrocaval Ureter, Retroperitoneal Fibrosis, Rhabdomyolysis, Rhabdomyolysis related to Bariatric Surgery, Rheumatoid Arthritis-Associated Renal Disease, Sarcoidosis Renal Disease, Salt Wasting, Renal and Cerebral, Schistosomiasis and Glomerular Disease, Schimke immuno-osseous dysplasia, Scleroderma Renal Crisis, Serpentine Fibula-Polycystic Kidney Syndrome, Exner Syndrome, Sickle Cell Nephropathy, Silica Exposure and Chronic Kidney Disease, Sri Lankan Farmers' Kidney Disease, Sjogren's Syndrome and Renal Disease, Synthetic Cannabinoid Use and Acute Kidney Injury, Kidney Disease Following Hematopoietic Cell Transplantation, Kidney Disease Related to Stem Cell Transplantation, TAFRO Syndrome, Tea and Toast Hyponatremia, Tenofovir-Induced Nephrotoxicity, Thin Basement Membrane Disease, Benign Familial Hematuria, Thrombotic Microangiopathy Associated with Monoclonal Gammopathy, Trench Nephritis, Trigonitis, Tuberculosis, Genitourinary, Tuberous Sclerosis, Tubular Dysgenesis, Immune Complex Tubulointerstitial Nephritis Due to Autoantibodies to the Proximal Tubule Brush Border, Tumor Lysis Syndrome, Uremia, Uremic Optic Neuropathy, Ureteritis Cystica, Ureterocele, Urethral Caruncle, Urethral Stricture, Urinary Incontinence, Urinary Tract Infection, Urinary Tract Obstruction, Urogenital Fistula, Uromodulin-Associated Kidney Disease, Vancomycin-Associated Cast Nephropathy, Vasomotor Nephropathy, Vesicointestinal Fistula, Vesicoureteral Reflux, VEGF Inhibition and Renal Thrombotic Microangiopathy, Volatile Anesthetics and Acute Kidney Injury, Von Hippel-Lindau Disease, Waldenstrom's Macroglobulinemic Glomerulonephritis, Warfarin-Related Nephropathy, Wasp Stings and Acute Kidney Injury, Wegener's Granulomatosis, Granulomatosis with Polyangiitis, West Nile Virus and Chronic Kidney Disease, Wunderlich syndrome, Zellweger Syndrome, Cerebrohepatorenal Syndrome, or Alport Syndrome. In some embodiments, the kidney disease is Alport Syndrome.

The term “gene” refers to a nucleic acid fragment that expresses a protein, including regulatory sequences preceding (5′ non-coding sequences) and following (3′ non-coding sequences) the coding sequence. “Native gene” refers to a gene as found in nature with its own regulatory sequences. “Chimeric gene” or “chimeric construct” refers to any gene or a construct, not a native gene, comprising regulatory and coding sequences that are not found together in nature. Accordingly, a chimeric gene or chimeric construct may comprise regulatory sequences and coding sequences that are derived from different sources, or regulatory sequences and coding sequences derived from the same source, but arranged in a manner different than that found in nature. “Endogenous gene” refers to a native gene in its natural location in the genome of an organism. A “foreign” gene refers to a gene not normally found in the host organism, but which is introduced into the host organism by gene transfer. Foreign genes can comprise native genes inserted into a non-native organism, or chimeric genes. A “transgene” is a gene that has been introduced into the genome by a transformation procedure.

The terms “polynucleotide”, “nucleotide sequence”, “nucleic acid”, “nucleic acid molecule”, “nucleic acid sequence”, and “oligonucleotide” refer to a series of nucleotide bases (also called “nucleotides”) in DNA and RNA, and mean any chain of two or more nucleotides. The polynucleotides can be chimeric mixtures or derivatives or modified versions thereof, single-stranded or double-stranded. The oligonucleotide can be modified at the base moiety, sugar moiety, or phosphate backbone, for example, to improve stability of the molecule, its hybridization parameters, etc. The antisense oligonucleotide may comprise a modified base moiety which is selected from the group including, but not limited to, 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid, 5-methyl-2-thiouracil, 3-(3-amino-3 —N-2-carboxypropyl) uracil, a thio-guanine, and 2,6-diaminopurine. A nucleotide sequence typically carries genetic information, including the information used by cellular machinery to make proteins and enzymes. These terms include double- or single-stranded genomic and cDNA, RNA, any synthetic and genetically manipulated polynucleotide, and both sense and antisense polynucleotides. This includes single- and double-stranded molecules, i.e., DNA-DNA, DNA-RNA and RNA-RNA hybrids, as well as “protein nucleic acids” (PNAs) formed by conjugating bases to an amino acid backbone. This also includes nucleic acids containing carbohydrate or lipids. Exemplary DNAs include single-stranded DNA (ssDNA), double-stranded DNA (dsDNA), plasmid DNA (pDNA), genomic DNA (gDNA), complementary DNA (cDNA), antisense DNA, chloroplast DNA (ctDNA or cpDNA), microsatellite DNA, mitochondrial DNA (mtDNA or mDNA), kinetoplast DNA (kDNA), provirus, lysogen, repetitive DNA, satellite DNA, and viral DNA. Exemplary RNAs include single-stranded RNA (ssRNA), double-stranded RNA (dsRNA), small interfering RNA (siRNA), messenger RNA (mRNA), precursor messenger RNA (pre-mRNA), small hairpin RNA or short hairpin RNA (shRNA), microRNA (miRNA), guide RNA (gRNA), transfer RNA (tRNA), antisense RNA (asRNA), heterogeneous nuclear RNA (hnRNA), coding RNA, non-coding RNA (ncRNA), long non-coding RNA (long ncRNA or 1ncRNA), satellite RNA, viral satellite RNA, signal recognition particle RNA, small cytoplasmic RNA, small nuclear RNA (snRNA), ribosomal RNA (rRNA), Piwi-interacting RNA (piRNA), polyinosinic acid, ribozyme, flexizyme, small nucleolar RNA (snoRNA), spliced leader RNA, viral RNA, and viral satellite RNA.

Polynucleotides described herein may be synthesized by standard methods known in the art, e.g., by use of an automated DNA synthesizer (such as those that are commercially available from Biosearch, Applied Biosystems, etc.). As examples, phosphorothioate oligonucleotides may be synthesized by the method of Stein et al., Nucl. Acids Res., 16, 3209, (1988), methylphosphonate oligonucleotides can be prepared by use of controlled pore glass polymer supports (Sarin et al., Proc. Natl. Acad. Sci. U.S.A. 85, 7448-7451, (1988)). A number of methods have been developed for delivering antisense DNA or RNA to cells, e.g., antisense molecules can be injected directly into the tissue site, or modified antisense molecules, designed to target the desired cells (antisense linked to peptides or antibodies that specifically bind receptors or antigens expressed on the target cell surface) can be administered systemically. Alternatively, RNA molecules may be generated by in vitro and in vivo transcription of DNA sequences encoding the antisense RNA molecule. Such DNA sequences may be incorporated into a wide variety of vectors that incorporate suitable RNA polymerase promoters such as the T7 or SP6 polymerase promoters. Alternatively, antisense cDNA constructs that synthesize antisense RNA constitutively or inducibly, depending on the promoter used, can be introduced stably into cell lines. However, it is often difficult to achieve intracellular concentrations of the antisense sufficient to suppress translation of endogenous mRNAs. Therefore a preferred approach utilizes a recombinant DNA construct in which the antisense oligonucleotide is placed under the control of a strong promoter. The use of such a construct to transfect target cells in the patient will result in the transcription of sufficient amounts of single stranded RNAs that will form complementary base pairs with the endogenous target gene transcripts and thereby prevent translation of the target gene mRNA. For example, a vector can be introduced in vivo such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequence encoding the antisense RNA can be by any promoter known in the art to act in mammalian, preferably human, cells. Such promoters can be inducible or constitutive. Any type of plasmid, cosmid, yeast artificial chromosome, or viral vector can be used to prepare the recombinant DNA construct that can be introduced directly into the tissue site.

The polynucleotides may be flanked by natural regulatory (expression control) sequences or may be associated with heterologous sequences, including promoters, internal ribosome entry sites (IRES) and other ribosome binding site sequences, enhancers, response elements, suppressors, signal sequences, polyadenylation sequences, introns, 5′- and 3′-non-coding regions, and the like. The nucleic acids may also be modified by many means known in the art. Non-limiting examples of such modifications include methylation, “caps”, substitution of one or more of the naturally occurring nucleotides with an analog, and internucleotide modifications, such as, for example, those with uncharged linkages (e.g., methyl phosphonates, phosphotriesters, phosphoroamidates, carbamates, etc.) and with charged linkages (e.g., phosphorothioates, phosphorodithioates, etc.). Polynucleotides may contain one or more additional covalently linked moieties, such as, for example, proteins (e.g., nucleases, toxins, antibodies, signal peptides, poly-L-lysine, etc.), intercalators (e.g., acridine, psoralen, etc.), chelators (e.g., metals, radioactive metals, iron, oxidative metals, etc.), and alkylators. The polynucleotides may be derivatized by formation of a methyl or ethyl phosphotriester or an alkyl phosphoramidate linkage. Furthermore, the polynucleotides herein may also be modified with a label capable of providing a detectable signal, either directly or indirectly. Exemplary labels include radioisotopes, fluorescent molecules, isotopes (e.g., radioactive isotopes), biotin, and the like.

“RNA transcript” refers to the product resulting from RNA polymerase-catalyzed transcription of a DNA sequence. When the RNA transcript is a complementary copy of the DNA sequence, it is referred to as the primary transcript, or it may be an RNA sequence derived from post-transcriptional processing of the primary transcript and is referred to as the mature RNA. “Messenger RNA (mRNA)” refers to the RNA that is without introns and can be translated into polypeptides by the cell. “cRNA” refers to complementary RNA, transcribed from a recombinant cDNA template. “cDNA” refers to DNA that is complementary to and derived from an mRNA template. The cDNA can be single-stranded or converted to double-stranded form using, for example, the Klenow fragment of DNA polymerase I.

A sequence “complementary” to a portion of an RNA, refers to a sequence having sufficient complementarity to be able to hybridize with the RNA, forming a stable duplex; in the case of double-stranded antisense nucleic acids, a single strand of the duplex DNA may thus be tested, or triplex formation may be assayed. The ability to hybridize will depend on both the degree of complementarity and the length of the antisense nucleic acid. Generally, the longer the hybridizing nucleic acid, the more base mismatches with an RNA it may contain and still form a stable duplex (or triplex, as the case may be). One skilled in the art can ascertain a tolerable degree of mismatch by use of standard procedures to determine the melting point of the hybridized complex.

The terms “nucleic acid” or “nucleic acid sequence”, “nucleic acid molecule”, “nucleic acid fragment” or “polynucleotide” may be used interchangeably with “gene”, “mRNA encoded by a gene” and “cDNA”.

The term “mRNA” or “mRNA molecule” refers to messenger RNA, or the RNA that serves as a template for protein synthesis in a cell. The sequence of a strand of mRNA is based on the sequence of a complementary strand of DNA comprising a sequence coding for the protein to be synthesized.

The term “siRNA” or “siRNA molecule” refers to small inhibitory RNA duplexes that induce the RNA interference (RNAi) pathway, where the siRNA interferes with the expression of specific genes with a complementary nucleotide sequence. siRNA molecules can vary in length (e.g., between 18-30 or 20-25 basepairs, inclusive) and contain varying degrees of complementarity to their target mRNA in the antisense strand. Some siRNA have unpaired overhanging bases on the 5′ or 3′ end of the sense strand and/or the antisense strand. The term siRNA includes duplexes of two separate strands, as well as single strands that can form hairpin structures comprising a duplex region.

The term “microRNAs” or “miRNA” refers to small non-coding RNAs that are transcribed as primary transcripts that are processed first in the nucleus by a first nuclease to liberate the precursor miRNA, and then in the cytoplasm by a second nuclease to produce the mature miRNA. In certain embodiments, the term “microRNAs” or “miRNA” refers to small non-coding RNAs that are transcribed as primary transcripts that are processed first in the nucleus by Drosha to liberate the precursor miRNA, and then in the cytoplasm by Dicer to produce the mature miRNA.

The term “linker” refers to a bond or a divalent chemical moiety that is bonded to (i.e., that connects) two separate monovalent chemical moieties (e.g., B and R in Formula (R)).

The term “RNA binder” refers to a compound or chemical moiety that is capable of binding to RNA (e.g., an RNA target). In certain embodiments, binding interactions between the RNA binder and RNA are based on structure. In certain embodiments, binding interactions between the RNA binder and RNA are based on structure and not the RNA sequence. In certain embodiments, the RNA binder and RNA form a ternary complex. In certain embodiments, the RNA binder is identified using a DNA-encoded library (DEL) as disclosed herein. In certain embodiments, the RNA binder is identified using Inforna or Inforna 2.0, as described in S. P. Velagapudi et al., Nat. Chem. Biol. 2014, 10(4):291-97 and M. D. Disney et al., ACS Chem. Biol. 2016, 11(6):1720-28, the contents of which are incorporated herein by reference. In certain embodiments, the RNA binder is identified using two-dimensional combinatorial screening (2DCS), as described in M. D. Disney et al., J. Am. Chem. Soc. 2008,130(33):11185-94 and International Patent Application No. PCT/US2018/000020, the contents of which are incorporated herein by reference. In certain embodiments, the RNA binder is identified using a DNA-encoded library (DEL), as described in R. I. Benhamou, et al., Proc. Natl. Acad. Sci. U.S.A. 2022,119(6) e2114971119, the contents of which are incorporated herein by reference. In certain embodiments, the RNA binder is identified using chemical cross-linking and isolation by pull-down (Chem-CLIP), as described in International Patent Application No. PCT/US2020/070189 and B. M. Suresh, et al., Proc. Natl. Acad. Sci. U.S.A. 2020,117(52):33197-203, the contents of which are incorporated herein by reference.

### DETAILED DESCRIPTION OF CERTAIN EMBODIMENTS

The aspects described herein are not limited to specific embodiments, systems, compositions, methods, or configurations, and as such can, of course, vary. The terminology used herein is for the purpose of describing particular aspects only and, unless specifically defined herein, is not intended to be limiting.

### Compounds

In one aspect, the present disclosure provides a compound of Formula (I):

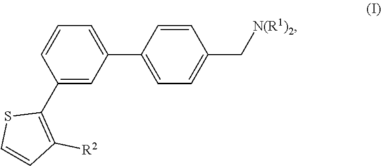

or a pharmaceutically acceptable salt, solvate, hydrate, polymorph, co-crystal, tautomer, stereoisomer, isotopically labeled compound, or prodrug thereof, wherein:
each occurrence of R¹ is independently hydrogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, or a nitrogen protecting group, or two occurrences of R¹ are joined together with their intervening atom to form an optionally substituted heterocyclic ring or optionally substituted heteroaryl ring;
R² is hydrogen, halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, or —B(OR^{A})₂; and
each occurrence of R^{A} is independently hydrogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two occurrences of R^{A} are joined together with their intervening atom to form an optionally substituted heterocyclic ring or optionally substituted heteroaryl ring.

In certain embodiments, each occurrence of R¹ is independently hydrogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, or a nitrogen protecting group, or two occurrences of R¹ are joined together with their intervening atom to form an optionally substituted heterocyclic ring or optionally substituted heteroaryl ring.

In certain embodiments, at least one occurrence of R¹ is hydrogen. In certain embodiments, at least one occurrence of R¹ is optionally substituted acyl. In certain embodiments, at least one occurrence of R¹ is optionally substituted alkyl. In certain embodiments, at least one occurrence of R¹ is optionally substituted alkenyl. In certain embodiments, at least one occurrence of R¹ is optionally substituted alkynyl. In certain embodiments, at least one occurrence of R¹ is optionally substituted heteroalkyl. In certain embodiments, at least one occurrence of R¹ is optionally substituted heteroalkenyl. In certain embodiments, at least one occurrence of R¹ is optionally substituted heteroalkynyl. In certain embodiments, at least one occurrence of R¹ is optionally substituted carbocyclyl. In certain embodiments, at least one occurrence of R¹ is optionally substituted heterocyclyl. In certain embodiments, at least one occurrence of R¹ is optionally substituted aryl. In certain embodiments, at least one occurrence of R¹ is optionally substituted heteroaryl. In certain embodiments, at least one occurrence of R¹ is a nitrogen protecting group. In certain embodiments, two occurrences of R¹ are joined together with their intervening atom to form an optionally substituted heterocyclic ring or optionally substituted heteroaryl ring. In certain embodiments, each occurrence of R¹ is optionally substituted alkyl. In certain embodiments, the moiety —N(R¹)₂ is of formula

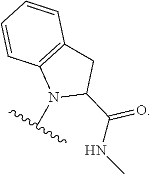 [SMILES: *N1c2ccccc2CC1C(=O)NC]

In certain embodiments, the moiety —N(R¹)₂ is of formula

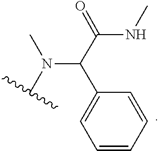

In certain embodiments, R² is hydrogen, halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, or —B(OR^{A})₂.

In certain embodiments, R² is hydrogen. In certain embodiments, R² is halogen. In certain embodiments, R² is optionally substituted alkenyl. In certain embodiments, R² is optionally substituted C₂₋₁₂ alkenyl. In certain embodiments, R² is optionally substituted C₂₋₆ alkenyl. In certain embodiments, R² is substituted or unsubstituted ethenyl, substituted or unsubstituted 1-propenyl, substituted or unsubstituted 2-propenyl, substituted or unsubstituted 1-butenyl, substituted or unsubstituted 2-butenyl, substituted or unsubstituted butadienyl, substituted or unsubstituted pentenyl, substituted or unsubstituted pentadienyl, or substituted or unsubstituted hexenyl. In certain embodiments, R² is optionally substituted alkynyl. In certain embodiments, R² is optionally substituted C₂₋₁₂ alkynyl. In certain embodiments, R² is optionally substituted C₂₋₆ alkynyl. In certain embodiments, R² is substituted or unsubstituted ethynyl, substituted or unsubstituted 1-propynyl, substituted or unsubstituted 2-propynyl, substituted or unsubstituted 1-butynyl, substituted or unsubstituted 2-butynyl, substituted or unsubstituted pentynyl, or substituted or unsubstituted hexynyl. In certain embodiments, R² is optionally substituted heteroalkyl. In certain embodiments, R² is optionally substituted heteroC₁₋₁₂ alkyl. In certain embodiments, R² is optionally substituted heteroC₁₋₆ alkyl. In certain embodiments, R² is optionally substituted heteroalkenyl. In certain embodiments, R² is optionally substituted heteroC₁₋₁₂ alkenyl. In certain embodiments, R² is optionally substituted heteroC₁₋₆ alkenyl. In certain embodiments, R² is optionally substituted heteroalkynyl. In certain embodiments, R² is optionally substituted heteroC₁₋₁₂ alkynyl. In certain embodiments, R² is optionally substituted heteroC₁₋₆ alkynyl. In certain embodiments, R² is optionally substituted carbocyclyl. In certain embodiments, R² is optionally substituted C₃₋₁₄ cycloalkyl. In certain embodiments, R² is optionally substituted heterocyclyl. In certain embodiments, R² is optionally substituted 5-10 membered heterocyclyl. In certain embodiments, R² is optionally substituted aryl. In certain embodiments, R² is optionally substituted 6-14 membered aryl. In certain embodiments, R² is optionally substituted heteroaryl. In certain embodiments, R² is optionally substituted 5-14 membered heteroaryl. In certain embodiments, R² is —CN. In certain embodiments, R² is —OR^{A}. In certain embodiments, R² is —SCN. In certain embodiments, R² is —SR^{A}. In certain embodiments, R² is —SSR^{A}. In certain embodiments, R² is —N3. In certain embodiments, R² is —NO. In certain embodiments, R² is —NO₂. In certain embodiments, R² is —C(═O)R^{A}. In certain embodiments, R² is —C(═O)OR^{A}. In certain embodiments, R² is —C(═O)SR^{A}. In certain embodiments, R² is —C(═O)N(R^{A})₂. In certain embodiments, R² is —C(═NR^{A})R^{A}. In certain embodiments, R² is —C(═NR^{A})OR^{A}. In certain embodiments, R² is —C(═NR^{A})SR^{A}. In certain embodiments, R² is —C(═NR^{A})N(R^{A})₂. In certain embodiments, R² is —S(═O)R^{A}. In certain embodiments, R² is —S(═O)OR^{A}. In certain embodiments, R² is —S(═O)SR^{A}. In certain embodiments, R² is —S(═O)N(R^{A})₂. In certain embodiments, R² is —S(═O)₂R^{A}. In certain embodiments, R² is —S(═O)₂OR^{A}. In certain embodiments, R² is —S(═O)₂SR^{A}. In certain embodiments, R² is —S(═O)₂N(R^{A})₂. In certain embodiments, R² is —OC(═O)R^{A}. In certain embodiments, R² is —OC(═O)OR^{A}. In certain embodiments, R² is —OC(═O)SR^{A}. In certain embodiments, R² is —OC(═O)N(R^{A})₂. In certain embodiments, R² is —OC(═NR^{A})R^{A}. In certain embodiments, R² is —OC(═NR^{A})OR^{A}. In certain embodiments, R² is —OC(═NR^{A})SR^{A}. In certain embodiments, R² is —OC(═NR^{A})N(R^{A})₂. In certain embodiments, R² is —OS(═O)R^{A}. In certain embodiments, R² is —OS(═O)OR^{A}. In certain embodiments, R² is —OS(═O)SR^{A}. In certain embodiments, R² is —OS(═O)N(R^{A})₂. In certain embodiments, R² is —OS(═O)₂R^{A}. In certain embodiments, R² is —OS(═O)₂OR^{A}. In certain embodiments, R² is —OS(═O)₂SR^{A}. In certain embodiments, R² is —OS(═O)₂N(R^{A})₂. In certain embodiments, R² is —ON(R^{A})₂. In certain embodiments, R² is —SC(═O)R^{A}. In certain embodiments, R² is —SC(═O)OR^{A}. In certain embodiments, R² is —SC(═O)SR^{A}. In certain embodiments, R² is —SC(═O)N(R^{A})₂. In certain embodiments, R² is —SC(═NR^{A})R^{A}. In certain embodiments, R² is —SC(═NR^{A})OR^{A}. In certain embodiments, R² is —SC(═NR^{A})SR^{A}. In certain embodiments, R² is —SC(═NR^{A})N(R^{A})₂. In certain embodiments, R² is —NR^{A}C(═O)R^{A}. In certain embodiments, R² is —NR^{A}C(═O)OR^{A}. In certain embodiments, R² is —NR^{A}C(═O)SR^{A}. In certain embodiments, R² is —NR^{A}C(═O)N(R^{A})₂. In certain embodiments, R² is —NR^{A}C(═NR^{A})R^{A}. In certain embodiments, R² is —NR^{A}C(═NR^{A})OR^{A}. In certain embodiments, R² is —NR^{A}C(═NR^{A})SR^{A}. In certain embodiments, R² is —NR^{A}C(═NR^{A})N(R^{A})₂. In certain embodiments, R² is —NR^{A}S(═O)R^{A}. In certain embodiments, R² is —NR^{A}S(═O)OR^{A}. In certain embodiments, R² is —NR^{A}S(═O)SR^{A}. In certain embodiments, R² is —NR^{A}S(═O)N(R^{A})₂. In certain embodiments, R² is —NR^{A}S(═O)₂R^{A}. In certain embodiments, R² is —NR^{A}S(═O)₂OR^{A}. In certain embodiments, R² is —NR^{A}S(═O)₂SR^{A}. In certain embodiments, R² is —NR^{A}S(═O)₂N(R^{A})₂. In certain embodiments, R² is —Si(R^{A})₃. In certain embodiments, R² is —Si(R^{A})₂OR^{A}. In certain embodiments, at least one of occurrence of R² is —Si(R^{A})(OR^{A})₂. In certain embodiments, R² is —Si(OR^{A})₃. In certain embodiments, R² is —OSi(R^{A})₃. In certain embodiments, R² is —OSi(R^{A})₂OR^{A}. In certain embodiments, R² is —OSi(R^{A})(OR^{A})₂. In certain embodiments, R² is —OSi(OR^{A})₃. In certain embodiments, R² is —B(OR^{A})₂.

In certain embodiments, R² is optionally substituted alkyl. In certain embodiments, R² is optionally substituted C₁₋₁₂ alkyl. In certain embodiments, R² is optionally substituted C₁₋₆ alkyl. In certain embodiments, R² is unsubstituted C₁₋₆ alkyl. In certain embodiments, R² is substituted C₁₋₆ alkyl. In certain embodiments, R² is substituted or unsubstituted methyl, substituted or unsubstituted ethyl, substituted or unsubstituted n-propyl, substituted or unsubstituted isopropyl, substituted or unsubstituted n-butyl, substituted or unsubstituted tert-butyl, substituted or unsubstituted sec-butyl, substituted or unsubstituted isobutyl, substituted or unsubstituted n-pentyl, substituted or unsubstituted 3-pentanyl, substituted or unsubstituted amyl, substituted or unsubstituted neopentyl, substituted or unsubstituted 3-methyl-2-butanyl, substituted or unsubstituted tert-amyl, substituted or unsubstituted n-hexyl, substituted or unsubstituted n-heptyl, or substituted or unsubstituted n-octyl. In certain embodiments, R² is unsubstituted methyl, unsubstituted ethyl, unsubstituted n-propyl, unsubstituted isopropyl, unsubstituted n-butyl, unsubstituted tert-butyl, unsubstituted sec-butyl, unsubstituted isobutyl, unsubstituted n-pentyl, unsubstituted 3-pentanyl, unsubstituted amyl, unsubstituted neopentyl, unsubstituted 3-methyl-2-butanyl, unsubstituted tert-amyl, unsubstituted n-hexyl, unsubstituted n-heptyl, or unsubstituted n-octyl. In certain embodiments, R² is substituted methyl, substituted ethyl, substituted n-propyl, substituted isopropyl, substituted n-butyl, substituted tert-butyl, substituted sec-butyl, substituted isobutyl, substituted n-pentyl, substituted 3-pentanyl, substituted amyl, substituted neopentyl, substituted 3-methyl-2-butanyl, substituted tert-amyl, substituted n-hexyl, substituted n-heptyl, or substituted n-octyl. In certain embodiments, R² is unsubstituted n-octyl. In certain embodiments, R² is substituted n-octyl.

In certain embodiments, R² is —N(R^{A})₂. In certain embodiments, R² is —NH(optionally substituted alkyl). In certain embodiments, R² is —NH(optionally substituted C₁₋₁₂ alkyl). In certain embodiments, R² is —NH(optionally substituted C₁₋₆ alkyl). In certain embodiments, R² is —NH(unsubstituted C₁₋₆ alkyl). In certain embodiments, R² is —NH(substituted C₁₋₆ alkyl). In certain embodiments, R² is —NH(substituted or unsubstituted methyl). In certain embodiments, R² is —NH(substituted or unsubstituted ethyl). In certain embodiments, R² is —NH(R^{A}). In certain embodiments, R² is —NH(R^{A}). In certain embodiments, R² is —NH(R^{A}). In certain embodiments, R² is —NH(R^{A}). In certain embodiments, R² is —NH(R^{A}). In certain embodiments, R² is —NH(R^{A}). In certain embodiments, R² is —NH(R^{A}). In certain embodiments, R² is —NH(substituted or unsubstituted n-propyl). In certain embodiments, R² is —NH(substituted or unsubstituted isopropyl). In certain embodiments, R² is —NH(substituted or unsubstituted n-butyl). In certain embodiments, R² is —NH(substituted or unsubstituted tert-butyl). In certain embodiments, R² is —NH(substituted or unsubstituted sec-butyl). In certain embodiments, R² is —NH(substituted or unsubstituted isobutyl). In certain embodiments, R² is —NH(substituted or unsubstituted n-pentyl). In certain embodiments, R² is —NH(substituted or unsubstituted 3-pentanyl). In certain embodiments, R² is —NH(substituted or unsubstituted amyl). In certain embodiments, R² is —NH(substituted or unsubstituted neopentyl). In certain embodiments, R² is —NH(substituted or unsubstituted 3-methyl-2-butanyl). In certain embodiments, R² is —NH(substituted or unsubstituted tert-amyl). In certain embodiments, R² is —NH(substituted or unsubstituted n-hexyl). In certain embodiments, R² is —NH₂.

In certain embodiments, R² is of formula

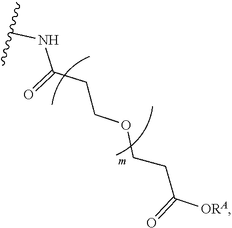

wherein m is 0, 1, 2, 3, 4, 5, 6, 7, 8, 9, or 10. In certain embodiments, m is 0, 1, 2, 3, 4, 5, 6, 7, 8, 9, or 10. In certain embodiments, m is 0. In certain embodiments, m is 1. In certain embodiments, m is 2. In certain embodiments, m is 3. In certain embodiments, m is 4. In certain embodiments, m is 5. In certain embodiments, m is 6. In certain embodiments, m is 7. In certain embodiments, m is 8. In certain embodiments, m is 9. In certain embodiments, m is 10. In certain embodiments, R² is of formula

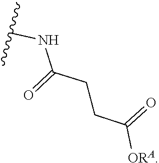

In certain embodiments, R² is of formula

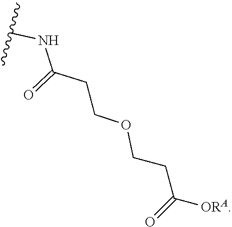

In certain embodiments. R² is of formula

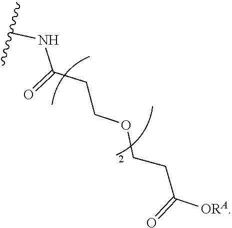

In certain embodiments. R² is of formula

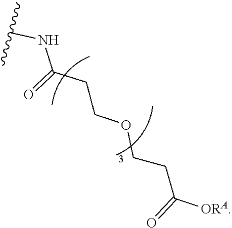

In certain embodiments, R² is of formula

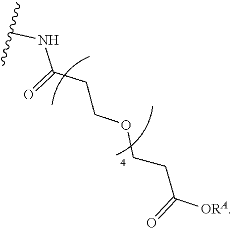

In certain embodiments, R² is of formula

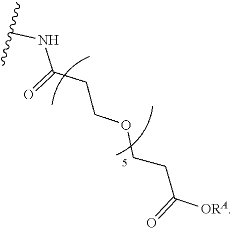

In certain embodiments, R² is of formula

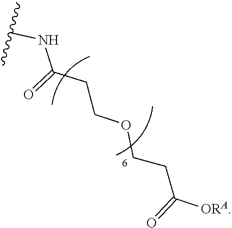

In certain embodiments. R² is of formula

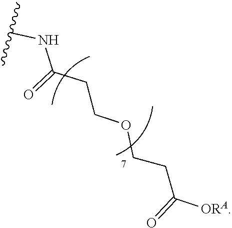

In certain embodiments, R² is of formula

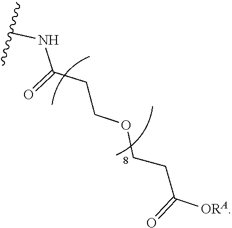

In embodiments, R² is of formula

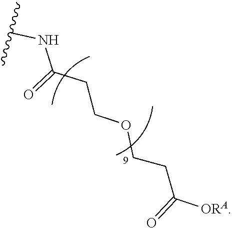

In certain embodiments. R² is of formula

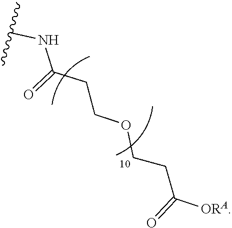

In certain embodiments. R² is of formula

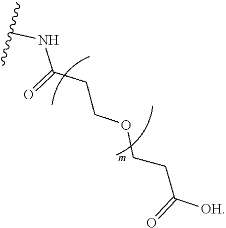

In certain embodiments, R² is of formula

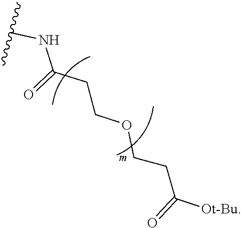

In certain embodiments, R² is of formula

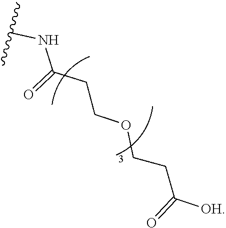

In certain embodiments, R² is of formula

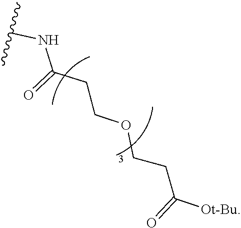

In certain embodiments, each occurrence of R¹ is optionally substituted alkyl, and R² is optionally substituted alkyl. In certain embodiments, the moiety —N(R¹)₂ is of formula

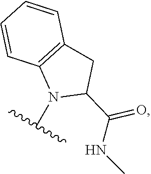 [SMILES: *N1c2ccccc2CC1C(=O)NC]

and R² is optionally substituted alkyl. In certain embodiments, the moiety —N(R¹)₂ is of formula

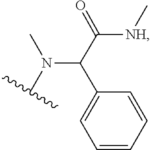

and R² is optionally substituted alkyl. In certain embodiments, each occurrence of R¹ is optionally substituted alkyl, and R² is unsubstituted n-octyl. In certain embodiments, the moiety —N(R¹)₂ is of formula

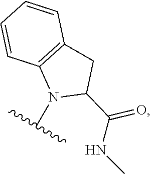 [SMILES: *N1c2ccccc2CC1C(=O)NC]

and R² is unsubstituted n-octyl. In certain embodiments, the moiety —N(R¹)₂ is of formula

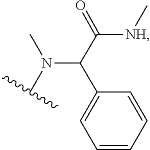

and R² is unsubstituted n-octyl. In certain embodiments, each occurrence of R¹ is optionally substituted alkyl, and R² is substituted n-octyl. In certain embodiments, the moiety —N(R¹)₂ is of formula

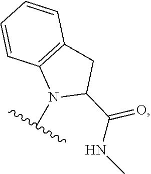 [SMILES: *N1c2ccccc2CC1C(=O)NC]

and R² is substituted n-octyl. In certain embodiments, the moiety —N(R¹)₂ is of formula

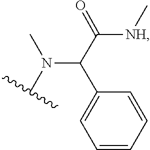

and R² is substituted n-octyl. In certain embodiments, each occurrence of R¹ is optionally substituted alkyl, and R² is —N(R^{A})₂. In certain embodiments, the moiety —N(R¹)₂ is of formula

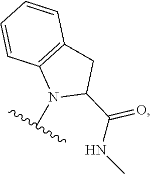 [SMILES: *N1c2ccccc2CC1C(=O)NC]

and R² is —N(R^{A})₂. In certain embodiments, the moiety —N(R¹)₂ is of formula

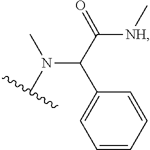

and R² is —N(R^{A})₂. In certain embodiments, each occurrence of R¹ is optionally substituted alkyl, and R² is —NH₂. In certain embodiments, the moiety —N(R¹)₂ is of formula

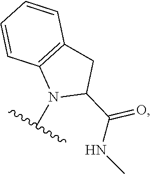 [SMILES: *N1c2ccccc2CC1C(=O)NC]

and R² is —NH₂. In certain embodiments, the moiety —N(R¹)₂ is of formula

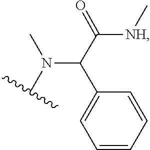

and R² is —NH₂. In certain embodiments, each occurrence of R¹ is optionally substituted alkyl, and R² is of formula

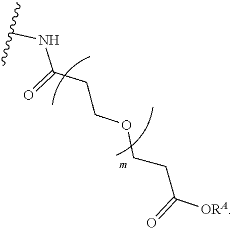

In certain embodiments, the moiety —N(R¹)₂ is of formula

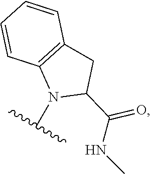 [SMILES: *N1c2ccccc2CC1C(=O)NC]

and R² is of formula

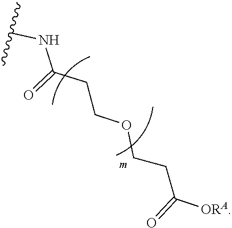

In certain embodiments, the moiety —N(R¹)₂ is of formula

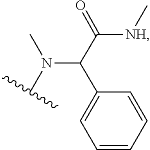

and R² is of formula

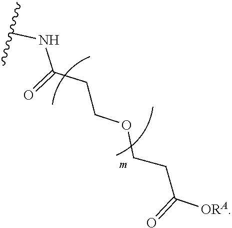

In certain embodiments, each occurrence of R¹ is optionally substituted alkyl, and R² is of formula

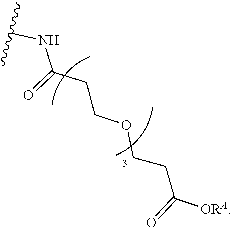

In certain embodiments, the moiety —N(R¹)₂ is of formula

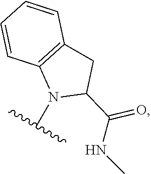 [SMILES: *N1c2ccccc2CC1C(=O)NC]

and R² is of formula

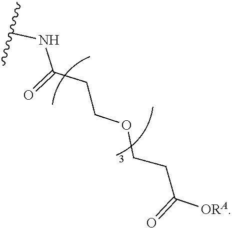

In certain embodiments, the moiety —N(R¹)₂ is of formula

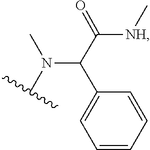

and R² is of formula

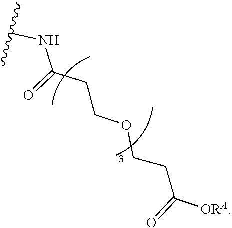

In certain embodiments, each occurrence of R¹ is optionally substituted alkyl, and R² is of formula

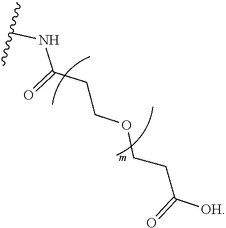

In certain embodiments, the moiety —N(R¹)₂ is of formula

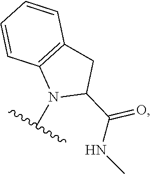 [SMILES: *N1c2ccccc2CC1C(=O)NC]

and R² is of formula

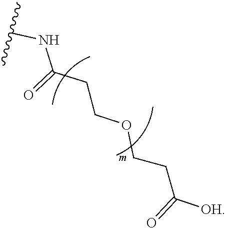

In certain embodiments, the moiety —N(R¹)₂ is of formula

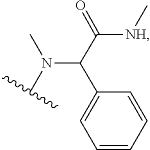

and R² is of formula

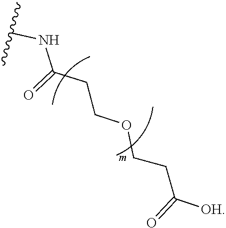

In certain embodiments, each occurrence of R¹ is optionally substituted alkyl, and R² is of formula

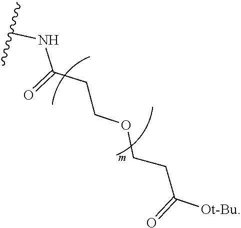

In certain embodiments, the moiety —N(R¹)₂ is of formula

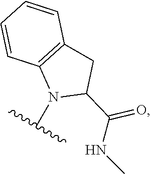 [SMILES: *N1c2ccccc2CC1C(=O)NC]

and R² is of formula

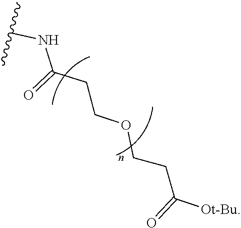

In certain embodiments, the moiety —N(R¹)₂ is of formula

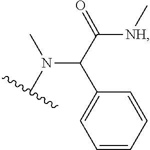

and R² is of formula

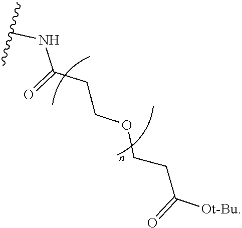

In certain embodiments, each occurrence of R¹ is optionally substituted alkyl, and R² is of formula

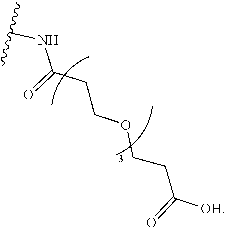

In certain embodiments, the moiety —N(R¹)₂ is of formula

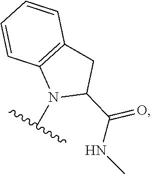 [SMILES: *N1c2ccccc2CC1C(=O)NC]

and R² is of formula

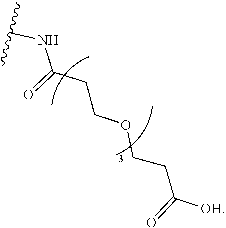

In certain embodiments, the moiety —N(R¹)₂ is of formula

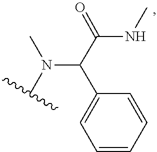

and R² is of formula

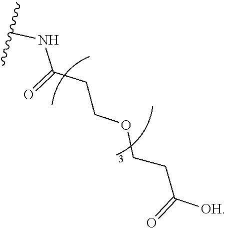

In certain embodiments, each occurrence of R¹ is optionally substituted alkyl, and R² is of formula

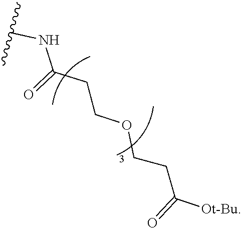

In certain embodiments, the moiety —N(R¹)₂ is of formula

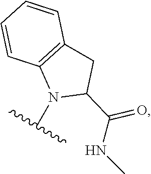 [SMILES: *N1c2ccccc2CC1C(=O)NC]

and R² is of formula

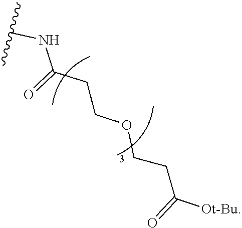

In certain embodiments, the moiety —N(R¹)₂ is of formula

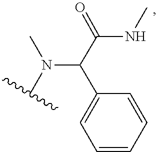

and R² is of formula

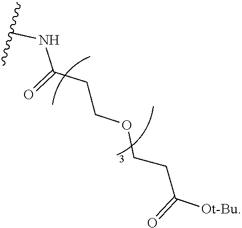

In certain embodiments, each occurrence of R^{A} is independently hydrogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two occurrences of R^{A} are joined together with their intervening atom to form an optionally substituted heterocyclic ring or optionally substituted heteroaryl ring. In certain embodiments, at least one occurrence of R^{A} is independently hydrogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two occurrences of R^{A} are joined together with their intervening atom to form an optionally substituted heterocyclic ring or optionally substituted heteroaryl ring. In certain embodiments, at least one occurrence of R^{A} is hydrogen. In certain embodiments, at least one occurrence of R^{A} is optionally substituted acyl. In certain embodiments, at least one occurrence of R^{A} is optionally substituted C₁₋₁₂ alkyl. In certain embodiments, at least one occurrence of R^{A} is optionally substituted C₁₋₆ alkyl. In certain embodiments, at least one occurrence of R^{A} is unsubstituted C₁₋₆ alkyl. In certain embodiments, at least one occurrence of R^{A} is substituted C₁₋₆ alkyl. In certain embodiments, at least one occurrence of R^{A} is substituted or unsubstituted methyl, substituted or unsubstituted ethyl, substituted or unsubstituted n-propyl, substituted or unsubstituted isopropyl, substituted or unsubstituted n-butyl, substituted or unsubstituted tert-butyl, substituted or unsubstituted sec-butyl, substituted or unsubstituted isobutyl, substituted or unsubstituted n-pentyl, substituted or unsubstituted 3-pentanyl, substituted or unsubstituted amyl, substituted or unsubstituted neopentyl, substituted or unsubstituted 3-methyl-2-butanyl, substituted or unsubstituted tert-amyl, or substituted or unsubstituted n-hexyl. In certain embodiments, at least one occurrence of R^{A} is optionally substituted C₂₋₁₂ alkenyl. In certain embodiments, at least one occurrence of R^{A} is optionally substituted C₂₋₆ alkenyl. In certain embodiments, at least one occurrence of R^{A} is substituted or unsubstituted ethenyl, substituted or unsubstituted 1-propenyl, substituted or unsubstituted 2-propenyl, substituted or unsubstituted 1-butenyl, substituted or unsubstituted 2-butenyl, substituted or unsubstituted butadienyl, substituted or unsubstituted pentenyl, substituted or unsubstituted pentadienyl, or substituted or unsubstituted hexenyl. In certain embodiments, at least one occurrence of R^{A} is optionally substituted C212 alkynyl. In certain embodiments, at least one occurrence of R^{A} is optionally substituted C₂₋₆ alkynyl. In certain embodiments, at least one occurrence of R^{A} is substituted or unsubstituted ethynyl, substituted or unsubstituted 1-propynyl, substituted or unsubstituted 2-propynyl, substituted or unsubstituted 1-butynyl, substituted or unsubstituted 2-butynyl, substituted or unsubstituted pentynyl, or substituted or unsubstituted hexynyl. In certain embodiments, at least one occurrence of R^{A} is optionally substituted heteroC₁₋₁₂ alkyl. In certain embodiments, at least one occurrence of R^{A} is optionally substituted heteroC₁₋₆ alkyl. In certain embodiments, at least one occurrence of R^{A} is optionally substituted heteroC₁₋₁₂ alkenyl. In certain embodiments, at least one occurrence of R^{A} is optionally substituted heteroC₁₋₆ alkenyl. In certain embodiments, at least one occurrence of R^{A} is optionally substituted heteroC₁₋₁₂ alkynyl. In certain embodiments, at least one occurrence of R^{A} is optionally substituted heteroC₁₋₆ alkynyl. In certain embodiments, at least one occurrence of R^{A} is optionally substituted C₃₋₁₄ cycloalkyl. In certain embodiments, at least one occurrence of R^{A} is optionally substituted 5-10 membered heterocyclyl. In certain embodiments, at least one occurrence of R^{A} is optionally substituted 6-14 membered aryl. In certain embodiments, at least one occurrence of R^{A} is optionally substituted 5-14 membered heteroaryl. In certain embodiments, at least one occurrence of R^{A} is a nitrogen protecting group when attached to a nitrogen atom. In certain embodiments, at least one occurrence of R^{A} is an oxygen protecting group when attached to an oxygen atom. In certain embodiments, at least one occurrence of R^{A} is a sulfur protecting group when attached to a sulfur atom. In certain embodiments, at least two occurrences of R^{A} are joined together with their intervening atom to form an optionally substituted 5-10 membered heterocyclic ring. In certain embodiments, at least two occurrences of R^{A} are joined together with their intervening atom to form an optionally substituted 5-14 membered heteroaryl ring.

In certain embodiments, the compound of Formula (I) is of formula:

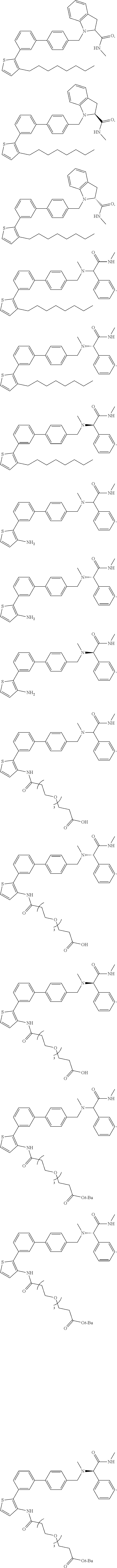

or a pharmaceutically acceptable salt, solvate, hydrate, polymorph, co-crystal, tautomer, stereoisomer, isotopically labeled compound, or prodrug thereof.

In another aspect, the present disclosure provides a compound of Formula (II):

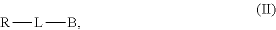

or a pharmaceutically acceptable salt, solvate, hydrate, polymorph, co-crystal, tautomer, stereoisomer, isotopically labeled compound, or prodrug thereof, wherein:
R is an RNase L recruiter of Formula (III):

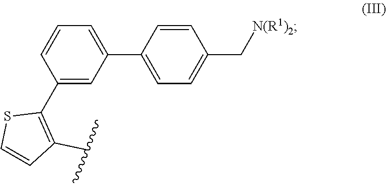

L is a linker;
B is an RNA binder; and
each occurrence of R¹ is independently hydrogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, or a nitrogen protecting group, or two occurrences of R¹ are joined together with their intervening atom to form an optionally substituted heterocyclic ring or optionally substituted heteroaryl ring.

In certain embodiments, each occurrence of R¹ is independently hydrogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, or a nitrogen protecting group, or two occurrences of R¹ are joined together with their intervening atom to form an optionally substituted heterocyclic ring or optionally substituted heteroaryl ring.

In certain embodiments, at least one occurrence of R¹ is hydrogen. In certain embodiments, at least one occurrence of R¹ is optionally substituted acyl. In certain embodiments, at least one occurrence of R¹ is optionally substituted alkyl. In certain embodiments, at least one occurrence of R¹ is optionally substituted alkenyl. In certain embodiments, at least one occurrence of R¹ is optionally substituted alkynyl. In certain embodiments, at least one occurrence of R¹ is optionally substituted heteroalkyl. In certain embodiments, at least one occurrence of R¹ is optionally substituted heteroalkenyl. In certain embodiments, at least one occurrence of R¹ is optionally substituted heteroalkynyl. In certain embodiments, at least one occurrence of R¹ is optionally substituted carbocyclyl. In certain embodiments, at least one occurrence of R¹ is optionally substituted heterocyclyl. In certain embodiments, at least one occurrence of R¹ is optionally substituted aryl. In certain embodiments, at least one occurrence of R¹ is optionally substituted heteroaryl. In certain embodiments, at least one occurrence of R¹ is a nitrogen protecting group. In certain embodiments, two occurrences of R¹ are joined together with their intervening atom to form an optionally substituted heterocyclic ring or optionally substituted heteroaryl ring. In certain embodiments, each occurrence of R¹ is optionally substituted alkyl. In certain embodiments, the moiety —N(R¹)₂ is of formula

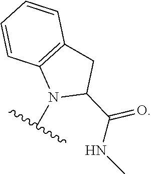 [SMILES: *N1c2ccccc2CC1C(=O)NC]

In certain embodiments, the moiety —N(R¹)₂ is of formula

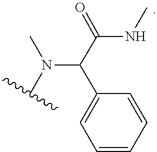

In certain embodiments, R is of formula

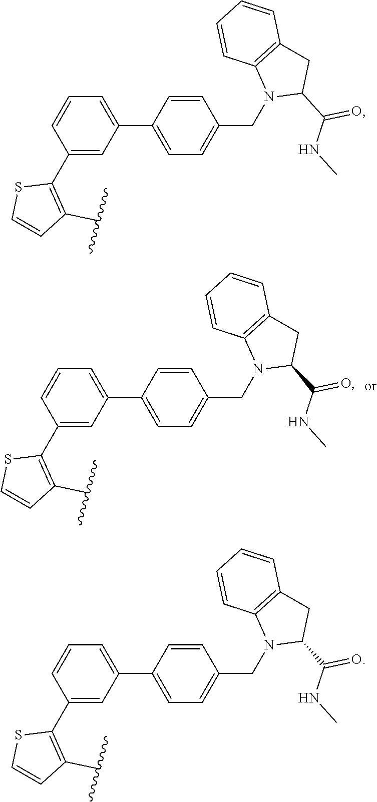 [SMILES: *c1ccsc1-c1cccc(-c2ccc(CN3c4ccccc4CC3C(=O)NC)cc2)c1 ; *c1ccsc1-c1cccc(-c2ccc(CN3c4ccccc4C[C@H]3C(=O)NC)cc2)c1 ; *c1ccsc1-c1cccc(-c2ccc(CN3c4ccccc4C[C@@H]3C(=O)NC)cc2)c1]

In certain embodiments, R is of formula

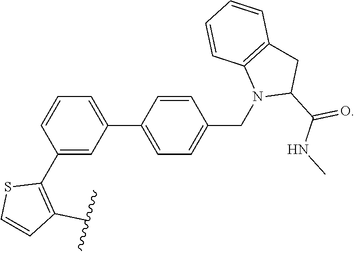 [SMILES: *c1ccsc1-c1cccc(-c2ccc(CN3c4ccccc4CC3C(=O)NC)cc2)c1]

In certain embodiments, R is of formula

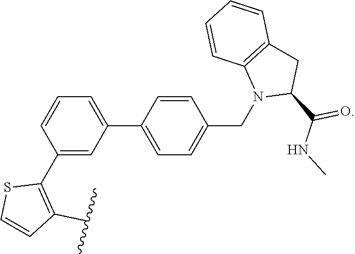 [SMILES: *c1ccsc1-c1cccc(-c2ccc(CN3c4ccccc4C[C@H]3C(=O)NC)cc2)c1]

In certain embodiments, R is of formula

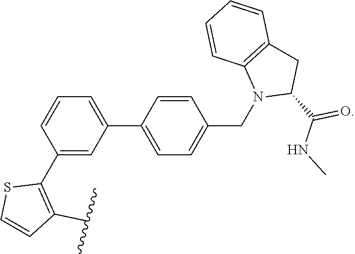 [SMILES: *c1ccsc1-c1cccc(-c2ccc(CN3c4ccccc4C[C@@H]3C(=O)NC)cc2)c1]

In certain embodiments, R is of formula

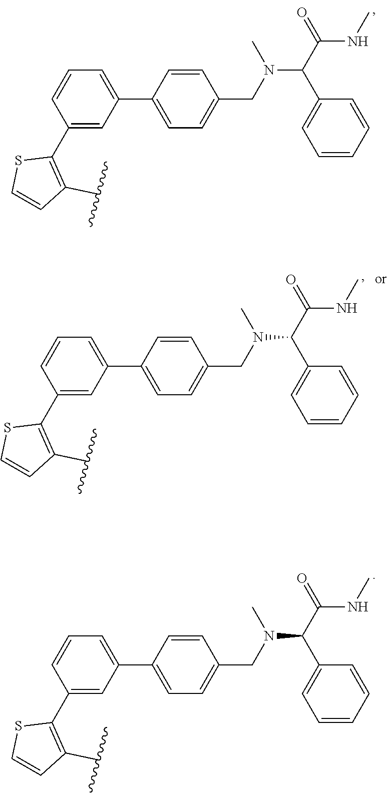 [SMILES: *c1ccsc1-c1cccc(-c2ccc(CN(C)C(C(=O)NC)c3ccccc3)cc2)c1 ; *c1ccsc1-c1cccc(-c2ccc(CN(C)[C@H](C(=O)NC)c3ccccc3)cc2)c1 ; *c1ccsc1-c1cccc(-c2ccc(CN(C)[C@@H](C(=O)NC)c3ccccc3)cc2)c1]

In certain embodiments, R is of formula

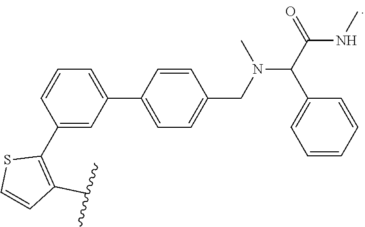 [SMILES: *c1ccsc1-c1cccc(-c2ccc(CN(C)C(C(=O)NC)c3ccccc3)cc2)c1]

In certain embodiments, R is of formula

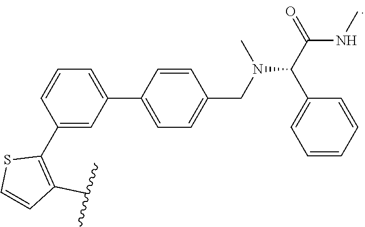 [SMILES: *c1ccsc1-c1cccc(-c2ccc(CN(C)[C@H](C(=O)NC)c3ccccc3)cc2)c1]

In certain embodiments, R is of formula

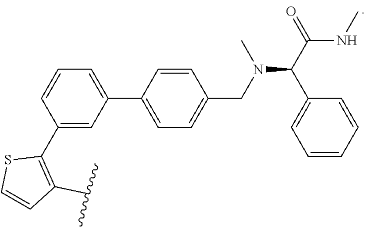 [SMILES: *c1ccsc1-c1cccc(-c2ccc(CN(C)[C@@H](C(=O)NC)c3ccccc3)cc2)c1]

In certain embodiments, L is a linker. In certain embodiments, L is a bond, optionally substituted alkylene, optionally substituted alkenylene, optionally substituted alkynylene, optionally substituted heteroalkylene, optionally substituted heteroalkenylene, optionally substituted heteroalkynylene, optionally substituted heterocyclylene, optionally substituted carbocyclylene, optionally substituted arylene, optionally substituted heteroarylene, or a combination thereof. In certain embodiments, L is a bond. In certain embodiments, L is optionally substituted alkylene. In certain embodiments, L is optionally substituted alkenylene. In certain embodiments, L is optionally substituted alkynylene. In certain embodiments, L is optionally substituted heteroalkylene. In certain embodiments, L is optionally substituted heteroalkenylene. In certain embodiments, L is optionally substituted heteroalkynylene. In certain embodiments, L is optionally substituted heterocyclylene. In certain embodiments, L is optionally substituted carbocyclylene. In certain embodiments, L is optionally substituted arylene. In certain embodiments, L is optionally substituted heteroarylene. In certain embodiments, L is optionally substituted alkylene, optionally substituted heteroalkylene, or a combination thereof.

In certain embodiments, L comprises a moiety of formula

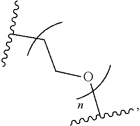

wherein n is 0, 1, 2, 3, 4, 5, 6, 7, 8, 9, or 10. In certain embodiments, n is 0, 1, 2, 3, 4, 5, 6, 7, 8, 9, or 10. In certain embodiments, n is 0. In certain embodiments, n is 1. In certain embodiments, n is 2. In certain embodiments, n is 3. In certain embodiments, n is 4. In certain embodiments, n is 5. In certain embodiments, n is 6. In certain embodiments, n is 7. In certain embodiments, n is 8. In certain embodiments, n is 9. In certain embodiments, n is 10.

In certain embodiments, L comprises a moiety of formula

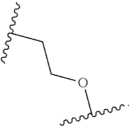 [SMILES: *CCO*]

In certain embodiments, L comprises moiety of formula

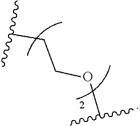

In certain embodiments, L comprises a moiety of formula

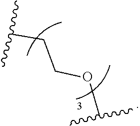

In certain embodiments, L comprises a moiety of formula

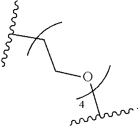

In certain embodiments, L comprises a moiety of formula

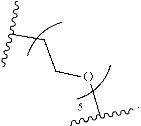

In certain embodiments, L comprises a moiety of formula

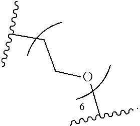

In certain embodiments, L comprises a moiety of formula

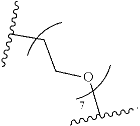

In certain embodiments, L comprises a moiety of formula

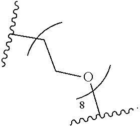

In certain embodiments, L comprises a moiety of formula

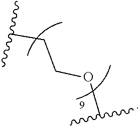

In certain embodiments, L comprises moiety of formula

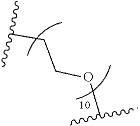

In certain embodiments, L comprises a moiety of formula

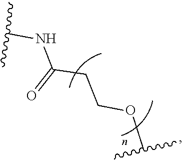

wherein n is 0, 1, 2, 3, 4, 5, 6, 7, 8, 9, or 10. In certain embodiments, L comprises a moiety of formula

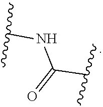 [SMILES: *NC(*)=O]

In certain embodiments, L comprises a moiety of formula

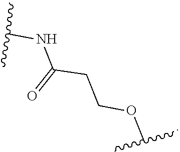 [SMILES: *NC(=O)CCO*]

In certain embodiments, L comprises a moiety of formula

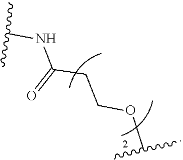

In certain embodiments, L comprises a moiety of formula

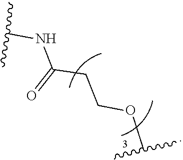

In certain embodiments, L comprises a moiety of formula

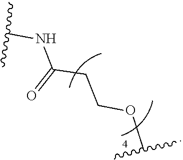

In certain embodiments, L comprises a moiety of formula

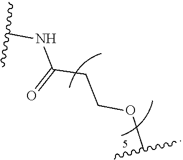

In certain embodiments, L comprises a moiety of formula

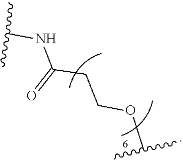

In certain embodiments, L comprises a moiety of formula

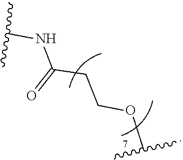

In certain embodiments, L comprises a moiety of formula

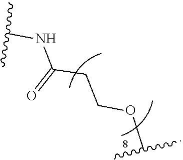

In certain embodiments, L comprises a moiety of formula

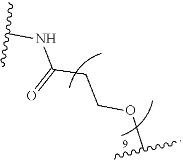

In certain embodiments, L comprises a moiety of formula

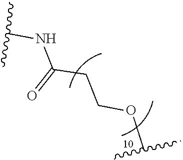

In certain embodiments, L comprises a moiety of formula

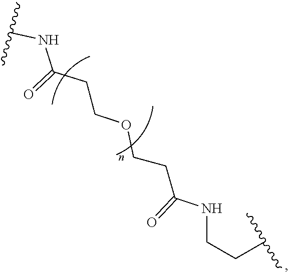

wherein n is 0, 1, 2, 3, 4, 5, 6, 7, 8, 9, or 10. In certain embodiments, L comprises a moiety of formula

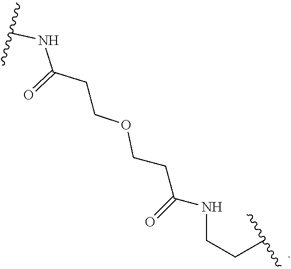 [SMILES: *CCNC(=O)CCOCCC(=O)N*]

In certain embodiments, L comprises a moiety of formula O

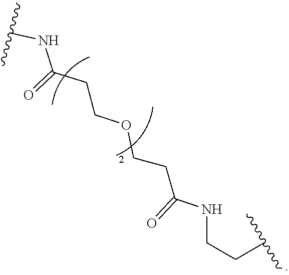

In certain embodiments, L comprises a moiety of formula

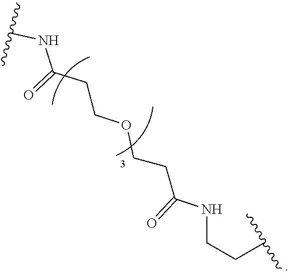

In certain embodiments, L comprises a moiety of formula

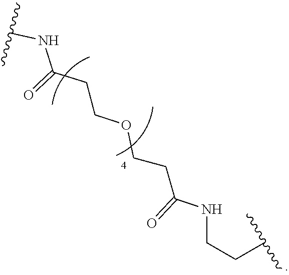

In certain embodiments, L comprises a moiety of formula

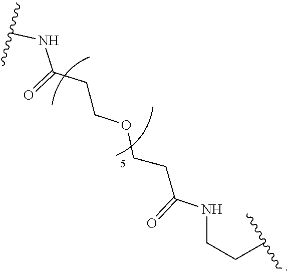

In certain embodiments, L comprises a moiety of formula

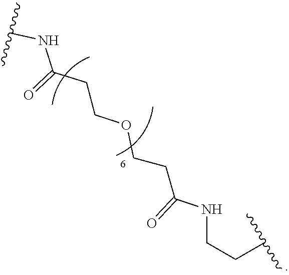

In certain embodiments, L comprises a moiety of formula

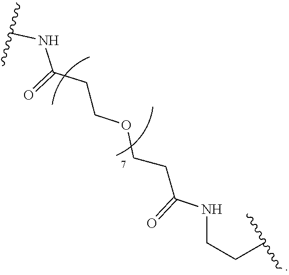

In certain embodiments, L comprises a moiety of formula

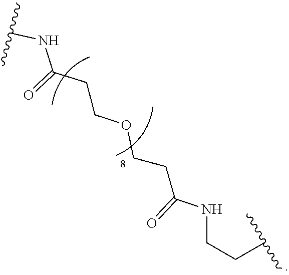

In certain embodiments, L comprises a moiety of formula

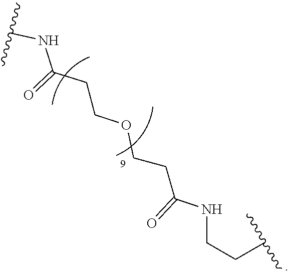

In certain embodiments, L comprises a moiety of formula

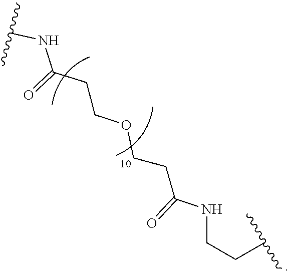

In certain embodiments, each occurrence of R¹ is optionally substituted alkyl, and L is optionally substituted alkylene, optionally substituted heteroalkylene, or a combination thereof. In certain embodiments, the moiety —N(R¹)₂ is of formula

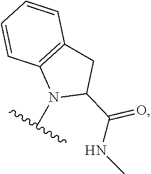 [SMILES: *N1c2ccccc2CC1C(=O)NC]

and L is optionally substituted alkylene, optionally substituted heteroalkylene, or a combination thereof. In certain embodiments, the moiety —N(R¹)₂ is of formula

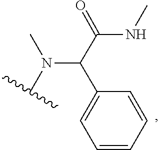

and L is optionally substituted alkylene, optionally substituted heteroalkylene, or a combination thereof. In certain embodiments, each occurrence of R¹ is optionally substituted alkyl, and L comprises a moiety of formula

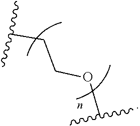

In certain embodiments, the moiety —N(R¹)₂ is of formula

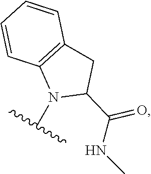 [SMILES: *N1c2ccccc2CC1C(=O)NC]

and L comprises a moiety of formula

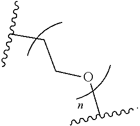

In certain embodiments, the moiety —N(R¹)₂ is of formula

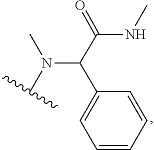

and L comprises a moiety of formula

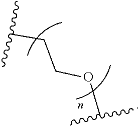

In certain embodiments, each occurrence of R¹ is optionally substituted alkyl, and L comprises a moiety of formula

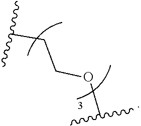

In certain embodiments, the moiety —N(R¹)₂ is of formula

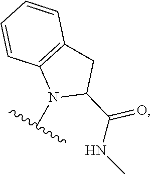 [SMILES: *N1c2ccccc2CC1C(=O)NC]

and L comprises a moiety formula

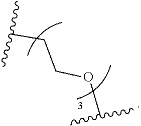

In certain embodiments, the moiety —N(R¹)₂ is of formula

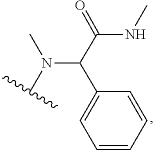

L comprises a moeity of formula

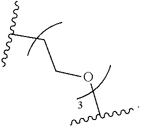

In certain embodiments, each occurrence of R¹ is optionally substituted alkyl, and L comprises a moiety of formula

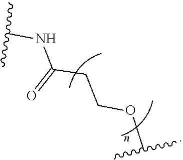

In certain embodiments, the —N(R¹)₂ is of formula

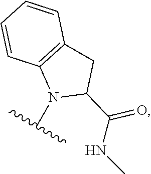 [SMILES: *N1c2ccccc2CC1C(=O)NC]

and L comprises a moiety of formula

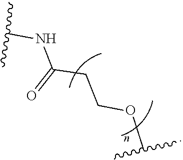

In certain embodiments, the moiety —N(R¹)₂ is of formula

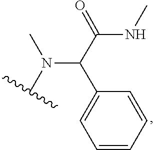

and L comprises a moiety of formula

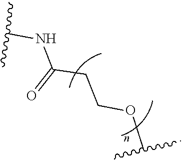

In certain embodiments, each occurrence of R¹ is optionally substituted alkyl, and L comprises a moiety of formula

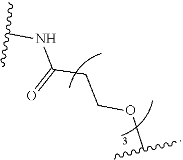

In certain embodiments, the moiety —N(R¹)₂ is of formula

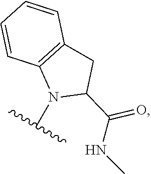 [SMILES: *N1c2ccccc2CC1C(=O)NC]

and L comprises a moiety of formula

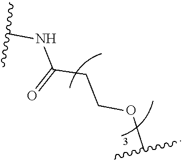

In certain embodiments, the moiety —N(R¹)₂ is of formula

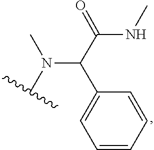

and L comprises a moiety of formula

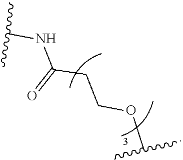

In certain embodiments, each occurrence of R¹ is optionally substituted alkyl, and L comprises a moiety of formula

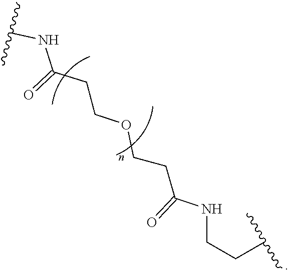

In certain embodiments, the moiety —N(R¹)₂ is of formula

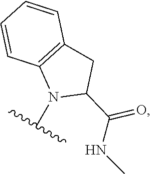 [SMILES: *N1c2ccccc2CC1C(=O)NC]

and L comprises a moiety of formula

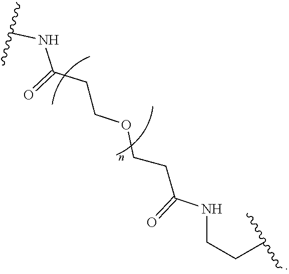

In certain embodiments, the moiety —N(R¹)₂ is of formula

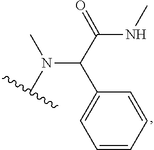

and L comprises a moiety of formula

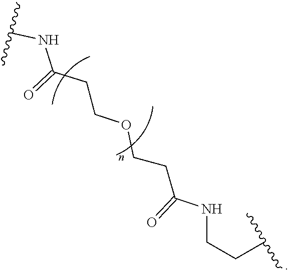

In certain embodiments, each occurrence of R¹ is optionally substituted alkyl, and L comprises a moiety of formula

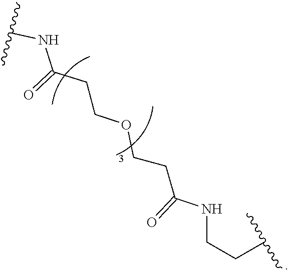

In certain embodiments, the moiety —N(R¹)₂ is of formula

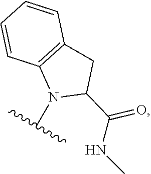 [SMILES: *N1c2ccccc2CC1C(=O)NC]

and L comprises a moiety of formula

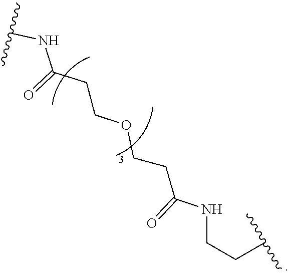

In certain embodiments, the moiety —N(R¹)₂ is of formula

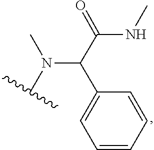

and L comprises a moiety of formula

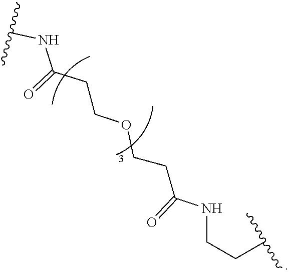

In certain embodiments, B is an RNA binder. In certain embodiments, binding interactions between the RNA binder and a corresponding RNA are based on structure. In certain embodiments, binding interactions between the RNA binder and the corresponding RNA are based on structure and not the RNA sequence. In certain embodiments, the RNA binder and corresponding RNA form a ternary complex. In certain embodiments, the RNA binder binds to a Dicer processing site in the corresponding RNA. In certain embodiments, the RNA binder is dovitinib, or a pharmaceutically acceptable salt, solvate, hydrate, polymorph, co-crystal, tautomer, stereoisomer, isotopically labeled compound, or prodrug thereof. In certain embodiments, B is an RNA binder of Formula (IV):

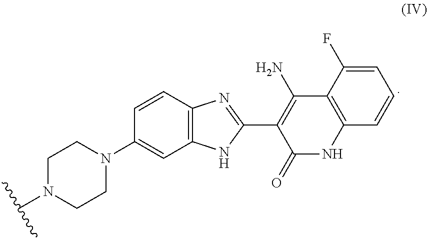 [SMILES: *N1CCN(c2ccc3nc(-c4c(N)c5c(F)cccc5[nH]c4=O)[nH]c3c2)CC1]

In certain embodiments, each occurrence of R¹ is optionally substituted alkyl, and B is of formula

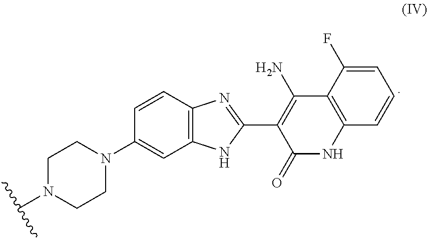 [SMILES: *N1CCN(c2ccc3nc(-c4c(N)c5c(F)cccc5[nH]c4=O)[nH]c3c2)CC1]

In certain embodiments, the moiety —N(R¹)₂ is of formula

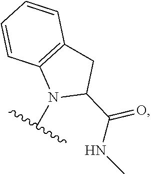 [SMILES: *N1c2ccccc2CC1C(=O)NC]

and B is of formula

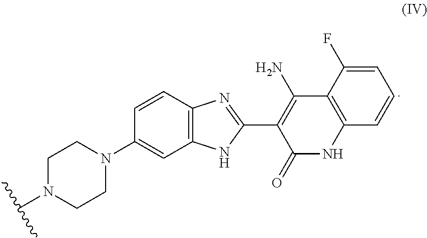 [SMILES: *N1CCN(c2ccc3nc(-c4c(N)c5c(F)cccc5[nH]c4=O)[nH]c3c2)CC1]

In certain embodiments, the moiety —N(R¹)₂ is of formula

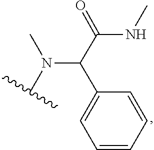

and B is of formula

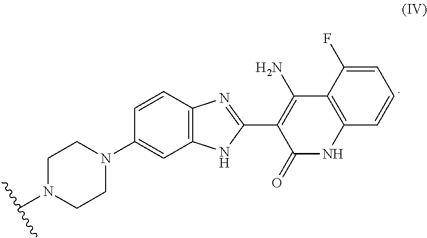 [SMILES: *N1CCN(c2ccc3nc(-c4c(N)c5c(F)cccc5[nH]c4=O)[nH]c3c2)CC1]

In certain embodiments, each occurrence of R¹ is optionally substituted alkyl, B is of formula

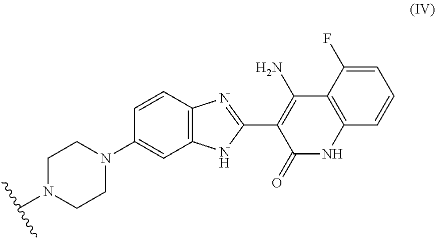 [SMILES: *N1CCN(c2ccc3nc(-c4c(N)c5c(F)cccc5[nH]c4=O)[nH]c3c2)CC1]

and L is optionally substituted alkylene, optionally substituted heteroalkylene, or a combination thereof. In certain embodiments, the moiety —N(R¹)₂ is of formula

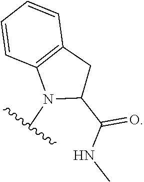 [SMILES: *N1c2ccccc2CC1C(=O)NC]

B is of formula

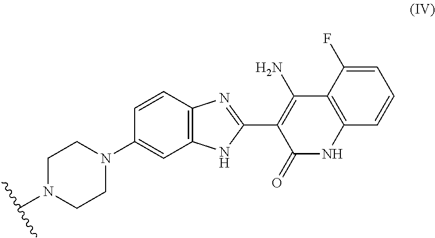 [SMILES: *N1CCN(c2ccc3nc(-c4c(N)c5c(F)cccc5[nH]c4=O)[nH]c3c2)CC1]

and L is optionally substituted alkylene, optionally substituted heteroalkylene, or a combination thereof. In certain embodiments, the moiety —N(R¹)₂ is of formula

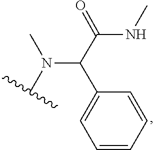

B is of ferule

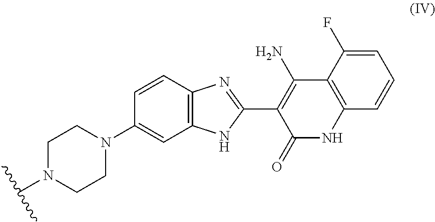 [SMILES: *N1CCN(c2ccc3nc(-c4c(N)c5c(F)cccc5[nH]c4=O)[nH]c3c2)CC1]

and L is optionally substituted alkylene, optionally substituted heteroalkylene, or a combination thereof. In certain embodiments, each occurrence of R¹ is optionally substituted alkyl, B is of formula

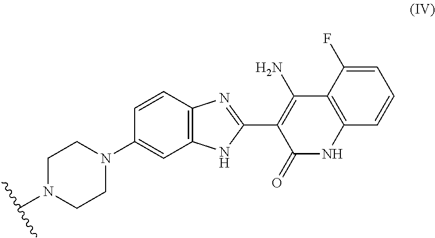 [SMILES: *N1CCN(c2ccc3nc(-c4c(N)c5c(F)cccc5[nH]c4=O)[nH]c3c2)CC1]

and L comprises a moiety of formula

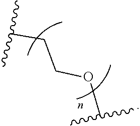

In certain embodiments, the moiety —N(R¹)₂ is of formula

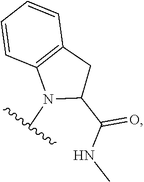 [SMILES: *N1c2ccccc2CC1C(=O)NC]

B is of formula

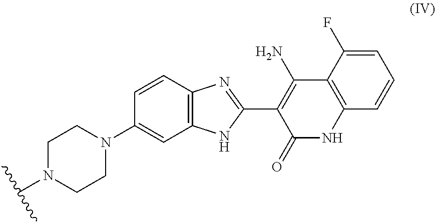 [SMILES: *N1CCN(c2ccc3nc(-c4c(N)c5c(F)cccc5[nH]c4=O)[nH]c3c2)CC1]

and L comprises a moiety of formula

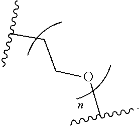

In certain embodiments, the moiety —N(R¹)₂ is of formula

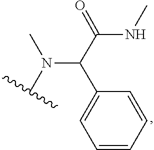

B is of formula

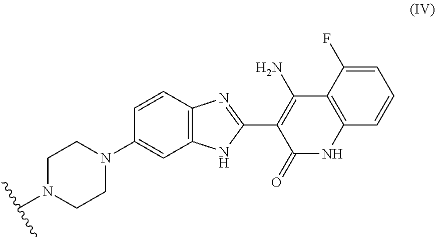 [SMILES: *N1CCN(c2ccc3nc(-c4c(N)c5c(F)cccc5[nH]c4=O)[nH]c3c2)CC1]

and L comprises a moiety of formula

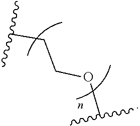

In certain embodiments, each occurrence of R¹ is optionally substituted alkyl, B is of formula

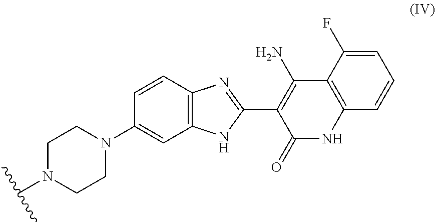 [SMILES: *N1CCN(c2ccc3nc(-c4c(N)c5c(F)cccc5[nH]c4=O)[nH]c3c2)CC1]

and L comprises a moiety of formula

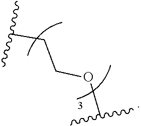

In certain embodiments, the moiety —N(R¹)₂ is of formula

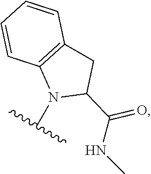 [SMILES: *N1c2ccccc2CC1C(=O)NC]

B is of formula

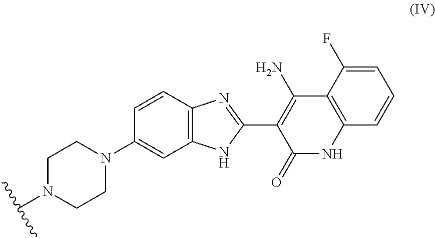 [SMILES: *N1CCN(c2ccc3nc(-c4c(N)c5c(F)cccc5[nH]c4=O)[nH]c3c2)CC1]

and L comprises a moiety of formula

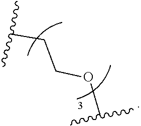

In certain embodiments, the moiety —N(R¹)₂ is of formula

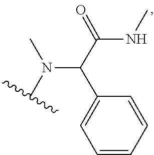

B is of formula

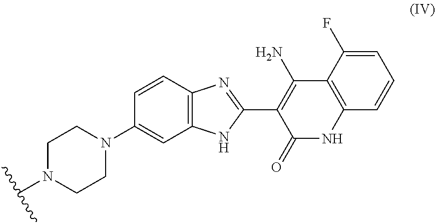 [SMILES: *N1CCN(c2ccc3nc(-c4c(N)c5c(F)cccc5[nH]c4=O)[nH]c3c2)CC1]

and L comprises a moiety of formula

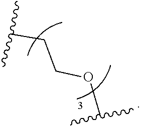

In certain embodiments, each occurrence of R¹ is optionally substituted alkyl, B is of formula

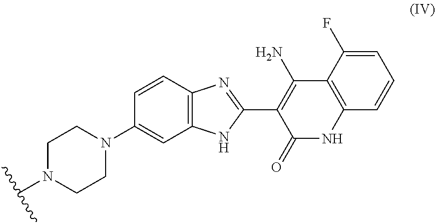 [SMILES: *N1CCN(c2ccc3nc(-c4c(N)c5c(F)cccc5[nH]c4=O)[nH]c3c2)CC1]

and L comprises a moiety of formula

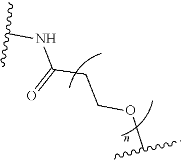

In certain embodiments, the moiety —N(R¹)₂ is of formula

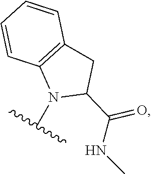 [SMILES: *N1c2ccccc2CC1C(=O)NC]

B is of formula

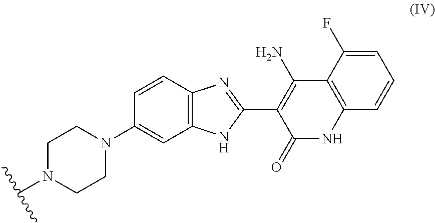 [SMILES: *N1CCN(c2ccc3nc(-c4c(N)c5c(F)cccc5[nH]c4=O)[nH]c3c2)CC1]

and L comprises a moiety of formula

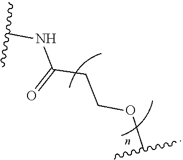

In certain embodiments, the moiety —N(R¹)₂ is of formula

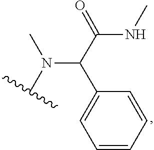

B is of formula

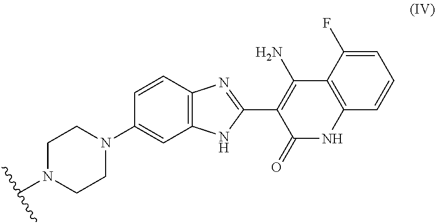 [SMILES: *N1CCN(c2ccc3nc(-c4c(N)c5c(F)cccc5[nH]c4=O)[nH]c3c2)CC1]

and L comprises a moiety of formula

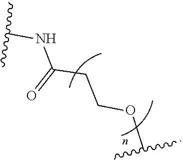

In certain embodiments, each occurrence of R¹ is optionally substituted alkyl, B is of formula

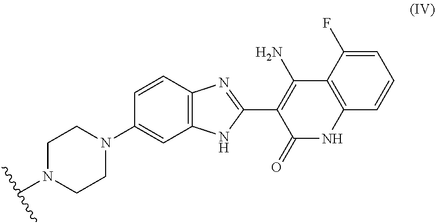 [SMILES: *N1CCN(c2ccc3nc(-c4c(N)c5c(F)cccc5[nH]c4=O)[nH]c3c2)CC1]

and L comprises a moiety of formula

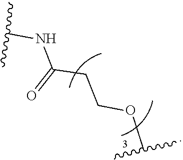

In certain embodiments, the —N(R¹)₂ is of formula

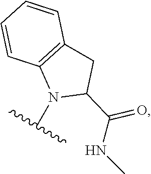 [SMILES: *N1c2ccccc2CC1C(=O)NC]

B is of formula

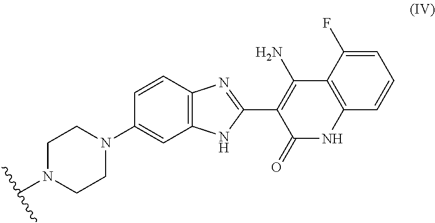 [SMILES: *N1CCN(c2ccc3nc(-c4c(N)c5c(F)cccc5[nH]c4=O)[nH]c3c2)CC1]

and L comprises a moiety of formula

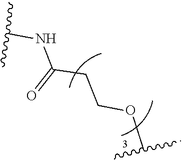

In certain embodiments, the moiety —N(R¹)₂ is of formula

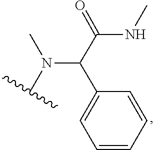

B is of formula

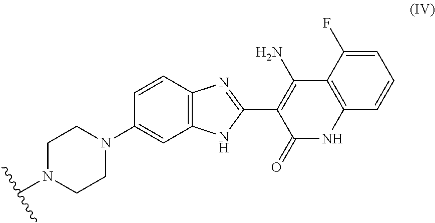 [SMILES: *N1CCN(c2ccc3nc(-c4c(N)c5c(F)cccc5[nH]c4=O)[nH]c3c2)CC1]

and L comprises a moiety of formula

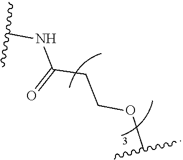

In certain embodiments, each occurrence of R¹ is optionally substituted alkyl, B is of form

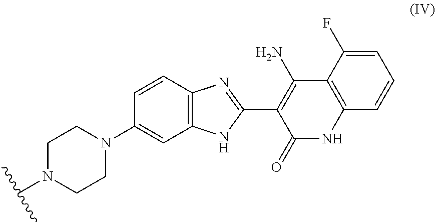 [SMILES: *N1CCN(c2ccc3nc(-c4c(N)c5c(F)cccc5[nH]c4=O)[nH]c3c2)CC1]

and L comprises a moiety of formula

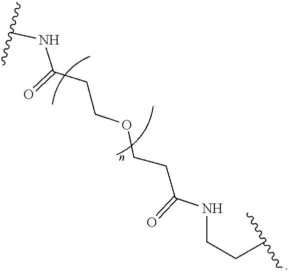

In certain embodiments, the moiety —N(R¹)₂ is of formula

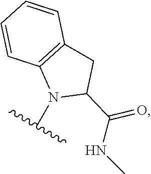 [SMILES: *N1c2ccccc2CC1C(=O)NC]

B is of formula

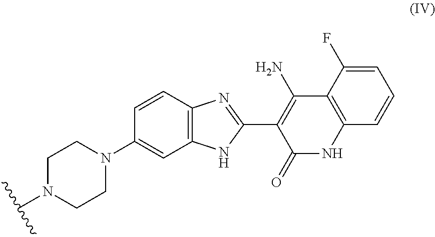 [SMILES: *N1CCN(c2ccc3nc(-c4c(N)c5c(F)cccc5[nH]c4=O)[nH]c3c2)CC1]

and L comprises a moiety of formula

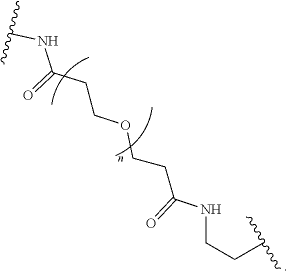

In certain embodiments, the moiety —N(R¹)₂ is of formula

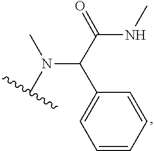

B is of formula

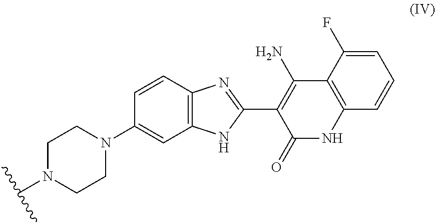 [SMILES: *N1CCN(c2ccc3nc(-c4c(N)c5c(F)cccc5[nH]c4=O)[nH]c3c2)CC1]

and L comprises a moiety of formula

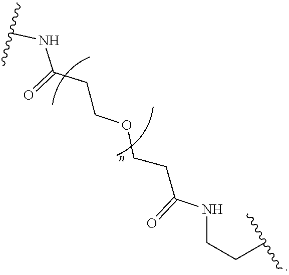

In certain embodiments, each occurrence of R¹ is optionally substituted alkyl, B is of formula

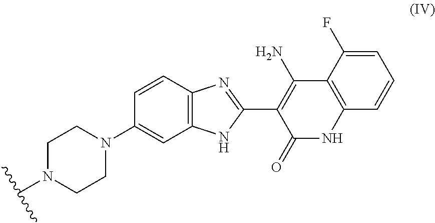 [SMILES: *N1CCN(c2ccc3nc(-c4c(N)c5c(F)cccc5[nH]c4=O)[nH]c3c2)CC1]

and L comprises a moiety of formula

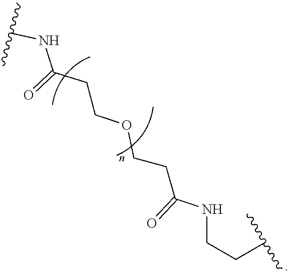

In certain embodiments, the moiety —N(R¹)₂ is of formula

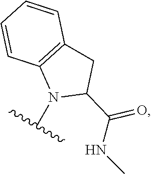 [SMILES: *N1c2ccccc2CC1C(=O)NC]

B is of formula

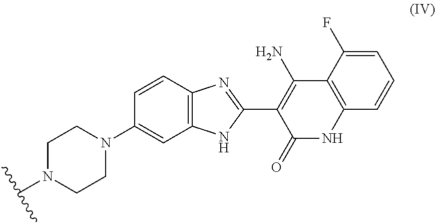 [SMILES: *N1CCN(c2ccc3nc(-c4c(N)c5c(F)cccc5[nH]c4=O)[nH]c3c2)CC1]

and L comprises a moiety of formula

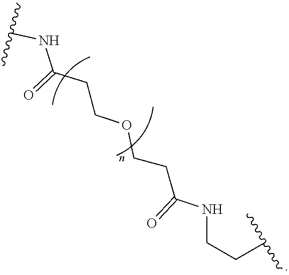

In certain embodiments, the moiety —N(R¹)₂ is of formula

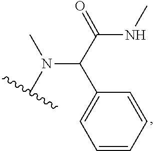

B is of formula

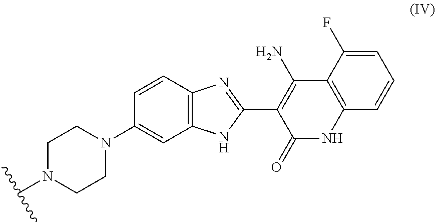 [SMILES: *N1CCN(c2ccc3nc(-c4c(N)c5c(F)cccc5[nH]c4=O)[nH]c3c2)CC1]

and L comprises a moiety of formula

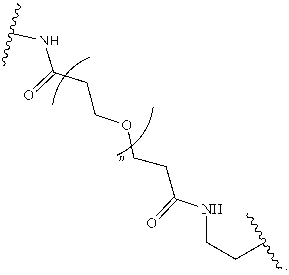

In certain embodiments, the compound of formula (II) is of formula:

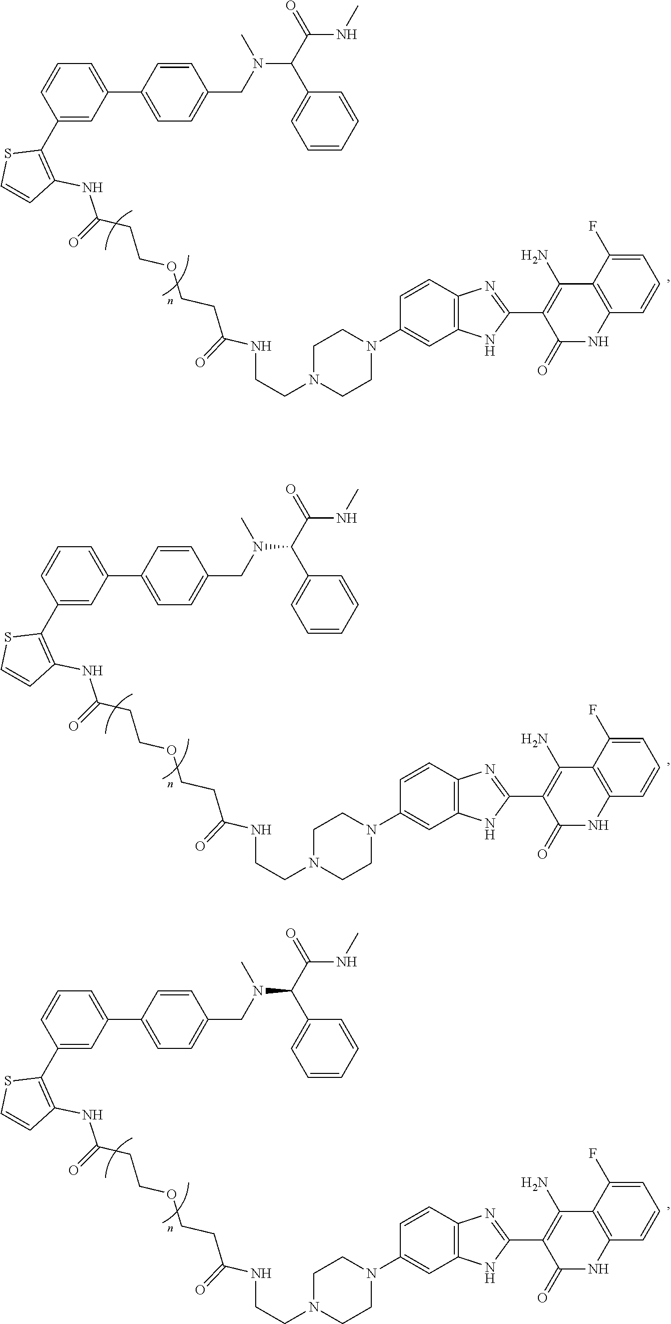

or a pharmaceutically acceptable salt, solvate, hydrate, polymorph, co-crystal, tautomer, stereoisomer, isotopically labeled compound, or prodrug thereof. In certain embodiments, the compound of Formula (II) is of formula:

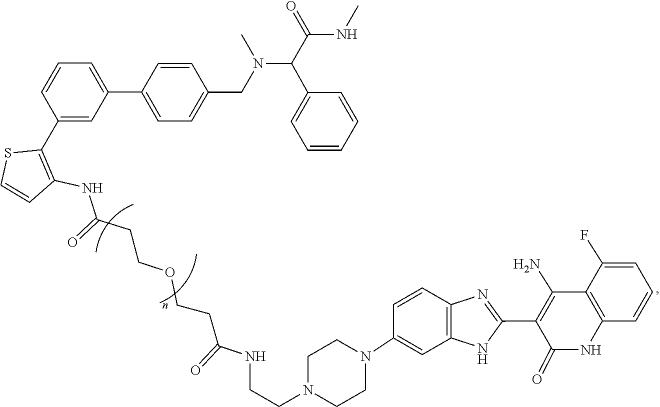

or a pharmaceutically acceptable salt, solvate, hydrate, polymorph, co-crystal, tautomer, stereoisomer, isotopically labeled compound, or prodrug thereof. In certain embodiments, the compound of Formula (II) is of formula:

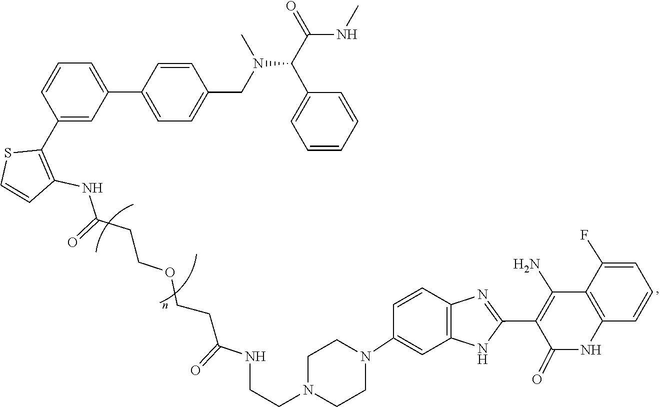

or a pharmaceutically acceptable salt, solvate, hydrate, polymorph, co-crystal, tautomer, stereoisomer, isotopically labeled compound, or prodrug thereof. In certain embodiments, the compound of Formula (II) is of formula:

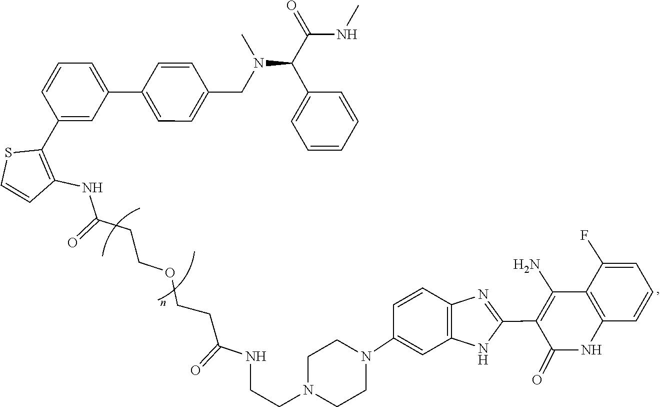

or a pharmaceutically acceptable salt, solvate, hydrate, polymorph, co-crystal, tautomer, stereoisomer, isotopically labeled compound, or prodrug thereof.

In certain embodiments, the compound of Formula (II) is of formula:

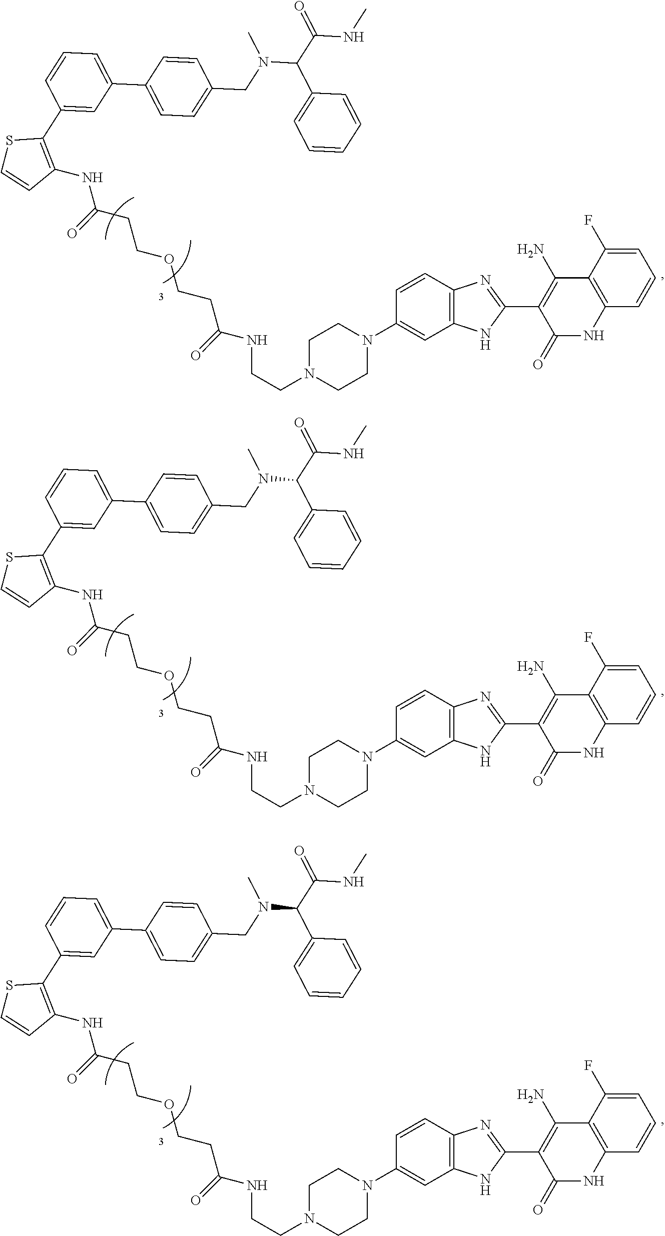

or a pharmaceutically acceptable salt, solvate, hydrate, polymorph, co-crystal, tautomer, stereoisomer, isotopically labeled compound, or prodrug thereof. In certain embodiments, the compound of Formula (II) is of formula:

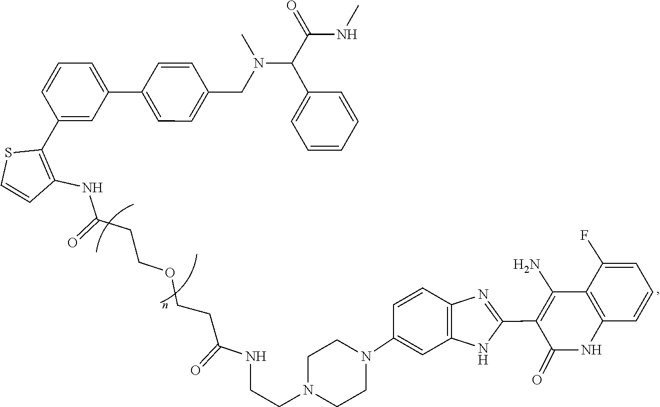

or a pharmaceutically acceptable salt, solvate, hydrate, polymorph, co-crystal, tautomer, stereoisomer, isotopically labeled compound, or prodrug thereof. In certain embodiments, the compound of Formula (II) is of formula:

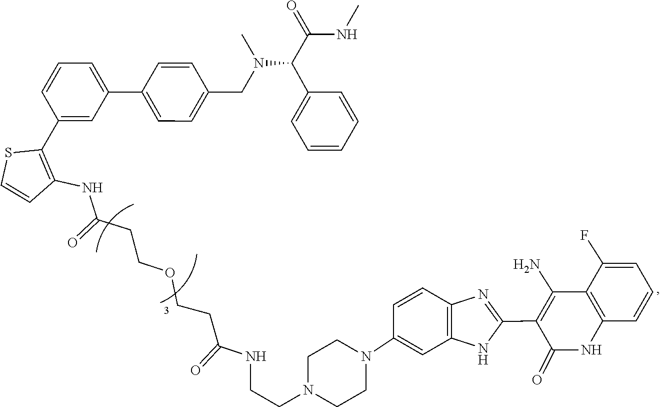

or a pharmaceutically acceptable salt, solvate, hydrate, polymorph, co-crystal, tautomer, stereoisomer, isotopically labeled compound, or prodrug thereof. In certain embodiments, the compound of Formula (II) is of formula:

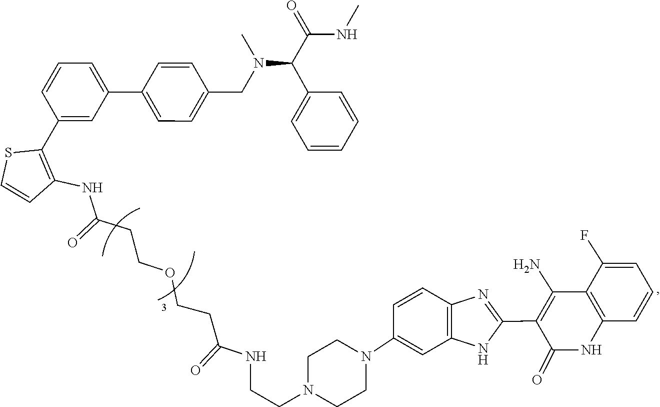

or a pharmaceutically acceptable salt, solvate, hydrate, polymorph, co-crystal, tautomer, stereoisomer, isotopically labeled compound, or prodrug thereof.

In certain embodiments, a provided compound (a compound described herein, a compound of the present disclosure) is a compound of any of the formulae herein (e.g., Formulae (I) or (II)), or pharmaceutically acceptable salt, solvate, hydrate, polymorph, co-crystal, tautomer, stereoisomer, isotopically labeled compound, or prodrug thereof. In certain embodiments, a provided compound is a compound of any of the formulae herein (e.g., Formulae (I) or (II)), or a pharmaceutically acceptable salt or tautomer thereof. In certain embodiments, a provided compound is a compound of any of the formulae herein (e.g., Formulae (I) or (II)), or a pharmaceutically acceptable salt thereof. In certain embodiments, a provided compound is a compound of any of the formulae herein (e.g., Formulae (I) or (II)), or a salt thereof.

### Pharmaceutical Compositions and Kits

In one aspect, the present disclosure provides pharmaceutical compositions comprising a provided compound. In some embodiments, the pharmaceutical composition comprises one or more excipients. In certain embodiments, the pharmaceutical compositions described herein comprise a provided compound and an excipient.

In certain embodiments, the pharmaceutical composition comprises an effective amount of the provided compound. In certain embodiments, the effective amount is a therapeutically effective amount. In certain embodiments, the effective amount is a prophylactically effective amount. In certain embodiments, the effective amount is an amount effective for treating a disease or disorder associated with associated with microRNA-21 (miR-21) (e.g., proliferative disease, kidney disease) in a subject in need thereof. In certain embodiments, the effective amount is an amount effective for preventing a disease or disorder associated with microRNA-21 (miR-21) (e.g., proliferative disease, kidney disease) in a subject in need thereof. In certain embodiments, the effective amount is an amount effective for reducing the risk of developing a disease or disorder associated with microRNA-21 (miR-21) (e.g., proliferative disease, kidney disease) in a subject in need thereof. In certain embodiments, the effective amount is an amount effective for binding a precursor to microRNA-21 (pre-miR-21) in a subject in need thereof or in a cell, tissue, or biological sample. In certain embodiments, the effective amount is an amount effective for downregulating mature microRNA-21 (miR-21).

In certain embodiments, the subject is an animal. In certain embodiments, the subject is a human. In certain embodiments, the subject is a human aged 18 years or older. In certain embodiments, the subject is a human aged 12-18 years, exclusive. In certain embodiments, the subject is a human aged 2-12 years, inclusive. In certain embodiments, the subject is a human younger than 2 years. In certain embodiments, the subject is a non-human animal. In certain embodiments, the subject is a mammal. In certain embodiments, the subject is a non human mammal. In certain embodiments, the subject is a domesticated animal, such as a dog, cat, cow, pig, horse, sheep, or goat. In certain embodiments, the subject is a companion animal, such as a dog or cat. In certain embodiments, the subject is a livestock animal, such as a cow, pig, horse, sheep, or goat. In certain embodiments, the subject is a zoo animal. In another embodiment, the subject is a research animal, such as a rodent (e.g., mouse, rat), dog, pig, or non-human primate. In certain embodiments, the animal is a genetically engineered animal. In certain embodiments, the animal is a transgenic animal (e.g., transgenic mice and transgenic pigs). In certain embodiments, the subject is a fish or reptile.

In certain embodiments, the effective amount is an amount effective for downregulating mature microRNA-21 (miR-21) (e.g., by at least about 10%, at least about 20%, at least about 30%, at least about 40%, at least about 50%, at least about 60%, at least about 70%, at least about 80%, at least about 90%, at least 95%, at least 98%, at least 99%, or at least about 100%). In certain embodiments, the effective amount is an amount effective for downregulating mature microRNA-21 by a range between a percentage described in this paragraph and another percentage described in this paragraph, inclusive.

In certain embodiments, the pharmaceutical composition is for use in treating a disease or disorder associated with microRNA-21 (miR-21) (e.g., proliferative disease, kidney disease) in a subject in need thereof. In certain embodiments, the pharmaceutical composition is for use in preventing a disease or disorder associated with microRNA-21 (miR-21) (e.g., proliferative disease, kidney disease) in a subject in need thereof. In certain embodiments, the pharmaceutical composition is for use in binding a precursor to microRNA-21 (pre-miR-21) in a subject in need thereof or in a cell, tissue, or biological sample. In certain embodiments, the pharmaceutical composition is for use in downregulating mature microRNA-21 in a subject in need thereof or in a cell, tissue, or biological sample.

A provided compound or pharmaceutical composition, as described herein, can be administered in combination with one or more additional pharmaceutical agents (e.g., therapeutically and/or prophylactically active agents). The provided compounds or pharmaceutical compositions can be administered in combination with additional pharmaceutical agents that improve their activity (e.g., activity (e.g., potency and/or efficacy) in treating a disease or disorder associated with miR-21 (e.g., proliferative disease, kidney disease) in a subject in need thereof, in preventing a disease or disorder associated with miR-21 (e.g., proliferative disease, kidney disease) in a subject in need thereof, and/or in reducing the risk of developing a disease or disorder associated with miR-21 (e.g., proliferative disease, kidney disease) in a subject in need thereof), improve bioavailability, improve safety, reduce drug resistance, reduce and/or modify metabolism, inhibit excretion, and/or modify distribution in a subject or cell. It will also be appreciated that the additional pharmaceutical agents employed may achieve a desired effect for the same disorder, and/or it may achieve different effects. In certain embodiments, a pharmaceutical composition described herein including a provided compound described herein and an additional pharmaceutical agent exhibit a synergistic effect that is absent in a pharmaceutical composition including one of the provided compounds and the additional pharmaceutical agent, but not both. In some embodiments, the additional pharmaceutical agent achieves a desired effect for the same disorder. In some embodiments, the additional pharmaceutical agent achieves different effects.

The provided compound or pharmaceutical composition can be administered concurrently with, prior to, or subsequent to one or more additional pharmaceutical agents, which are different from the compound or pharmaceutical composition and may be useful as, e.g., combination therapies. Pharmaceutical agents include therapeutically active agents. Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides, synthetic proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, the additional pharmaceutical agent is a pharmaceutical agent useful for treating and/or preventing a disease or disorder associated with miR-21 (e.g., proliferative disease, kidney disease).

Each additional pharmaceutical agent may be administered at a dose and/or on a time schedule determined for that pharmaceutical agent. The additional pharmaceutical agents may also be administered together with each other and/or with the compound or pharmaceutical composition described herein in a single dose or administered separately in different doses. The particular combination to employ in a regimen will take into account compatibility of the compound described herein with the additional pharmaceutical agent(s) and/or the desired therapeutic and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized individually.

The additional pharmaceutical agents include, but are not limited to, anti-proliferative agents, anti-cancer agents, anti-angiogenesis agents, steroidal or non-steroidal anti-inflammatory agents, immunosuppressants, anti-bacterial agents, anti-viral agents, cardiovascular agents, cholesterol-lowering agents, anti-diabetic agents, anti-allergic agents, contraceptive agents, pain-relieving agents, anesthetics, anti-coagulants, inhibitors of an enzyme, steroidal agents, steroidal or antihistamine, antigens, vaccines, antibodies, decongestant, sedatives, opioids, analgesics, anti-pyretics, hormones, and prostaglandins. In certain embodiments, the additional pharmaceutical agents include, but are not limited to, cardiovascular agents, cholesterol-lowering agents, anti-diabetic agents, anti-coagulants, steroidal agents, hormones, and prostaglandins. In certain embodiments, the additional pharmaceutical agent is a cardiovascular agent. In certain embodiments, the additional pharmaceutical agent is a cholesterol-lowering agent. In certain embodiments, the additional pharmaceutical agent is an anti-diabetic agent. In certain embodiments, the additional pharmaceutical agent is an anti-coagulant. In certain embodiments, the additional pharmaceutical agent is a steroidal agent. In certain embodiments, the additional pharmaceutical agent is a hormone. In certain embodiments, the additional pharmaceutical agent is a prostaglandin.

In certain embodiments, the provided compound or pharmaceutical composition is a solid. In certain embodiments, the provided compound or pharmaceutical composition is a powder. In certain embodiments, the provided compound or pharmaceutical composition can be dissolved in a liquid to make a solution. In certain embodiments, the provided compound or pharmaceutical composition is dissolved in water to make an aqueous solution. In certain embodiments, the pharmaceutical composition is a liquid for parental injection. In certain embodiments, the pharmaceutical composition is a liquid for oral administration (e.g., ingestion). In certain embodiments, the pharmaceutical composition is a liquid (e.g., aqueous solution) for intravenous injection. In certain embodiments, the pharmaceutical composition is a liquid (e.g., aqueous solution) for subcutaneous injection.

Pharmaceutical compositions described herein can be prepared by any method known in the art of pharmacology. In general, such preparatory methods include the steps of bringing the composition comprising a provided compound (i.e., the “active ingredient”) into association with a carrier and/or one or more other accessory ingredients, and then, if necessary and/or desirable, shaping and/or packaging the product into a desired single- or multi-dose unit.

Pharmaceutical compositions can be prepared, packaged, and/or sold in bulk, as a single unit dose, and/or as a plurality of single unit doses. A “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage, such as one-half or one-third of such a dosage.

Relative amounts of the provided compound, pharmaceutically acceptable excipient, agent, and/or any additional ingredients in a pharmaceutical composition described herein will vary, depending upon the identity, size, and/or condition of the subject treated and further depending upon the route by which the pharmaceutical composition is to be administered. The pharmaceutical composition may comprise between 0.1% and 100% (w/w) agent, inclusive.

Pharmaceutically acceptable excipients used in manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients and accessory ingredients, such as cocoa butter and suppository waxes, coloring agents, coating agents, sweetening, flavoring, and perfuming agents, may also be present in the pharmaceutical composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween® 20), polyoxyethylene sorbitan monostearate (Tween® 60), polyoxyethylene sorbitan monooleate (Tween® 80), sorbitan monopalmitate (Span® 40), sorbitan monostearate (Span® 60), sorbitan tristearate (Span® 65), glyceryl monooleate, sorbitan monooleate (Span® 80), polyoxyethylene esters (e.g., polyoxyethylene monostearate (Myrj® 45), polyoxyethylene hydrogenated castor oil, polyethoxylated castor oil, polyoxymethylene stearate, and Solutol®), sucrose fatty acid esters, polyethylene glycol fatty acid esters (e.g., Cremophor®), polyoxyethylene ethers, (e.g., polyoxyethylene lauryl ether (Brij® 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic® F-68, poloxamer P-188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant® Plus, Phenonip®, methylparaben, Germall® 115, Germaben® II, Neolone®, Kathon®, and Euxyl®.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral pharmaceutical compositions can include adjuvants such as wetting agents, emulsifying and suspending agents, sweetening, flavoring, and perfuming agents. In certain embodiments for parenteral administration, the conjugates described herein are mixed with solubilizing agents such as Cremophor®, alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension, or emulsion in a nontoxic parenterally acceptable diluent or solvent, for example, as a solution in 1,3-butanediol. Among the acceptable vehicles and solvents that can be employed are water, Ringer's solution, U.S.P., and isotonic sodium chloride solution. In addition, sterile, fixed oils are conventionally employed as a solvent or suspending medium. For this purpose any bland fixed oil can be employed including synthetic mono- or di-glycerides. In addition, fatty acids such as oleic acid are used in the preparation of injectables.

The injectable formulations can be sterilized, for example, by filtration through a bacterial-retaining filter, or by incorporating sterilizing agents in the form of sterile solid pharmaceutical compositions which can be dissolved or dispersed in sterile water or other sterile injectable medium prior to use.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form may be accomplished by dissolving or suspending the drug in an oil vehicle.

Pharmaceutical compositions for rectal or vaginal administration are typically suppositories which can be prepared by mixing the conjugates described herein with suitable non-irritating excipients or carriers such as cocoa butter, polyethylene glycol, or a suppository wax which are solid at ambient temperature but liquid at body temperature and therefore melt in the rectum or vaginal cavity and release the active ingredient.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may include a buffering agent.

Solid compositions of a similar type can be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the art of pharmacology. They may optionally comprise opacifying agents and can be of a composition that they release the active ingredient(s) only, or preferentially, in a certain part of the intestinal tract, optionally, in a delayed manner. Examples of encapsulating compositions which can be used include polymeric substances and waxes.

The active ingredient can be in a micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the active ingredient can be admixed with at least one inert diluent such as sucrose, lactose, or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of encapsulating agents which can be used include polymeric substances and waxes.

Dosage forms for topical and/or transdermal administration of a compound described herein may include ointments, pastes, creams, lotions, gels, powders, solutions, sprays, inhalants, and/or patches. Generally, the active ingredient is admixed under sterile conditions with a pharmaceutically acceptable carrier or excipient and/or any needed preservatives and/or buffers as can be required. Additionally, the present disclosure contemplates the use of transdermal patches, which often have the added advantage of providing controlled delivery of an active ingredient to the body. Such dosage forms can be prepared, for example, by dissolving and/or dispensing the active ingredient in the proper medium. Alternatively or additionally, the rate can be controlled by either providing a rate controlling membrane and/or by dispersing the active ingredient in a polymer matrix and/or gel.

Formulations suitable for topical administration include, but are not limited to, liquid and/or semi-liquid preparations such as liniments, lotions, oil-in-water and/or water-in-oil emulsions such as creams, ointments, and/or pastes, and/or solutions and/or suspensions. Topically administrable formulations may, for example, comprise from about 1% to about 10% (w/w) active ingredient, although the concentration of the active ingredient can be as high as the solubility limit of the active ingredient in the solvent. Formulations for topical administration may further comprise one or more of the additional ingredients described herein.

Suitable devices for use in delivering injectable pharmaceutical compositions described herein include short needle devices. Injectable pharmaceutical compositions can be administered by devices which limit the effective penetration length of a needle into the skin. Alternatively or additionally, conventional syringes can be used in the classical mantoux method of administration. Jet injection devices which deliver liquid formulations via a liquid jet injector and/or via a needle. Ballistic powder/particle delivery devices which use compressed gas to accelerate the compound in powder form are suitable.

A pharmaceutical composition described herein can be prepared, packaged, and/or sold in a formulation suitable for pulmonary administration via the buccal cavity. Such a formulation may comprise dry particles which comprise the active ingredient and which have a diameter in the range from about 0.5 to about 7 nanometers, or from about 1 to about 6 nanometers. Such pharmaceutical compositions are conveniently in the form of dry powders for administration using a device comprising a dry powder reservoir to which a stream of propellant can be directed to disperse the powder and/or using a self-propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nanometers and at least 95% of the particles by number have a diameter less than 7 nanometers. Alternatively, at least 95% of the particles by weight have a diameter greater than 1 nanometer and at least 90% of the particles by number have a diameter less than 6 nanometers. Dry powder pharmaceutical compositions may include a solid fine powder diluent such as sugar and are conveniently provided in a unit dose form.

Low boiling propellants generally include liquid propellants having a boiling point of below 65° F. at atmospheric pressure. Generally, the propellant may constitute 50 to 99.9% (w/w) of the pharmaceutical composition, and the active ingredient may constitute 0.1 to 20% (w/w) of the pharmaceutical composition. The propellant may further comprise additional ingredients such as a liquid non-ionic and/or solid anionic surfactant and/or a solid diluent (which may have a particle size of the same order as particles comprising the active ingredient).

Pharmaceutical compositions described herein formulated for pulmonary delivery may provide the active ingredient in the form of droplets of a solution and/or suspension. Such formulations can be prepared, packaged, and/or sold as aqueous and/or dilute alcoholic solutions and/or suspensions, optionally sterile, comprising the active ingredient, and may conveniently be administered using any nebulization and/or atomization device. Such formulations may further comprise one or more additional ingredients including a flavoring agent such as saccharin sodium, a volatile oil, a buffering agent, a surface active agent, and/or a preservative such as methylhydroxybenzoate. The droplets provided by this route of administration may have an average diameter in the range from about 0.1 to about 200 nanometers.

Formulations described herein as being useful for pulmonary delivery are useful for intranasal delivery of a pharmaceutical composition described herein. Another formulation suitable for intranasal administration is a coarse powder comprising the active ingredient and having an average particle from about 0.2 to 500 micrometers. Such a formulation is administered by rapid inhalation through the nasal passage from a container of the powder held close to the nares.

Formulations for nasal administration may, for example, comprise from about as little as 0.1% (w/w) to as much as 100% (w/w) of the active ingredient, and may comprise one or more of the additional ingredients described herein. A pharmaceutical composition described herein can be prepared, packaged, and/or sold in a formulation for buccal administration. Such formulations may, for example, be in the form of tablets and/or lozenges made using conventional methods, and may contain, for example, 0.1 to 20% (w/w) active ingredient, the balance comprising an orally dissolvable and/or degradable composition and, optionally, one or more of the additional ingredients described herein. Alternately, formulations for buccal administration may comprise a powder and/or an aerosolized and/or atomized solution and/or suspension comprising the active ingredient. Such powdered, aerosolized, and/or aerosolized formulations, when dispersed, may have an average particle and/or droplet size in the range from about 0.1 to about 200 nanometers, and may further comprise one or more of the additional ingredients described herein.

A pharmaceutical composition described herein can be prepared, packaged, and/or sold in a formulation for ophthalmic administration. Such formulations may, for example, be in the form of eye drops including, for example, a 0.1-1.0% (w/w) solution and/or suspension of the active ingredient in an aqueous or oily liquid carrier or excipient. Such drops may further comprise buffering agents, salts, and/or one or more other of the additional ingredients described herein. Other opthalmically-administrable formulations which are useful include those which comprise the active ingredient in microcrystalline form and/or in a liposomal preparation. Ear drops and/or eye drops are also contemplated as being within the scope of this disclosure.

Although the descriptions of pharmaceutical compositions provided herein are principally directed to pharmaceutical compositions which are suitable for administration to humans, it will be understood by the skilled artisan that such pharmaceutical compositions are generally suitable for administration to animals of all sorts. Modification of pharmaceutical compositions suitable for administration to humans in order to render the pharmaceutical compositions suitable for administration to various animals is well understood, and the ordinarily skilled veterinary pharmacologist can design and/or perform such modification with ordinary experimentation.

Provided compounds are typically formulated in dosage unit form for ease of administration and uniformity of dosage. It will be understood, however, that the total daily usage of the pharmaceutical compositions described herein will be decided by a physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex, and diet of the subject; the time of administration, route of administration, and rate of excretion of the specific active ingredient employed; the duration of the treatment; drugs used in combination or coincidental with the specific active ingredient employed; and like factors well known in the medical arts.

The provided compounds and pharmaceutical compositions provided herein can be administered by any route, including enteral (e.g., oral), parenteral, intravenous, intramuscular, intraarticular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, sublingual; by intratracheal instillation, bronchial instillation, and/or inhalation; and/or as an oral spray, nasal spray, and/or aerosol. Specifically, contemplated routes are intraarticular administration, oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration).

The exact amount of a provided compound required to achieve an effective amount will vary from subject to subject, depending, for example, on species, age, and general condition of a subject, severity of the side effects or disorder, identity of the particular compound of the disclosure, mode of administration, and the like. An effective amount may be included in a single dose (e.g., single oral dose) or multiple doses (e.g., multiple oral doses). In certain embodiments, when multiple doses are administered to a subject or applied to a biological sample, tissue, or cell, any two doses of the multiple doses include different or substantially the same amounts of an agent described herein.

In certain embodiments, a pharmaceutical composition comprising a provided compound is administered, orally or parenterally, at dosage levels of each pharmaceutical composition sufficient to deliver from about 0.001 mg/kg to about 200 mg/kg in one or more dose administrations for one or several days (depending on the mode of administration). In certain embodiments, the effective amount per dose varies from about 0.001 mg/kg to about 200 mg/kg, about 0.001 mg/kg to about 100 mg/kg, about 0.01 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, of subject body weight per day, one or more times a day, to obtain the desired therapeutic and/or prophylactic effect. In certain embodiments, the compounds described herein may be at dosage levels sufficient to deliver from about 0.001 mg/kg to about 200 mg/kg, from about 0.001 mg/kg to about 100 mg/kg, from about 0.01 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, and more preferably from about 1 mg/kg to about 25 mg/kg, of subject body weight per day, one or more times a day, to obtain the desired therapeutic and/or prophylactic effect. The desired dosage may be delivered three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations). In certain embodiments, the pharmaceutical composition described herein is administered at a dose that is below the dose at which the agent causes non-specific effects.

In certain embodiments, the pharmaceutical composition is administered at a dose of about 0.001 mg to about 1000 mg per unit dose. In certain embodiments, the pharmaceutical composition is administered at a dose of about 0.01 mg to about 200 mg per unit dose. In certain embodiments, the pharmaceutical composition is administered at a dose of about 0.01 mg to about 100 mg per unit dose. In certain embodiments, pharmaceutical composition is administered at a dose of about 0.01 mg to about 50 mg per unit dose. In certain embodiments, the pharmaceutical composition is administered at a dose of about 0.01 mg to about 10 mg per unit dose. In certain embodiments, the pharmaceutical composition is administered at a dose of about 0.1 mg to about 10 mg per unit dose.

Dose ranges as described herein provide guidance for the administration of provided compounds or pharmaceutical compositions to an adult. The amount to be administered to, for example, a child or an adolescent can be determined by a medical practitioner or person skilled in the art and can be lower or the same as that administered to an adult. In certain embodiments, a dose described herein is a dose to an adult human whose body weight is 70 kg.

In certain embodiments, when multiple doses are administered to a subject or applied to a tissue or cell, the frequency of administering the multiple doses to the subject or applying the multiple doses to the tissue or cell may be, in non-limiting examples, three doses a day, two doses a day, one dose a day, one dose every other day, one dose every third day, one dose every week, one dose every two weeks, one dose every three weeks, or one dose every four weeks, or even slow dose controlled delivery over a selected period of time using a drug delivery device. In certain embodiments, when multiple doses are administered to a subject or applied to a biological sample, tissue, or cell, the duration between the first dose and last dose of the multiple doses is one day, two days, four days, one week, two weeks, three weeks, one month, two months, three months, four months, six months, nine months, one year, two years, three years, four years, five years, seven years, ten years, fifteen years, twenty years, or the lifetime of the subject, tissue, or cell. In certain embodiments, the duration between the first dose and last dose of the multiple doses is three months, six months, or one year. In certain embodiments, the duration between the first dose and last dose of the multiple doses is the lifetime of the subject, tissue, or cell.

Also encompassed by the present disclosure are kits (e.g., pharmaceutical packs). In certain embodiments, the kit comprises a provided compound or pharmaceutical composition described herein, and instructions for using the compound or pharmaceutical composition. In certain embodiments, the kit comprises a first container, wherein the first container includes the compound or pharmaceutical composition. In some embodiments, the kit further comprises a second container. In certain embodiments, the second container includes an excipient (e.g., an excipient for dilution or suspension of the compound or pharmaceutical composition). In certain embodiments, the second container includes an additional pharmaceutical agent. In some embodiments, the kit further comprises a third container. In certain embodiments, the third container includes an additional pharmaceutical agent. In some embodiments, the provided compound or pharmaceutical composition included in the first container and the excipient or additional pharmaceutical agent included in the second container are combined to form one unit dosage form. In some embodiments, the provided compound or pharmaceutical composition included in the first container, the excipient included in the second container, and the additional pharmaceutical agent included in the third container are combined to form one unit dosage form. In certain embodiments, each of the first, second, and third containers is independently a vial, ampule, bottle, syringe, dispenser package, tube, or inhaler.

In certain embodiments, the instructions are for administering the provided compound or pharmaceutical composition to a subject (e.g., a subject in need of treatment or prevention of a disease described herein). In certain embodiments, the instructions are for contacting a biological sample or cell with the provided compound or pharmaceutical composition. In certain embodiments, the instructions comprise information required by a regulatory agency, such as the U.S. Food and Drug Administration (FDA) or the European Agency for the Evaluation of Medicinal Products (EMA). In certain embodiments, the instructions comprise prescribing information.

In certain embodiments, the kits and instructions provide for treating a disease or disorder associated with miR-21 (e.g., proliferative disease, kidney disease) in a subject in need thereof. In certain embodiments, the kits and instructions provide for preventing a disease or disorder associated with miR-21 (e.g., proliferative disease, kidney disease) in a subject in need thereof. In certain embodiments, the kits and instructions provide for reducing the risk of developing a disease or disorder associated with miR-21 (e.g., proliferative disease, kidney disease) in a subject in need thereof. In certain embodiments, the kits and instructions provide for binding a precursor to microRNA-21 (pre-miR-21) in a subject in need thereof or in a cell, tissue, or biological sample. In certain embodiments, the kits and instructions provide for downregulating mature microRNA-21.

A kit described herein may include one or more additional pharmaceutical agents described herein as a separate pharmaceutical composition.

Another object of the present disclosure is the use of a compound as described herein in the manufacture of a medicament for use in the treatment of a disorder or disease described herein. Another object of the present disclosure is the use of a compound as described herein for use in the treatment of a disorder or disease described herein.

### Methods of Treatment and Prevention

In another aspect, the present disclosure provides methods of treating or preventing a disease in a subject in need thereof, comprising administering to the subject in need thereof a provided compound or pharmaceutical composition. In certain embodiments, the present disclosure provides methods of treating a disease in a subject in need thereof, comprising administering to the subject in need thereof a provided compound or pharmaceutical composition. In certain embodiments, the present disclosure provides methods of preventing a disease in a subject in need thereof, comprising administering to the subject in need thereof a provided compound or pharmaceutical composition. In certain embodiments, the disease is associated with microRNA-21 (miR-21) (e.g., proliferative disease, kidney disease).

In another aspect, the present disclosure provides a provided compound or pharmaceutical composition for use in treating or preventing a disease in a subject in need thereof. In another aspect, the present disclosure provides a provided compound or pharmaceutical composition for use in treating a disease in a subject in need thereof. In another aspect, the present disclosure provides a provided compound or pharmaceutical composition for use in preventing a disease in a subject in need thereof. In certain embodiments, the disease is associated with microRNA-21 (miR-21) (e.g., proliferative disease, kidney disease)

In another aspect, the present disclosure provides a provided compound or pharmaceutical composition for use in the manufacture of a medicament for treatment or prevention of a disease in a subject in need thereof. In another aspect, the present disclosure provides a provided compound or pharmaceutical composition for use in the manufacture of a medicament for treatment of a disease in a subject in need thereof. In another aspect, the present disclosure provides a provided compound or pharmaceutical composition for use in the manufacture of a medicament for prevention of a disease in a subject in need thereof. In certain embodiments, the disease is associated with microRNA-21 (miR-21) (e.g., proliferative disease, kidney disease)

In certain embodiments, the disease is associated with microRNA-21 (miR-21) (e.g., proliferative disease, kidney disease). In certain embodiments, the disease is a proliferative disease (e.g., breast cancer, prostate cancer, lung cancer). In certain embodiments, the proliferative disease is breast cancer (e.g., triple-negative breast cancer (TNBC)). In certain embodiments, the proliferative disease is triple-negative breast cancer (TNBC). In certain embodiments, the proliferative disease is prostate cancer. In certain embodiments, the proliferative disease is lung cancer (e.g., non-small cell lung cancer (NSCLC)). In certain embodiments, the proliferative disease is non-small cell lung cancer (NSCLC). In certain embodiments, the disease is a kidney disease (e.g., Alport Syndrome). In certain embodiments, the kidney disease is Alport Syndrome.

### Methods of Binding RNase L, Binding an RNA Target, and Inhibiting Cell Proliferation or Inducing Apoptosis

In another aspect, the present disclosure provides methods of effecting degradation of an RNA target in a subject in need thereof or in a cell, tissue, or biological sample, comprising administering to the subject in need thereof or contacting the cell, tissue, or biological sample with an effective amount of a heterobifunctional compound, or a pharmaceutically acceptable salt, solvate, hydrate, polymorph, co-crystal, tautomer, stereoisomer, isotopically labeled compound, or prodrug thereof, comprising a compound of Formula (I), or a pharmaceutically acceptable salt, solvate, hydrate, polymorph, co-crystal, tautomer, stereoisomer, isotopically labeled compound, or prodrug thereof, capable of binding to RNase L, and an RNA binder capable of binding to the RNA target. In certain embodiments, the compound of Formula (I) and the RNA binder are connected by a linker.

In some embodiments, a heterobifunctional compound comprises: a radical of a compound of Formula (I), or a radical of a compound that includes a compound of Formula (I), and a radical of an RNA binder, or a radical of a compound that includes an RNA binder.

In another aspect, the present disclosure provides methods of preparing a heterobifunctional compound, or a pharmaceutically acceptable salt, solvate, hydrate, polymorph, co-crystal, tautomer, stereoisomer, isotopically labeled compound, or prodrug thereof, capable of effecting degradation of an RNA target, comprising combining: a radical of a compound of Formula (I), or a pharmaceutically acceptable salt, solvate, hydrate, polymorph, co-crystal, tautomer, stereoisomer, isotopically labeled compound, or prodrug thereof, or a radical of a linker connected to the compound of Formula (I), or pharmaceutically acceptable salt, solvate, hydrate, polymorph, co-crystal, tautomer, stereoisomer, isotopically labeled compound, or prodrug thereof, wherein the compound of Formula (I), or pharmaceutically acceptable salt, solvate, hydrate, polymorph, co-crystal, tautomer, stereoisomer, isotopically labeled compound, or prodrug thereof, is capable of binding to RNase L; and a radical of an RNA binder, or a radical of a linker connected to the RNA binder, wherein the RNA binder is capable of binding to the RNA target.

In another aspect, the present disclosure provides methods of binding RNase L in a subject in need thereof or in a cell, tissue, or biological sample, comprising administering to the subject in need thereof or contacting the cell, tissue, or biological sample with an effective amount of a provided compound or pharmaceutical composition. In certain embodiments, the present disclosure provides methods of binding RNase L in a subject in need thereof, comprising administering to the subject in need thereof an effective amount of a provided compound or pharmaceutical composition. In certain embodiments, the present disclosure provides methods of binding RNase L in a cell, tissue, or biological sample, comprising contacting the cell, tissue, or biological sample with an effective amount of a provided compound or pharmaceutical composition.

In another aspect, the present disclosure provides methods of binding an RNA target in a subject in need thereof or in a cell, tissue, or biological sample, comprising administering to the subject in need thereof or contacting the cell, tissue, or biological sample with an effective amount of a provided compound or pharmaceutical composition. In certain embodiments, the present disclosure provides methods of binding an RNA target in a subject in need thereof, comprising administering to the subject in need thereof an effective amount of a provided compound or pharmaceutical composition. In certain embodiments, the present disclosure provides methods of binding an RNA target in a cell, tissue, or biological sample, comprising contacting the cell, tissue, or biological sample with an effective amount of a provided compound or pharmaceutical composition. In another aspect, the present disclosure provides methods of inhibiting cell proliferation in a subject in need thereof or in a cell, tissue, or biological sample, comprising administering to the subject in need thereof or contacting the cell, tissue, or biological sample with an effective amount of a provided compound or pharmaceutical composition. In certain embodiments, the present disclosure provides methods of inhibiting cell proliferation in a subject in need thereof comprising administering to the subject in need thereof an effective amount of a provided compound or pharmaceutical composition. In certain embodiments, the present disclosure provides methods of inhibiting cell proliferation in a cell, tissue, or biological sample, comprising contacting the cell, tissue, or biological sample with an effective amount of a provided compound or pharmaceutical composition. In certain embodiments, the method comprises inhibiting cell proliferation (e.g., by at least about 10%, at least about 20%, at least about 30%, at least about 40%, at least about 50%, at least about 60%, at least about 70%, at least about 80%, at least about 90%, at least 95%, at least 98%, at least 99%, or at least about 100%).

In certain embodiments, binding RNase L comprises activating RNase L (e.g., by at least about 10%, at least about 20%, at least about 30%, at least about 40%, at least about 50%, at least about 60%, at least about 70%, at least about 80%, at least about 90%, at least about 100%, at least about 150%, at least about 200%, at least about 300%, at least about 400%, at least about 500%, at least about 600%, at least about 700%, at least about 800%, at least about 900%, or at least about 1000%). In certain embodiments, binding RNase L comprises inducing RNase L dimerization (e.g., by at least about 10%, at least about 20%, at least about 30%, at least about 40%, at least about 50%, at least about 60%, at least about 70%, at least about 80%, at least about 90%, at least about 100%, at least about 150%, at least about 200%, at least about 300%, at least about 400%, at least about 500%, at least about 600%, at least about 700%, at least about 800%, at least about 900%, or at least about 1000%).

In certain embodiments, the method further comprises recruiting RNase L. In certain embodiments, the method further comprises activating RNase L (e.g., by at least about 10%, at least about 20%, at least about 30%, at least about 40%, at least about 50%, at least about 60%, at least about 70%, at least about 80%, at least about 90%, at least about 100%, at least about 150%, at least about 200%, at least about 300%, at least about 400%, at least about 500%, at least about 600%, at least about 700%, at least about 800%, at least about 900%, or at least about 1000%). In certain embodiments, the method further comprises inducing RNase L dimerization (e.g., by at least about 10%, at least about 20%, at least about 30%, at least about 40%, at least about 50%, at least about 60%, at least about 70%, at least about 80%, at least about 90%, at least about 100%, at least about 150%, at least about 200%, at least about 300%, at least about 400%, at least about 500%, at least about 600%, at least about 700%, at least about 800%, at least about 900%, or at least about 1000%).

In certain embodiments, the method comprises cleaving the RNA target (e.g., by at least about 10%, at least about 20%, at least about 30%, at least about 40%, at least about 50%, at least about 60%, at least about 70%, at least about 80%, at least about 90%, at least 95%, at least 98%, at least 99%, or at least about 100%). In certain embodiments, the method comprises decreasing an amount of the RNA target (e.g., by at least about 10%, at least about 20%, at least about 30%, at least about 40%, at least about 50%, at least about 60%, at least about 70%, at least about 80%, at least about 90%, at least 95%, at least 98%, at least 99%, or at least about 100%).

In certain embodiments, the RNA target is a precursor to microRNA-21 (pre-miR-21). In certain embodiments, the method comprises decreasing an amount of pre-miR-21 (e.g., by at least about 10%, at least about 20%, at least about 30%, at least about 40%, at least about 50%, at least about 60%, at least about 70%, at least about 80%, at least about 90%, at least 95%, at least 98%, at least 99%, or at least about 100%). In certain embodiments, the method comprises cleaving pre-miR-21 (e.g., by at least about 10%, at least about 20%, at least about 30%, at least about 40%, at least about 50%, at least about 60%, at least about 70%, at least about 80%, at least about 90%, at least 95%, at least 98%, at least 99%, or at least about 100%).

In certain embodiments, the method further comprises downregulating mature microRNA-21 (miR-21) (e.g. miR-21-5p). In certain embodiments, miR-21 is downregulated (e.g., by at least about 10%, at least about 20%, at least about 30%, at least about 40%, at least about 50%, at least about 60%, at least about 70%, at least about 80%, at least about 90%, at least 95%, at least 98%, at least 99%, or at least about 100%). In certain embodiments, the downregulating is selective for miR-21 compared to other mature microRNA. In certain embodiments, the downregulating is selective for miR-21 compared to miR-101 (e.g., miR-101-3p), miR-135b (e.g., miR-135b-5p), miR-345 (e.g., miR-345-5p), miR-363 (e.g., miR-363-3p), and/or miR-378a (e.g., miR-378a-3p). In certain embodiments, the method further comprises decreasing an amount of miR-21 (e.g., by at least about 10%, at least about 20%, at least about 30%, at least about 40%, at least about 50%, at least about 60%, at least about 70%, at least about 80%, at least about 90%, at least 95%, at least 98%, at least 99%, or at least about 100%).

In certain embodiments, the method further comprises increasing an amount of programmed cell death protein 4 (PDCD4) (e.g., by at least about 10%, at least about 20%, at least about 30%, at least about 40%, at least about 50%, at least about 60%, at least about 70%, at least about 80%, at least about 90%, at least about 100%, at least about 150%, at least about 200%, at least about 300%, at least about 400%, at least about 500%, at least about 600%, at least about 700%, at least about 800%, at least about 900%, or at least about 1000%). In certain embodiments, increasing an amount of PDCD4 comprises derepression of PDCD4.

In certain embodiments, the method comprises selectively deactivating a miR-21-mediated cancer circuit. In certain embodiments, the method comprises not changing an amount of extracellular signal-regulated kinase (ERK) or an amount of phosphorylated extracellular signal-regulated kinase (pERK).

In certain embodiments, the cell, tissue, or biological sample is in vivo. In certain embodiments, the cell, tissue, or biological sample is in vitro.

### Methods of Identifying a Binder of an Effector Protein

In another aspect, the present disclosure provides methods of identifying a binder of an effector protein, comprising screening the effector protein against a DNA-encoded library (DEL) to identify a set of compounds specifically enriched for binding to the effector protein; synthesizing one or more compounds from the set of compounds specifically enriched for binding to the effector protein without an encoding DNA tag; determining effector protein activation by the one or more compounds from the set of compounds specifically enriched for binding to the effector protein synthesized without the encoding DNA tag; and identifying the one or more compounds from the set of compounds specifically enriched for binding to the effector protein synthesized without the encoding DNA tag as a binder of the effector protein.

In certain embodiments, the effector protein is RNase L. In certain embodiments, determining effector protein activation comprises an in vitro assay. In certain embodiments, the in vitro assay is fluorescence-based.

In certain embodiments, screening the effector protein against the DNA-encoded library (DEL) to identify the set of compounds specifically enriched for binding to the effector protein comprises identifying a set of compounds as binding to the effector protein; identifying one or more control sets of compounds; and removing the one or more control sets of compounds from the set of compounds identified as binding to the effector protein to identify the set of compounds specifically enriched for binding to the effector protein. In certain embodiments, the set of compounds identified as binding to the effector protein comprises a set of compounds enriched by a recombinant effector protein functionalized with a tag. In certain embodiments, one control set of compounds comprises a set of compounds enriched by the tag alone. In certain embodiments, a first control set of compounds comprises a set of compounds enriched by the tag alone. In certain embodiments, the tag is glutathione S-transferase (GST). In certain embodiments, one control set of compounds comprises a set of compounds enriched by a no target control (NTC). In certain embodiments, a first control set of compounds comprises a set of compounds enriched by a no target control (NTC). In certain embodiments, a second control set of compounds comprises a set of compounds enriched by a no target control (NTC).

In certain embodiments, the method further comprises calculating an enrichment score for one or more compounds from the set of compounds specifically enriched for binding to the effector protein. In certain embodiments, the enrichment score is calculated from decoding of the DNA tags by next generation sequencing (NGS). In certain embodiments, the enrichment score is calculated using corrected copy number, library size, NGS depth, and/or additional normalization factors.

### Methods of Preparation

In another aspect, the present disclosure provides methods of preparing a compound of Formula (II-a):

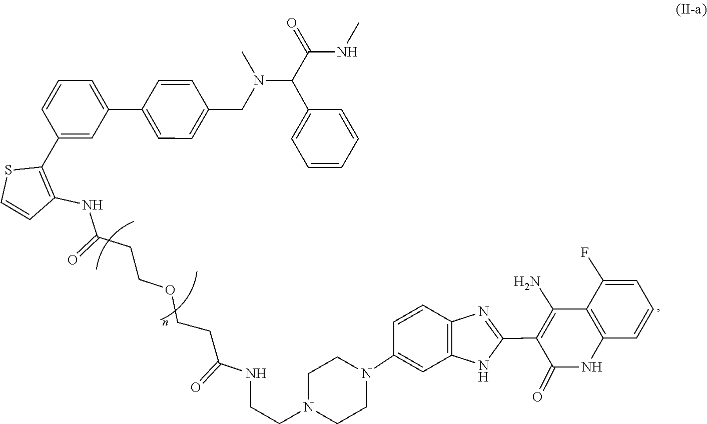

or a pharmaceutically acceptable salt, solvate, hydrate, polymorph, co-crystal, tautomer, stereoisomer, isotopically labeled compound, or prodrug thereof, comprising reacting a compound of formula:

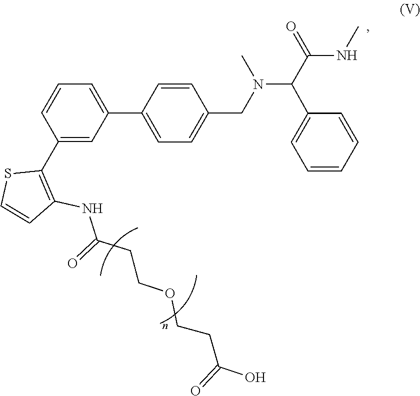

or salt thereof, with a compound of formula:

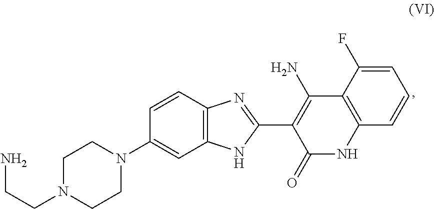 [SMILES: NCCN1CCN(c2ccc3nc(-c4c(N)c5c(F)cccc5[nH]c4=O)[nH]c3c2)CC1]

or a salt thereof, wherein:
n is 0,1,2,3,4,5,6,7,8,9, or 10.

In certain embodiments, n is 0, 1, 2, 3, 4, 5, 6, 7, 8, 9, or 10. In certain embodiments, n is 0. In certain embodiments, n is 1. In certain embodiments, n is 2. In certain embodiments, n is 3. In certain embodiments, n is 4. In certain embodiments, n is 5. In certain embodiments, n is 6. In certain embodiments, n is 7. In certain embodiments, n is 8. In certain embodiments, n is 9. In certain embodiments, n is 10.

In certain embodiments, the compound of Formula (II-a) is of formula:

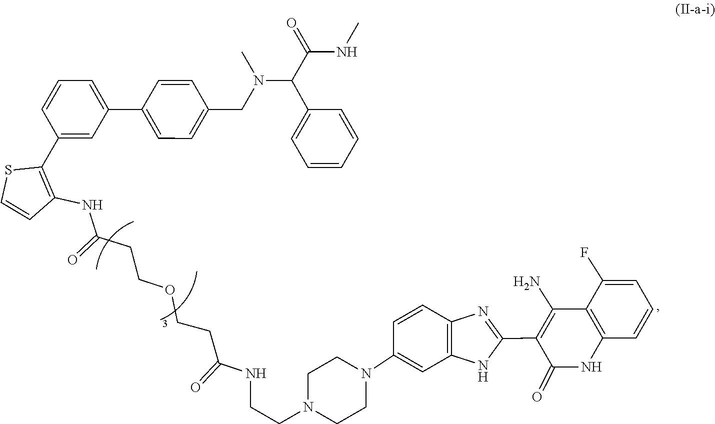

or a pharmaceutically acceptable salt, solvate, hydrate, polymorph, co-crystal, tautomer, stereoisomer, isotopically labeled compound, or prodrug thereof.

### EXAMPLES

In order that the present disclosure may be more fully understood, the following examples are set forth. The synthetic and biological examples described in this application are offered to illustrate the compounds, pharmaceutical compositions, and methods provided herein and are not to be construed in any way as limiting in their scope.

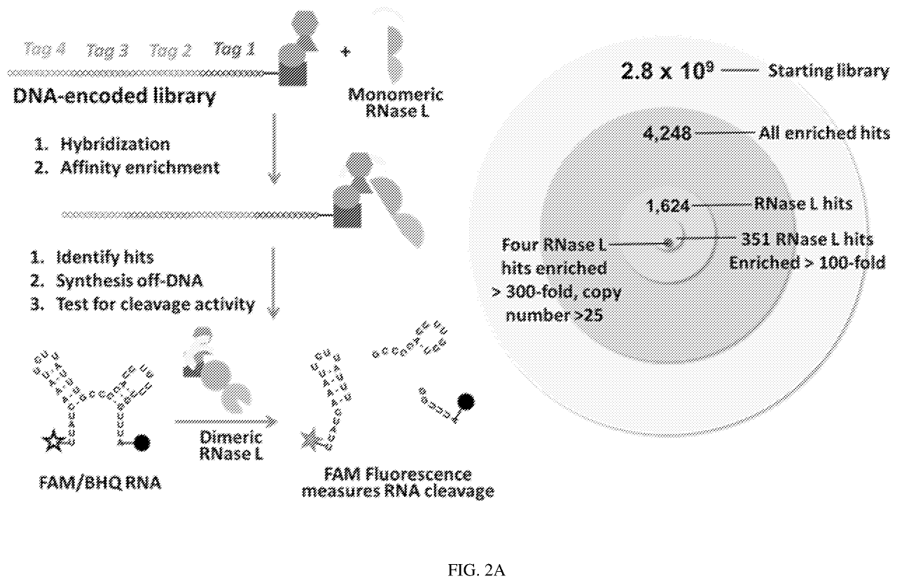

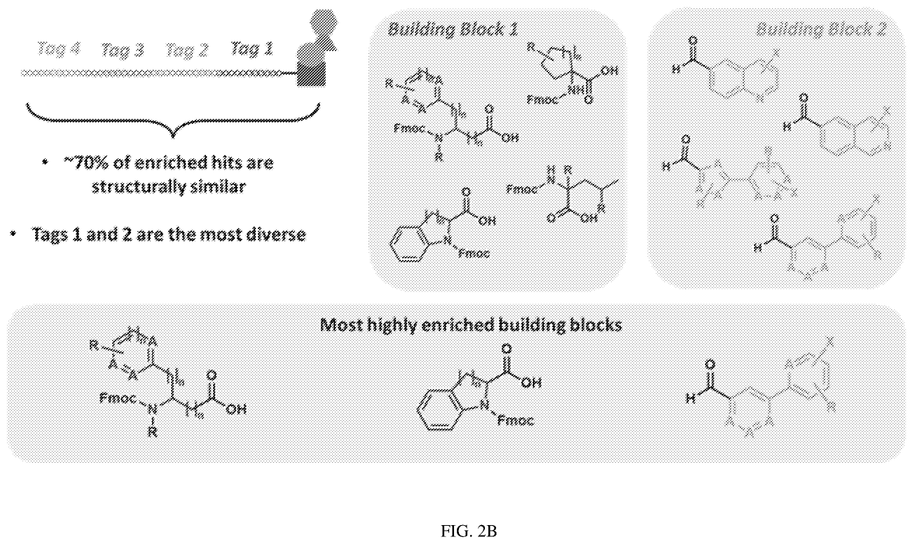

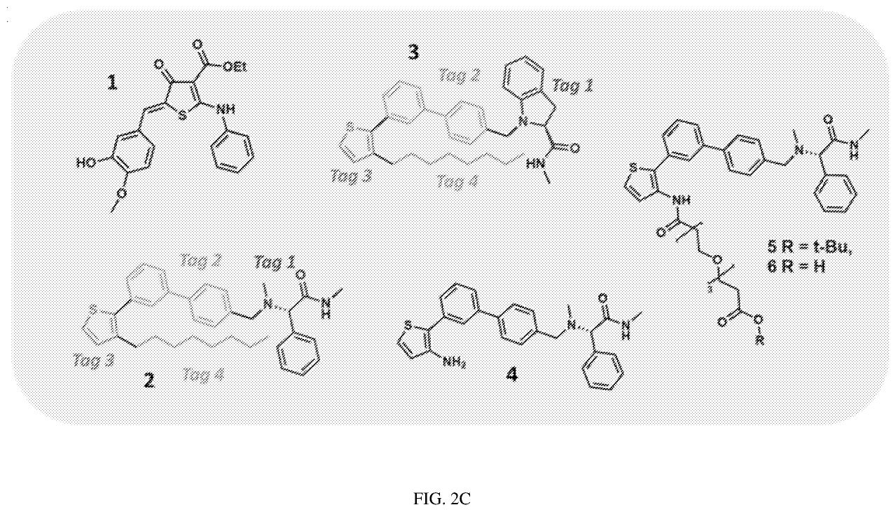

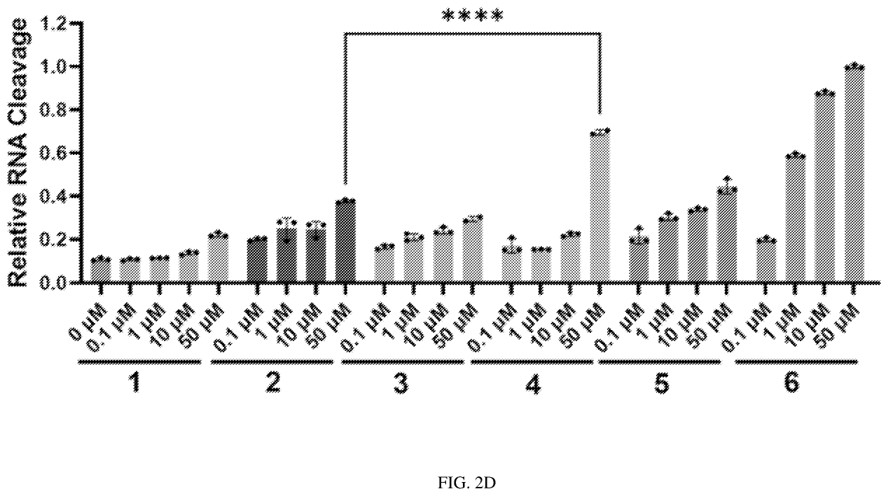

Monomeric RNase L was screened against the 2nd generation DELopen (WuXi AppTec) library¹⁵ (2.8 billion compounds) to identify new binders (FIG. 2A). After affinity enrichment, 4,248 compounds were identified across three conditions: (i) recombinant GST-RNase L-His6 (n=1,999 compounds); (ii) the GST tag alone (counter screen; n=380 compounds); and (iii) a no target control (NTC) (counter screen; n=2,244 compounds). Removing hits present in the two counter screens afforded 1,624 compounds specifically enriched for binding to RNase L. Of these, 351 compounds had an enrichment score of at least 100 for RNase L. The enrichment score was calculated from decoding of the DNA tags by next generation sequencing (NGS), considering corrected copy number, library size, NGS depth and other normalization factors, as completed by WuXi AppTec (see https://hits.wuxiapptec.com/delopen). A molecule with an enrichment score of 100 is 100 times more abundant in the sequencing data than the average molecule in the library, which has a score of 1. Analysis of the DEL identification codes showed most of the compounds enriched by RNase L belonged to the same compound family (FIG. 2B). Additionally, the largest positions of diversity were building blocks 1 and 2 (i.e., tag 1 and tag 2), showing a preference for highly heteroaromatic structures. Building blocks 3 and 4 were unchanged in the most highly enriched compounds, suggesting the thiophene may contribute to RNase L binding (FIG. 2B).

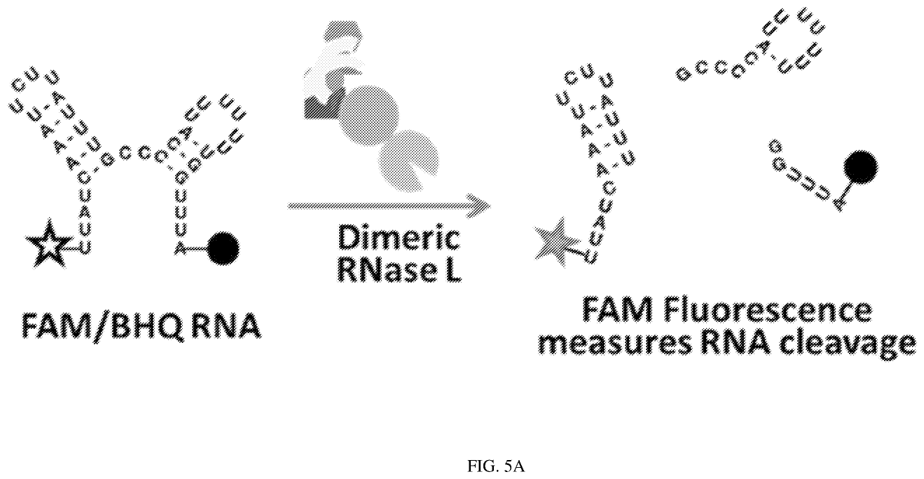

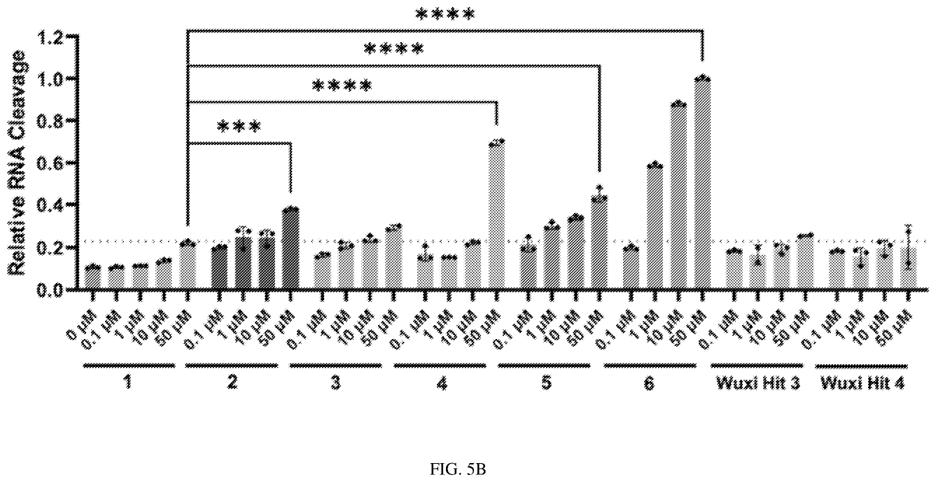

The four compounds with the highest confidence (enrichment scores>300 and copy number>25) were selected for further study. The compounds were re-synthesized without the encoding DNA tag and were studied for RNase L activation using a fluorescence-based in vitro assay, completed as previously described.¹⁶ In brief, the assay uses an dually labeled RNA [5′ fluorescein (6-FAM) and 3′ black hole quencher (BHQ)] that harbors multiple preferred substates for RNase L (FIG. 2A).¹⁶ In the presence of a binder that activates RNase L, the enzyme cleaves the RNA, resulting in an increased fluorescence signal. Here, a previously validated small molecule recruiter, 1, was used as a positive control.¹⁷⁻¹⁸ Two of the four tested compounds, 2 and 3, induced dose-dependent RNA cleavage to a greater extent than 1, indicating RNase L dimerization and activation (FIGS. 2C-D & 5). 2 and 3 are similar in structure, differing only by the first building block, phenylglycine in 2 and indoline in 3 (FIG. 2C). Data for the compounds not chosen is shown in FIG. 5. Taken together with the data gleaned from the DEL codes, these data suggest the aromatic rings and the sulfur atom play a role in binding of these small molecules to RNase L.

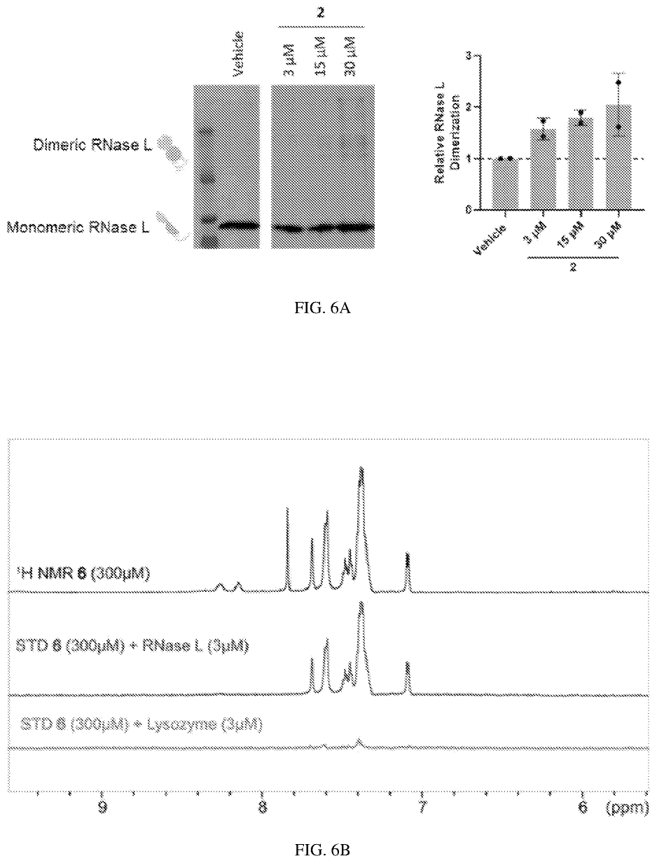

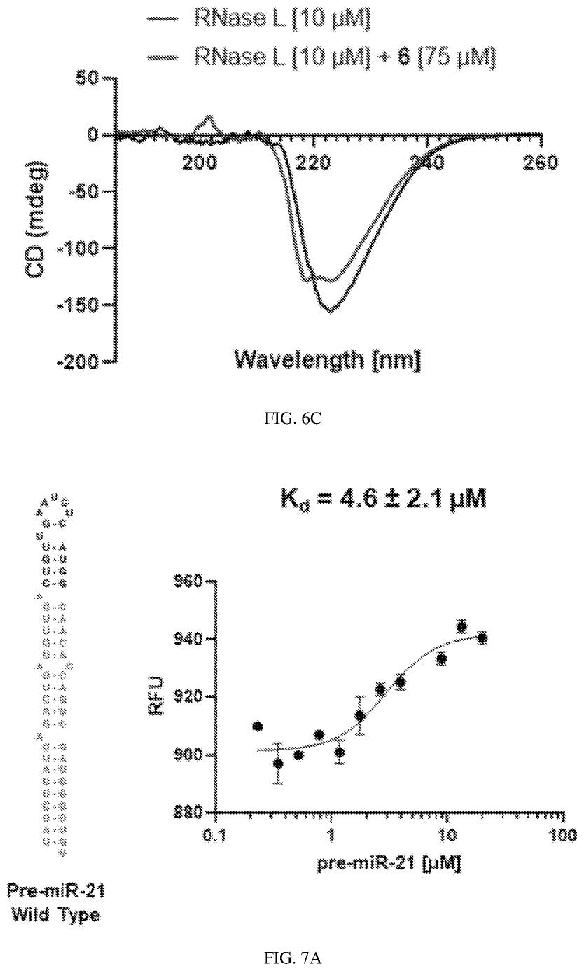

Prior to incorporating 2 into a next-generation RiboTAC, it was verified that 2 induces RNase L dimerization in vitro by Western blotting (FIG. 6A). The position within 2 suitable for conjugation was then determined. Previous studies in the ProTAC field have shown that the attachment site for a linker within the recruiting module should be chosen such that it does not affect binding.¹⁹ It was hypothesized that the long alkyl chain in 2 could be used as an attachment point, as this site is unlikely to affect RNase L-binding. Therefore, this position was derivatized and assessed for RNase L activation. Compound 4, which lacks the alkyl chain, showed enhanced RNA cleavage compared to 2 (p<0.0001), indicating the alkyl chain is not required for RNase L binding and dimerization (FIGS. 2C-D). A polyethylene glycol (PEG) linker terminated with a t-butyl ester was attached to yield 5 (FIG. 2C). Compound 5 also retained RNase L activation activity, indicating this compound could be used in the design of a RiboTAC (FIG. 2D).

To assess further the ability of this compound to bind to RNase L, saturation transfer difference (STD) nuclear magnetic resonance (NMR)²⁰ and circular dichroism (CD) spectroscopic studies were conducted with 6, a compound similar in structure to 5 and a potential intermediate in the synthesis of a resulting RiboTAC (FIGS. 2C & 6B-6C). STD-NMR spectra showed transfer of magnetization from the compound to RNase L, whereas no transfer was observed between 6 and the control protein lysozyme. Furthermore, CD spectra revealed an increase in signal intensity at 220 nm upon addition of 6, indicative of increased alpha-helical content and consistent with dimerization of RNase L.²¹

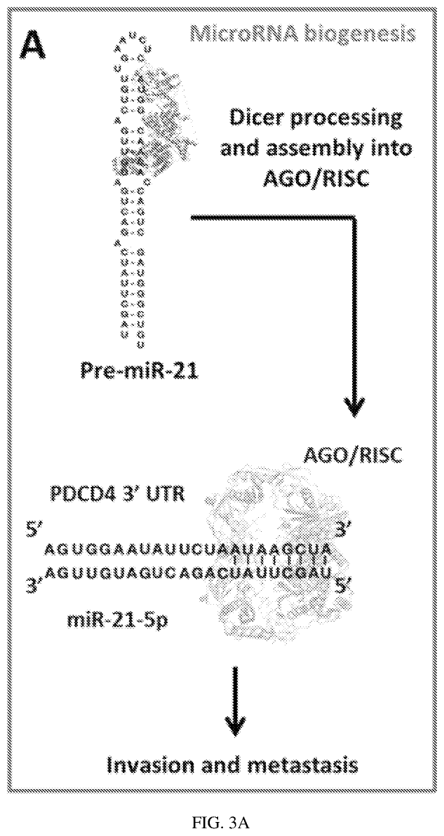

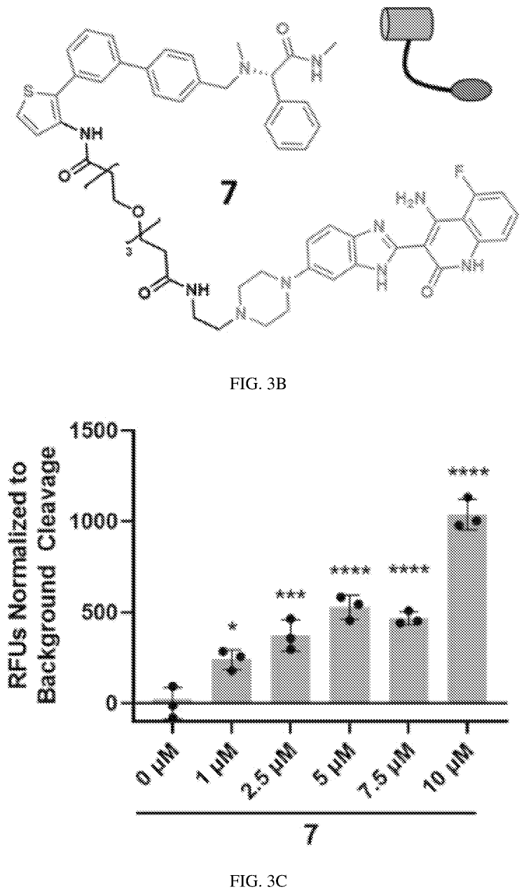

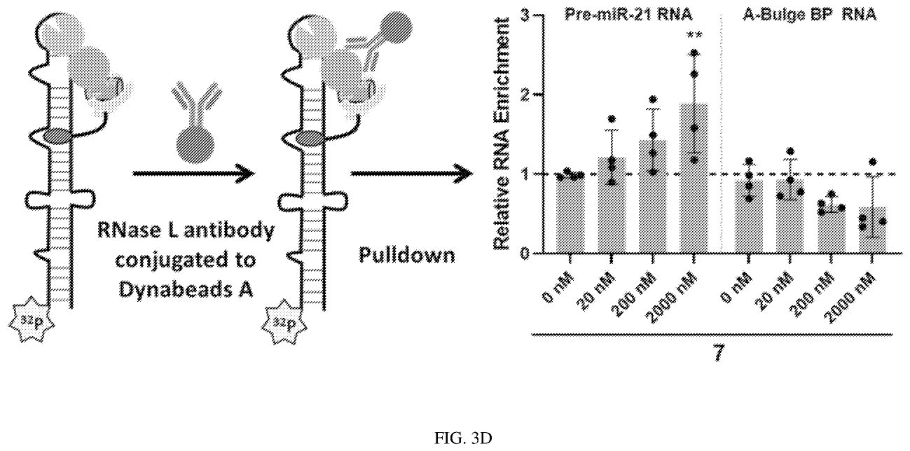

Next, the RNase L recruiter was examined as part of a previously validated RiboTAC that targets the precursor to microRNA-21 (pre-miR-21).¹⁸ MicroRNAs (miRNAs) are small non-coding RNAs that are transcribed as primary transcripts that are processed first in the nucleus by Drosha to liberate the precursor miRNA, and then in the cytoplasm by Dicer to produce the mature miRNA. The mature miRNA then associates with the Argonaute/RNA-induced silencing complex (AGO/RISC) and binds to the 3′ untranslated region (UTR) of complementary mRNAs to translationally repress or cleave them (FIG. 3A).²² In many cancers, miR-21 is highly expressed, including triple-negative breast cancer (TNBC), where aberrant expression triggers cancer-associated cellular phenotypes including migration.²³-24

To construct a pre-miR-21-targeting RiboTAC, 6 was attached to Dovitinib, a known kinase inhibitor that also binds to the Dicer processing site in pre-miR-21,¹⁸ yielding RiboTAC 7 (FIG. 3B). The in vitro interaction between the chimera (7) and premiR-21 was characterized using a battery of assays, including direct binding, pre-miR-21 cleavage, occupancy by Competitive Chemical Cross-linking and Isolation by Pull-down (C-Chem-CLIP) and co-immunoprecipitation assays.²⁵

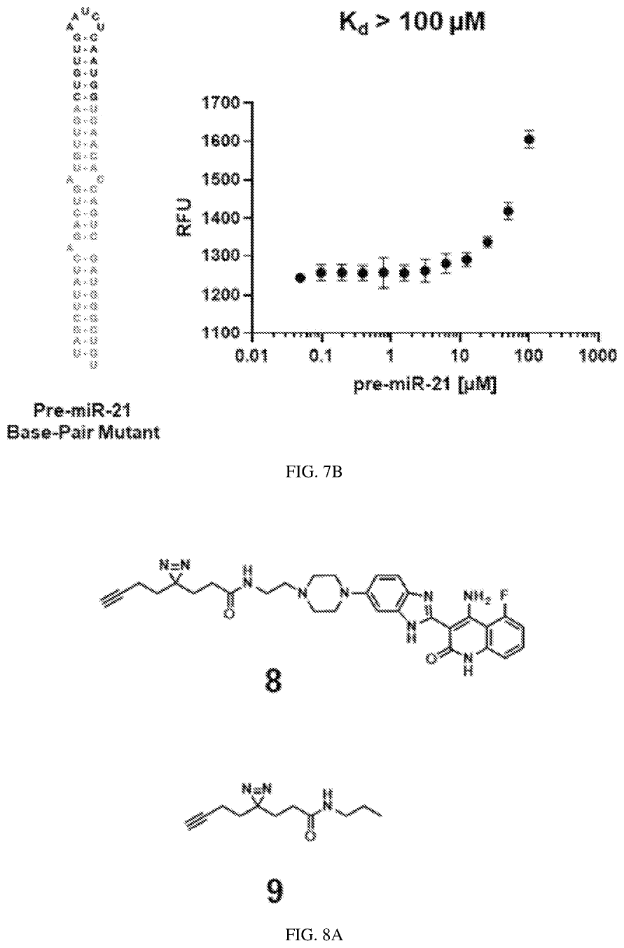

RiboTAC 7 bound to pre-miR-21 with a K_{d} of 4.6±2.1 μM, while no saturable binding (up to 100 μM) was observed to a mutant pre-miR-21 in which the A and U bulge binding sites¹⁸ were mutated to base pairs (FIG. 7). These data support that 7 recognizes the structure of pre-miR-21, not its sequence. Additionally, the K_{d} is similar to a previously reported Dovitinib-derived RiboTAC, indicating the new RNase L recruiter was not affecting Dovitinib's affinity for pre-miR-21.¹⁸

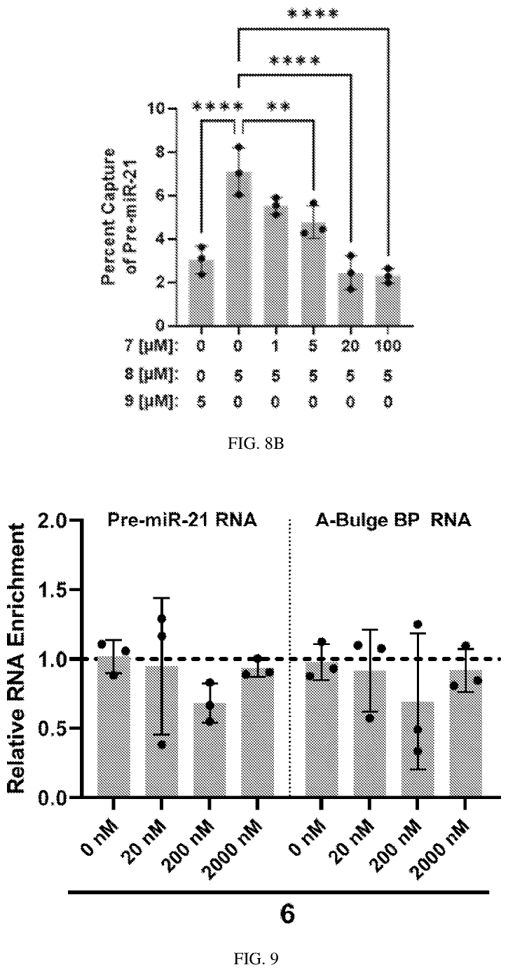

Direct target occupancy was further verified using C-Chem-CLIP. In this assay, the RNA-binding module was conjugated to a diazirine cross-linking module and an alkyne handle for purification, affording Chem-CLIP probe 8 (FIG. 8A).¹⁸ Radioactively labeled pre-miR-21 was incubated with a constant concentration of 8 and increasing amounts of 7. After cross-linking by irradiation with UV light, the amount of pre-miR-21 pulled down by 8 was measured by its radioactive signal. If the two compounds (7 and 8) share the same binding site, the amount of pre-miR-21 in the pulled down fraction is depleted. 7 dose-dependently reduced the amount of pre-miR-21 cross-linked to 8 (FIG. 8B). No pulldown was observed with a control Chem-CLIP probe that lacked the RNA-binding module (9) (FIG. 8A-8B). These data further support RiboTAC 7's specificity for the RNA's structure.

To confirm ternary complex formation, a pre-miR-21 construct dually labeled with 6-FAM and BHQ was incubated with 7 and RNase L. RiboTAC 7 induced dose-dependent cleavage of pre-miR-21, supporting that the chimera both interacts with premiR-21 and functions through an RNase L-mediated mechanism of action (FIG. 3C). Further, a co-immunoprecipitation assay using an inactive RNase L protein (mutation in catalytic site) showed that pre-miR-21, but not its base paired mutant, was pulled down with RNase L in the presence of 7 (FIG. 3D). Neither RNA was pulled down in the presence of 6, which lacks the RNA-binding module (FIG. 9). These data support that ternary complex formation is involved in the RiboTAC's mechanism of action.

RiboTAC 7 was next tested in the TNBC cell line MDA-MB-231 to investigate its ability to cleave pre-miR-21 and rescue miR-21-mediated cellular phenotypes. In TNBC cells, miR-21 represses expression of programmed cell death protein 4 (PDCD4).²⁶ Dysregulation of PDCD4 leads to an increase in the cell's invasive character and eventually to metastasis.²⁷

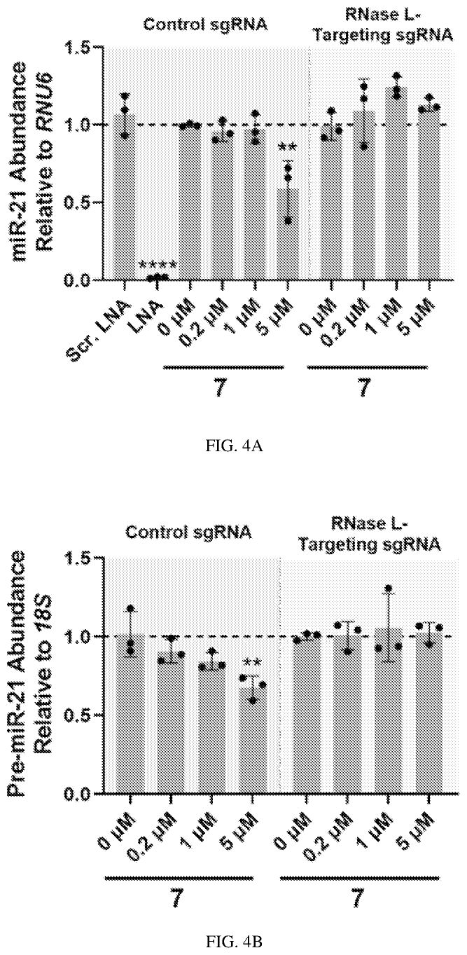

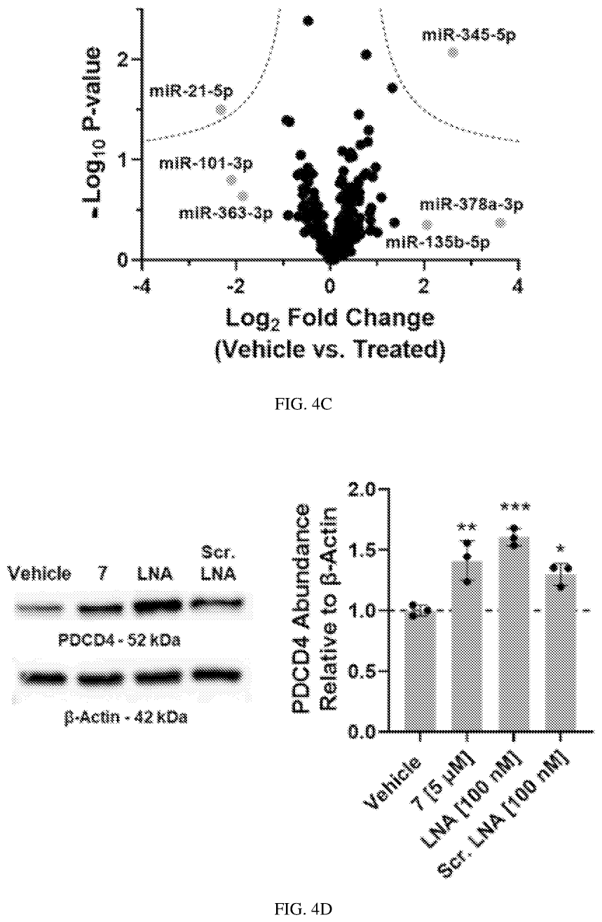

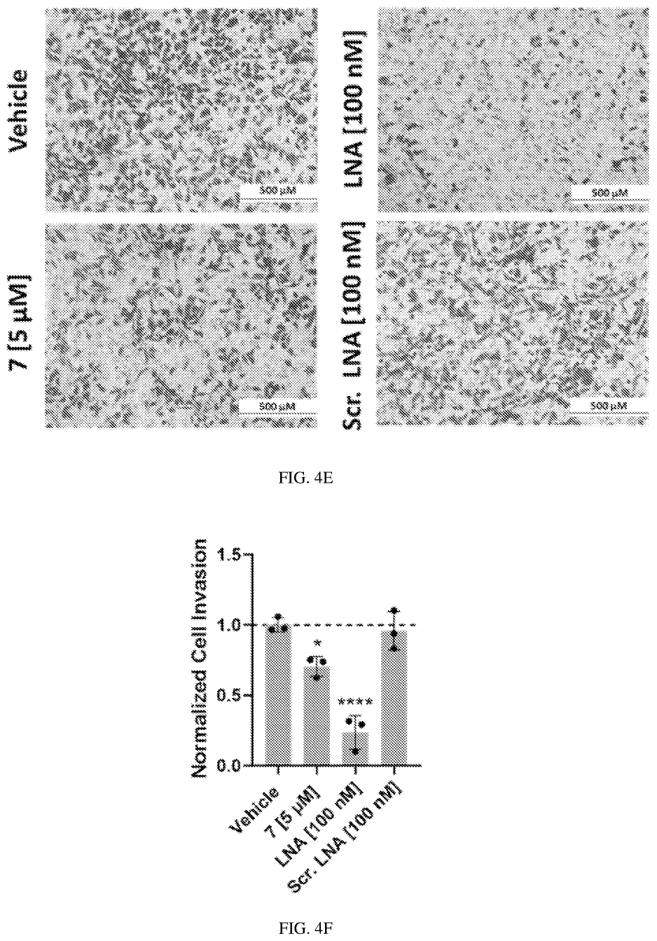

Two CRISPR-modified MDA-MB-231 cell lines with either a scrambled control guide RNA (“Control sgRNA”; RNase L is expressed) or with an RNase L-targeting guide RNA (“RNase L-Targeting sgRNA”; RNase L protein abundance is knocked down) were used to complete these studies. In CRISPR-Control sgRNA cells in which RNase L is expressed, 7 (5 μM) reduced mature and pre-miR-21 abundance by ˜40% and ˜30%, respectively (p<0.01; FIGS. 4A-B; “Control sgRNA”; left) after a 48 h treatment, similar to the previously reported Dovitinib-derived RiboTAC that uses 1 as the RNase L recruiter.¹⁸ Knock-down of RNase L by CRISPR¹⁸ rendered 7 inactive (FIG. 4A-B; “RNase L-Targeting sgRNA”; right), supporting an RNase L-mediated mechanism of action targeted to the miRNA precursor. miRNA profiling revealed 7 selectively down-regulated miR-21 across the transcriptome (FIG. 4C). The additional five miRs that were dysregulated by >2-fold are known hallmarks of breast cancer cells and are thus not off-targets of 7 (FIG. 4C).²⁸³¹ Collectively, these data support that 7 selectively cleaves pre-miR-21 in cells.

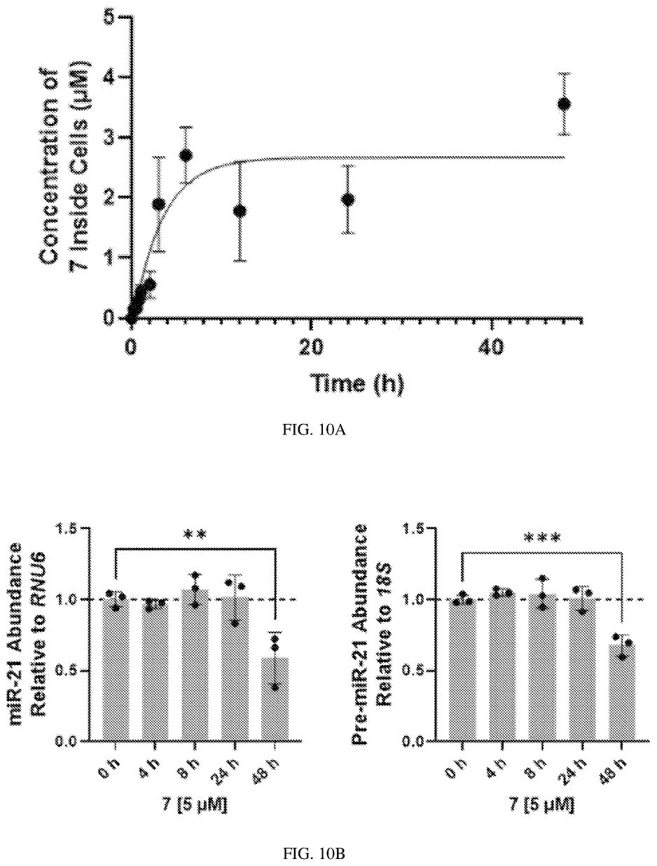

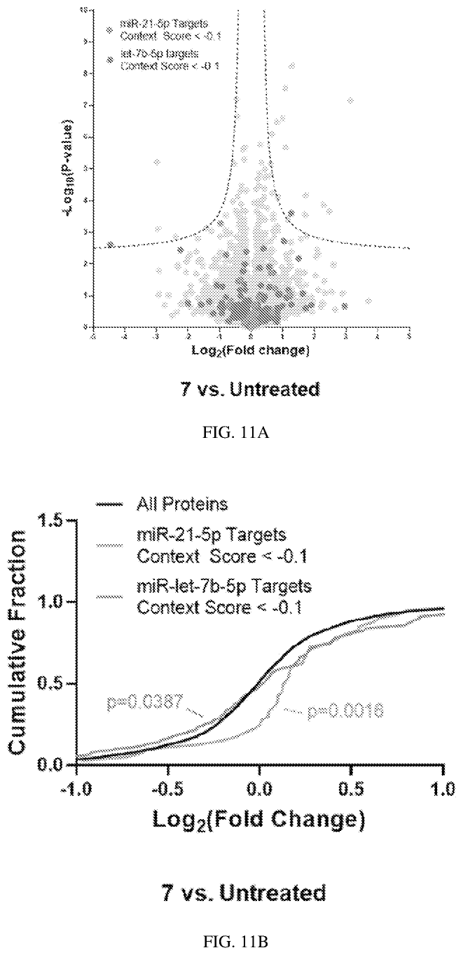

A time-course for RiboTAC degradation of pre-miR-21 was completed and compared to its cellular permeability. Permeability studies showed that RiboTAC 7 accumulated in cells at a concentration of 3.6±1.2 μM after 48 h, reaching a plateau after about 8 h and corresponding to an uptake half-life of 2.4 h (FIG. 10A). Additionally, degradation of pre-miR-21 is first detectable after 48 h of treatment with 7 (FIG. 10B).

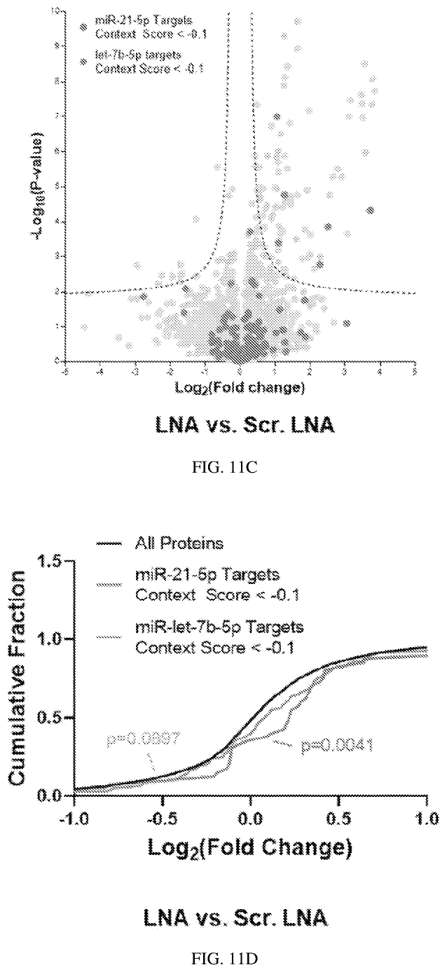

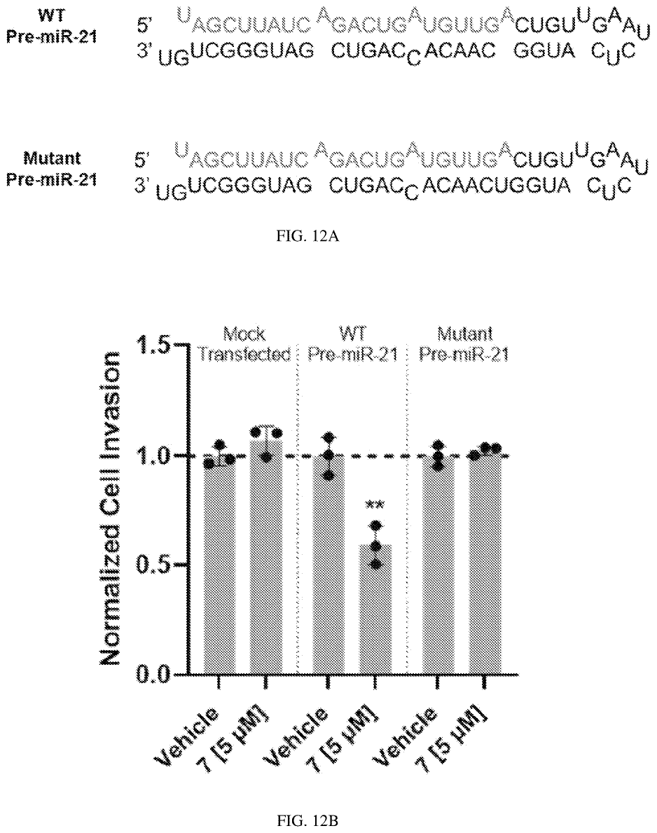

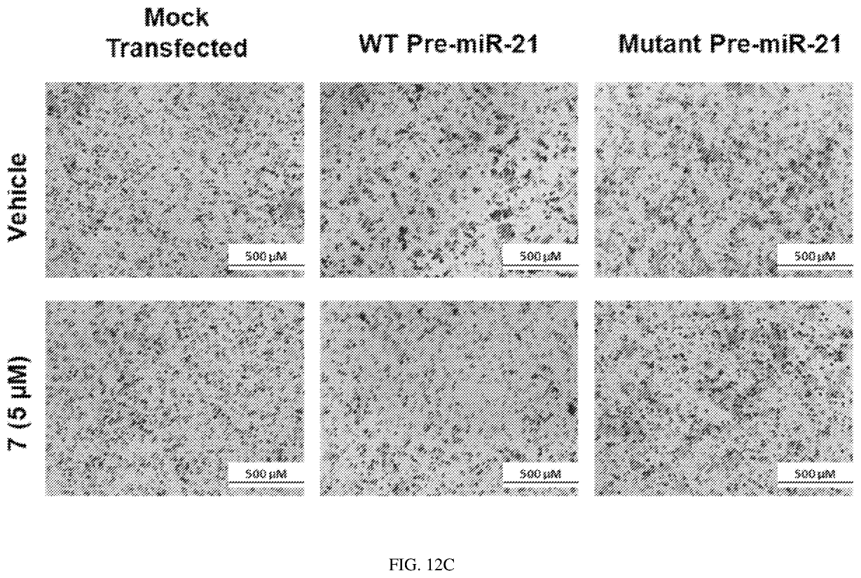

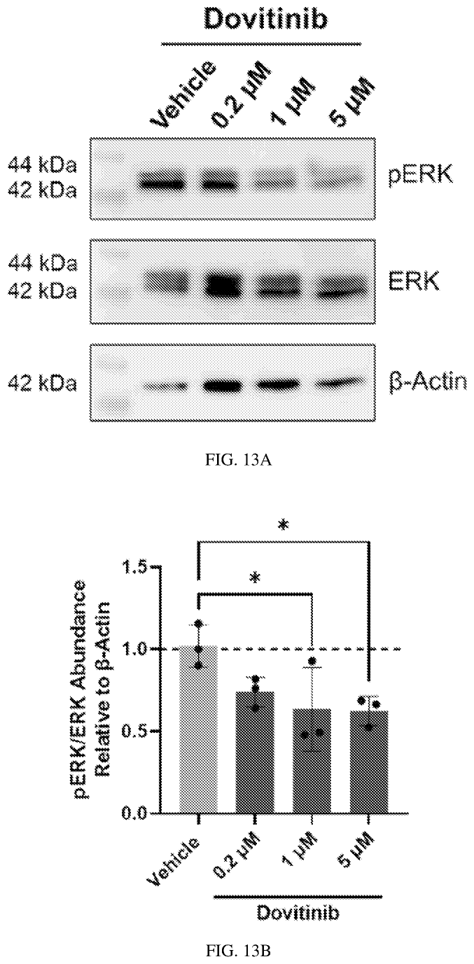

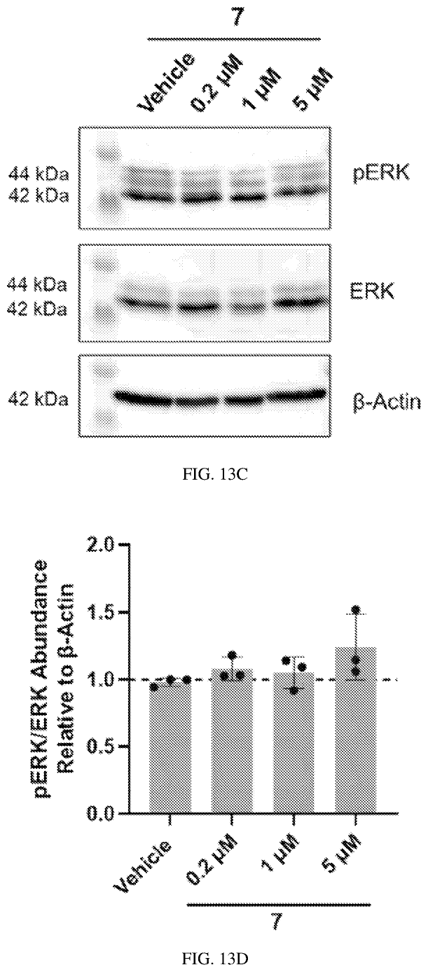

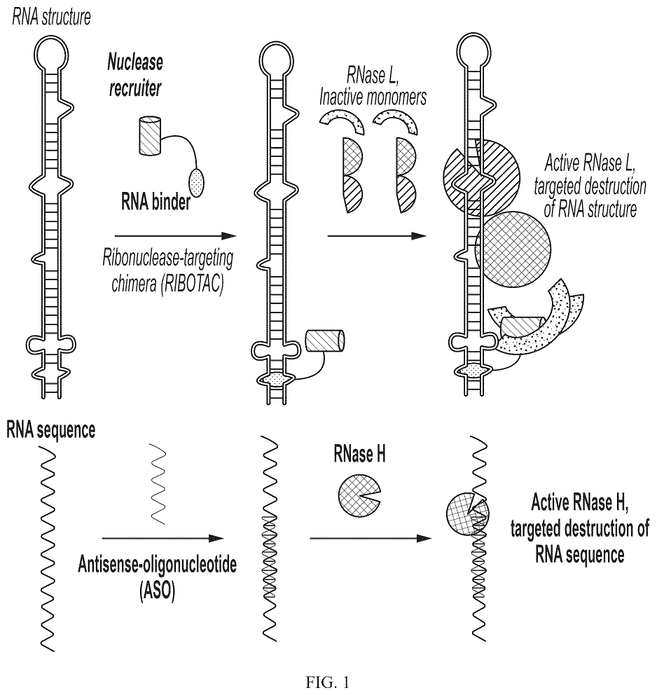

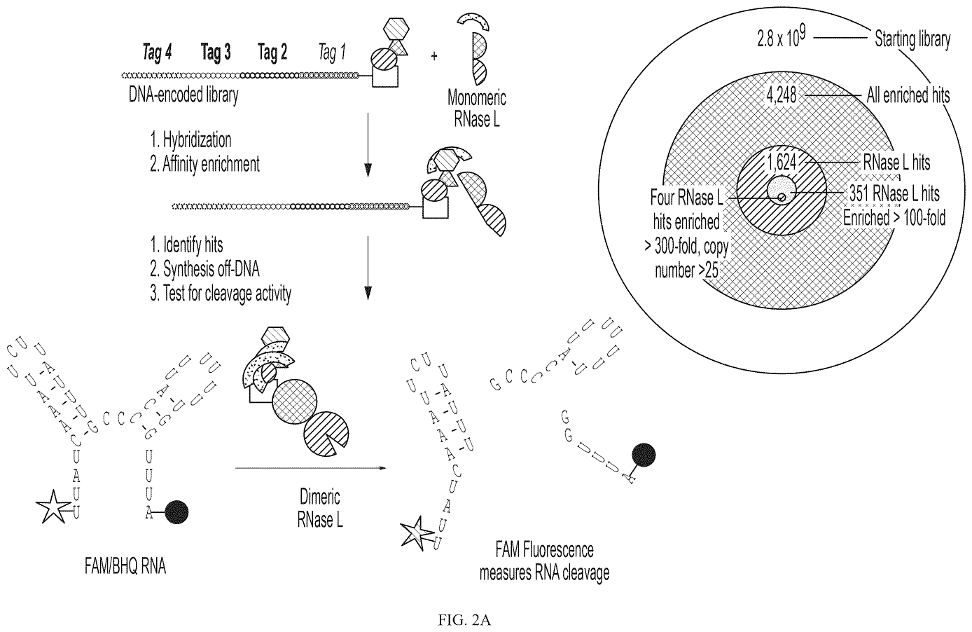

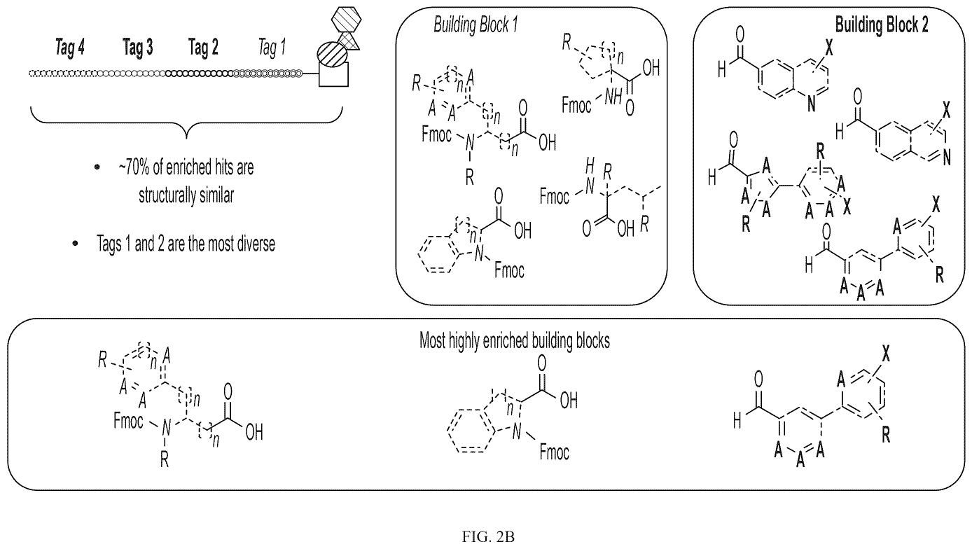

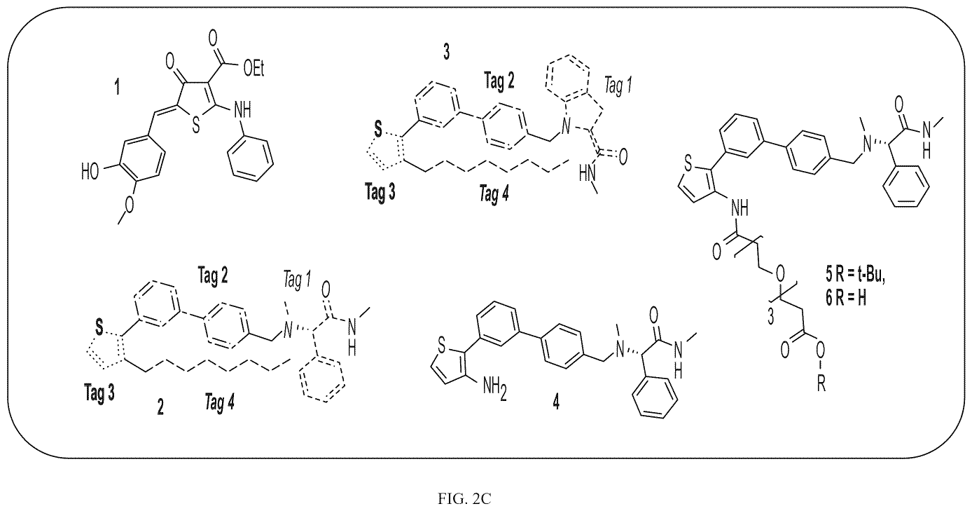

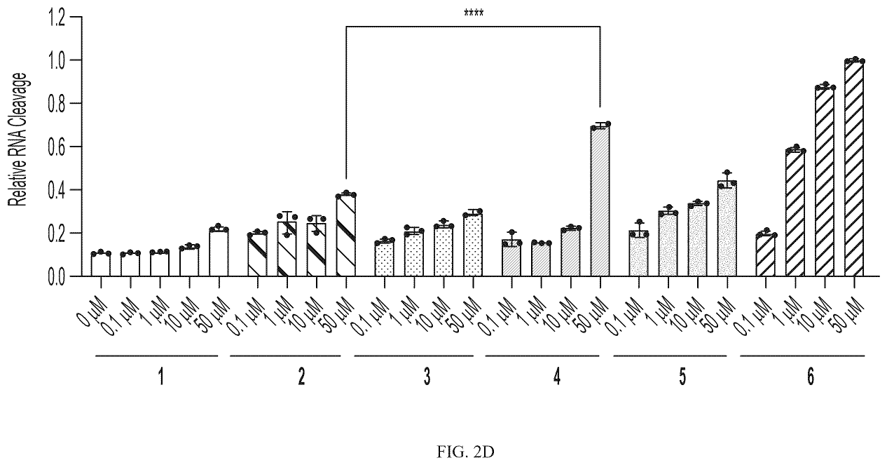

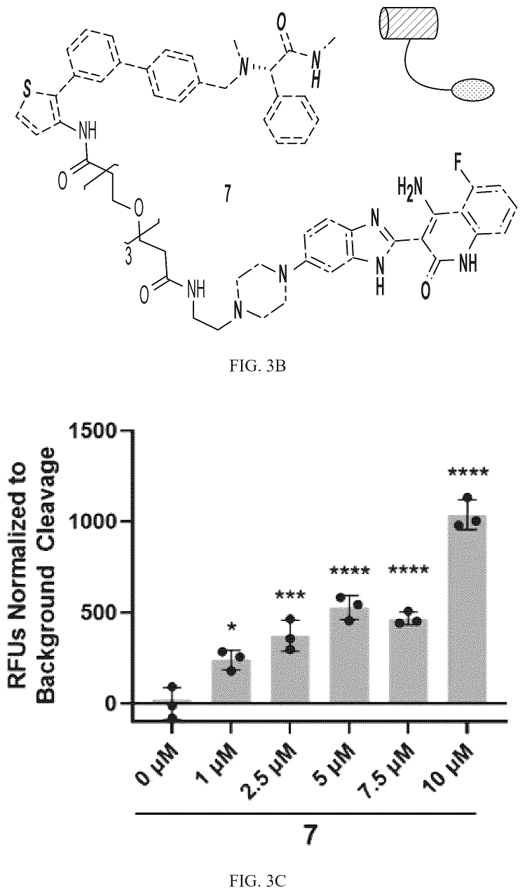

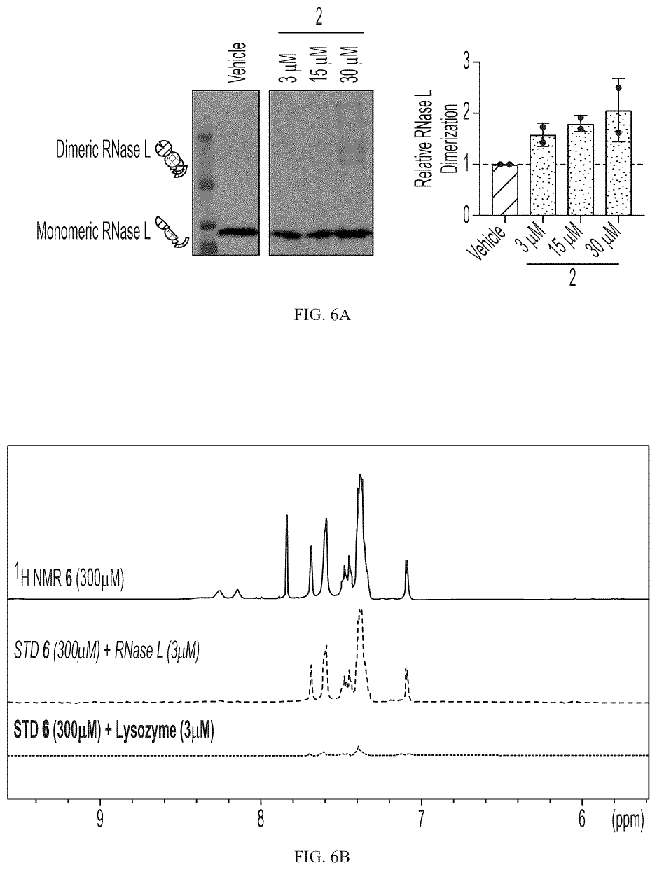

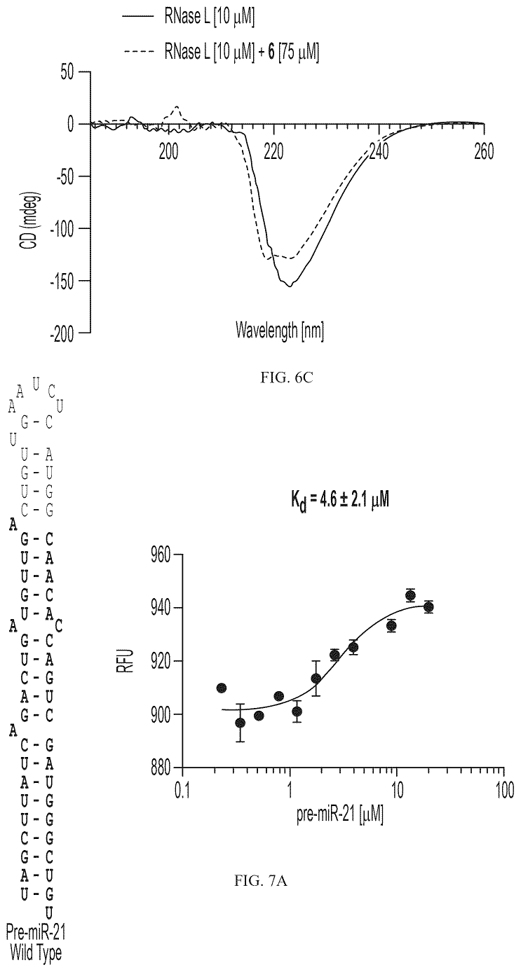

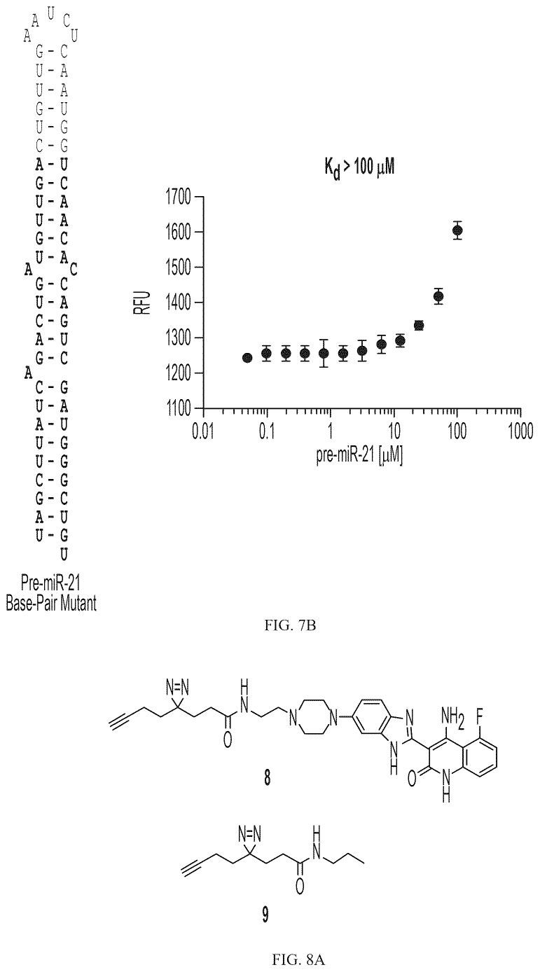

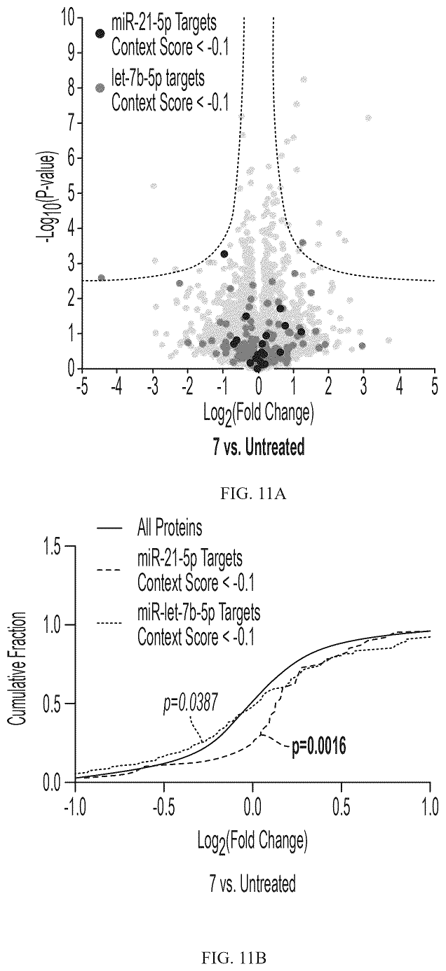

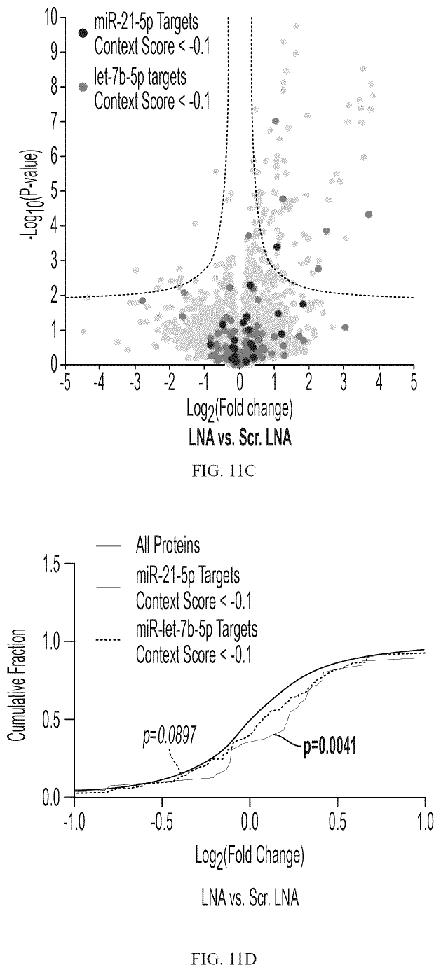

RiboTAC 7 was then assessed for its ability to affect protein levels by deactivation of a miR-21-mediated cancer circuit, i.e., derepression of PDCD4. Western blot analysis of PDCD4 showed protein levels were increased by ˜40% (p<0.01) after treatment with 7 (5 μM) in the CRISPR-Control sgRNA MDA-MB-231 cells (express RNase L; FIG. 4D). Proteome-wide analysis was also completed to study the effects of 7 and a locked nucleic acid (LNA) oligonucleotide that targets miR-21-5p on the proteome. The concentrations tested of both provided similar activity (5 nM of 7 and 100 nM of the LNA), as assessed by their effect on PDCD4 levels, ˜40% enhancement. Global analysis showed that of the 3176 proteins detected, 0.6% and 3.1% were affected by treatment with 7 and the LNA, respectively (FIG. 11A-11B). A cumulative distribution analysis was completed to study the effect on the abundance of proteins that are encoded by mRNAs with a miR-21-5p binding site in their 3′ UTRs, as determined by Human TargetScan v.7.2.³² This cumulative analysis showed a statistically significant increase in the abundance of these miR-21-repressed proteins upon treatment with 7 (p=0.0016) or the LNA (p=0.0041). Neither modality affected proteins that are direct targets of miRNA let-7b-5p, further supporting that RiboTAC 7 is selectively deactivating a miR-21-mediated cancer circuit in cells (FIG. 11C-11D).

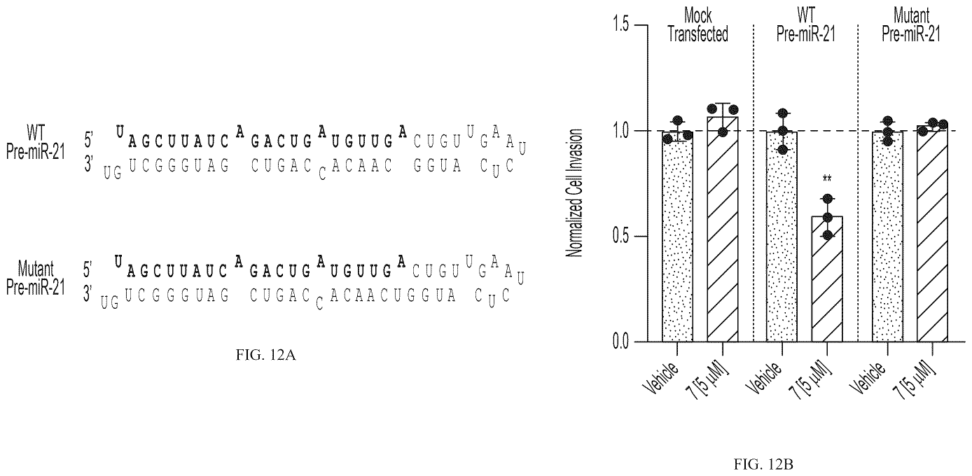

The effect of the RiboTAC on a miR-21-associated invasive phenotype was next assessed. Treatment with 5 μM of 7 reduced the invasiveness of MDA-MB-231 cells, by ˜30% (p<0.05) (FIG. 4E-F). Forced expression of pre-miR-21 induced an invasive phenotype in MCF-10A cells, a model of healthy breast epithelial cells that do not aberrantly express miR-21.³³ This phenotype was rescued by 7-treatment (FIG. 12). While forced expression of a mutant pre-miR-21 in which the Dovitinib-binding site is mutated also produced an invasive phenotype, the cells were insensitive to treatment with 7 (FIG. 12).

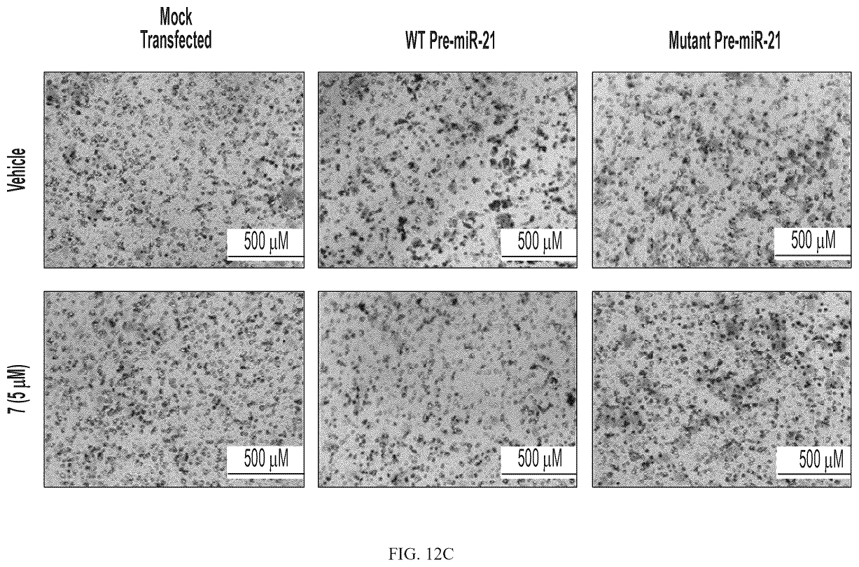

Although transcriptome- and proteome-wide studies did not suggest this alleviation of invasion is due to inhibition of RTKs, the RiboTAC mode of action was verified by studying its effect on RTK signaling, namely the phosphorylation of extracellular signal-regulated kinase (ERK) to afford pERK.³⁴⁻³⁵ Dovitinib dose-dependently inhibited phosphorylation of ERK with an IC50 of ˜1 μM (FIG. 13) in agreement with previous reports.³⁴⁻³⁵ In contrast, RiboTAC 7 had no effect on the levels of ERK or pERK (FIG. 13). Collectively, these data support 7's selectivity for the structured Dicer processing site in pre-miR-21 and that rescue of the invasive phenotype is due to reduction of miR-21.

Previous work to define heterocyclic ligands that activate RNase L were identified by using an enzymatic activity screen.¹⁶ As shown in the current approach, a simple, democratized DEL binding screen can identify various binders of RNase L. Although the compounds were identified by a simple binding screen, two of the four top compounds were validated to also activate RNase L. A top activator was then used as a ribonuclease recruiter in a RiboTAC, providing novel chemical matter that can be used to recruit effector domains. 1 and 4 both contain a thiophene-like ring substituted with an amine, suggesting this chemical motif may contribute to interactions with RNase L. Additionally, both recruiters demonstrate similar activities against miR-21-driven pathologies when incorporated into RiboTACs, leaving further opportunity for optimization of RNase L-recruiting small molecules. The RNA-binding module used herein is an RTK inhibitor, reprogrammed for activity and selectivity of an RNA offtarget.¹⁸ The off-target, miR-21, was identified from a selection platform that studies the RNA-binding capacity of small molecules and known drugs. Those studies¹⁸ as well as another³⁶ demonstrated that protein-targeted small molecules bind RNA and modulate RNA function in cells.

### REFERENCES

1. Burslem, G. M.; Crews, C. M., Proteolysis-targeting chimeras as therapeutics and tools for biological discovery. Cell 2020, 181 (1), 102-114.
2. Costales, M. G.; Matsumoto, Y.; Velagapudi, S. P.; Disney, M. D., Small molecule targeted recruitment of a nuclease to RNA. J. Am. Chem. Soc. 2018, 140 (22), 6741-6744.
3. Roberts, T. C.; Langer, R.; Wood, M. J. A., Advances in oligonucleotide drug delivery. Nat. Rev. Drug Discov. 2020, 19 (10), 673-694.
4. Velagapudi, S. P.; Gallo, S. M.; Disney, M. D., Sequence-based design of bioactive small molecules that target precursor microRNAs. Nat. Chem. Biol. 2014, 10 (4), 291-7.
5. Disney, M. D.; Labuda, L. P.; Paul, D. J.; Poplawski, S. G.; Pushechnikov, A.; Tran, T.; Velagapudi, S. P.; Wu, M.; Childs-Disney, J. L., Two-dimensional combinatorial screening identifies specific aminoglycoside-RNA internal loop partners. J. Am. Chem. Soc. 2008, 130 (33), 11185-94.
6. Sztuba-Solinska, J.; Shenoy, S. R.; Gareiss, P.; Krumpe, L. R. H.; Le Grice, S. F. J.; O'Keefe, B. R.; Schneekloth, J. S., Identification of biologically active, HIV TAR RNA-binding Small molecules using small molecule microarrays. J. Am. Chem. Soc. 2014, 136 (23), 8402-8410.
7. Chen, J. L.; Zhang, P.; Abe, M.; Aikawa, H.; Zhang, L.; Frank, A. J.; Zembryski, T.; Hubbs, C.; Park, H.; Withka, J.; Steppan, C.; Rogers, L.; Cabral, S.; Pettersson, M.; Wager, T. T.; Fountain, M. A.; Rumbaugh, G.; Childs-Disney, J. L.; Disney, M. D., Design, optimization, and study of small molecules that target tau pre-mRNA and affect splicing. J. Am. Chem. Soc. 2020, 142 (19), 8706-8727.
8. Andralojc, W.; Ravera, E.; Salmon, L.; Parigi, G.; Al—Hashimi, H. M.; Luchinat, C., Inter-helical conformational preferences of HIV-1 TAR-RNA from maximum occurrence analysis of NMR data and molecular dynamics simulations. Phys. Chem. Chem. Phys. 2016, 18 (8), 5743-5752.
9. Stelzer, A. C.; Frank, A. T.; Kratz, J. D.; Swanson, M. D.; Gonzalez-Hernandez, M. J.; Lee, J.; Andricioaei, I.; Markovitz, D. M.; Al-Hashimi, H. M., Discovery of selective bioactive small molecules by targeting an RNA dynamic ensemble. Nat. Chem. Biol. 2011, 7 (8), 553-559.
10. Shortridge, M. D.; Walker, M. J.; Pavelitz, T.; Chen, Y.; Yang, W.; Varani, G., A macrocyclic peptide ligand binds the oncogenic microRNA-21 precursor and suppresses Dicer processing. ACS Chem. Biol. 2017, 12 (6), 1611-1620.
11. Han, Y.; Donovan, J.; Rath, S.; Whitney, G.; Chitrakar, A.; Korennykh, A., Structure of human RNase L reveals the basis for regulated RNA decay in the IFN response. Science 2014, 343 (6176), 1244-1248.
12. Washenberger, C. L.; Han, J.-Q.; Kechris, K. J.; Jha, B. K.; Silverman, R. H.; Barton, D. J., Hepatitis C virus RNA: dinucleotide frequencies and cleavage by RNase L. Virus Res. 2007, 130 (1), 85-95.
13. Mannocci, L.; Leimbacher, M.; Wichert, M.; Scheuermann, J.; Neri, D., 20 years of DNA-encoded chemical libraries. Chem Commun. 2011, 47 (48), 12747-12753.
14. Disch, J. S.; Duffy, J. M.; Lee, E. C. Y.; Gikunju, D.; Chan, B.; Levin, B.; Monteiro, M. I.; Talcott, S. A.; Lau, A. C.; Zhou, F.; Kozhushnyan, A.; Westlund, N. E.; Mullins, P. B.; Yu, Y.; von Rechenberg, M.; Zhang, J.; Arnautova, Y. A.; Liu, Y.; Zhang, Y.; McRiner, A. J.; Keefe, A. D.; Kohlmann, A.; Clark, M. A.; Cuozzo, J. W.; Huguet, C.; Arora, S., Bispecific Estrogen Receptor a Degraders Incorporating Novel Binders Identified Using DNA-Encoded Chemical Library Screening. J. Med. Chem. 2021, 64 (8), 5049-5066.
15. Lerner, R. A.; Brenner, S., DNA-Encoded compound libraries as open source: a powerful pathway to new drugs. Angew. Chem. Int. Ed. Engl. 2017, 56 (5), 1164-1165.
16. Thakur, C. S.; Jha, B. K.; Dong, B.; Gupta, J. D.; Silverman, K. M.; Mao, H.; Sawai, H.; Nakamura, A. O.; Banerjee, A. K.; Gudkov, A.; Silverman, R. H., Small-molecule activators of RNase L with broad-spectrum antiviral activity. Proc. Natl. Acad. Sci. U.S.A 2007, 104 (23), 9585-9590.
17. Costales, M. G.; Aikawa, H.; Li, Y.; Childs-Disney, J. L.; Abegg, D.; Hoch, D. G.; Velagapudi, S. P.; Nakai, Y.; Khan, T.; Wang, K. W.; Yildirim, I.; Adibekian, A.; Wang, E. T.; Disney, M. D., Small-molecule targeted recruitment of a nuclease to cleave an oncogenic RNA in a mouse model of metastatic cancer. Proc. Natl. Acad. Sci. U.S.A 2020, 117 (5), 2406-2411.
18. Zhang, P.; Liu, X.; Abegg, D.; Tanaka, T.; Tong, Y.; Benhamou, R. I.; Baisden, J.; Crynen, G.; Meyer, S. M.; Cameron, M. D.; Chatterjee, A. K.; Adibekian, A.; Childs-Disney, J. L.; Disney, M. D., Reprogramming of protein-targeted small-molecule medicines to RNA by ribonuclease recruitment. J. Am. Chem. Soc. 2021, 143 (33), 13044-13055.
19. Troup, R. I.; Fallan, C.; Baud, M. G. J., Current strategies for the design of PROTAC linkers: a critical review. Explor. Target Antitumor Ther. 2020, 1 (5), 273-312.
20. Daou, S.; Talukdar, M.; Tang, J.; Dong, B.; Banerjee, S.; Li, Y.; Duffy, N. M.; Ogunjimi, A. A.; Gaughan, C.; Jha, B. K.; Gish, G.; Tavernier, N.; Mao, D.; Weiss, S. R.; Huang, H.; Silverman, R. H.; Sicheri, F., A phenolic small molecule inhibitor of RNase L prevents cell death from ADAR1 deficiency. Proc. Natl. Acad. Sci. U.S.A 2020, 117 (40), 24802-24812.
21. Greenfield, N. J., Using circular dichroism spectra to estimate protein secondary structure. Nat. Protoc. 2006, 1 (6), 2876-2890.
22. Bartel, D. P., MicroRNAs: genomics, biogenesis, mechanism, and function. Cell 2004, 116 (2), 281-97.
23. Krichevsky, A. M.; Gabriely, G., miR-21: a small multifaceted RNA. J. Cell. Mol. Med. 2009, 13 (1), 39-53.
24. Feng, Y.-H.; Tsao, C.-J., Emerging role of microRNA-21 in cancer. Biomed. Rep. 2016, 5 (4), 395-402.
25. Guan, L.; Disney, M. D., Covalent small-molecule-RNA complex formation enables cellular profiling of small-molecule-RNA interactions. Angew. Chem. Int. Ed. Engl. 2013, 52 (38), 10010-10013.
26. Frankel, L. B.; Christoffersen, N. R.; Jacobsen, A.; Lindow, M.; Krogh, A.; Lund, A. H., Programmed Cell Death 4 (PDCD4) is an important functional target of the microRNA miR-21 in breast cancer cells. J. Biol. Chem. 2008, 283 (2), 1026-1033.
27. Zhu, S.; Wu, H.; Wu, F.; Nie, D.; Sheng, S.; Mo, Y.-Y., MicroRNA-21 targets tumor suppressor genes in invasion and metastasis. Cell Res. 2008, 18 (3), 350-359.
28. Wang, Y.; Chen, T.; Huang, H.; Jiang, Y.; Yang, L.; Lin, Z.; He, H.; Liu, T.; Wu, B.; Chen, J.; Kamp, D. W.; Liu, G., miR-363-3p inhibits tumor growth by targeting PCNA in lung adenocarcinoma. Oncotarget 2017, 8 (12), 20133-20144.
29. Toda, H.; Kurozumi, S.; Kijima, Y.; Idichi, T.; Shinden, Y.; Yamada, Y.; Arai, T.; Maemura, K.; Fujii, T.; Horiguchi, J.; Natsugoe, S.; Seki, N., Molecular pathogenesis of triple-negative breast cancer based on microRNA expression signatures: antitumor miR-204-5p targets APIS3. J. Hum. Genet. 2018, 63 (12), 1197-1210.
30. Niu, M.; Shan, M.; Liu, Y.; Song, Y.; Han, J.-g.; Sun, S.; Liang, X.-s.; Zhang, G.-q., DCTPP1, an Oncogene Regulated by miR-378a-3p, Promotes Proliferation of Breast Cancer via DNA Repair Signaling Pathway. Front. Oncol. 2021, 11, 641931.
31. Di, Y.; Jiang, Y.; Shen, X.; Liu, J.; Gao, Y.; Cai, H.; Sun, X.; Ning, D.; Liu, B.; Lei, J.; Jin, S., Downregulation of miR-135b-5p Suppresses Progression of Esophageal Cancer and Contributes to the Effect of Cisplatin. Front. Oncol. 2021, 11, 679348.
32. Agarwal, V.; Bell, G. W.; Nam, J.-W.; Bartel, D. P., Predicting effective microRNA target sties in mammalian mRNAs. eLife 2015, 4, e05005.
33. Yan, L. X.; Wu, Q. N.; Zhang, Y.; Li, Y. Y.; Liao, D. Z.; Hou, J. H.; Fu, J.; Zeng, M. S.; Yun, J. P.; Wu, Q. L.; Zeng, Y. X.; Shao, J. Y., Knockdown of miR-21 in human breast cancer cell lines inhibits proliferation, in vitro migration and in vivotumor growth. Breast Cancer Res. 2011, 13 (1), R2.
34. Kang, C. W.; Jang, K. W.; Sohn, J.; Kim, S.-M.; Pyo, K.-H.; Kim, H.; Yun, M. R.; Kang, H. N.; Kim, H. R.; Lim, S. M.; Moon, Y. W.; Paik, S.; Kim, D. J.; Kim, J. H.; Cho, B. C., Antitumor Activity and Acquired Resistance Mechanism of Dovitinib (TK1258) in RET-Rearranged Lung Adenocarcinoma. Mol. Cancer Ther. 2015, 14 (10), 2238-2248.
35. Gaur, S.; Chen, L.; Ann, V.; Lin, W.-C.; Wang, Y.; Chang, V. H. S.; Hsu, N. Y.; Shia, H.-S.; Yen, Y., Dovitinib synergizes with oxaliplatin in suppressing cell proliferation and inducing apoptosis in colorectal cancer cells regardless of RASRAF mutation status. Mol. Cancer Ther. 2014, 13 (21).
36. Velagapudi, S. P.; Costales, M. G.; Vummidi, B. R.; Nakai, Y.; Angelbello, A. J.; Tran, T.; Haniff, H. S.; Matsumoto, Y.; Wang, Z. F.; Chatterjee, A. K.; Childs-Disney, J. L.; Disney, M. D., Approved anti-cancer drugs target oncogenic non-coding RNAs. Cell Chem. Biol. 2018, 25 (9), 1086-1094.
37. Ishida, T.; Ciulli, A., E3 ligase ligands for PROTACs: how they were found and how to discover new ones. SLAS Discov. 2021, 26 (4), 484-502.
38. Spradlin, J. N.; Hu, X.; Ward, C. C.; Brittain, S. M.; Jones, M. D.; Ou, L.; To, M.; Proudfoot, A.; Ornelas, E.; Woldegiorgis, M.; Olzmann, J. A.; Bussiere, D. E.; Thomas, J. R.; Tallarico, J. A.; McKenna, J. M.; Schirle, M.; Maimone, T. J.; Nomura, D. K., Harnessing the anti-cancer natural product nimbolide for targeted protein degradation. Nat. Chem. Biol. 2019, 15 (7), 747-755.
39. Zhang, X.; Crowley, V. M.; Wucherpfennig, T. G.; Dix, M. M.; Cravatt, B. F., Electrophilic PROTACs that degrade nuclear proteins by engaging DCAF16. Nat. Chem. Biol. 2019, 15 (7), 737-746.

### Example 1: In Vitro Methods

Production of Inactive RNase L Protein. The inactive RNase L plasmid (pGEX-6p-GSTInactive RNase L) encodes RNase L with a single codon mutation (H672N) in the catalytic site4 that renders the ribonuclease inactive (constructed by Atum). Protein was expressed by transforming this plasmid into BL21 AI Escherichia coli (FisherScientific) followed by culturing in autoinduction media (LB medium supplemented with 1 mM MgSO4, 1×5052, 1×NPS, 100 μg/mL ampicillin) for 5 h at 37° C. with shaking. The culture was then cooled to 18° C., induced with 200 μM IPTG and cultured at 37° C. for an additional 24 h. Cells were pelleted by centrifugation and lysed by sonication in 1× Inactive RNase L Buffer (20 mM Na+ HEPES, pH 7.4, 300 mM NaCl, 5 mM MgCl2, 0.1 mM EDTA, and 5 mM DTT). The lysate was then incubated with Pierce™ glutathione agarose resin (3 mL, ThermoFisher) for 3 h at 4° C. The resin was then pelleted by centrifugation, and the supernatant was removed. After washing three times with 1× Inactive RNase L Buffer, the resin was incubated with PreScission Protease (30 μL, GE Healthcare—Life Sciences) in 3 mL of 1× Inactive RNase L Buffer overnight at 4° C. Purified inactive RNase L was eluted with 1× Inactive RNase L Buffer, leaving the GST tag resin-bound. The purified protein was stored in 1× Inactive RNase L Storage Buffer at −80° C.

Production of Active GST-RNase L-His Protein. The active RNase L plasmid (pGEX-4T-GSTRNase L-6×His) was prepared by modifying the previously described pGEX-4T-GST-RNase L expression plasmid^{3, 5} to add a 6×His tag to the C-terminus (Genscript). Active RNase L protein was produced by transforming the pGEX-4T-GST-RNase L-6×His plasmid into Rosetta DE3 competent E. coli (Novagen). E. coli were then cultured in autoinduction medium (LB medium supplemented with 1 mM MgSO4, 1×5052, 1×NPS, 100 μg/mL ampicillin) for 36 h at room temperature with shaking. Cells were pelleted and lysed by sonication in 1× Lysis Buffer (32 mM Na2HPO4, 8 mM KH2PO4, pH 7.4, 20 mM MgCl2, 550 mL NaCl, 10 mM KCl, 4 mM EDTA, 40 mM β-mercaptoethanol, 0.1 mM ATP, and 40% (w/v) glycerol). Briefly, the lysate was loaded onto Pierce™ glutathione agarose resin (3 mL, ThermoFisher), self-packed in a gravity column. After washing the resin with 1× Lysis Buffer (50 mL), RNasse L was eluted from the column using 1× Glutathione Elution Buffer (50 mM Tris, pH 8.8, 20 mM glutathione, 100 mM KCl, 2 mM EDTA, 10 mM βmercaptoethanol, 10 mM MgCl2, 0.2 mM ATP, and 0.1% (v/v) Triton-X). To remove spontaneously cleaved GST tags, the eluted protein was purified using a second gravity column packed with His60 Ni Superflow resin (Takara). In brief, the buffer of the eluted protein was exchanged to 1× Equilibration Buffer (50 mM Na2HPO4, pH 7.4, 20 mM imidazole, and 300 mM NaCl) by using an Amicon Ultra-15 10K centrifugal filter (Millipore) and then loaded onto the His60 column. The resin was then washed with 50 mL of 1×His60 Wash Buffer (50 mM Na2HPO4, pH 7.4, 40 mM imidazole, and 300 mM NaCl), and protein was eluted in 1×His60 Elution Buffer (50 mM Na2HPO4, pH 7.4, 500 mM imidazole, and 300 mM NaCl). The buffer of the purified recombinant protein was exchanged into 1× Active RNase L Storage Buffer (20 mM HEPES, pH 7.4, 70 mM NaCl, 2 mM MgCl2, 1 mM DTT, and 25% (v/v) glycerol) and stored at −80° C.

DEL Screening. DEL screening was performed using a 2nd generation DELopen selection kit (9 DEL libraries with a total diversity of 2.8 billion compounds) supplied by WuXi AppTec. Screening was carried out according to the manufacturer's protocol (https://hits.wuxiapptec.com/assets/pdf/DELopen_Protocol.pdf). After three iterative rounds of selection, the samples were returned to WuXi AppTec's facility for further processing including PCR amplification, quantitation of qPCR, and sequencing. All samples passed the standard DELopen quality control criteria and proceeded forward to the sequencing step using an Illumina NovaSeq Platform. Enrichment scores were then calculated using WuXi's proprietary pipeline where the raw data are normalized to enrichment score following the derived algorithm from Kuai. et al., 6 with consideration given to correct copy number, library size, NGS depth, and additional normalization factors. Enrichment scores are assigned based on a compound's abundance in the sequencing data compared to the average molecule in the library, i.e., a compound with an enrichment score of 100 is 100 times more abundant than the average library molecule.

FRET-Based In Vitro RNA Cleavage Assay. Samples were prepared as previously described.³ Briefly, an RNA construct with multiple preferred RNase L cleavage sites⁷ (5′ 6-FAM-UUAUCAAAUUCUUAUUUGCCCCAUUUUUUUGGUUUA-3′ BHQ; 100 nM final concentration) was folded in 1× RNase L Assay Buffer (25 mM Tris-HCl, pH 7.4, and 100 mM NaCl) for 1 min at 95° C. followed by snap cooling on ice for 10 min. Following cooling, β-mercaptoethanol (7 mM), ATP (50 μM), and MgCl2 (1 mM) were added. Compound or DMSO (1%; vehicle) was added to the folded RNA, and the samples were incubated for 15 min. Recombinant GST-RNase L-His was then added to the solution (50 nM final concentration), affording a total volume of 33 μL. Technical triplicates (10 μL) were dispensed into 384-well low volume non-binding plate (Corning 4515), and the samples were incubated in the dark for 1 h. Fluorescence was measured using a Tecan infinite M1000 Pro plate reader using an excitation wavelength of 485 nm, emission wavelength of 520 nm, and gain of 100. Data are reported as “Relative RNA Cleavage”, normalizing fluorescence to the highest level of cleavage observed—upon addition of 50 μM of 6, which was set to 1.

Saturation Transfer Difference (STD) NMR. NMR spectra were acquired at 25° C. on a 600 MHz Bruker Avance III spectrometer equipped with a cryoprobe. Samples were prepared by drying 6 (120 nmol) to a solid and resuspending in deuterated DMSO (4 μL). This solution was then added to 1× Inactive RNase L Storage Buffer (400 μL). Inactive RNase L or lysozyme was then added to the solution to afford final concentrations of 3 μM protein and 300 μM compound. The STD spectra were recorded as an interleaved experiment (n=2) with one experiment including a saturating pulse at −40 ppm for 2 s and one at 0.8 ppm for 2 s. Both spectra were processed with identical phasing before calculating the difference spectra (A) using TopSpin 4.0.6 (Bruker).

Circular Dichroism (CD) Spectroscopy. Inactive RNase L (10 μM) was prepared in 1×CD Buffer (80 mM K2HPO4, 20 mM KH2PO4, pH 7.0, and 50 mM KF). CD wavelength scans were completed from 190 to 260 nm. Compound was added to the inactive RNase L sample (75 μM; 0.1% DMSO final concentration), and CD spectra were collected. The spectra were plotted and compared in GraphPad Prism.

Binding Affinity Measurements. Binding assays were completed as previously described.8 Briefly, WT pre-miR-21 (5′-UAGCUUAUCAGCUGAUGUUGACUGUUGAAUCUCAUGG CAACACCAGUCGAUGGGCUGU-3′) or a mutant pre-miR-21 with the A-bulge and U-bulges replaced with base pairs (5′-UAGCUUAUCAGCUGAUGUUGACUGUUGAAUCUCAAUG GUCAACACCAGUCGAUGGGCUGU-3′) was folded in 1× Assay Buffer (8 mM Na2HPO4, pH 7.0, 185 mM NaCl, and 1 mM EDTA) by heating at 95° C. for 1 min followed by cooling on ice for 10 min. After cooling, BSA was added to a final concentration of 40 μg/mL. RiboTAC 7 was then added to the solution to a final concentration of 0.5 μM. The RNA solution was serial diluted with 1× Assay Buffer supplemented with 40 μg/mL BSA and 0.5 μM of 7. The solutions were incubated in the dark for 30 min at room temperature before being transferred to a black 384-well plate (Corning 4514, low-volume, non-binding) Fluorescence intensity was measured using a Tecan infinite M1000 Pro (Gain: 100) with excitation wavelength of 380 nm and emission wavelength of 425 nm. Binding affinity was calculated using Equation 1, as previously described:8

$$I = {I}_{0} + 0.5 Δε \{ ( {[ FL ]}_{0} + {[ R N A ]}_{D} + {K}_{d} ) - {( {( {[ FL ]}_{0} + {[ R N A ]}_{0} + {K}_{d} )}^{2} - {{4 [ FL ]}_{0} [ R N A ]}_{0} )}^{0.5} \} \qquad ( Eq . 1 )$$

where I and I0 are the measured fluorescence intensity of compound with or without RNA, respectively, Δε is the difference between the fluorescence intensity with infinite concentration of RNA versus without RNA, [FL]₀ and [RNA]₀ are the concentrations of small molecule and RNA, respectively, and Kd is the dissociation constant.

In Vitro C-Chem-CLIP. Growth medium (RPMI 1640 medium supplemented with 300 mg/L Lglutamine & 25 mM HEPES; Corning, catalog #10-041-CV)] was inactivated by heating to 95° C. for 10 min followed by slowly cooling to room temperature. Pre-miR-21 was radioactively labeled using [γ-32P]ATP and T4 polynucleotide kinase and purified on a 15% denaturing gel, as previously described.^{1, 9} The RNA (˜5000 cpm per sample) was folded in the inactivated growth medium by heating at 95° C. for 2 min and cooled to room temperature. RiboTAC 7 (1, 5, 20, or 100 μM) or DMSO (vehicle) was added to the folded RNA and incubated at 37° C. for 30 min. Then Chem-CLIP probe 8 (5 μM) or negative control probe 9 was added to the mixture and incubated at 37° C. for another 30 min. The samples were cross-linked under UV light (365 nm) for 10 min.

Copper-catalyzed click chemistry¹⁰ was used to click the probes in each sample to biotinazide (1265-5, Click Chemistry Tools). The click reaction was carried out through the addition of click chemistry reagents [10 mM CuSO4 (1 μL); 50 mM tris-hydroxypropyltriazolylmethylamine (THPTA; 1 μL); 10 mM biotin-azide (1 μL); and 250 mM sodium ascorbate, pH 7.0 (1 μL). The reaction mixtures were incubated at 37° C. for 2 h. Streptavidin beads (Invitrogen; 15 μL of slurry per sample) were then added to each sample, which were then incubated for an additional 30 min at room temperature to pull-down cross-linked RNA. The samples were washed three times with 1×PBST (phosphate buffered saline containing 0.1% (v/v) Tween-20). Radioactive signal from both the bound RNA on the magnetic beads and the unbound RNA in the wash solutions were measured using a Beckman rya Coulter LS6500 Liquid Scintillation Counter. The percentage pulldown by Chem-CLIP probe was calculated according to equation 2:

$$Cbound / ( Cbound + Cunbound ) \qquad ( Eq . 2 )$$

Co-Immunoprecipitation of pre-miR-2I and RNase L. Approximately 20,000 cpm of 32P 5′-end labeled WT or mutant pre-miR-21 or an RNA construct where the A-bulge was mutated to a base pair (5′-UAGCUUAUCAGCUGAUGUUGACUGUUGAAUCUCAUGGUCAACACCAGUCGA UGGGCUGU-3′) was folded in 1× RNase L Assay Buffer for 1 min at 95° C. and cooled on ice for 10 min. Following cooling, β-mercaptoethanol (7 mM), ATP (50 μM), and MgCl2 (1 mM) were added to the solution. The solution was aliquoted, and compounds were incubated in solution for 15 min (1% DMSO final concentration). Inactive RNase L (100 nM final concentration) was added (22 μL total volume per sample), and the reactions were incubated for an additional 1 h at room temperature before being tumbled overnight with Dynabeads Protein A (10 μL per sample, Life Technologies) bound to an anti-RNase L primary antibody (Cell Signaling Technology, D4B4J, 1:10), at 4° C. Beads were then washed with 1×PBS supplemented with 0.02% (v/v) Tween-20. Radioactive signal in the supernatant and associated with the beads was quantified by a Beckman Coulter LS6500 Liquid Scintillation Counter. Relative RNA enrichment was calculated by comparing the radioactive signal associated with beads compared to the total radioactive signal from the beads plus the supernatant.

### Example 2: Cellular Methods

Cell Culture. Cells were cultured at 37° C. with 5% CO2 and used in experiments at passage number 20 or less. MDA-MB-231 cells (HTB-26, ATCC) were cultured in RPMI 1640 medium with L-glutamine and 25 mM HEPES supplemented with 1× Antibiotic/Antimycotic solution (Corning) and 10% fetal bovine serum (FBS; Sigma, catalog #12003C). MCF-10A cells (CRL-10317, ATCC) were cultured in 1×DMEM/F12 with L-glutamine & 15 mM HEPES (Corning) supplemented with 20% FBS, 1× Antibiotic/Antimycotic solution, 20 ng/mL human epidermal growth factor (Pepro Tech, Inc.), 10 μg/mL insulin (Sigma-Aldrich), and 0.5 mg/mL hydrocortisone (Pfaltz & Bauer).

MDA-MB-231 cells were treated with compound (0.1% DMSO final concentration) for 24 h, followed by removal of the compound-containing medium and redosing in growth medium for an additional 24 h (total treatment period of 48 h), with the exception of invasion assays in which the cells were treated overnight. For the time-course experiment, MDA-MB-231 cells were treated for the indicated lengths of time. MCF-10A cells, also used in invasion assays, were treated with compound overnight (0.1% DMSO final concentration).

LNA-21, a locked nucleic acid targeting miR-21-5p (#YI04100689-DFA, Power Inhibitor) and the scrambled control LNA (#YI00199006-DFA, Power Inhibitor) were purchased from Qiagen and treated in medium at 100 nM in all cellular experiments.

Generation of the RNase L Knockdown MDA-MB-231 Cell Line. RNase L knockdown and control MDA-MB-231 cell lines were generated by CRISPR as previously described.¹ Briefly, lentiviral constructs containing Cas9 and gRNAs against RNASEL or a negative control were purchased from Transomic Technologies, Inc. [catalog #TEVH-1249025-pCLIP-ALL-hCMVPuro (KD+) and TELA1015 (KD−)] and packaged by transfecting HEK293T cells (ATCC, catalog #CRL-11268) using Lipofectamine 2000 transfection reagent (Invitrogen; catalog #11668500) and Cellecta packaging mix (Cellecta, catalog #CPCP-K2A) in Opti-MEM (Gibco, catalog #31985070). Virus supernatants were harvested 72 h post-transfection and filtered through a 0.45 μm PES filter (mdi Membrane Technologies Inc., catalog #SYPL0602MNXX204). The harvested viral particles were then transduced into MDA-MB-231 cells in the presence of 6 μg/mL polybrene (Millipore, catalog #TR-1003-G).

RNA Isolation and Quantitative Real-Time Polymerase Chain Reaction (RT-qPCR). MDAMB-231 cells were cultured as described in “Cell Culture.” After compound treatment, total RNA was extracted using a Quick-RNA Miniprep Kit (Zymo) per the manufacturer's instructions. Reverse transcription (RT) for pre-miRNAs was completed using 200 ng total RNA and a qScript cDNA Synthesis kit (QuantaBio), following the manufacturer's instructions. RT for mature miRNAs was completed using 250 ng total RNA and the Mir-X miRNA First Strand Synthesis Kit (Takara) per the manufacturer's instructions. RT-qPCR was performed on a QuantStudio5™ Real-Time PCR Instrument (Applied Biosystems) using Power SYBR Green Master Mix (Applied Biosystems), and the resulting data were analyzed using the ΔΔCt method.

For global miRNA profiling experiments, RT was completed using 2 μg total RNA and a Mir-X miRNA First Strand Synthesis Kit (Takara) in a total volume of 20 μL. The RT reaction was then plated into the wells of a 384-well qPCR plate containing pre-diluted forward and reverse primers for the miRNA of interest. RT-qPCR was performed on a QuantStudio5™ Real-Time PCR Instrument (Applied Biosystems) using Power SYBR Green Master Mix (Applied Biosystems). Data were analyzed using the ΔΔCt method,¹¹ followed by calculation of log 2 fold changes and −log 10 p-values. The software Perseus was used to calculate the false discovery rate (FDR) of 1% with a group variance of S0(0.1).

RiboTAC Cellular Uptake Quantification. MDA-MB-231 cells were cultured as described in “Cell Culture” with a final cell density of 13,500 cells per well. Cells were treated with 7 (5 μM) for 0, 0.25, 0.5, 0.75, 1, 2, 3, 6, 12, 24, and 48 hours. Following treatment, cells were rapidly washed with 1×PBS to remove any unincorporated compound. Cells were then detached using 75 μl trypsin, immediately homogenized and flash frozen. The cell homogenate was analyzed using a Sciex 6500 mass spectrometer operated in multiple reaction monitoring mode with the transition m/z=1078.3/900.3. Homogenate drug levels were determined by comparison to a standard curve prepared in untreated cell homogenate. Calculated concentrations, (ng drug)/(ml homogenate) or μM drug inside cell, were fit to a one-phase association model using GraphPad Prism.

Quantification of Protein Levels by Western Blotting. MDA-MB-231 cells were treated as described in “Cell Culture.” After treatment, the medium was removed, and the cells were washed with 1× Dulbecco's phosphate-buffered saline (DPBS). Total protein was extracted using Mammalian Protein Extraction Reagent (M-PER, Thermo Scientific) according to the manufacturer's protocol. Protein concentration was measured using a Pierce Micro BCA Protein Assay kit (Fisher Scientific). Approximately 20 μg total protein was loaded onto a Tris-glycine gel with a 5% polyacrylamide stacking layer and a 10% polyacrylamide resolving layer. The gel was run at 140 V for ˜1.5 h in 1× Running Buffer (25 mM Tris base, pH 8.3, 190 mM glycine, and 0.1% (w/v) SDS). After electrophoresis, the protein was transferred to a PVDF membrane using 1× Transfer Buffer (25 mM Tris base, pH 8.3, 190 mM glycine, and 20% (v/v) methanol). The membrane was then washed with 1×TBST (Tris-buffered saline with 0.1% (v/v) Tween 20) and blocked in a solution of 5% (w/v) milk in 1×TBST for 30 min with shaking at room temperature. The membrane was then incubated overnight at 4° C. in a solution of primary antibody diluted in 1×TBST containing 5% (w/v) milk. The membrane was washed three additional times with 1×TBST and then incubated for 1 h at room temperature in a solution of anti-rabbit IgG horseradishperoxidase secondary antibody conjugate (Cell Signaling Technology, 7074S, 1:5000 dilution) in 1×TBST containing 5% (w/v) milk. The membrane was washed three times with 1×TBST and protein was visualized using SuperSignal West Pico Plus Chemiluminescent Substrate (Pierce Biotechnology) per the manufacturer's protocol. Blots were imaged using an Amersham ImageQuant 800 Western blot imager.

The membrane was then stripped using 1× Stripping Buffer (200 mM glycine, pH 2.2, 1% (v/v) Tween-20, and 0.1% (w/v) SDS) and blocked for 30 min at room temperature with a solution of 5% (w/v) milk in 1×TBST. The membrane was incubated overnight at 4° C. in a solution of anti-β-actin (Cell Signaling Technology, 3700S) primary antibody (1:5000 dilution) in 1×TBST containing 5% (w/v) milk. The membrane was washed three times with 1×TBST and incubated in a solution of anti-mouse IgG horseradish-peroxidase secondary antibody conjugate (Cell Signaling Technology, 7076S, 1:10000 dilution) in 1×TBST containing 5% (w/v) milk for 1 h at room temperature. Following incubation, the membrane was washed three times with 1×TBST, and protein expression was visualized using SuperSignal West Pico Plus Chemiluminescent Substrate (Life Technologies) per the manufacturer's protocol and imaged with an Amersham ImageQuant 800 Western blot imager.

Western blots were quantified using ImageJ (National Institutes of Health).¹² Protein expression was normalized to 13-actin abundance.

Antibodies and dilutions: Anti-PDCD4 primary antibody (Cell Signaling Technology, 9535S,1:1000 dilution). Anti-pERK primary antibody (Cell Signaling Technology, 9101S, 1:1000 dilution). Anti-ERK primary antibody (Cell Signaling Technology, 9102S 1:1000 dilution). Anti-β-actin primary antibody (Cell Signaling Technology, 3700S, 1:5000 dilution).

Global proteomics analysis via LC-MS/MS. MDA-MB-231 cells modified with CRISPR and a control guide RNA were treated as described in “Cell Culture.” After treatment, the medium was removed, and the cells were washed with 1×DPBS. After washing, the cells were scraped from the culture plates, and the resulting suspension was centrifuged. Cell pellets were resuspended in 1×DPBS, and protein concentration was determined using a Pierce Micro BCA Protein Assay Kit (ThermoScientific). Urea (9 M in 100 mM NH4HCO3, pH 8) was added to a final concentration of 6 M to 30 μg lysate (15 μL prepared in 1×DPBS). Proteins were reduced with 10 mM tris(2-carboxyethyl)phosphine hydrochloride (TCEP, 20× freshly prepared in water) for 30 min at room temperature, followed by alkylation with 25 mM iodoacetamide (250 mM, fresh prepared in water) for 30 min at room temperature in the dark. Samples were then diluted to 2 M urea with 50 mM NH4HCO3 (pH 8) and digested with trypsin (Thermo scientific, 1.5 μL of 0.5 μg/μL) overnight at 37° C., in the presence of 1 mM CaCl₂). Peptides were desalted over a self-packed C18 spin column, dried, and analyzed via LC-MS/MS. Analysis was completed using the MaxQuant software (V2.0.3.0), as described previously.¹

Boyden Chamber Invasion Assay. Hanging cell culture inserts (for 24-well plates, 8.0 μm) were pre-coated with 0.3 mg/mL Matrigel (Corning, 356234). MDA-MB-231 cells (75,000 per insert) were then seeded into the hanging inserts in serum free growth medium, with or without compound (0.1% final DMSO concentration). The inserts were then placed into 24-well plates containing complete. After 16-24 h, the medium was removed, and the hanging inserts were washed with 1×DPBS. Cells were fixed for 20 min at room temperature with 4% (w/v) paraformaldehyde in 1×DPBS. The inserts were washed twice with 1×DPBS and then stained with 0.1% (w/v) crystal violet in 20% methanol (v/v) for 20 min. The inserts were then washed twice with Nanopure water and once with 1×DPBS. Cells on the surface of the Matrigel (noninvasive) were removed with cotton swabs and invading cells were imaged under a Leica DMI3000 B upright fluorescent microscope (the number of invasive cells in 4 fields of view per sample).

MCF-10A cells were first transfected with either a plasmid expressing pre-miR-21 (Addgene; catalog #21114) or a plasmid encoding a pre-miR-21 mutant (custom purchased from GenScript USA, Inc, FIG. 8) using jetPRIME (Polyplus), per the manufacturer's protocol (3.2 μg of plasmid per 60 mm dish). After transfection, cells (50,000 per insert) were seeded into the hanging inserts in serum free growth medium, with or without compound (0.1% final DMSO concentration). The assay was then completed as described for the MDA-MB-231 cells.

### Example 3: Synthetic Experimental Procedures

General Methods. Reagents and solvents purchased from commercial suppliers were used directly without further purification. Reactions were monitored by thin layer chromatography (TLC, Agela Technologies) or by liquid chromatography-mass spectrometry (LC/MS; Agilent 1260 Infinity LC system coupled to an Agilent 6130 TOF(HR-ESI) equipped with a Poroshell 120 EC-C18 column; 50 mm×4.6 mm, 2.7 μm). TLC bands were visualized under UV light (254 nm).

Products requiring further purification were purified by either silica gel column chromatography or by high-performance liquid chromatography (HPLC). HPLC purification was completed using a Waters 1525 Binary HPLC Pump equipped with a 2489 UV/Visible Detector and a SunFire® Prep C18 OBD™ 5 μm column (19×150 mm) using a flow rate of 5 mL/min. The purity of products was analyzed by HPLC (Waters 2487 and 1525) equipped with a SunFire® C18 3.5 μm column (4.6×150 mm) using a flow rate of 1 mL/min. The linear gradient used for purification and analysis of purity analysis were from 100% H2O+0.1% (v/v) trifluoroacetic acid (TFA) to 100% MeOH+0.1% TFA (v/v) over 60 min unless otherwise noted.

NMR spectra were acquired using a Bruker Ascend™ 600 (600 MHz for 1H and 150 MHz for 13C) or a Bruker 400 UltraShield™ (400 MHz for 1H, 100 MHz for 13C, 376 MHz for 19F). Residual solvents are used as internal standards, and chemical shifts are reported in ppm. Coupling constants (J values) are reported in Hz.

Mass spectra were acquired by an Agilent 1260 Infinity LC system coupled to an Agilent 6130 TOF(HR-ESI) or a 4800 Plus MALDI TOF/TOF Analyzer. Specific optical rotations were measured using a Rudolph Research Analytical AUTOPOL IV polarimeter.

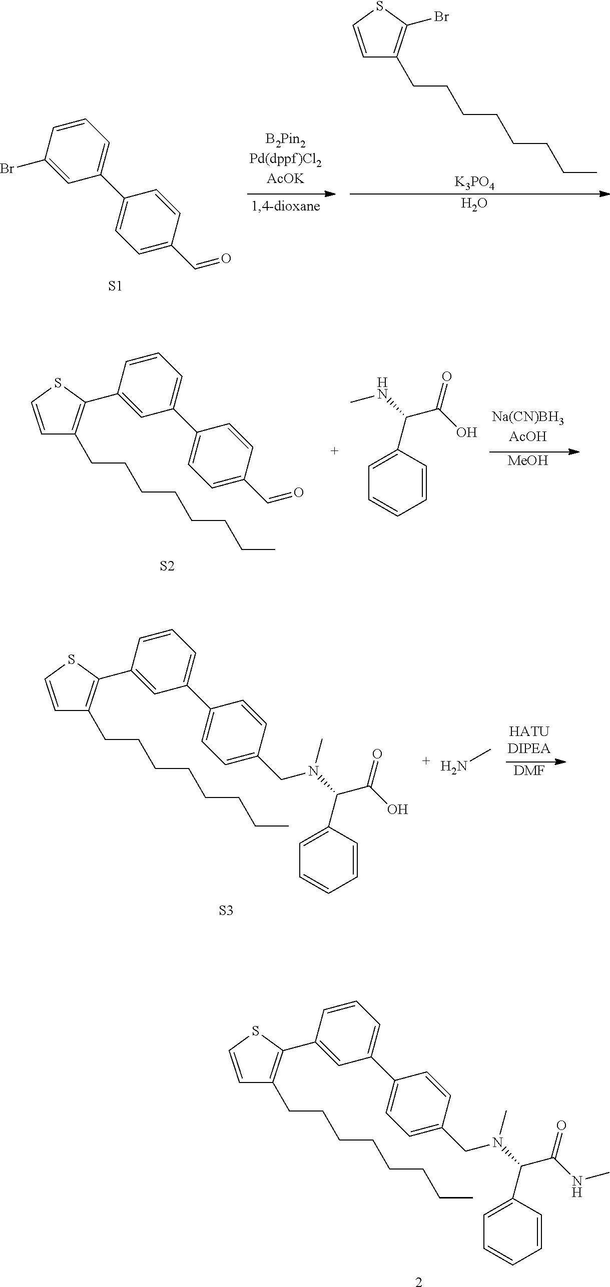

Synthesis of 2. 3′-Bromo-[1,1′-biphenyl]-4-carbaldehyde (S1, 200 mg, 0.766 mmol). bis(pinacolato)diboron (291 mg, 1.15 mmol). potassium acetate (112 mg, 1.15 mmol), and anhydrous 1,4-dioxane (3.8 mL) were added to a round bottom flask and stirred at room temperature for 5 min. The mixture was purged with nitrogen, and a 1,1′-[bis(diphenylphosphino)ferrocene] dichloropalladium dichloromethane adduct (62.6 mg, 0.0766 mmol) was added. The reaction mixture was stirred at 90° C. overnight. After checking for completion of the reaction by LC/MS, 2-bromo-3-octylthiophene (174 μL, 0.766 mmol) and tripotassium phosphate (487 mg, 2.29 mmol) in H2O (2.5 mL) were added, and the reaction was stirred for 2 h. After checking reaction completion by LC/MS, the reaction mixture was filtered and washed with EtOAc, and the filtrate was evaporated. The residue was purified by silica gel column chromatography (0-35% EtOAc in hexane) and by C18 silica column chromatography (0-100% MeOH in H2O) to obtain target material 3′-(3-octylthiophen-2-yl)-[1,1′-biphenyl]-4-carbaldehyde (S2, 110 mg, 38% yield) as a pale-yellow oil. 1H NMR (400 MHz, CDCl3): δ=10.1 (s, 1H), 7.96 (m, 2H), 7.78 (m, 2H), 7.69 (m, 1H), 7.59 (dt, J=7.3, 1.7 Hz 1H), 7.50 (m, 2H), 7.26 (d, J=5.2 Hz, 1H), 7.01 (d, J=5.2 Hz, 1H), 2.69 (m, 2H), 1.63 (m, 2H), 1.25 (m, 10H), 0.85 (t, J=6.9 Hz, 3H). 13C NMR (101 MHz, CDCl3): δ=191.9, 146.9, 140.0, 139.2, 137.2, 135.8, 135.4, 130.4, 129.7, 129.5, 129.3, 128.5, 127.8, 126.3, 124.1, 31.9, 31.2, 29.6, 29.5, 29.3, 28.8, 22.7, 14.2.

S2 (30 mg, 0.079 mmol) was added to a solution of N-methyl-L-phenylglycine (13 mg, 0.079 mmol) and acetic acid (0.03 mL) in methanol (0.8 mL). Sodium cyanoborohydride (15 mg, 0.24 mmol) was slowly added to the solution. The solution was stirred overnight at room temperature, then a portion of H2O was added to quench excess reducing reagent. The solvent was evaporated, and the residue was purified by silica gel column chromatography (0-20% MeOH in CH2Cl2). The target material (S)-2-(methyl((3′-(3-octylthiophen-2-yl)-[1,1′-biphenyl]-4-yl)methyl)amino)-2-phenylacetic acid (S3) was obtained (25 mg, 60% yield) as a colorless amorphous solid. 1H NMR (400 MHz, CD3OD): δ=7.71 (d, J=8.1 Hz, 2H), 7.61 (m, 6H), 7.49 (t, J=7.7 Hz, 1H), 7.42 (m, 4H), 7.31 (d, J=5.2 Hz, 1H), 6.99 (d, J=5.2 Hz, 1H), 4.57 (s, 1H), 4.38 (m, 1H), 4.27 (d, J=12.6 Hz, 1H), 2.67 (m, 2H), 2.44 (s, 3H), 1.58 (m, 2H), 1.20 (m, 10H), 0.83 (t, J=6.9 Hz, 3H). 13C-NMR (400 MHz, CD3OD): δ=172.1, 143.4, 141.7, 140.1, 138.5, 137.0, 133.0, 131.0, 130.8, 130.7, 130.3, 130.2, 129.9, 129.0, 128.6, 127.1, 125.0, 75.8, 60.5, 38.2, 33.0, 32.0, 30.3, 29.5, 23.7, 14.5. HR-MS (m/z): Calculated for C34H40NO2S+[M+H]+, 526.2774; found, 526.2770. [α]D23=+29.4° (10 mg/mL in CHCl3).

A solution of 1-[bis(dimethylamino)methylene]-1H-1,2,3-triazolo[4,5-b]pyridinium 3-oxide hexafluorophosphate (HATU, 9.4 mg, 0.025 mmol) in anhydrous DMF (0.55 mL) was added to S3 (8.7 mg, 0.017 mmol). N,N-diisopropylethylamine (DIPEA; 14.4 μL, 0.083 μmol) was then added, and the mixture was stirred for 30 min. N-methylamine in 2 M THE solution (66 μL, 0.033 mmol) was added, and the reaction mixture was stirred at room temperature for 16 h. The product was purified directly by reverse-phase HPLC (40-100% MeOH in H2O, with 0.1% TFA) to afford (6.2 mg, 70% yield) of (S)—N-methyl-2-(methyl((3′-(3-octylthiophen-2-yl)-[1,1′-biphenyl]-4-yl)methyl)amino)-2-phenylacetamide (2). 1H NMR (400 MHz, CD3OD): δ=8.52 (s, 1H), 7.79 (m, 2H), 7.65 (m, 4H), 7.60 (m, 2H), 7.54 (m, 4H), 7.45 (dt, J=7.7, 1.4 Hz, 1H), 7.34 (d, J=5.2 Hz, 1H), 7.34 (d, J=5.2 Hz, 1H), 7.02 (d, J=5.2 Hz, 1H), 4.96 (s, 1H), 4.54 (br. s, 1H), 4.34 (d, J=12.3 Hz, 1H), 2.80 (s, 3H), 2.70 (m, 2H), 2.54 (s, 3H), 1.61 (m, 2H), 1.24 (m, 10H), 0.85 (t, J=7.0 Hz, 3H). 13C NMR (101 MHz, CD3OD): δ=168.2, 144.1, 141.6, 140.1, 138.5, 137.1, 133.4, 132.0, 131.4, 130.8, 130.7, 130.4, 130.0, 129.5, 129.0, 128.9, 128.8, 127.1, 125.1, 72.7, 38.4, 33.0, 32.0, 30.3, 29.4, 26.7, 23.7, 14.4. HR-MS (m/z): Calculated for C35H43N2OS+[M+H]+, 539.3091; found, 539.3081. [α]D23=35.9° (10 mg/mL in MeOH).

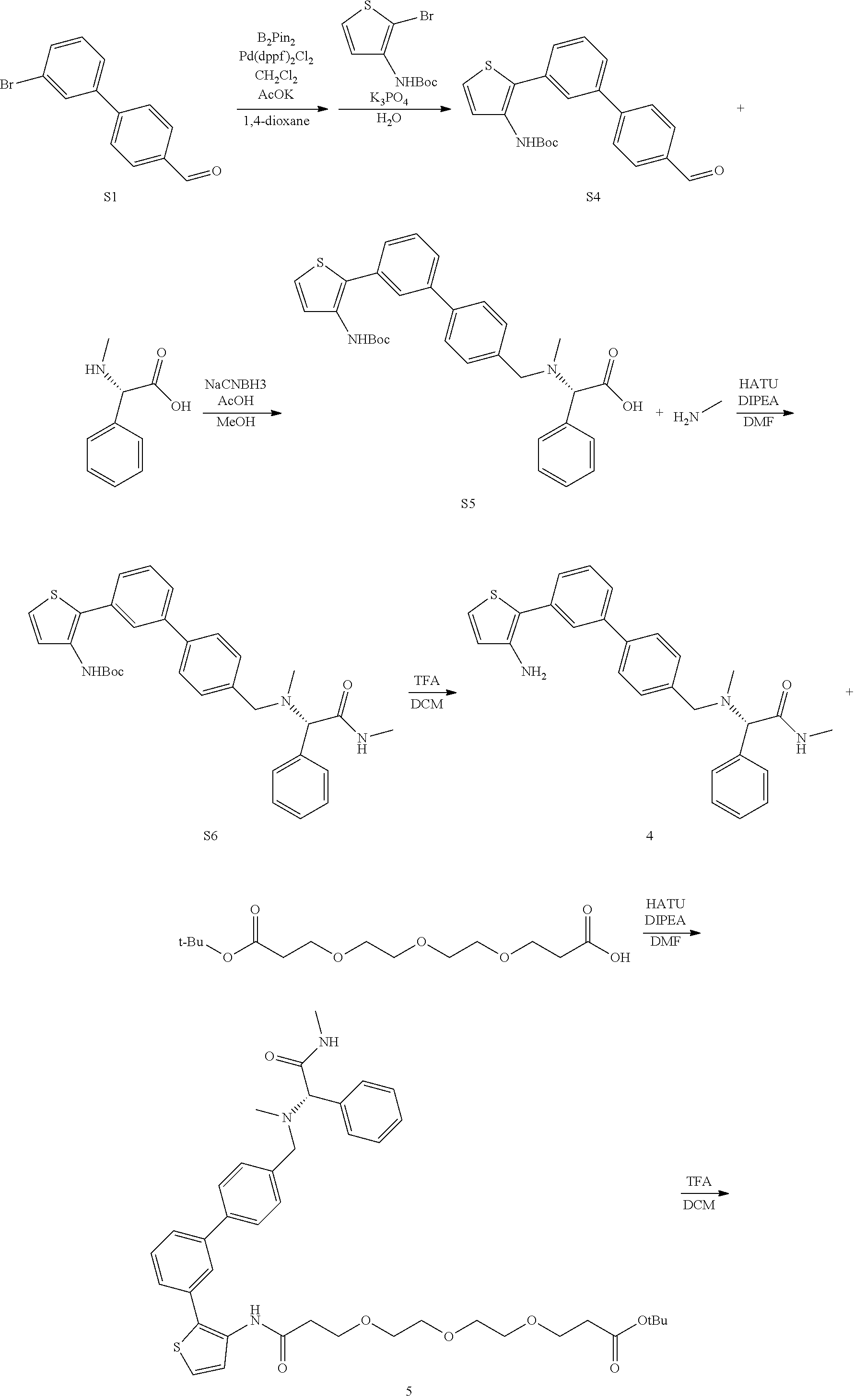

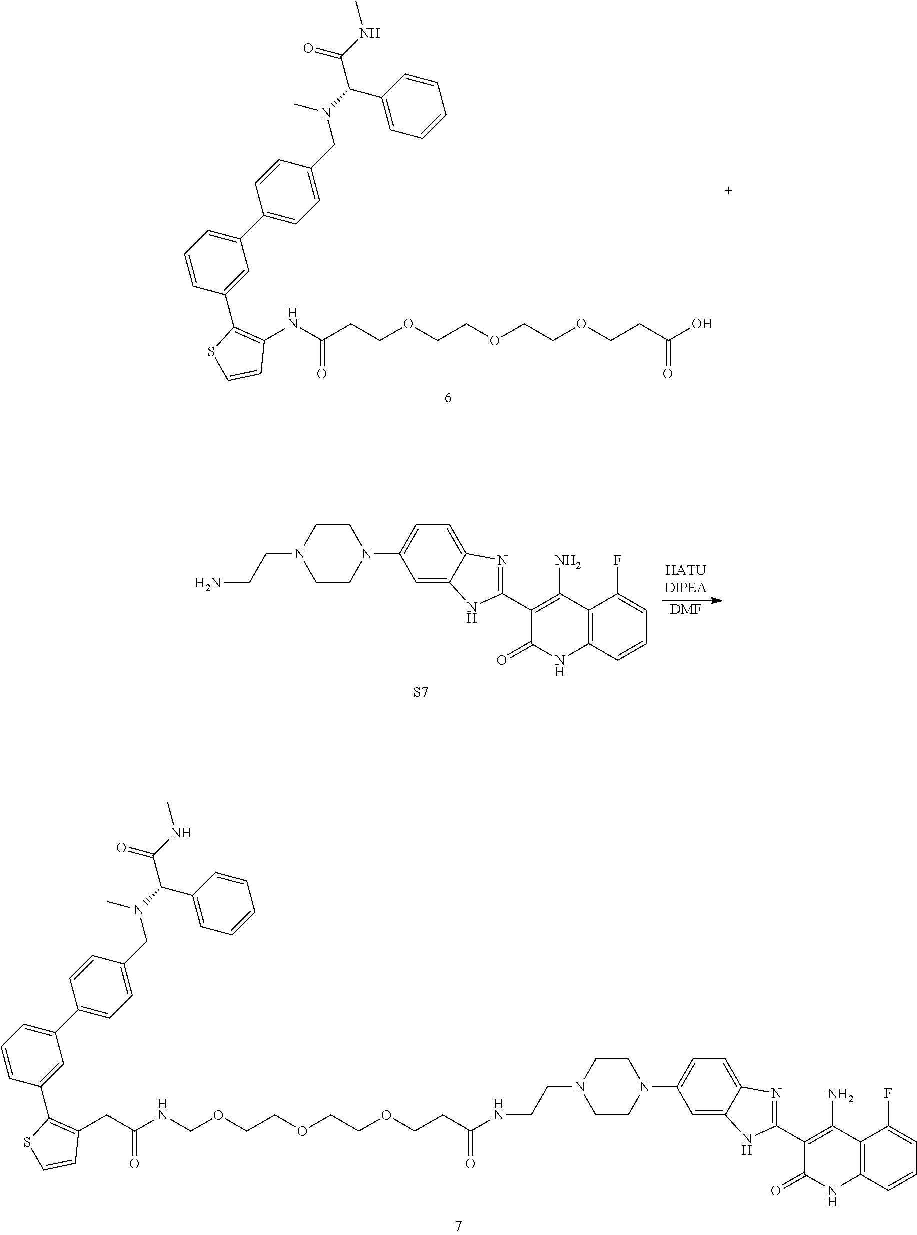

Synthesis of RiboTAC 7. S1 (500 mg, 1.91 mmol). bis(pinacolato)diboron (729 mg, 2.87 mmol), potassium acetate (281 mg, 2.87 mmol), and anhydrous 1,4dioxane (9.5 mL) were added to a round bottom flask and stirred at room temperature for 5 min, The mixture was purged with nitrogen, and a 1,1′-[bis(diphenylphosphino)ferrocene] dichloropalladium dichloromethane adduct (156 mg, 0.191 mmol) was added. The reaction mixture was stirred. at 90° C. overnight. After checking for completion of the reaction by LC/MS, tert-butyl (2-bromothiophen-3-yl)carbamate (523 mg, 1.91 mmol) and tripotassium phosphate (1.21 g, 5.74 mmol) in H2O (6.3 mL) were added, and the reaction mixture was stirred for an additional 2 h at 90° C. After checking for reaction completion by LC/MS, the reaction mixture was filtered and washed with EtOAc, and the filtrate was evaporated. The residue was purified by silica gel column chromatography (0-35% EtOAc in hexane) and by C18 silica column chromatography (0-100% MeOH in H2O) to obtain target material tert-butyl (2-(4′-formyl-[1,1′-biphenyl]-3-yl)thiophen-3-yl)carbamate (S4, 460 mg, 63% yield) as a white solid. 1H NMR (600 MHz, CD3C1): 5=9.93 (s, 1H), 7.84 (d, J=8.2 Hz, 2H), 7.65 (d, J=8.2 Hz, 2H), 7.61 (m, 1H), 7.45 (m, 4H), 7.16 (m, 1H), 6.56 (brs, 1H), 1.38 (s, 9H). 13C NMR (150 MHz, CD3C1): 5=191.8, 153.0, 146.4, 140.7, 135.4, 133.7, 132.2, 130.3, 129.9, 128.5, 127.7, 126.7, 125.0, 123.8, 80.7, 53.5, 28.3.

A portion of tert-butyl (2-(4′-formyl-[1,1′-biphenyl]-3-yl)thiophen-3-yl)carbamate (S4, 300 mg, 0.791 mmol) was added to a solution of N-methyl-L-phenylglycine (261 mg, 1.58 mmol) and acetic acid (0.26 mL) in methanol (7.9 mL). Sodium cyanoborohydride (149 mg, 2.37 mmol) was slowly added to the solution, and it was stirred overnight at room temperature. The next morning, a portion of H2O was added to quench excess reducing reagent. The solvent was evaporated, and the residue was purified by silica gel column chromatography (0-20% DCM in MeOH). The target material (S)-2-(((3′-(3-((tert-butoxycarbonyl)amino)thiophen-2-yl)-[1,1′-biphenyl]-4-yl)methyl)(methyl)amino)-2-phenylacetic acid (S5) was obtained (250 mg, 60% yield) as a pale yellow amorphous solid. 1H NMR (600 MHz, DMSO-d6): δ=7.64 (m, 3H), 7.49 (m, 5H), 7.36 (m, 5H), 7.22 (d, J=5.3 Hz, 1H), 7.06 (brs, 1H), 4.53 (s, 1H), 4.34 (m, 1H), 4.22 (m, 1H), 3.20 (s, 1H), 2.35 (s, 3H), 1.32 (s, 9H). 13C NMR (150 MHz, DMSO-d6): 5=170.1, 155.0, 142.2, 140.4, 134.0, 132.1, 131.7, 131.2, 129.7, 129.6, 129.2, 129.1, 128.9, 127.3, 126.5, 126.3, 125.9, 122.8, 79.7, 74.2, 59.1, 36.7, 27.3; HR-MS (m/z): Calculated for C31H33N2O4S+[M+H]+, 529.2156; found, 529.2155.

A solution of HATU (184 mg, 0.483 mmol) in anhydrous DMF (3.2 mL) was added to S5 (170 mg, 0.322 mmol). DIPEA (280 μL, 1.60 mmol) was then added, and the mixture was stirred or 30 min. N-methylamine in 2 M THE solution (1.28 mL, 0.644 mmol) was added and the reaction mixture was stirred at room temperature overnight. The reaction mixture was purified directly by reversed-phase HPLC (40-100% MeOH in H2O, with 0.1% TFA) to afford (140 mg, 80.3% yield) of the tert-butyl (S)-(2-(4′-((methyl(2-(methylamino)-2-oxo-1-phenylethyl)amino)methyl)-[1,1′-biphenyl]-3-yl)thiophen-3-yl)carbamate (S6) as a pale yellow amorphous solid. 1H NMR (400 MHz, CDCl3): δ=7.48 (m, 6H), 7.28 (m, 8H), 7.16 (m, 1H), 6.54 (brs, 1H), 4.06 (brs, 1H), 3.54 (brs, 1H), 3.35 (m, 2H), 2.79 (d, J=4.8 Hz, 3H), 2.08 (s, 3H), 1.39 (s, 9H). 13C NMR (150 MHz, DMSO-d6): δ=153.0, 141.8, 133.4, 132.1, 129.7, 129.2, 128.5, 128.2, 127.6, 127.5, 127.3, 126.4, 123.6, 80.7, 74.6, 59.2, 39.9, 28.3, 26.1; HR-MS (m/z): Calculated for C32H36N303S+[M+H]+, 542.2472; found, 542.2471.

A solution of S6 (140 mg, 0.258 mmol) in CH2Cl2 (0.7 mL) and TFA (0.3 mL) were mixed and stirred at room temperature for 1 h. After checking for reaction completion by LC/MS, the mixture was purified by reversed-phase column chromatography (10-100% MeOH in H2O, with 0.1% TFA). The target material (S)-2-(((3′-(3-aminothiophen-2-yl)-[1,1′-biphenyl]-4-yl)methyl)-(methyl)amino)-N-methyl-2-phenylacetamide (4) was obtained (64 mg, 56% yield) as a pale yellow amorphous solid. 1H NMR (400 MHz, CD3OD): δ=7.73 (s, 1H), 7.59 (m, 2H), 7.45 (m, 7H), 7.34 (m, 3H), 7.17 (d, J=5.3 Hz, 1H), 6.72 (d, J=5.3 Hz, 1H), 3.99 (s, 1H), 3.56 (d, J=13.3 Hz, 1H), 3.42 (d, J=13.3 Hz, 1H), 3.30 (s, 1H), 2.76 (s, 3H), 2.10 (s, 3H). 13C NMR (100 MHz, CD3OD): δ=174.9, 142.9, 142.6, 141.2, 138.9, 138.3, 136.6, 130.7, 130.5, 130.1, 129.5, 129.3, 128.0, 127.2, 126.8, 125.8, 124.4, 123.8, 117.5, 76.0, 60.1, 40.2, 26.3; HR-MS (m/z): Calculated for C27H28N30S+[M+H]+, 422.1948; found, 422.1948.

A solution of HATU (25 mg, 0.068 mmol) in anhydrous DMF (0.45 mL) was added to carboxy-PEG3-t-butyl ester (25 mg, 0.091 mmol). DIPEA (39 μL, 0.22 mmol) was then added and the mixture was stirred for 30 min. Next, compound 4 (20 mg, 0.045 mmol) was added, and the reaction mixture was stirred at room temperature for 1 h. The reaction mixture was purified directly by reversed-phase HPLC (40-100% MeOH in H2O, with 0.1% TFA) to afford (35 mg, 98% yield) of the tert-butyl (S)-3-(2-(2-(3-((2-(4′-((methyl(2-(methylamino)-2-oxo-1-phenylethyl)-amino)methyl)-[1,1′-biphenyl]-3-yl)thiophen-3-yl)amino)-3-oxopropoxy)ethoxy)-ethoxy) propanoate (5) as a colorless oil. 1H NMR (600 MHz, CD3OD): δ=7.74 (s, 1H), 7.59 (m, 3H), 7.48 (m, 6H), 7.34 (m, 4H), 7.30 (m, 1H), 3.98 (s, 1H), 3.71 (t, J=5.9 Hz, 2H), 3.56 (m, 3H), 3.45 (m, 2H), 3.40 (m, 7H), 3.30 (m, 1H), 2.76 (s, 3H), 2.57 (t, J=5.9 Hz, 2H), 2.36 (t, J=6.2 Hz, 2H), 2.10 (s, 3H) 1.40 (s, 9H). 13C NMR (150 MHz, CD3OD): δ=174.9, 172.9, 172.7, 142.8, 140.7, 139.3, 138.4, 134.7, 132.7, 132.4, 130.67, 130.61, 130.0, 129.5, 129.2, 128.4, 128.0, 127.9, 127.4, 124.4, 81.6, 76.1, 71.3, 71.19, 71.16, 68.0, 67.7, 60.0, 40.2, 37.8, 37.0, 28.3, 26.3; HR-MS (m/z): Calculated for C41H52N307S+[M+H]+, 730.3520; found, 730.3547.

A solution of 5 (140 mg, 0.258 mmol) in CH2C12 (0.7 mL) and TFA (0.3 mL) were mixed and stirred at room temperature for 1 h. After checking for reaction completion by LC/MS, the mixture was evaporated to afford (S)-3-(2-(2-(3-((2-(4′-((methyl(2-(methylamino)-2-oxo-1-phenylethyl)amino)methyl)-[1,1′-biphenyl]-3-yl)thiophen-3-yl)amino)-3-oxopropoxy)ethoxy)ethoxy)propanoic acid (6), without further purification. 1H NMR (600 MHz, CD3OD): δ=7.74 (s, 1H), 7.59 (m, 3H), 7.48 (m, 6H), 7.34 (m, 5H), 7.30 (m, 1H), 3.99 (s, 1H), 3.73 (t, J=5.8 Hz, 2H), 3.62 (t, J=6.5 Hz, 2H), 3.57 (d, J=13.3 Hz, 1H), 3.45 (m, 8H), 3.30 (m, 1H), 2.76 (s, 3H), 2.60 (t, J=5.8 Hz, 2H), 2.39 (t, J=5.9 Hz, 2H), 2.11 (s, 3H). 13C NMR (150 MHz, CD3OD): δ=178.1, 173.6, 171.8, 141.4, 139.4, 137.9, 137.0, 133.4, 131.7, 131.0, 129.3, 129.2, 128.6, 128.1, 127.8, 127.0, 126.6, 126.5, 126.2, 126.0, 123.0, 74.7, 69.8, 69.6, 69.6, 69.4, 68.0, 66.7, 58.6, 38.8, 36.2, 24.9; HR-MS (m/z): Calculated for C37H44N307S+[M+H]+, 674.2894; found, 674.2896.

A solution of HATU (25 mg, 0.068 mmol) in anhydrous DMF (0.40 mL) was added to 6 (30 mg, 0.045 mmol). DIPEA (39 μL, 0.23 mmol) was added and the mixture was stirred for 30 min. Next, 4-amino-3-(6-(4-(2-aminoethyl)piperazin-1-yl)-1H-benzo[d]imidazol-2-yl)-5-fluoroquinolin-2(1H)-one (S7)1 (19 mg, 0.045 mmol) in anhydrous DMF (0.4 mL) was added, and the reaction mixture was stirred at room temperature overnight. Additional S7 (6.3 mg, 0.015 mmol) was added, and stirring continued overnight. The reaction mixture was purified directly by reverse phase HPLC (20-100% MeOH in H2O, with 0.1% TFA) to afford (30 mg, 52% yield) of the (S)—N-(2-(4-(2-(4-amino-5-fluoro-2-oxo-1,2-dihydroquinolin-3-yl)-1H-benzo[d]imidazol-6-yl)piperazin-1-yl)ethyl)-3-(2-(2-(3-((2-(4′-((methyl(2-(methylamino)-2-oxo-1-phenylethyl)amino)methyl)-[1,1′-biphenyl]-3-yl)thiophen-3-yl)amino)-3-oxopropoxy)ethoxy)ethoxy)propanamide (7) as a yellow solid. 1H NMR (400 MHz, CD3OD): δ=8.57 (s, 1H), 7.72 (m. 3H). 7.56 (m, 12H), 7.39 (m. 1H). 7.30 (m, 1H). 7.15 (m. 3H). 6.98 (m, 1H), 5.01 (m. 1H), 4.44 (br. s, 2H). 4.31 (m, 1H), 3.76 (t. J=5.9 Hz. 2H), 3.68 (t, J=5.9 Hz. 2H), 3.62 (t, J=5.7 Hz, 2H), 3.52 (m. 8H), 3.45 (s. 1H), 3.35 (t. J=5.8 Hz. 2H), 2.80 (m, 3H). 2.60 (t, J=5.9 Hz. 2H), 2.53 (br. s. 3H). 2.45 (t, J=5.9 Hz, 2H). 13C NMR (101 MHz, CD3OD): δ=175.6, 173.1, 168.2, 163.4, 163.2, 162.3, 161.9, 160.9, 155.0, 149.4, 148.9, 143.7, 141.8, 141.6, 141.6, 135.7, 135.0, 134.3, 133.3, 133.1, 132.5, 132.0, 131.4, 130.8, 130.8, 129.6, 128.9, 128.7, 127.9, 127.8, 127.5, 124.7, 119.2, 117.4, 116.3, 115.9, 113.7, 113.6, 113.4, 109.9, 109.7, 104.3, 104.2, 101.9, 91.0, 72.7, 71.3, 71.2, 68.1, 67.9, 57.9, 53.6, 38.3, 37.7, 37.3, 35.6, 26.7, 19F NMR (376 MHz, CD3OD): δ=−77.1, −116.5. HR-MS (m/z): Calculated for C59H66FN10O7S+[M+H]+. 1077.4815; found, 1077.4819. [α]D22=+9.7° (10 mg/mL in MeOH).

### References (Examples)

(1) Zhang, P.; Liu, X.; Abegg, D.; Tanaka, T.; Tong, Y.; Benhamou, R. I.; Baisden, J.; Crynen, G.; Meyer, S. M.; Cameron, M. D.; et al. Reprogramming of protein-targeted small-molecule medicines to RNA by ribonuclease recruitment. J. Am. Chem. Soc. 2021, 143 (33), 13044-13055.
(2) Agarwal, V.; Bell, G. W.; Nam, J.-W.; Bartel, D. P. Predicting effective microRNA target sites in mammalian mRNAs. eLife 2015, 4, e05005.
(3) Costales, M. G.; Aikawa, H.; Li, Y.; Childs-Disney, J. L.; Abegg, D.; Hoch, D. G.; Pradeep Velagapudi, S.; Nakai, Y.; Khan, T.; Wang, K. W.; et al. Small-molecule targeted recruitment of a nuclease to cleave an oncogenic RNA in a mouse model of metastatic cancer. Proc. Natl. Acad. Sci. U.S.A 2020, 117 (5), 2406-2411.
(4) Han, Y.; Donovan, J.; Rath, S.; Whitney, G.; Chitrakar, A.; Korennykh, A. Structure of human RNase L reveals the basis for regulated RNA decay in the IFN rResponse. Science 2014, 343 (6176), 1244-1248.
(5) Dong, B.; Silverman, R. H. A bipartite model of 2-5A-dependent RNase L. J. Biol. Chem. 1997, 272 (35), 22236-22242.
(6) Kuai, L.; O'Keeffe, T.; Arico-Muendel, C. Randomness in DNA encoded library selection data can be modeled for more reliable enrichment calculation. SLAS Discov. 2018, 23 (5), 405-416.
(7) Thakur, C. S.; Jha, B. K.; Dong, B.; Das Gupta, J.; Silverman, K. M.; Mao, H.; Sawai, H.; Nakamura, A. O.; Banerjee, A. K.; Gudkov, A.; et al. Small-molecule activators of RNase L with broad-spectrum antiviral activity. Proc. Natl. Acad. Sci. U.S.A 2007, 104 (23), 9585-9590.
(8) Velagapudi, S. P.; Luo, Y.; Tran, T.; Haniff, H. S.; Nakai, Y.; Fallahi, M.; Martinez, G. J.; Childs-Disney, J. L.; Disney, M. D. Defining RNA-small molecule affinity landscapes enables design of a small molecule inhibitor of an oncogenic concoding RNA. ACS Cent. Sci. 2017, 3 (3), 205-216.
(9) Angelbello, A. J.; Disney, M. D. Bleomycin can cleave an oncogenic noncoding RNA. ChemBioChem 2018,19 (1), 43-47.
(10) Rostovtsev, V. V.; Green, L. G.; Fokin, V. V.; Sharpless, K. B. A Stepwise Huisgen cycloaddition process: copper(I)-catalyzed regioselective “ligation” of azides and terminal aAlkynes. Angew. Chem. Int. Ed. 2002, 41 (14), 2596-2599.
(11) Livak, K. J.; Schmittgen, T. D. Analysis of relative gene expression data using real-time quantitative PCR and the 2-ΔΔCT Method. Methods 2001, 25 (4), 402-408.
(12) Schneider, C. A.; Rasband, W. S.; Eliceiri, K. W. NIH Image to ImageJ: 25 years of image analysis. Nat. Methods 2012, 9 (7), 671-675.

TABLE S1
Sequences of primers used.

| Primer | Sequence (5′ to 3′) | Supplier |
|---|---|---|
| U6 Fwd | MiR-X miRNA First | Takara Bio |
| | Strand Synthesis | |
| | Kit | |
| U6 Rev | MiR-X miRNA First | Takara Bio |
| | Strand Synthesis | |
| | Kit | |
| hsa-miR-21 | TAGCTTATCAGACTGATGTT | IDT |
| Fwd | GA | |
| mRQ Universal | MiR-X miRNA First | Takara Bio |
| Rev | Strand Synthesis Kit | |
| 18S Fwd | GTAACCCGTTGAACCCCATT | IDT |
| 18S Rev | CCATCCAATCGGTAGTAGCG | IDT |
| pre-miR-21 Fwd | CTGATGTTGACTGTTGAATC | IDT |
| pre-miR-21 Rev | GCCCATCGACTGGTGTTGCC | IDT |

*Sequences of primers used in Global microRNA profiling have been previously described.³

### INCORPORATION BY REFERENCE

The present application refers to various issued patent, published patent applications, scientific journal articles, and other publications, all of which are incorporated herein by reference. The details of one or more embodiments of the invention are set forth herein. Other features, objects, and advantages of the invention will be apparent from the Detailed Description, the Figures, the Examples, and the Claims.

### EQUIVALENTS AND SCOPE

In the articles such as “a,” “an,” and “the” may mean one or more than one unless indicated to the contrary or otherwise evident from the context. Embodiments or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claims that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the embodiments. Because such embodiments are deemed to be known to one of ordinary skill in the art, they may be excluded even if the exclusion is not set forth explicitly herein. Any particular embodiment of the invention can be excluded from any embodiment, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended embodiments. Those of ordinary skill in the art will appreciate that various changes and modifications to this description may be made without departing from the spirit or scope of the present invention, as defined in the following claims.

What is claimed is:

1. A compound of Formula (I): 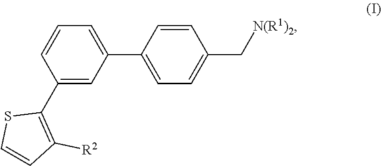
or a pharmaceutically acceptable salt, solvate, hydrate, polymorph, co-crystal, tautomer, stereoisomer, isotopically labeled compound, or prodrug thereof, wherein:
each occurrence of R¹ is independently hydrogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, or a nitrogen protecting group, or two occurrences of R¹ are joined together with their intervening atom to form an optionally substituted heterocyclic ring or optionally substituted heteroaryl ring;
R² is hydrogen, halogen, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, —CN, —OR^{A}, —SCN, —SR^{A}, —SSR^{A}, —N₃, —NO, —N(R^{A})₂, —NO₂, —C(═O)R^{A}, —C(═O)OR^{A}, —C(═O)SR^{A}, —C(═O)N(R^{A})₂, —C(═NR^{A})R^{A}, —C(═NR^{A})OR^{A}, —C(═NR^{A})SR^{A}, —C(═NR^{A})N(R^{A})₂, —S(═O)R^{A}, —S(═O)OR^{A}, —S(═O)SR^{A}, —S(═O)N(R^{A})₂, —S(═O)₂R^{A}, —S(═O)₂OR^{A}, —S(═O)₂SR^{A}, —S(═O)₂N(R^{A})₂, —OC(═O)R^{A}, —OC(═O)OR^{A}, —OC(═O)SR^{A}, —OC(═O)N(R^{A})₂, —OC(═NR^{A})R^{A}, —OC(═NR^{A})OR^{A}, —OC(═NR^{A})SR^{A}, —OC(═NR^{A})N(R^{A})₂, —OS(═O)R^{A}, —OS(═O)OR^{A}, —OS(═O)SR^{A}, —OS(═O)N(R^{A})₂, —OS(═O)₂R^{A}, —OS(═O)₂OR^{A}, —OS(═O)₂SR^{A}, —OS(═O)₂N(R^{A})₂, —ON(R^{A})₂, —SC(═O)R^{A}, —SC(═O)OR^{A}, —SC(═O)SR^{A}, —SC(═O)N(R^{A})₂, —SC(═NR^{A})R^{A}, —SC(═NR^{A})OR^{A}, —SC(═NR^{A})SR^{A}, —SC(═NR^{A})N(R^{A})₂, —NR^{A}C(═O)R^{A}, —NR^{A}C(═O)OR^{A}, —NR^{A}C(═O)SR^{A}, —NR^{A}C(═O)N(R^{A})₂, —NR^{A}C(═NR^{A})R^{A}, —NR^{A}C(═NR^{A})OR^{A}, —NR^{A}C(═NR^{A})SR^{A}, —NR^{A}C(═NR^{A})N(R^{A})₂, —NR^{A}S(═O)R^{A}, —NR^{A}S(═O)OR^{A}, —NR^{A}S(═O)SR^{A}, —NR^{A}S(═O)N(R^{A})₂, —NR^{A}S(═O)₂R^{A}, —NR^{A}S(═O)₂OR^{A}, —NR^{A}S(═O)₂SR^{A}, —NR^{A}S(═O)₂N(R^{A})₂, —Si(R^{A})₃, —Si(R^{A})₂OR^{A}, —Si(R^{A})(OR^{A})₂, —Si(OR^{A})₃, —OSi(R^{A})₃, —OSi(R^{A})₂OR^{A}, —OSi(R^{A})(OR^{A})₂, —OSi(OR^{A})₃, or —B(OR^{A})₂; and
each occurrence of R^{A} is independently hydrogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, a nitrogen protecting group when attached to a nitrogen atom, an oxygen protecting group when attached to an oxygen atom, or a sulfur protecting group when attached to a sulfur atom, or two occurrences of R^{A} are joined together with their intervening atom to form an optionally substituted heterocyclic ring or optionally substituted heteroaryl ring.

2. The compound of claim 1, wherein two occurrences of R¹ are joined together with their intervening atom to form an optionally substituted heterocyclic ring or optionally substituted heteroaryl ring.

3. The compound of any one of claims 1 and 2, or a pharmaceutically acceptable salt, solvate, hydrate, polymorph, co-crystal, tautomer, stereoisomer, isotopically labeled compound, or prodrug thereof, wherein the moiety —N(R¹)₂ is of formula:

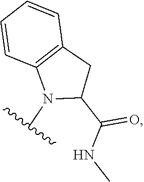 [SMILES: *N1c2ccccc2CC1C(=O)NC]

4. The compound of claim 1, or a pharmaceutically acceptable salt, solvate, hydrate, polymorph, co-crystal, tautomer, stereoisomer, isotopically labeled compound, or prodrug thereof, wherein at least one occurrence of R¹ is optionally substituted alkyl.

5. The compound of any one of claims 1 and 4, or a pharmaceutically acceptable salt, solvate, hydrate, polymorph, co-crystal, tautomer, stereoisomer, isotopically labeled compound, or prodrug thereof, wherein the moiety —N(R¹)₂ is of formula:

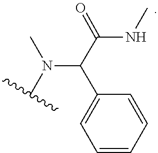

6. The compound of any one of claims 1-5, or a pharmaceutically acceptable salt, solvate, hydrate, polymorph, co-crystal, tautomer, stereoisomer, isotopically labeled compound, or prodrug thereof, wherein R² is optionally substituted alkyl.

7. The compound of any one of claims 1-6, or a pharmaceutically acceptable salt, solvate, hydrate, polymorph, co-crystal, tautomer, stereoisomer, isotopically labeled compound, or prodrug thereof, wherein R² is unsubstituted n-octyl.

8. The compound of any one of claims 1-5, or a pharmaceutically acceptable salt, solvate, hydrate, polymorph, co-crystal, tautomer, stereoisomer, isotopically labeled compound, or prodrug thereof, wherein R² is —N(R^{A})₂.

9. The compound of any one of claims 1-5 and 8, or a pharmaceutically acceptable salt, solvate, hydrate, polymorph, co-crystal, tautomer, stereoisomer, isotopically labeled compound, or prodrug thereof, wherein R² is —NH(R^{A}).

10. The compound of any one of claims 1-5, 8, and 9, or a pharmaceutically acceptable salt, solvate, hydrate, polymorph, co-crystal, tautomer, stereoisomer, isotopically labeled compound, or prodrug thereof, wherein R² is —NH(optionally substituted alkyl).

11. The compound of any one of claims 1-5 and 8-10, or a pharmaceutically acceptable salt, solvate, hydrate, polymorph, co-crystal, tautomer, stereoisomer, isotopically labeled compound, or prodrug thereof, wherein R² is of formula: 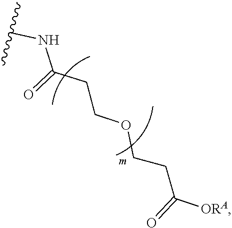
wherein m is 0, 1, 2, 3, 4, 5, 6, 7, 8, 9, or 10.

12. The compound of any one of claims 1-5 and 8-11, or a pharmaceutically acceptable salt, solvate, hydrate, polymorph, co-crystal, tautomer, stereoisomer, isotopically labeled compound, or prodrug thereof, wherein R² is of formula:

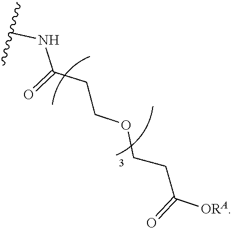

13. The compound of any one of claims 1-5 and 8-12, or a pharmaceutically acceptable salt, solvate, hydrate, polymorph, co-crystal, tautomer, stereoisomer, isotopically labeled compound, or prodrug thereof, wherein R² is of formula:

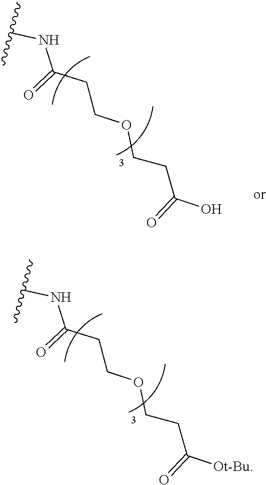

14. The compound of any one of claims 1-5, 8, and 9, or a pharmaceutically acceptable salt, solvate, hydrate, polymorph, co-crystal, tautomer, stereoisomer, isotopically labeled compound, or prodrug thereof, wherein R² is —NH₂.

15. The compound of any one of claims 1-14, or a pharmaceutically acceptable salt, solvate, hydrate, polymorph, co-crystal, tautomer, stereoisomer, isotopically labeled compound, or prodrug thereof, wherein the compound of Formula (I) is of formula: 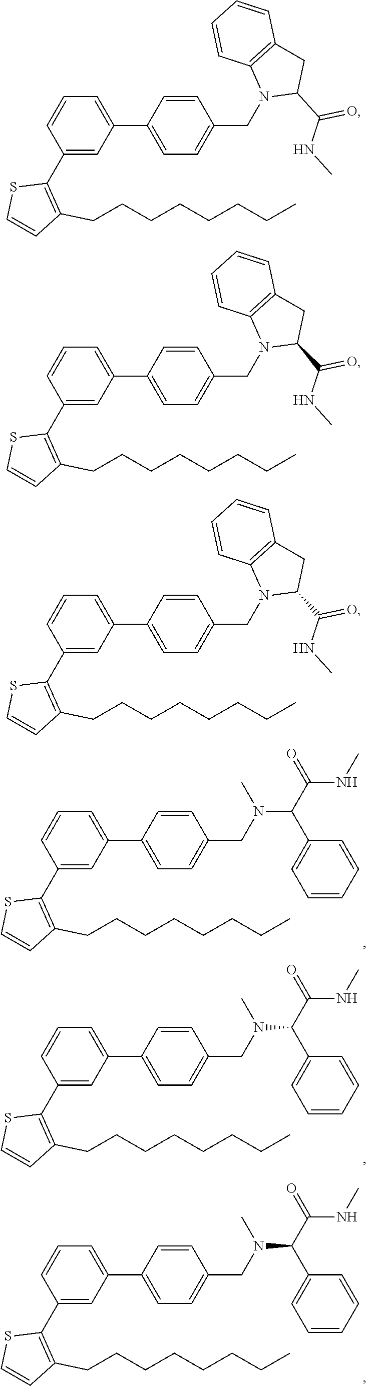 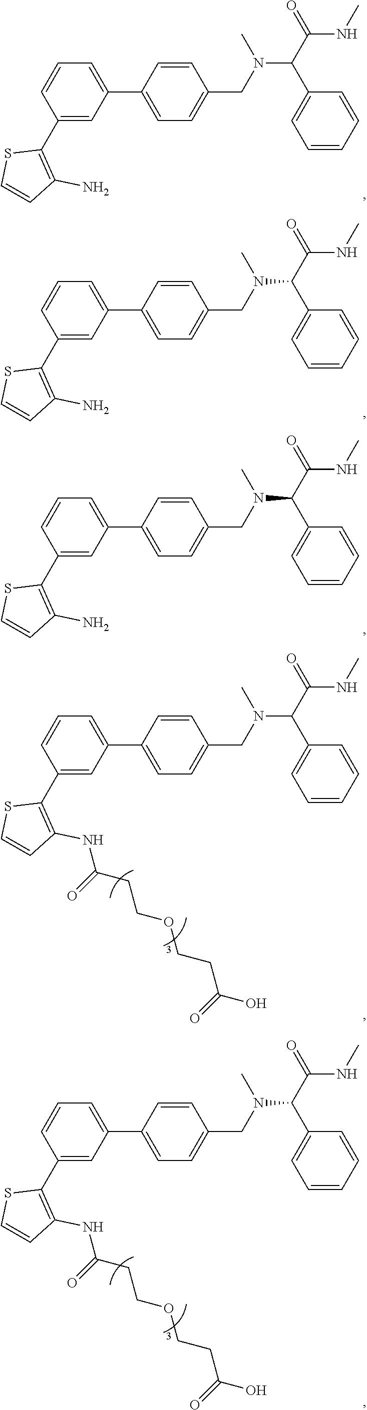 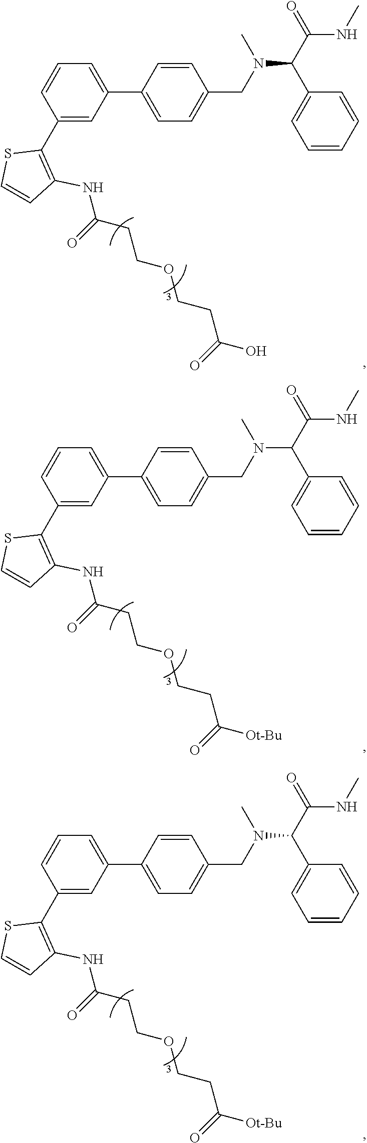 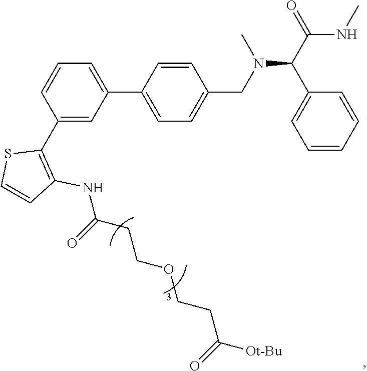
or a pharmaceutically acceptable salt, solvate, hydrate, polymorph, co-crystal, tautomer, stereoisomer, isotopically labeled compound, or prodrug thereof.

16. A compound of Formula (II): 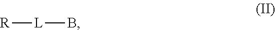
or a pharmaceutically acceptable salt, solvate, hydrate, polymorph, co-crystal, tautomer, stereoisomer, isotopically labeled compound, or prodrug thereof, wherein:
R is an RNase L recruiter of Formula (III): 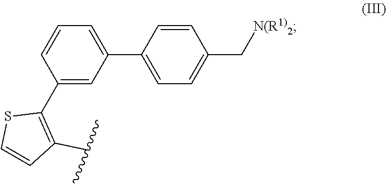
L is a linker;
B is an RNA binder; and
each occurrence of R¹ is independently hydrogen, optionally substituted acyl, optionally substituted alkyl, optionally substituted alkenyl, optionally substituted alkynyl, optionally substituted heteroalkyl, optionally substituted heteroalkenyl, optionally substituted heteroalkynyl, optionally substituted carbocyclyl, optionally substituted heterocyclyl, optionally substituted aryl, optionally substituted heteroaryl, or a nitrogen protecting group, or two occurrences of R¹ are joined together with their intervening atom to form an optionally substituted heterocyclic ring or optionally substituted heteroaryl ring.

17. The compound of claim 16, wherein two occurrences of R¹ are joined together with their intervening atom to form an optionally substituted heterocyclic ring or optionally substituted heteroaryl ring.

18. The compound of any one of claims 16 and 17, or a pharmaceutically acceptable salt, solvate, hydrate, polymorph, co-crystal, tautomer, stereoisomer, isotopically labeled compound, or prodrug thereof, wherein the moiety —N(R¹)₂ is of formula:

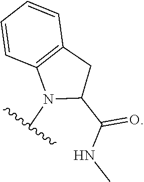 [SMILES: *N1c2ccccc2CC1C(=O)NC]

19. The compound of claim 16, or a pharmaceutically acceptable salt, solvate, hydrate, polymorph, co-crystal, tautomer, stereoisomer, isotopically labeled compound, or prodrug thereof, wherein at least one occurrence of R¹ is optionally substituted alkyl.

20. The compound of any one of claims 16 and 19, or a pharmaceutically acceptable salt, solvate, hydrate, polymorph, co-crystal, tautomer, stereoisomer, isotopically labeled compound, or prodrug thereof, wherein the moiety —N(R¹)₂ is of formula:

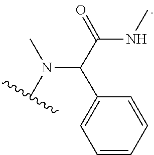

21. The compound of any one of claims 16, 19, and 20, or a pharmaceutically acceptable salt, solvate, hydrate, polymorph, co-crystal, tautomer, stereoisomer, isotopically labeled compound, or prodrug thereof, wherein R is of formula:

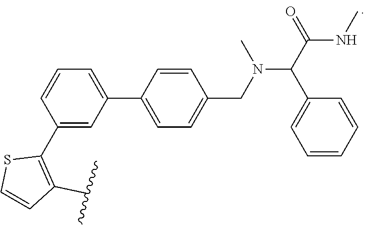 [SMILES: *c1ccsc1-c1cccc(-c2ccc(CN(C)C(C(=O)NC)c3ccccc3)cc2)c1]

22. The compound of any one of claims 16-21, or a pharmaceutically acceptable salt, solvate, hydrate, polymorph, co-crystal, tautomer, stereoisomer, isotopically labeled compound, or prodrug thereof, wherein L is a bond, optionally substituted alkylene, optionally substituted alkenylene, optionally substituted alkynylene, optionally substituted heteroalkylene, optionally substituted heteroalkenylene, optionally substituted heteroalkynylene, optionally substituted heterocyclylene, optionally substituted carbocyclylene, optionally substituted arylene, optionally substituted heteroarylene, or a combination thereof.

23. The compound of any one of claims 16-22, or a pharmaceutically acceptable salt, solvate, hydrate, polymorph, co-crystal, tautomer, stereoisomer, isotopically labeled compound, or prodrug thereof, wherein L is optionally substituted alkylene, optionally substituted heteroalkylene, or a combination thereof.

24. The compound of any one of claims 16-23, or a pharmaceutically acceptable salt, solvate, hydrate, polymorph, co-crystal, tautomer, stereoisomer, isotopically labeled compound, or prodrug thereof, wherein L comprises a moiety of formula: 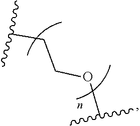
wherein n is 0, 1, 2, 3, 4, 5, 6, 7, 8, 9, or 10.

25. The compound of any one of claims 16-24, or a pharmaceutically acceptable salt, solvate, hydrate, polymorph, co-crystal, tautomer, stereoisomer, isotopically labeled compound, or prodrug thereof, wherein L comprises a moiety of formula:

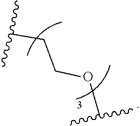

26. The compound of any one of claims 16-25, or a pharmaceutically acceptable salt, solvate, hydrate, polymorph, co-crystal, tautomer, stereoisomer, isotopically labeled compound, or prodrug thereof, wherein L comprises a moiety of formula: 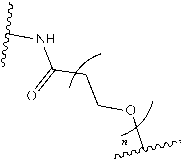
wherein n is 0, 1, 2, 3, 4, 5, 6, 7, 8, 9, or 10.

27. The compound of any one of claims 16-26, or a pharmaceutically acceptable salt, solvate, hydrate, polymorph, co-crystal, tautomer, stereoisomer, isotopically labeled compound, or prodrug thereof, wherein L comprises a moiety of formula:

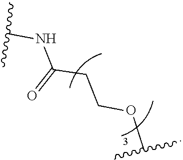

28. The compound of any one of claims 16-27, or a pharmaceutically acceptable salt, solvate, hydrate, polymorph, co-crystal, tautomer, stereoisomer, isotopically labeled compound, or prodrug thereof, wherein L is of formula: 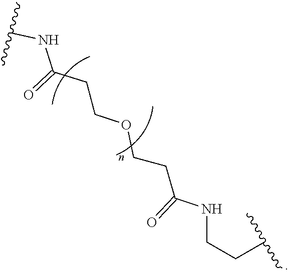
wherein n is 0, 1, 2, 3, 4, 5, 6, 7, 8, 9, or 10.

29. The compound of any one of claims 16-28, or a pharmaceutically acceptable salt, solvate, hydrate, polymorph, co-crystal, tautomer, stereoisomer, isotopically labeled compound, or prodrug thereof, wherein L is of formula:

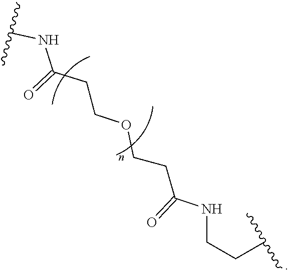

30. The compound of any one of claims 16-29, wherein B is an RNA binder of Formula (IV):

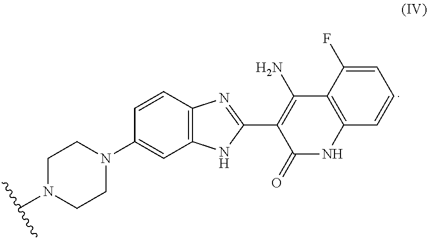 [SMILES: *N1CCN(c2ccc3nc(-c4c(N)c5c(F)cccc5[nH]c4=O)[nH]c3c2)CC1]

31. The compound of any one of claims 16 and 19-30, wherein the compound is of formula: 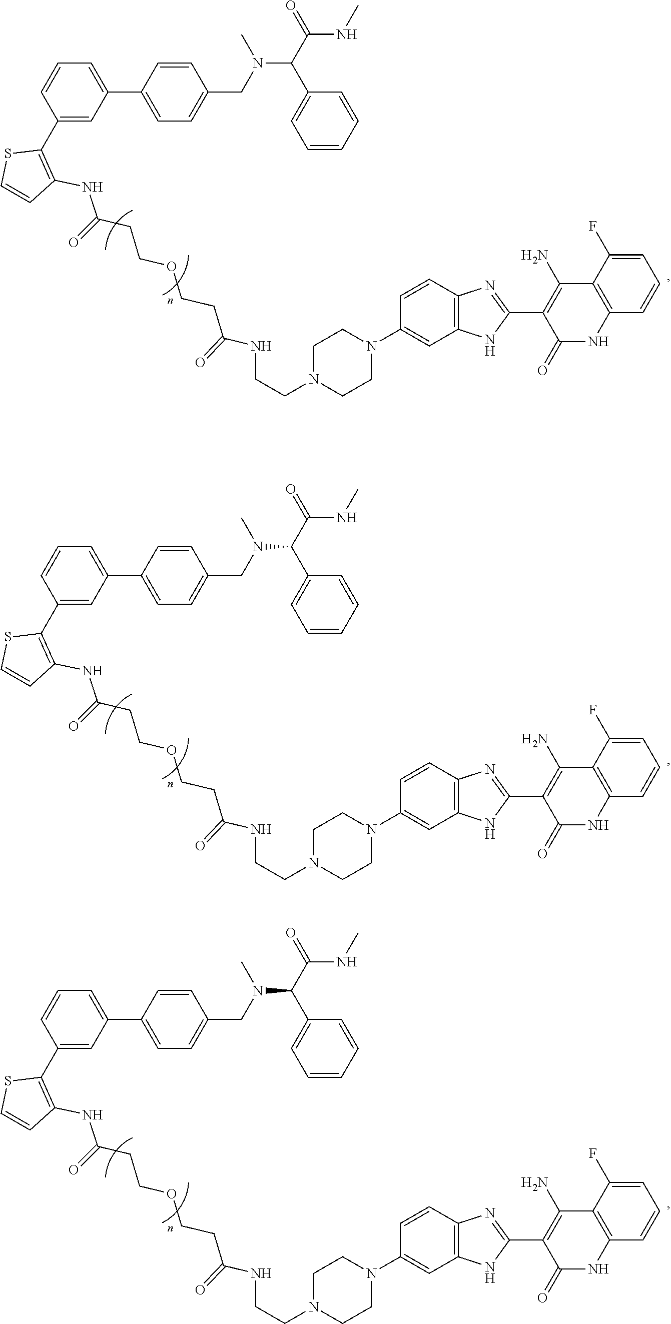
or a pharmaceutically acceptable salt, solvate, hydrate, polymorph, co-crystal, tautomer, stereoisomer, isotopically labeled compound, or prodrug thereof.

32. The compound of any one of claims 16 and 19-31, wherein the compound is of formula: 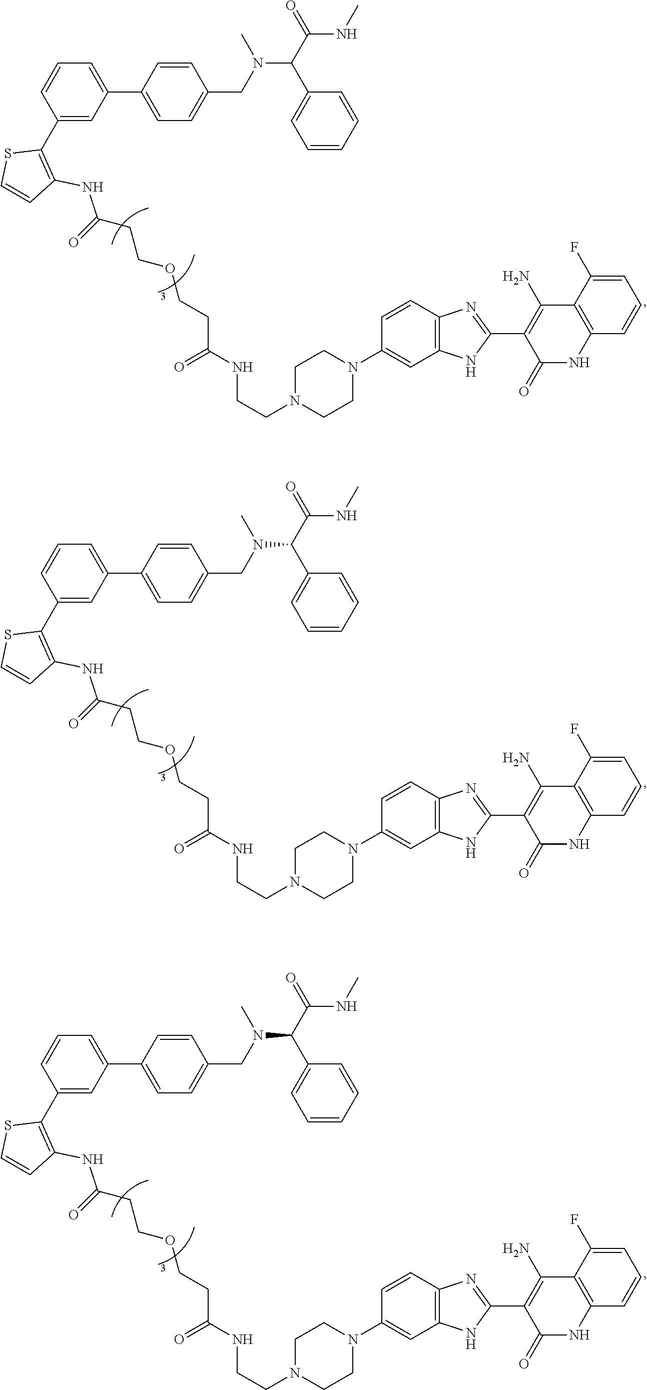
or a pharmaceutically acceptable salt, solvate, hydrate, polymorph, co-crystal, tautomer, stereoisomer, isotopically labeled compound, or prodrug thereof.

33. A composition comprising the compound of any one of claims 1-32, or a pharmaceutically acceptable salt, solvate, hydrate, polymorph, co-crystal, tautomer, stereoisomer, isotopically labeled compound, or prodrug thereof, and an excipient.

34. A method of effecting degradation of an RNA target in a subject in need thereof or in a cell, tissue, or biological sample, comprising administering to the subject in need thereof or contacting the cell, tissue, or biological sample with an effective amount of:
a heterobifunctional compound, or a pharmaceutically acceptable salt, solvate, hydrate, polymorph, co-crystal, tautomer, stereoisomer, isotopically labeled compound, or prodrug thereof, comprising:
a compound of any one of claims 1-15, or a pharmaceutically acceptable salt, solvate, hydrate, polymorph, co-crystal, tautomer, stereoisomer, isotopically labeled compound, or prodrug thereof, capable of binding to RNase L; and
an RNA binder capable of binding to the RNA target.

35. A method of preparing a heterobifunctional compound, or a pharmaceutically acceptable salt, solvate, hydrate, polymorph, co-crystal, tautomer, stereoisomer, isotopically labeled compound, or prodrug thereof, capable of effecting degradation of an RNA target, comprising combining:
a radical of a compound of any one of claims 1-15, or a pharmaceutically acceptable salt, solvate, hydrate, polymorph, co-crystal, tautomer, stereoisomer, isotopically labeled compound, or prodrug thereof, or a radical of a linker connected to the compound of any one of claims 1-15, or pharmaceutically acceptable salt, solvate, hydrate, polymorph, co-crystal, tautomer, stereoisomer, isotopically labeled compound, or prodrug thereof, wherein the compound of any one of claims 1-15, or pharmaceutically acceptable salt, solvate, hydrate, polymorph, co-crystal, tautomer, stereoisomer, isotopically labeled compound, or prodrug thereof, is capable of binding to RNase L; and
a radical of an RNA binder, or a radical of a linker connected to the RNA binder, wherein the RNA binder is capable of binding to the RNA target.

36. A method of binding RNase L in a subject in need thereof or in a cell, tissue, or biological sample, comprising administering to the subject in need thereof or contacting the cell, tissue, or biological sample with an effective amount of:
a compound of any one of claims 1-32, or a pharmaceutically acceptable salt, solvate, hydrate, polymorph, co-crystal, tautomer, stereoisomer, isotopically labeled compound, or prodrug thereof;
or the composition of claim 33.

37. The method of any one of claims 34-36, wherein binding RNase L comprises activating RNase L.

38. The method of any one of claims 34-37, wherein binding RNase L comprises inducing RNase L dimerization.

39. A method of binding an RNA target in a subject in need thereof or in a cell, tissue, or biological sample, comprising administering to the subject in need thereof or contacting the cell, tissue, or biological sample with an effective amount of:
a compound of any one of claims 16-32, or a pharmaceutically acceptable salt, solvate, hydrate, polymorph, co-crystal, tautomer, stereoisomer, isotopically labeled compound, or prodrug thereof;
or the composition of claim 33.

40. A method of inhibiting cell proliferation or inducing apoptosis in a subject in need thereof or in a cell, tissue, or biological sample, comprising administering to the subject in need thereof or contacting the cell, tissue, or biological sample with an effective amount of:
a compound of any one of claims 1-32, or a pharmaceutically acceptable salt, solvate, hydrate, polymorph, co-crystal, tautomer, stereoisomer, isotopically labeled compound, or prodrug thereof;
or the composition of claim 33.

41. The method, compound for use, pharmaceutically acceptable salt, solvate, hydrate, polymorph, co-crystal, tautomer, stereoisomer, isotopically labeled compound, or prodrug thereof for use, or composition for use of any one of claims 34-39 further comprising recruiting RNase L.

42. The method, compound for use, pharmaceutically acceptable salt, solvate, hydrate, polymorph, co-crystal, tautomer, stereoisomer, isotopically labeled compound, or prodrug thereof for use, or composition for use of any one of claims 34-39 and 41, further comprising cleaving the RNA target.

43. The method, compound for use, pharmaceutically acceptable salt, solvate, hydrate, polymorph, co-crystal, tautomer, stereoisomer, isotopically labeled compound, or prodrug thereof for use, or composition for use of any one of claims 34-39, 41, and 42, further comprising effecting degradation of the RNA target.

44. The method, compound for use, pharmaceutically acceptable salt, solvate, hydrate, polymorph, co-crystal, tautomer, stereoisomer, isotopically labeled compound, or prodrug thereof for use, or composition for use of any one of claims 34-39 and 41-43, wherein the RNA target is a precursor to microRNA-21 (pre-miR-21).

45. The method, compound for use, pharmaceutically acceptable salt, solvate, hydrate, polymorph, co-crystal, tautomer, stereoisomer, isotopically labeled compound, or prodrug thereof for use, or composition for use of any one of claims 34-39 and 41-44, further comprising decreasing an amount of pre-miR-21.

46. The method, compound for use, pharmaceutically acceptable salt, solvate, hydrate, polymorph, co-crystal, tautomer, stereoisomer, isotopically labeled compound, or prodrug thereof for use, or composition for use of any one of claims 34-39 and 41-45, further comprising cleaving pre-miR-21.

47. The method, compound for use, pharmaceutically acceptable salt, solvate, hydrate, polymorph, co-crystal, tautomer, stereoisomer, isotopically labeled compound, or prodrug thereof for use, or composition for use of any one of claims 34-39 and 41-46, further comprising downregulating mature microRNA-21 (miR-21).

48. The method, compound for use, pharmaceutically acceptable salt, solvate, hydrate, polymorph, co-crystal, tautomer, stereoisomer, isotopically labeled compound, or prodrug thereof for use, or composition for use of claim 47, wherein the downregulating is selective for miR-21 compared to other mature microRNA.

49. The method, compound for use, pharmaceutically acceptable salt, solvate, hydrate, polymorph, co-crystal, tautomer, stereoisomer, isotopically labeled compound, or prodrug thereof for use, or composition for use of any one of claims 34-39 and 41-48, further comprising decreasing an amount of miR-21.

50. The method, compound for use, pharmaceutically acceptable salt, solvate, hydrate, polymorph, co-crystal, tautomer, stereoisomer, isotopically labeled compound, or prodrug thereof for use, or composition for use of any one of claims 34-39 and 41-49, further comprising increasing an amount of programmed cell death protein 4 (PDCD4).

51. The method, compound for use, pharmaceutically acceptable salt, solvate, hydrate, polymorph, co-crystal, tautomer, stereoisomer, isotopically labeled compound, or prodrug thereof for use, or composition for use of claim 50, wherein increasing the amount of PDCD4 comprises derepression of PDCD4.

52. The method, compound for use, pharmaceutically acceptable salt, solvate, hydrate, polymorph, co-crystal, tautomer, stereoisomer, isotopically labeled compound, or prodrug thereof for use, or composition for use of any one of claims 34-39 and 41-51, further comprising selectively deactivating a miR-21-mediated cancer circuit.

53. The method, compound for use, pharmaceutically acceptable salt, solvate, hydrate, polymorph, co-crystal, tautomer, stereoisomer, isotopically labeled compound, or prodrug thereof for use, or composition for use of any one of claims 34-39 and 41-52, further comprising not changing an amount of extracellular signal-regulated kinase (ERK) or an amount of phosphorylated extracellular signal-regulated kinase (pERK).

54. A method of treating or preventing a disease in a subject in need thereof, comprising administering to the subject in need thereof an effective amount of:
the compound of any one of claims 1-32, or a pharmaceutically acceptable salt, solvate, hydrate, polymorph, co-crystal, tautomer, stereoisomer, isotopically labeled compound, or prodrug thereof;
or the composition of claim 33.

55. The compound of any one of claims 1-32, or a pharmaceutically acceptable salt, solvate, hydrate, polymorph, co-crystal, tautomer, stereoisomer, isotopically labeled compound, or prodrug thereof, or the composition of claim 33, for use in treating a disease in a subject in need thereof.

56. The compound of any one of claims 1-32, or a pharmaceutically acceptable salt, solvate, hydrate, polymorph, co-crystal, tautomer, stereoisomer, isotopically labeled compound, or prodrug thereof, or the composition of claim 33, for use in the manufacture of a medicament for treatment of a disease in a subject in need thereof.

57. The method, compound for use, pharmaceutically acceptable salt, solvate, hydrate, polymorph, co-crystal, tautomer, stereoisomer, isotopically labeled compound, or prodrug thereof for use, or composition for use of any one of claims 54-56, wherein the disease is associated with microRNA-21 (miR-21).

58. The method, compound for use, pharmaceutically acceptable salt, solvate, hydrate, polymorph, co-crystal, tautomer, stereoisomer, isotopically labeled compound, or prodrug thereof for use, or composition for use of any one of claims 54-57, wherein the disease is a proliferative disease.

59. The method, compound for use, pharmaceutically acceptable salt, solvate, hydrate, polymorph, co-crystal, tautomer, stereoisomer, isotopically labeled compound, or prodrug thereof for use, or composition for use of claim 58, wherein the proliferative disease is cancer.

60. The method, compound for use, pharmaceutically acceptable salt, solvate, hydrate, polymorph, co-crystal, tautomer, stereoisomer, isotopically labeled compound, or prodrug thereof for use, or composition for use of claim 59, wherein the cancer is breast cancer.

61. The method, compound for use, pharmaceutically acceptable salt, solvate, hydrate, polymorph, co-crystal, tautomer, stereoisomer, isotopically labeled compound, or prodrug thereof for use, or composition for use of any one of claims 59 and 60, wherein the cancer is triple-negative breast cancer (TNBC).

62. A method of identifying a binder of an effector protein, comprising:
screening the effector protein against a DNA-encoded library (DEL) to identify a set of compounds specifically enriched for binding to the effector protein;
synthesizing one or more compounds from the set of compounds specifically enriched for binding to the effector protein without an encoding DNA tag;
determining effector protein activation by the one or more compounds from the set of compounds specifically enriched for binding to the effector protein synthesized without the encoding DNA tag; and
identifying the one or more compounds from the set of compounds specifically enriched for binding to the effector protein synthesized without the encoding DNA tag as a binder of the effector protein.

63. The method of claim 62, wherein the effector protein is RNase L.

64. The method of any one of claims 62 and 63, wherein determining effector protein activation comprises an in vitro assay.

65. The method of claim 64, wherein the in vitro assay is fluorescence-based.

66. The method of any one of claims 62-65, wherein screening the effector protein against the DNA-encoded library (DEL) to identify the set of compounds specifically enriched for binding to the effector protein comprises:
identifying a set of compounds as binding to the effector protein;
identifying one or more control sets of compounds; and
removing the one or more control sets of compounds from the set of compounds identified as binding to the effector protein to identify the set of compounds specifically enriched for binding to the effector protein.

67. The method of claim 66, wherein the set of compounds identified as binding to the effector protein comprises a set of compounds enriched by a recombinant effector protein functionalized with a tag.

68. The method of claim 67, wherein one control set of compounds comprises a set of compounds enriched by the tag alone.

69. The method of any one of claims 67 and 68, wherein the tag is glutathione S-transferase (GST).

70. The method of any one of claims 66-69, wherein one control set of compounds comprises a set of compounds enriched by a no target control (NTC).

71. The method of any one of claims 66-70, further comprising calculating an enrichment score for one or more compounds from the set of compounds specifically enriched for binding to the effector protein.

72. The method of claim 71, wherein the enrichment score is calculated from decoding of the DNA tags by next generation sequencing (NGS).

73. The method of any one of claims 71 and 72, wherein the enrichment score is calculated using corrected copy number, library size, NGS depth, and/or additional normalization factors.

74. A method of preparing a compound of Formula (II-a): 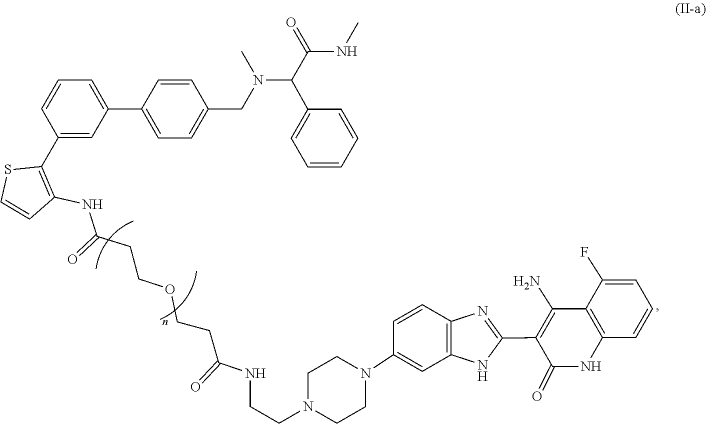
or a pharmaceutically acceptable salt, solvate, hydrate, polymorph, co-crystal, tautomer, stereoisomer, isotopically labeled compound, or prodrug thereof, comprising reacting a compound of formula: 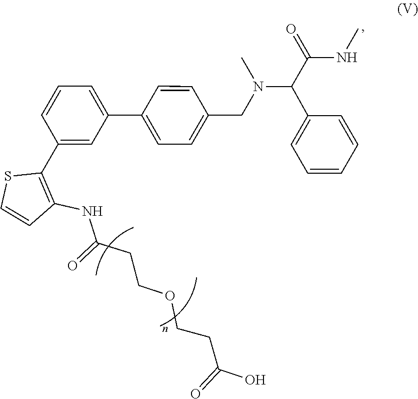
or salt thereof, with a compound of formula: 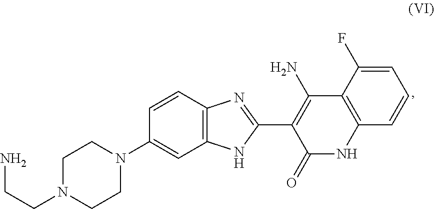 [SMILES: NCCN1CCN(c2ccc3nc(-c4c(N)c5c(F)cccc5[nH]c4=O)[nH]c3c2)CC1]
or a salt thereof, wherein:
n is 0, 1, 2, 3, 4, 5, 6, 7, 8, 9, or 10.

75. The method of claim 74, wherein the compound of Formula (II-a) is of formula: 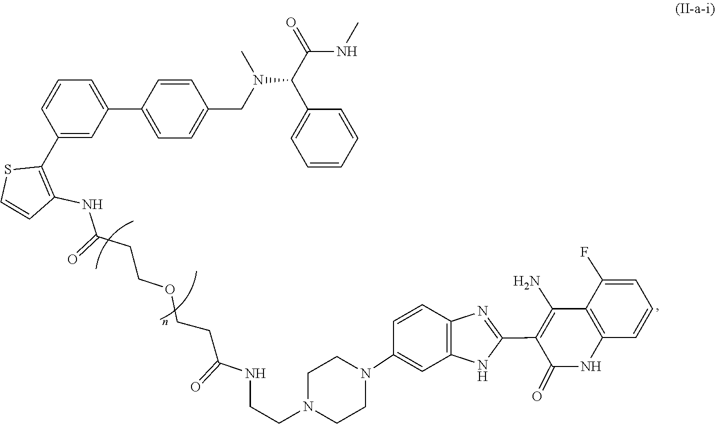
or a pharmaceutically acceptable salt, solvate, hydrate, polymorph, co-crystal, tautomer, stereoisomer, isotopically labeled compound, or prodrug thereof.

76. A kit comprising:
the compound of any one of claims 1-32, or a pharmaceutically acceptable salt, solvate, hydrate, polymorph, co-crystal, tautomer, stereoisomer, isotopically labeled compound, or prodrug thereof, or the composition of claim 33;
and instructions for its use.


SEQUENCE LISTING (16 sequences)

SEQ ID NO: 1 - RNA, 36 nt, synthetic construct
  source 1..36: mol_type = other RNA
ttatcaaattcttatttgccccatttttttggttta

SEQ ID NO: 2 - RNA, 17 nt, synthetic construct
  source 1..17: mol_type = other RNA
ttatcaaattcttattt

SEQ ID NO: 3 - RNA, 13 nt, synthetic construct
  source 1..13: mol_type = other RNA
gccccattttttt

SEQ ID NO: 4 - RNA, 59 nt, Homo sapiens
  source 1..59: mol_type = other RNA
tagcttatcagactgatgttgactgttgaatctcatggcaacaccagtcgatgggctgt

SEQ ID NO: 5 - RNA, 22 nt, synthetic construct
  source 1..22: mol_type = other RNA
agtggaatattctaataagcta

SEQ ID NO: 6 - RNA, 22 nt, Homo sapiens
  source 1..22: mol_type = other RNA
tagcttatcagactgatgttga

SEQ ID NO: 7 - RNA, 61 nt, synthetic construct
  source 1..61: mol_type = other RNA
tagcttatcagactgatgttgactgttgaatctcaatggtcaacaccagtcgatgggctgt

SEQ ID NO: 8 - RNA, 60 nt, synthetic construct
  source 1..60: mol_type = other RNA
tagcttatcagactgatgttgactgttgaatctcatggtcaacaccagtcgatgggctgt

SEQ ID NO: 9 - RNA, 58 nt, Homo sapiens
  source 1..58: mol_type = other RNA
tagcttatcagctgatgttgactgttgaatctcatggcaacaccagtcgatgggctgt

SEQ ID NO: 10 - RNA, 60 nt, synthetic construct
  source 1..60: mol_type = other RNA
tagcttatcagctgatgttgactgttgaatctcaatggtcaacaccagtcgatgggctgt

SEQ ID NO: 11 - RNA, 59 nt, synthetic construct
  source 1..59: mol_type = other RNA
tagcttatcagctgatgttgactgttgaatctcatggtcaacaccagtcgatgggctgt

SEQ ID NO: 12 - DNA, 22 bp, synthetic construct
  source 1..22: mol_type = other DNA
tagcttatcagactgatgttga

SEQ ID NO: 13 - DNA, 20 bp, synthetic construct
  source 1..20: mol_type = other DNA
gtaacccgttgaaccccatt

SEQ ID NO: 14 - DNA, 20 bp, synthetic construct
  source 1..20: mol_type = other DNA
ccatccaatcggtagtagcg

SEQ ID NO: 15 - DNA, 20 bp, synthetic construct
  source 1..20: mol_type = other DNA
ctgatgttgactgttgaatc

SEQ ID NO: 16 - DNA, 20 bp, synthetic construct
  source 1..20: mol_type = other DNA
gcccatcgactggtgttgcc